(19) United States
(12) Patent Application Publication
(10) Pub. No.: US 2019/0292179 A1
(43) Pub. Date: Sep. 26, 2019

(54) TGF Beta RECEPTOR ANTAGONISTS

(71) Applicant: BRISTOL-MYERS SQUIBB COMPANY, Princeton, NJ (US)

(72) Inventors: Robert M. Borzilleri, Carversville, PA (US); Peiyung Liu, Marlborough, MA (US); Andrew J. Tebben, New Hope, PA (US); Upender Velaparthi, Princeton Junction, NJ (US); Hasibur Rahaman, Bangalore (IN); Gopikishan Tonukunuru, Bangalore (IN); Jayakumar Sankara Warrier, Bangalore (IN)

(21) Appl. No.: 16/316,714

(22) PCT Filed: Jul. 19, 2017
(86) PCT No.: PCT/US2017/042697

§ 371 Date: Jan. 10, 2019

Related U.S. Application Data
(60) Provisional application No. 62/364,995, filed on Jul. 21, 2016.

(51) Int. Cl. C07D 471/04; A61K 31/437; A61K 31/5383; A61K 31/416; A61K 31/5025; A61K 39/395; C07D 403/04; C07D 487/04; C07D 498/04

(52) U.S. Cl. CPC C07D 471/04; A61K 31/437; A61K 31/5383; A61K 31/416; C07D 498/04; A61K 39/39558; C07D 403/04; C07D 487/04; A61K 31/5025

(57) ABSTRACT

The invention relates generally to compounds that modulate the activity of TGFβR-1 and TGFβR-2, pharmaceutical compositions containing said compounds and methods of treating proliferative disorders and disorders of dysregulated apoptosis, such as cancer, utilizing the compounds of the invention.

### CROSS-REFERENCE TO RELATED APPLICATIONS

This application claims the benefit of U.S. Provisional Application No. 62/364,995, filed Jul. 21, 2016, the disclosure of which is incorporated herein by reference in its entirety.

### FIELD OF THE INVENTION

The invention relates generally to compounds that modulate the activity of TGFβR-1 and TGFβR-2, pharmaceutical compositions containing said compounds and methods of treating proliferative disorders and disorders of dysregulated apoptosis, such as cancer, utilizing the compounds of the invention.

### BACKGROUND OF THE INVENTION

TGFβ is a multifunctional cytokine that regulates a wide variety of biological processes that include cell proliferation and differentiation, migration and adhesion, extracellular matrix modification including tumor stroma and immunosuppression, angiogenesis and desmoplasia (Ling and Lee, Current Pharmaceutical Biotech. 2011, 12:2190-2202), processes supporting tumor progression and late stage disease.

The active form of TGFβ is a dimer that signals through the formation of a membrane bound heterotetramer composed of the serine threonine type 1 and type 2 receptors, TGFβR-1 (ALK5) and TGFβR-2, respectively. Upon binding of two type 1 and two type 2 receptors, the type 2 constitutively activated receptors phosphorylate the type 1 receptors in the glycine and serine rich “GS region” activating a signaling cascade through the intracellular signaling effector molecules, Smad2 or Smad3. TGFβR-1 phosphorylates the receptor Smad2 and/or Smad3 (RSmads) that form a complex with Smad4 (Shi and Massague, Cell 2003, 113:685-700). These complexes then translocate to the nucleus where they elicit a wide variety of transcriptional responses resulting in altered gene expression (Weiss and Attisano, WIREs Developmental Biology, 2013, 2:47-63). The TGFβ proteins are prototypic members of a large family of related factors in mammals with a number of these also identified in other phyla. Generally, two groups have been characterized, the TGFβ-like and BMP-like ligands. In addition, in vertebrates, seven type 1 receptors and five type 2 receptors have been identified. An additional layer of complexity in ligand/receptor binding is the potential of co-receptors known as type 3 that facilitate ligand binding to the type 1 and 2 receptor complex. These type 3 receptors, also known as Betaglycan and Endoglin are comprised of large extracellular domains and short cytoplasmic tails and bind different TGFβ family members (Bernabeu et al., Biochem Biophys Acta 2009, 1792:954-73). Although type 3 receptors facilitate signaling, cleavage of the extracellular domain can generate soluble proteins that sequester ligands and can potentially inhibit signaling (Bernabeu et al., Biochem Biophys Acta 2009, 1792:954-73). While multiple redundancies in this large family present challenges to identifying a selective inhibitor, TGFβR-1 and -2 are relatively selective targets for TGFβ ligand engagement.

Alteration in TGFβ signaling are associated with a wide variety of human disorders including fibrosis, inflammatory, skeletal, muscular and cardiovascular disorders as well as cancer (Harradine, et al, 2006, Annals of Medicine 38:403-14). In human cancer, TGFβ signaling alterations can occur in the germline or arise spontaneously in various cancer types. TGFβ is also a potent inducer of angiogenesis, which provides a critical support system for solid tumors as well as a mechanism for tumor cell dissemination (Buijs et al., 2011, Curr Pharmaceutical Biotech, 12:2121-37). Therefore multiple strategies to inhibit TGFβ signaling have been exploited in various disease states.

### SUMMARY OF THE INVENTION

In a first aspect of the present invention, there is provided a compound having the structure of formula (Ia) or (Ib):

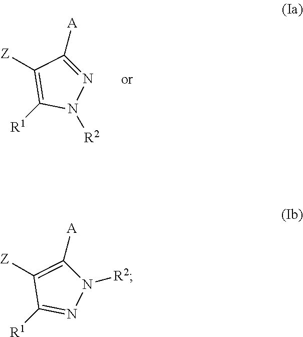

or a pharmaceutically acceptable salt, prodrug, or N-oxide thereof, or a solvate or hydrate thereof,

wherein

A is phenyl substituted with 0-3 R⁴ groups or pyridyl substituted with 0-3 R⁴ groups;

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

Z is a 10-12 membered heterobicyclic ring substituted with 0-2 R^{Z} groups containing 1-4 heteroatoms selected from —O—, —S— or —N—;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect, there is provided a pharmaceutical composition comprising a compound of the invention or a pharmaceutically acceptable salt thereof and one or more pharmaceutically acceptable carriers, diluents or excipients.

In another aspect, there is provided a compound of the invention or a pharmaceutically acceptable salt thereof for use in therapy. In particular, for use in the treatment of a disease or condition for which a TGFβR antagonist is indicated.

In another aspect, there is provided a method of treating cancers, fibrosis, inflammatory, skeletal, muscular and cardiovascular disorders which comprise administering to a subject in need thereof a therapeutically effective amount of a TGFβR antagonist.

### DETAILED DESCRIPTION OF THE INVENTION

In a first aspect of the present invention, there is provided a compound having the structure of formula (Ia) or (Ib):

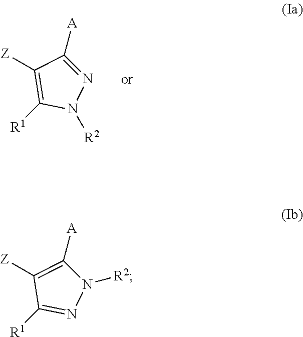

or a pharmaceutically acceptable salt, prodrug, or N-oxide thereof, or a solvate or hydrate thereof,

wherein

A is phenyl substituted with 0-3 R⁴ groups or pyridyl substituted with 0-3 R⁴ groups;

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

Z is a 10-12 membered heterobicyclic ring substituted with 0-2 R^{Z} groups containing 1-4 heteroatoms selected from —O—, —S— or —N—;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In a second aspect within the scope of the first aspect of the invention, there is provided a compound of formula (Ia)

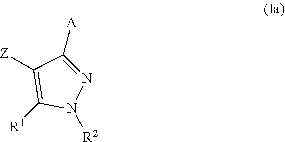

or a pharmaceutically acceptable salt, prodrug, or N-oxide thereof, or a solvate or hydrate thereof,

wherein

A is phenyl substituted with 0-3 R⁴ groups or pyridyl substituted with 0-3 R⁴ groups;

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

Z is a 10-12 membered heterobicyclic ring substituted with 0-2 R^{Z} groups containing 1-4 heteroatoms selected from —O—, —S— or —N—;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In a third aspect within the scope of the first and second aspects of the invention, there is provided a compound of formula (Ia) wherein

A is phenyl substituted with 0-2 R⁴ groups or pyridyl substituted with 0-2 R⁴ groups;

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

Z is a 10-12 membered heterobicyclic ring substituted with 0-2 R^{Z} groups containing 1-4 heteroatoms selected from —O—, —S— or —N—;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In a fourth aspect within the scope of the first, second and third aspects of the invention, there is provided a compound of formula (Ia) wherein

Z is

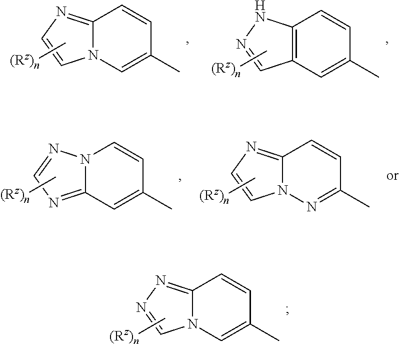

and n is 0, 1 or 2.

In a fifth aspect of the invention, there is provided a compound of formula (Ia) wherein

Z is

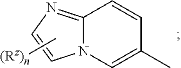

and n is 0, 1 or 2.

In a sixth aspect of the invention, there is provided a compound of formula (Ia) wherein

Z is

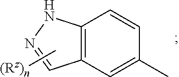

and n is 0, 1 or 2.

In a seventh aspect of the invention, there is provided a compound of formula (Ia) wherein

Z is

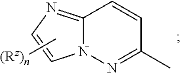

and n is 0, 1 or 2.

In another aspect of the invention within the scope of the prior seven aspects, there is provided a compound of the following structure

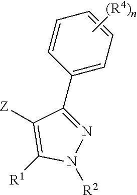

or a pharmaceutically acceptable salt, prodrug, or N-oxide thereof, or a solvate or hydrate thereof.

In another aspect of the invention within the scope of the prior aspects, there is provided a compound of the following structure

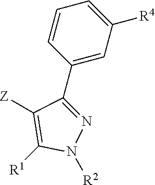

or a pharmaceutically acceptable salt, prodrug, or N-oxide thereof, or a solvate or hydrate thereof.

In another aspect of the invention within the scope of the prior aspects, there is provided a compound of the following structure

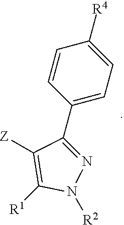

or a pharmaceutically acceptable salt, prodrug, or N-oxide thereof, or a solvate or hydrate thereof.

In another aspect of the invention within the scope of the prior aspects, there is provided a compound of the following structure

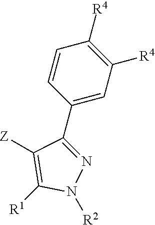

or a pharmaceutically acceptable salt, prodrug, or N-oxide thereof, or a solvate or hydrate thereof.

In another aspect of the invention within the scope of the prior aspects, there is provided a compound of the following structure

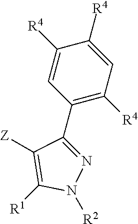

or a pharmaceutically acceptable salt, prodrug, or N-oxide thereof, or a solvate or hydrate thereof.

In another aspect of the invention within the scope of the prior aspects, there is provided a compound of the following structure

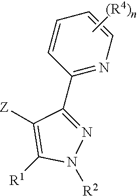

or a pharmaceutically acceptable salt, prodrug, or N-oxide thereof, or a solvate or hydrate thereof.

In another aspect of the invention within the scope of the prior aspects, there is provided a compound of the following structure

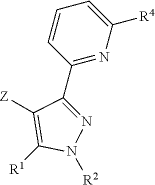

or a pharmaceutically acceptable salt, prodrug, or N-oxide thereof, or a solvate or hydrate thereof.

In another aspect of the invention within the scope of the prior aspects, there is provided a compound of the following structure

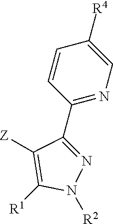

or a pharmaceutically acceptable salt, prodrug, or N-oxide thereof, or a solvate or hydrate thereof.

In another aspect of the invention within the scope of the prior aspects, there is provided a compound of the following structure

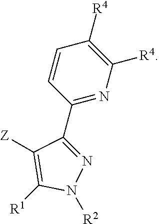

In another aspect of the invention, there is provided a compound of the following structure

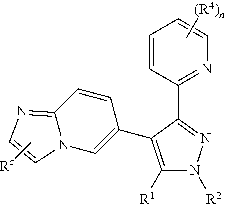

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect of the invention, there is provided a compound of the following structure

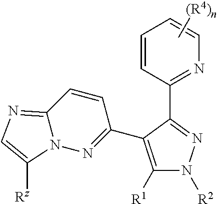

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl R² is hydrogen, C₁-C₆ alkyl, C₁-C₆ haloalkyl, C₁-C₆ alkyloxy or C₃-C₈ cycloalkyl; or R¹ and R² combined with the atoms to which they are attached form a five- to eight-membered ring.

In another aspect of the invention, there is provided a compound of the following structure

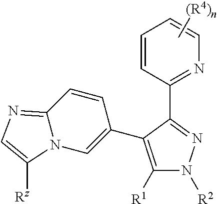

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect of the invention, there is provided a compound of the following structure

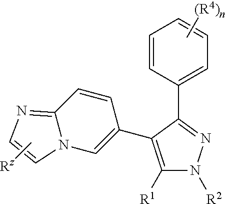

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect of the invention, there is provided a compound of the following structure

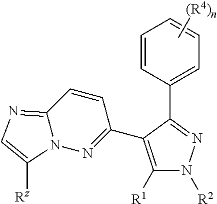

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect of the invention, there is provided a compound of the following structure

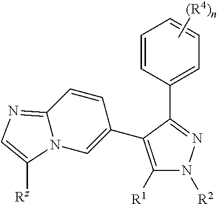

or a pharmaceutically acceptable salt, prodrug or N-oxide thereof, or solvate or hydrate thereof,

wherein

each R⁴ is independently hydrogen, halogen, C₁-C₆ alkyl, C₁-C₆ haloalkyl, C₁-C₆ alkoxy, C₁-C₆ haloalkoxy, —NO₂, or —CN;

each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;

each R^{z1} and R^{z2} is independently hydrogen, C₁-C₆ alkyl or C₃-C₆ cycloalkyl;

R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;

each R^{1a} and R^{1b} is independently hydrogen, C₁-C₆ alkyl or C₃-C₆ cycloalkyl

R² is hydrogen, C₁-C₆ alkyl, C₁-C₆ haloalkyl, C₁-C₆ alkyloxy or C₃-C₈ cycloalkyl;

or R¹ and R² combined with the atoms to which they are attached form a five- to eight-membered ring.

In another aspect, there is provided a compound selected from the exemplified examples within the scope of the first aspect, or a pharmaceutically acceptable salt, tautomer or stereoisomer thereof.

In another aspect, there is provided a compound selected from any subset list of compounds within the scope of any of the above aspects.

### Other Embodiments of the Invention

In another embodiment, the invention provides a pharmaceutical composition, comprising a pharmaceutically acceptable carrier and a therapeutically effective amount of at least one of the compounds of the invention or a stereoisomer, a tautomer, a pharmaceutically acceptable salt, or a solvate thereof.

In another embodiment, the invention provides a process for making a compound of the invention or a stereoisomer, a tautomer, a pharmaceutically acceptable salt, or a solvate thereof.

In another embodiment, the invention provides a method for the treatment and/or prophylaxis of various types of cancer, comprising administering to a patient in need of such treatment and/or prophylaxis a therapeutically effective amount of one or more compounds of the invention, alone, or, optionally, in combination with another compound of the invention and/or at least one other type of therapeutic agent.

In another embodiment, the invention provides a method for the treatment and/or prophylaxis of various types of cancer, including without limitation, small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In another embodiment, the invention provides a method for the treatment and/or prophylaxis of various types of cancer, including without limitation, small cell lung cancer, non-small cell lung cancer, triple-negative breast cancer, colorectal cancer, prostate cancer, melanoma, pancreatic cancer, multiple myeloma, T-acute lymphoblastic leukemia or AML.

In another embodiment, the invention provides a method for the treatment and/or prophylaxis of Marfan's syndrome and associated diseases, disorders and conditions associated with aberrant TGF-β expression.

In another embodiment, the invention provides a method for the treatment and/or prophylaxis of fibrosis such as hepatic or pulmonary fibrosis.

In another embodiment, the invention provides a compound of the present invention for use in therapy.

In another embodiment, the invention provides a combined preparation of a compound of the present invention and additional therapeutic agent(s) for simultaneous, separate or sequential use in therapy.

### Therapeutic Applications

The compounds of formula (I) of the invention are TGFβR antagonists and have potential utility in the treatment of diseases and conditions for which a TGFβR antagonist is indicated.

In one embodiment there is provided a method for the treatment of a disease or condition, for which a TGFβR antagonists is indicated, in a subject in need thereof which comprises administering a therapeutically effective amount of compound of formula (I) or a pharmaceutically acceptable salt thereof.

In another embodiment there is provided a method for treatment of a chronic autoimmune and/or inflammatory condition, in a subject in need thereof which comprises administering a therapeutically effective amount of one or more compounds of formula (I) or a pharmaceutically acceptable salt thereof.

In a further embodiment there is provided a method for treatment of cancer in a subject in need thereof which comprises administering a therapeutically effective amount of one or more compounds of formula (I) or a pharmaceutically acceptable salt thereof.

In one embodiment the subject in need thereof is a mammal, particularly a human.

TGFβR antagonists are believed to be useful in the treatment of a variety of diseases or conditions related to systemic or tissue inflammation, inflammatory responses to infection or hypoxia, cellular activation and proliferation, lipid metabolism, fibrosis and in the prevention and treatment of viral infections.

TGFβR antagonists may be useful in the treatment of fibrotic conditions such as idiopathic pulmonary fibrosis, renal fibrosis, post-operative stricture, keloid formation, scleroderma and cardiac fibrosis.

TGFβR antagonists may be useful in the treatment of cancer, including hematological, epithelial including lung, breast and colon carcinomas, midline carcinomas, mesenchymal, hepatic, renal and neurological tumours.

The term “diseases or conditions for which a TGFβR antagonists is indicated” is intended to include any of or all of the above disease states.

While it is possible that for use in therapy, a compound of formula (I) as well as pharmaceutically acceptable salts thereof may be administered as the compound itself, it is more commonly presented as a pharmaceutical composition.

Pharmaceutical compositions may be presented in unit dose forms containing a predetermined amount of active ingredient pep unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Types of cancers that may be treated with the compounds of this invention include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

Compounds of the invention are useful for the treatment of certain types of cancer by themselves or in combination or co-administration with other therapeutic agents or radiation therapy. Thus, in one embodiment, the compounds of the invention are co-administered with radiation therapy or a second therapeutic agent with cytostatic or antineoplastic activity. Suitable cytostatic chemotherapy compounds include, but are not limited to (i) antimetabolites; (ii) DNA-fragmenting agents, (iii) DNA-crosslinking agents, (iv) intercalating agents (v) protein synthesis inhibitors, (vi) topoisomerase I poisons, such as camptothecin or topotecan; (vii) topoisomerase II poisons, (viii) microtubule-directed agents, (ix) kinase inhibitors (x) miscellaneous investigational agents (xi) hormones and (xii) hormone antagonists. It is contemplated that compounds of the invention may be useful in combination with any known agents falling into the above 12 classes as well as any future agents that are currently in development. In particular, it is contemplated that compounds of the invention may be useful in combination with current Standards of Care as well as any that evolve over the foreseeable future. Specific dosages and dosing regimens would be based on physicians' evolving knowledge and the general skill in the art.

Further provided herein are methods of treatment wherein compounds of the invention are administered with one or more immuno-oncology agents. The immuno-oncology agents used herein, also known as cancer immunotherapies, are effective to enhance, stimulate, and/or up-regulate immune responses in a subject. In one aspect, the administration of a compound of the invention with an immuno-oncology agent has a synergic effect in inhibiting tumor growth.

In one aspect, the compound(s) of the invention are sequentially administered prior to administration of the immuno-oncology agent. In another aspect, compound(s) of the invention are administered concurrently with the immunology-oncology agent. In yet another aspect, compound(s) of the invention are sequentially administered after administration of the immuno-oncology agent.

In another aspect, compounds of the invention may be co-formulated with an immuno-oncology agent.

Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY, NGFR.

In another aspect, the immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In one aspect, T cell responses can be stimulated by a combination of a compound of the invention and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of the invention for the treatment of cancer include antagonists of inhibitory receptors on NK cells or agonists of activating receptors on NK cells. For example, compounds of the invention can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, compounds of the invention can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224

In another aspect, the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO008/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO009/009116) and MK-4166 (WO11/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO009/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO 11/109400).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional embodiments. It is also understood that each individual element of the embodiments is its own independent embodiment. Furthermore, any element of an embodiment is meant to be combined with any and all other elements from any embodiment to describe an additional embodiment.

### Pharmaceutical Compositions and Dosing

The invention also provides pharmaceutically acceptable compositions which comprise a therapeutically effective amount of one or more of the compounds of Formula I, formulated together with one or more pharmaceutically acceptable carriers (additives) and/or diluents, and optionally, one or more additional therapeutic agents described above. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained release formulation; (3) topical application, for example, as a cream, ointment, or a controlled release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the patient being treated and the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, troches and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide.

Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

Generally, oral, intravenous, intracerebroventricular and subcutaneous doses of the compounds of this invention for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for a compound of the present invention to be administered alone, it is preferable to administer the compound as a pharmaceutical formulation (composition).

### Definitions

Unless specifically stated otherwise herein, references made in the singular may also include the plural. For example, “a” and “an” may refer to either one, or one or more.

Unless otherwise indicated, any heteroatom with unsatisfied valences is assumed to have hydrogen atoms sufficient to satisfy the valences.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention.

Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

When a substituent is noted as “optionally substituted”, the substituents are selected from, for example, substituents such as alkyl, cycloalkyl, aryl, heterocyclo, halo, hydroxy, alkoxy, oxo, alkanoyl, aryloxy, alkanoyloxy, amino, alkylamino, arylamino, arylalkylamino, disubstituted amines in which the 2 amino substituents are selected from alkyl, aryl or arylalkyl; alkanoylamino, aroylamino, aralkanoylamino, substituted alkanoylamino, substituted arylamino, substituted aralkanoylamino, thiol, alkylthio, arylthio, arylalkylthio, alkylthiono, arylthiono, arylalkylthiono, alkylsulfonyl, arylsulfonyl, arylalkylsulfonyl, sulfonamido, e.g. —SO₂NH₂, substituted sulfonamido, nitro, cyano, carboxy, carbamyl, e.g. —CONH₂, substituted carbamyl e.g. —CONHalkyl, —CONHaryl, —CONHarylalkyl or cases where there are two substituents on the nitrogen selected from alkyl, aryl or arylalkyl; alkoxycarbonyl, aryl, substituted aryl, guanidino, heterocyclyl, e.g., indolyl, imidazolyl, furyl, thienyl, thiazolyl, pyrrolidyl, pyridyl, pyrimidyl, pyrrolidinyl, piperidinyl, morpholinyl, piperazinyl, homopiperazinyl and the like, and substituted heterocyclyl, unless otherwise defined.

For purposes of clarity and in accordance with standard convention in the art, the symbol

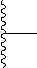

is used in formulas and tables to show the bond that is the point of attachment of the moiety or substituent to the core/nucleus of the structure.

Additionally, for purposes of clarity, where a substituent has a dash (-) that is not between two letters or symbols; this is used to indicate a point of attachment for a substituent. For example, —CONH₂ is attached through the carbon atom.

Additionally, for purposes of clarity, when there is no substituent shown at the end of a solid line, this indicates that there is a methyl (CH₃) group connected to the bond.

As used herein, the term “alkyl” or “alkylene” is intended to include both branched and straight-chain saturated aliphatic hydrocarbon groups having the specified number of carbon atoms. For example, “C₁-C₆ alkyl” denotes alkyl having 1 to 6 carbon atoms. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “alkenyl” denotes a straight- or branch-chained hydrocarbon radical containing one or more double bonds and typically from 2 to 20 carbon atoms in length. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” denotes a straight- or branch-chained hydrocarbon radical containing one or more triple bonds and typically from 2 to 20 carbon atoms in length. For example, “C₂-C₈ alkenyl” contains from two to eight carbon atoms. Representative alkynyl groups include, but are not limited to, for example, ethynyl, 1-propynyl, 1-butynyl, heptynyl, octynyl and the like.

The term “alkoxy” or “alkyloxy” refers to an —O-alkyl group. “C₁₋₆ alkoxy” (or alkyloxy), is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

The term “aryl”, either alone or as part of a larger moiety such as “aralkyl”, “aralkoxy”, or aryloxyalkyl”, refers to monocyclic, bicyclic and tricyclic ring systems having a total of five to 15 ring members, wherein at least one ring in the system is aromatic and wherein each ring in the system contains three to seven ring members. In certain embodiments of the invention, “aryl” refers to an aromatic ring system which includes, but not limited to phenyl, biphenyl, indanyl, 1-naphthyl, 2-naphthyl and terahydronaphthyl. The term “aralkyl” or “arylalkyl” refers to an alkyl residue attached to an aryl ring. Non-limiting examples include benzyl, phenethyl and the like. The fused aryls may be connected to another group either at a suitable position on the cycloalkyl ring or the aromatic ring. For example:

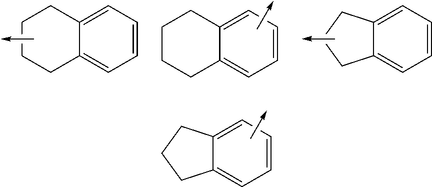

Arrowed lines drawn from the ring system indicate that the bond may be attached to any of the suitable ring atoms.

The term “cycloalkyl” refers to cyclized alkyl groups. C₃₋₆ cycloalkyl is intended to include C₃, C₄, C₅, and C₆ cycloalkyl groups. Example cycloalkyl groups include, but are not limited to, cyclopropyl, cyclobutyl, cyclopentyl, cyclohexyl, and norbomyl.

Branched cycloalkyl groups such as 1-methylcyclopropyl and 2-methylcyclopropyl are included in the definition of “cycloalkyl”. The term “cycloalkenyl” refers to cyclized alkenyl groups. C₄₋₆ cycloalkenyl is intended to include C₄, C₅, and C₆ cycloalkenyl groups. Example cycloalkenyl groups include, but are not limited to, cyclobutenyl, cyclopentenyl, and cyclohexenyl.

The term “cycloalkylalkyl” refers to a cycloalkyl or substituted cycloalkyl bonded to an alkyl group connected to the core of the compound.

“Halo” or “halogen” includes fluoro, chloro, bromo, and iodo. “Haloalkyl” is intended to include both branched and straight-chain saturated aliphatic hydrocarbon groups having the specified number of carbon atoms, substituted with 1 or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with 1 or more fluorine atoms.

“Haloalkoxy” or “haloalkyloxy” represents a haloalkyl group as defined above with the indicated number of carbon atoms attached through an oxygen bridge. For example, “C₁₋₆ haloalkoxy”, is intended to include C₁, C₂, C₃, C₄, C₅, and C₆ haloalkoxy groups. Examples of haloalkoxy include, but are not limited to, trifluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

The term “benzyl,” as used herein, refers to a methyl group on which one of the hydrogen atoms is replaced by a phenyl group.

As used herein, the term “heterocycle,” “heterocyclyl,” or “heterocyclic group” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. When the term “heterocycle” is used, it is intended to include heteroaryl.

Examples of heterocycles include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

As used herein, the term “bicyclic heterocycle” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocycle, a 6-membered heterocycle or a carbocycle (provided the first ring is not benzo when the second ring is a carbocycle).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocycle exceeds 1, then these heteroatoms are not adjacent to one another. It is preferred that the total number of S and O atoms in the heterocycle is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydro-quinolinyl, 2,3-dihydro-benzofuranyl, chromanyl, 1,2,3,4-tetrahydro-quinoxalinyl and 1,2,3,4-tetrahydro-quinazolinyl.

As used herein, the term “aromatic heterocyclic group” or “heteroaryl” is intended to mean stable monocyclic and polycyclic aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

Bridged rings are also included in the definition of heterocycle. A bridged ring occurs when one or more, preferably one to three, atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge.

The term “heterocyclylalkyl” refers to a heterocyclyl or substituted heterocyclyl bonded to an alkyl group connected to the core of the compound.

The term “counter ion” is used to represent a negatively charged species such as chloride, bromide, hydroxide, acetate, and sulfate or a positively charged species such as sodium (Na+), potassium (K+), ammonium (RnNHm+ where n=0-4 and m=0-4) and the like.

The term “electron withdrawing group” (EWG) refers to a substituent which polarizes a bond, drawing electron density towards itself and away from other bonded atoms. Examples of EWGs include, but are not limited to, CF₃, CF₂CF₃, CN, halogen, haloalkyl, NO₂, sulfone, sulfoxide, ester, sulfonamide, carboxamide, alkoxy, alkoxyether, alkenyl, alkynyl, OH, C(O)alkyl, CO₂H, phenyl, heteroaryl, —O-phenyl, and —O— heteroaryl. Preferred examples of EWG include, but are not limited to, CF₃, CF₂CF₃, CN, halogen, SO₂(C₁₋₄ alkyl), CONH(C₁₋₄ alkyl), CON(C₁₋₄ alkyl)₂, and heteroaryl. More preferred examples of EWG include, but are not limited to, CF₃ and CN.

As used herein, the term “amine protecting group” means any group known in the art of organic synthesis for the protection of amine groups which is stable to an ester reducing agent, a disubstituted hydrazine, R4-M and R7-M, a nucleophile, a hydrazine reducing agent, an activator, a strong base, a hindered amine base and a cyclizing agent. Such amine protecting groups fitting these criteria include those listed in Wuts, P. G. M. and Greene, T. W. Protecting Groups in Organic Synthesis, 4th Edition, Wiley (2007) and The Peptides: Analysis, Synthesis, Biology, Vol. 3, Academic Press, New York (1981), the disclosure of which is hereby incorporated by reference. Examples of amine protecting groups include, but are not limited to, the following: (1) acyl types such as formyl, trifluoroacetyl, phthalyl, and p-toluenesulfonyl; (2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); (3) aliphatic carbamate types such as tert-butyloxycarbonyl (Boc), ethoxycarbonyl, diisopropylmethoxycarbonyl, and allyloxycarbonyl; (4) cyclic alkyl carbamate types such as cyclopentyloxycarbonyl and adamantyloxycarbonyl; (5) alkyl types such as triphenylmethyl and benzyl; (6) trialkylsilane such as trimethylsilane; (7) thiol containing types such as phenylthiocarbonyl and dithiasuccinoyl; and (8) alkyl types such as triphenylmethyl, methyl, and benzyl; and substituted alkyl types such as 2,2,2-trichloroethyl, 2-phenylethyl, and t-butyl; and trialkylsilane types such as trimethylsilane.

As referred to herein, the term “substituted” means that at least one hydrogen atom is replaced with a non-hydrogen group, provided that normal valencies are maintained and that the substitution results in a stable compound. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22nd Edition, Allen, L. V. Jr., Ed.; Pharmaceutical Press, London, UK (2012), the disclosure of which is hereby incorporated by reference.

In addition, compounds of formula I may have prodrug forms. Any compound that will be converted in vivo to provide the bioactive agent (i.e., a compound of formula I) is a prodrug within the scope and spirit of the invention. Various forms of prodrugs are well known in the art. For examples of such prodrug derivatives, see:
a) Bundgaard, H., ed., Design of Prodrugs, Elsevier (1985), and Widder, K. et al., eds., Methods in Enzymology, 112:309-396, Academic Press (1985);
b) Bundgaard, H., Chapter 5, “Design and Application of Prodrugs,” A Textbook of Drug Design and Development, pp. 113-191, Krosgaard-Larsen, P. et al., eds., Harwood Academic Publishers (1991);
c) Bundgaard, H., Adv. Drug Deliv. Rev., 8:1-38 (1992);
d) Bundgaard, H. et al., J. Pharm. Sci., 77:285 (1988);
e) Kakeya, N. et al., Chem. Pharm. Bull., 32:692 (1984); and
f) Rautio, J (Editor). Prodrugs and Targeted Delivery (Methods and Principles in Medicinal Chemistry), Vol 47, Wiley-VCH, 2011.

Compounds containing a carboxy group can form physiologically hydrolyzable esters that serve as prodrugs by being hydrolyzed in the body to yield formula I compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula I include C₁₋₆alkyl, C₁₋₆alkylbenzyl, 4-methoxybenzyl, indanyl, phthalyl, methoxymethyl, C₁₋₆ alkanoyloxy-C₁₋₆alkyl (e.g., acetoxymethyl, pivaloyloxymethyl or propionyloxymethyl), C₁₋₆alkoxycarbonyloxy-C₁₋₆alkyl (e.g., methoxycarbonyl-oxymethyl or ethoxycarbonyloxymethyl, glycyloxymethyl, phenylglycyloxymethyl, (5-methyl-2-oxo-1,3-dioxolen-4-yl)-methyl), and other well known physiologically hydrolyzable esters used, for example, in the penicillin and cephalosporin arts. Such esters may be prepared by conventional techniques known in the art.

Preparation of prodrugs is well known in the art and described in, for example, King, F. D., ed., Medicinal Chemistry: Principles and Practice, The Royal Society of Chemistry, Cambridge, UK (2^{nd} edition, reproduced, 2006); Testa, B. et al., Hydrolysis in Drug and Prodrug Metabolism. Chemistry, Biochemistry and Enzymology, VCHA and Wiley-VCH, Zurich, Switzerland (2003); Wermuth, C. G., ed., The Practice of Medicinal Chemistry, 3^{rd} edition, Academic Press, San Diego, Calif. (2008).

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD3 denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The term “solvate” means a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably refers to humans.

As used herein, the term “effective amount” means that amount of a drug or pharmaceutical agent, i.e., a compound of the invention, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. An effective amount can be administered in one or more administrations, applications or dosages and is not intended to be limited to a particular formulation or administration route. The term also includes within its scope amounts effective to enhance normal physiological function

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference.

The compounds of this invention may be prepared using the reactions and techniques described in this section. The reactions are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Third Edition, Wiley and Sons, 1999).

Compounds of Formula (I) may be prepared by reference to the methods illustrated in the following Schemes. As shown therein the end product is a compound having the same structural formula as Formula (I). It will be understood that any compound of Formula (I) may be produced by the schemes by the suitable selection of reagents with appropriate substitution. Solvents, temperatures, pressures, and other reaction conditions may readily be selected by one of ordinary skill in the art. Starting materials are commercially available or readily prepared by one of ordinary skill in the art. Constituents of compounds are as defined herein or elsewhere in the specification.

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth herein below, but rather is defined by the claims appended hereto.

### LIST OF ABBREVIATIONS

| | |
|---|---|
| AcCl | Acetyl chloride |
| BisPin | 4,4,4′,4′,5,5,5′,5′-octamethyl-2,2′-bi(1,3,2-dioxaborolane) |
| DCM | Dichloromethane |
| DAST | diethylaminosulfur trifluoride |
| DIPEA | N,N-diisopropylethylamine |
| DMF | N,N-Dimethylformamide |
| DMAP | 4-dimethylaminopyridine |
| DMSO | Dimethyl sulfoxide |
| HATU | 1-[Bis(dimethylamino)methylene]-1H-1,2,3-triazolo[4,5- |
| | b]pyridinium 3-oxid hexafluorophosphate |
| HPLC | High performance liquid chromatography |
| LCMS | Liquid chromatography-mass spectrometry |
| NBS | N-Bromosuccinimide |
| NIS | N-Iodosuccinimide |
| RT | Room temperature |
| ret. time | Retention time |
| SEM-Cl | 2-(Trimethylsilanyl)ethoxymethyl chloride |
| Selectfluor ® | 1-Chloromethyl-4-fluoro-1,4-diazoniabicyclo[2.2.2] |
| | octane bis(tetrafluoroborate) |
| TFA | Trifluoroacetic acid |
| TEA | Triethylamine |
| TBAF | Tetra-n-butylammonium fluoride |
| THF | Tetrahydrofuran |
| TEMPO | (2,2,6,6-Tetramethyl-piperidin-1-yl)oxyl |
| Xantphos | 4,5-Bis(diphenylphosphino)-9,9-dimethylxanthene |

Analytical LC-MS/HPLC retention time reported for each example and intermediate using one of the following general analytical LC-MS/HPLC conditions:

### LCMS Conditions:

Method A: Column: Waters Acquity UPLC BEH C18 (2.1×50 mm), 1.7μ; Mobile phase A: 0.1% TFA in water; Mobile phase B: 0.1% TFA in acetonitrile; Gradient=20-90% B over 1.1 minute, then a 0.6 minute hold at 90% B; Temperature: 50° C.; Flow rate: 0.7 mL/min; Detection: UV at 220 nm.
Method B: Column: Waters Acquity UPLC BEH C18 (2.1×50 mm) 1.7μ, Mobile phase A: 10 mM NH₄OAc:acetonitrile (95:5); Mobile phase B: 10 mM NH₄OAc:acetonitrile (5:95), Gradient=20-90% B over 1.1 minute, then a 0.6 minute hold at 90% B; Temperature: 50° C.; Flow rate: 0.7 mL/min; Detection: UV at 220 nm.
Method C: Column: Ascentis Express C18 (2.1×50 mm), 2.7μ; Mobile phase A: 10 mM NH₄OAc:acetonitrile (95:5), Mobile phase B: 10 mM NH₄OAc:acetonitrile (5:95), Gradient=0-100% B over 3 minutes; Temperature: 50° C.; Flow rate: 1.1 mL/min; Detection: UV at 220 nm.
Method D: Column: Ascentis Express C18 (2.1×50 mm), 2.7μ; Mobile phase A: 0.1% TFA: acetonitrile (95:5), Mobile phase B: 0.1% TFA:acetonitrile (5:95), Gradient=0-100% B over 3 minutes; Temperature: 50° C.; Flow rate: 1.1 mL/min; Detection: UV at 220 nm.
Method E: Column: Kinetex XB-C18 (3×75 mm) 2.6μ; Mobile phase A: 10 mM ammonium formate:acetonitrile (98:2), Mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

Preparative HPLC Conditions:

Method F: Column: Waters X-Bridge C18, 19×150 mm, 5μ; Mobile Phase A: 0.1% TFA in water; Mobile Phase B: acetonitrile; Gradient: 10-100% B over 25 minutes, then a 5 minute hold at 100% B; Flow: 15 mL/min.
Method G: Column: Inertsil ODS, 250×20 mm ID, 5μ; Mobile Phase A: 0.1% TFA in water; Mobile Phase B: acetonitrile; Gradient: 10-100% B over 25 minutes, then a 5 minute hold at 100% B; Flow: 17 mL/min.
Method H: Column: Inertsil ODS, 250×20 mm ID, 5μ; Mobile Phase A: 10 mM NH₄OAc in water; Mobile Phase B: Methanol; Gradient: 10-100% B over 25 minutes, then a 5 minute hold at 100% B; Flow: 17 mL/min.
Method I: Column: DAD-1 X-Bridge phenyl, 150×4.6 mm 5μ; DAD-2 Sunfire C18, 150×4.6 mm 5S; Mobile Phase A: 10 mM NH₄OAc in water; Mobile Phase B: acetonitrile; Gradient: 0-100% B over 18 minutes, then a 5 minute hold at 100% B; Flow: 1 mL/min.
Method J: Column: Sunfire C18, 150×4.6 mm, 5μ; Mobile Phase A: 0.05% TFA in water; Mobile Phase B: acetonitrile; Gradient: 0-100% B over 18 minutes, then a 5 minute hold at 100% B; Flow: 1 mL/min.
Method K: Column: Inertsil ODS, 150×4.6 mm ID, 5μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method L: Column: Sunfire C18, 150×19 mm ID, 5μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method M: Column: Synergy Polar, 250×21.2 mm ID, 4μ; Mobile Phase A: 0.1% TFA in water; Mobile Phase B: acetonitrile; Gradient: 0-100% B over 18 minutes, then a 5 minute hold at 100% B; Flow: 17 mL/min.
Method N: Column: Waters X-Bridge C18, 19×150 mm ID, 5μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method O: Column: Symmetry C8, 300×19 mm ID, 7μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method P: Column: X-Bridge Phenyl, 250×19 mm ID, 5μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method Q: Column: DAD1-X-Bridge Phenyl, 4.6×250 mm, 5μ; DAD2-X-Terra RP18, 4.6×250 mm, 5μ; Mobile Phase A: 0.05% TFA in water; Mobile Phase B: acetonitrile; Gradient: 0-100% B over 18 minutes, then a 5 minute hold at 100% B; Flow: 2 mL/min.
Method R: Column: Waters X-Select C18, 19×150 mm, 5μ; Mobile Phase A: 10 mM NH₄OAc in water; Mobile Phase B: acetonitrile; Gradient: 0-100% B over 18 minutes, then a 5 minute hold at 100% B; Flow: 17 mL/min.
Method S: SFC method: Injection Number 1, injection Volume 10 mL, co-solvent 0.2% in methanol, Column Chiral OJ-h (4.6×250 mm) ID, 5μ; Column Temperature; 21.1° C., CO₂ Flow Rate 2.1, co-solvent Flow Rate 0.9 mL/min, co-colvent % 30, Total Flow 3, Back Pressure 102 psi.
Method T: Column: DAD1-INERTSIL ODS, 4.6×250 mm, 5μ; DAD2-X-bridge phenyl, 4.6×250 mm, 5μ; Mobile Phase A: 10 mM NH₄OAc in water; Mobile Phase B: acetonitrile; Gradient: 0-100% B over 18 minutes, then a 5 minute hold at 100% B; Flow: 2 mL/min.

### EXPERIMENTAL

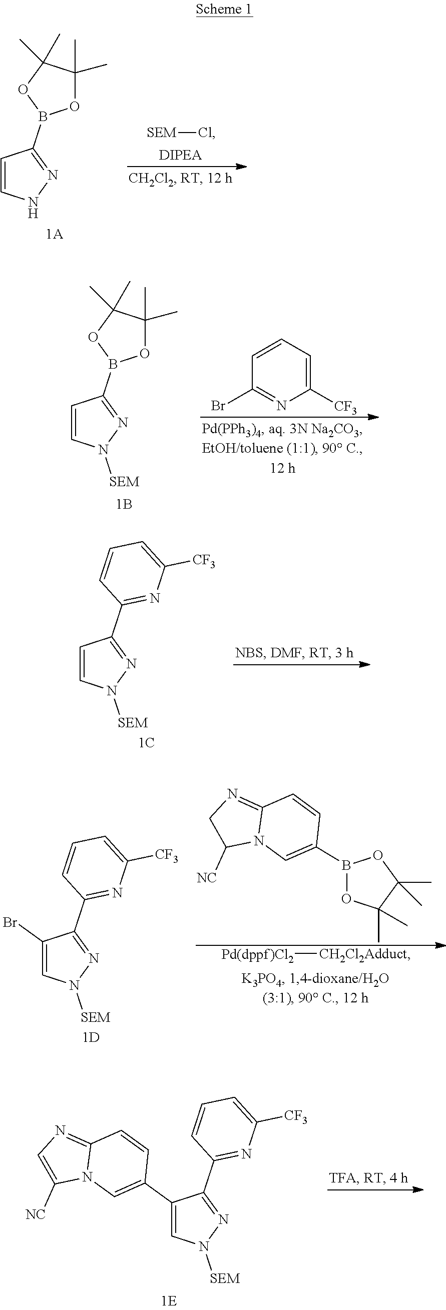

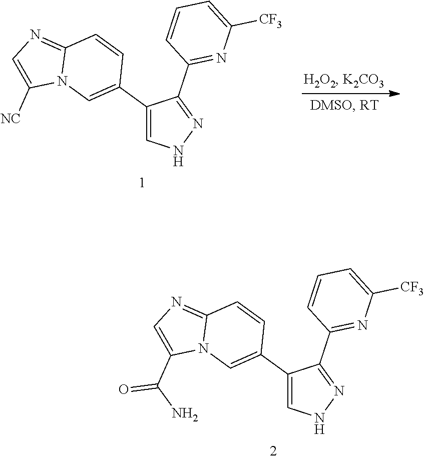 [SMILES: N#Cc1cnc2ccc(-c3c[nH]nc3-c3cccc(C(F)(F)F)n3)cn12 ; NC(=O)c1cnc2ccc(-c3c[nH]nc3-c3cccc(C(F)(F)F)n3)cn12]

### Intermediate 1B: 3-(4,4,5,5-tetramethyl-1,3,2-dioxaborolan-2-yl)-1-((2-(trimethylsilyl)ethoxy)methyl)-1H-pyrazole

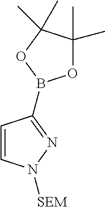 [SMILES: CC1(C)OB(c2ccn(COCC[Si](C)(C)C)n2)OC1(C)C]

To a solution of 1A (Source: COMBI-BLOCKS; 0.5 g, 2.58 mmol) in CH₂Cl₂ (10 mL) at 0° C. was added DIPEA (0.9 mL, 5.15 mmol) and the reaction mixture was stirred at that temperature for 30 min. At this point SEM-Cl (0.503 mL, 2.83 mmol) was added and the reaction mixture was slowly allowed to warm to RT and stirred for 16 h. The reaction mixture was diluted with DCM (50 mL) and quenched with H₂O (20 mL). The organic layer was separated. The aqueous layer was back-extracted with DCM (3×50 mL). The combined organic layers were washed with 10% aq. solution of NaHCO₃ (50 mL) and brine, dried over Na₂SO₄, filtered and the filtrate concentrated under reduced pressure to afford the crude product as a pale yellow liquid. The residue was purified via silica gel chromatography (24 g RediSep® column, eluting with 5% MeOH in CHCl₃). Fractions containing the desired product were combined and evaporated to afford intermediate 1B (310 mg, 37.1% yield) as a light yellow liquid. MS (ES): m/z=325.4 [M+H]⁺; HPLC Ret. Time 3.53 min. (HPLC Method E).

### Intermediate 1C: 2-(trifluoromethyl)-6-(1-((2-(trimethylsilyl)ethoxy)methyl)-1H-pyrazol-3-yl)pyridine

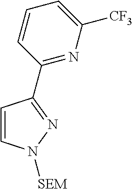 [SMILES: C[Si](C)(C)CCOCn1ccc(-c2cccc(C(F)(F)F)n2)n1]

To a solution of intermediate 1B (400 mg, 1.233 mmol) and 2-bromo-6-(trifluoromethyl)pyridine (418 mg, 1.850 mmol) in ethanol (4 mL)/toluene (4 mL) was added 3M aq. solution of Na₂CO₃ (1.850 mL, 3.70 mmol) and the reaction mixture was degassed for 5 min. To this mixture was then added Pd(PPh₃)₄ (143 mg, 0.123 mmol). The reaction mixture was degassed once again and heated at 90° C. for 12 h. The reaction mixture was cooled to RT and the volatiles were evaporated under reduced pressure to get a crude brown solid. The crude compound was purified via silica gel chromatography (20 g RediSep® column, eluted with 10% EtOAc in petroleum ether). Fractions containing the desired product were combined and evaporated to afford intermediate 1C (300 mg, 70.8% yield) as a light yellow solid. MS (ES): m/z=344.0 [M+H]⁺; HPLC Ret. Time 3.73 min. (HPLC Method E).

### Intermediate 1D: 2-(4-bromo-1-((2-(trimethylsilyl)ethoxy)methyl)-1H-pyrazol-3-yl)-6-(trifluoromethyl)pyridine

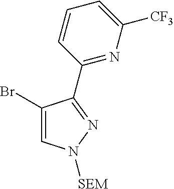 [SMILES: C[Si](C)(C)CCOCn1cc(Br)c(-c2cccc(C(F)(F)F)n2)n1]

To a solution of intermediate 1C (300 mg, 0.874 mmol) in DMF (10 mL) was added NBS (187 mg, 1.048 mmol) and the mixture was stirred overnight at RT. The reaction mixture was quenched with water (0.5 mL) and the solvent was removed under reduced pressure. The residue was dissolved in DCM and washed with H₂O. The aqueous layer was back-extracted with DCM. The combined organic layer was dried over Na₂SO₄, filtered and the filtrate was evaporated under reduced pressure to afford intermediate 1D (334 mg, 91% yield) as a light yellow solid. MS (ES): m/z=422.0 [M+H]⁺; HPLC Ret. Time 3.92 min. (HPLC Method E).

### Intermediate 1E: 6-(3-(6-(trifluoromethyl)pyridin-2-yl)-1-((2-(trimethylsilyl)ethoxy)methyl)-1H-pyrazol-4-yl)imidazo[1,2-a]pyridine-3-carbonitrile

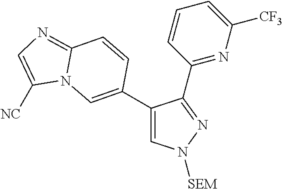 [SMILES: C[Si](C)(C)CCOCn1cc(-c2ccc3ncc(C#N)n3c2)c(-c2cccc(C(F)(F)F)n2)n1]

To a solution of intermediate 1D (320 mg, 1.190 mmol) and 6-(4,4,5,5-tetramethyl-1,3,2-dioxaborolan-2-yl)imidazo[1,2-a]pyridine-3-carbonitrile (reference: WO 2014/055955 A1) (335 mg, 0.793 mmol) in 1,4-dioxane (15 mL) and water (5.0 mL) was added K₃PO₄ (421 mg, 1.983 mmol) and the reaction mixture was degassed for 5 min. To the mixture was then added PdCl₂(dppf)-CH₂Cl₂ adduct (64.8 mg, 0.079 mmol) and the resulting mixture was degassed once again and heated at 90° C. for 12 h. The reaction mixture was cooled to RT and the volatiles were evaporated under reduced pressure to get a crude brown solid. The crude compound was purified via silica gel chromatography (40 g RediSep® column, eluted with 40% EtOAc in petroleum ether). Fractions containing the desired product were combined and evaporated under reduced pressure to afford intermediate 1E (353 mg, 92% yield) as a light yellow solid. MS (ES): m/z=485.2 [M+H]⁺; HPLC Ret. Time 3.40 min. (HPLC Method E).

### Example 1: 6-(3-(6-(trifluoromethyl)pyridin-2-yl)-1H-pyrazol-4-yl)imidazo[1,2-a]pyridine-3-carbonitrile

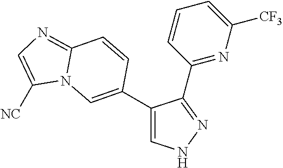 [SMILES: N#Cc1cnc2ccc(-c3c[nH]nc3-c3cccc(C(F)(F)F)n3)cn12]

A solution of intermediate 1E (100 mg, 0.206 mmol) in TFA (4 mL) was stirred at RT for 12 h. The reaction mixture was evaporated under reduced pressure to get the crude compound. The crude product was dissolved in DMF and purified via preparative HPLC (Condition N). Fractions containing the desired product were combined and dried under vacuum to afford Example 1 (11.5 mg, 31.5% yield). MS (ES): m/z=355.0 [M+H]⁺; HPLC Ret. Time 1.52 min. (HPLC Method C). ¹H NMR (400 MHz, DMSO-d₆) δ ppm 13.59 (s, 1H), 8.63 (s, 1H), 8.45 (s, 1H), 8.31 (s, 1H), 8.14-8.27 (m, 2H), 7.78-7.83 (m, 2H), 7.67-7.69 (m, 1H).

### Example 2: 6-(3-(6-(trifluoromethyl)pyridin-2-yl)-1H-pyrazol-4-yl)imidazo[1,2-a]pyridine-3-carboxamide

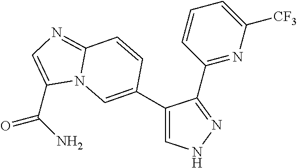 [SMILES: NC(=O)c1cnc2ccc(-c3c[nH]nc3-c3cccc(C(F)(F)F)n3)cn12]

To a stirred solution of Example 1 (40 mg, 0.113 mmol) and K₂CO₃ (54.6 mg, 0.395 mmol) in DMSO (3 mL) was added 30% aq. solution of H₂O₂ (0.346 mL, 3.39 mmol) at 0° C. The reaction mixture was then warmed to RT and stirred for 3 h. To the reaction was added water (10 mL) and the mixture was extracted with a solution of 10% MeOH in DCM (2×20 mL). The combined organic layers were dried over anhydrous Na₂SO₄, filtered and the filtrate was evaporated under reduced pressure to get semi-solid residue. The crude product was dissolved in DMF and purified via preparative HPLC (Condition N). Fractions containing the desired product were combined and evaporated under vacuum to afford Example 2. MS (ES): m/z=373.1 [M+H]⁺; HPLC Ret. Time 1.22 min. (HPLC Method C). ¹H NMR (400 MHz, DMSO-d₆) δ ppm 13.52 (s, 1H), 9.46 (s, 1H), 8.32 (s, 1H), 8.11-8.19 (m, 3H), 7.79-7.92 (m, 2H), 7.62 (d, J=9.2 Hz, 1H), 7.51 (d, J=9.2 Hz, 1H), 7.31 (bs, 1H).

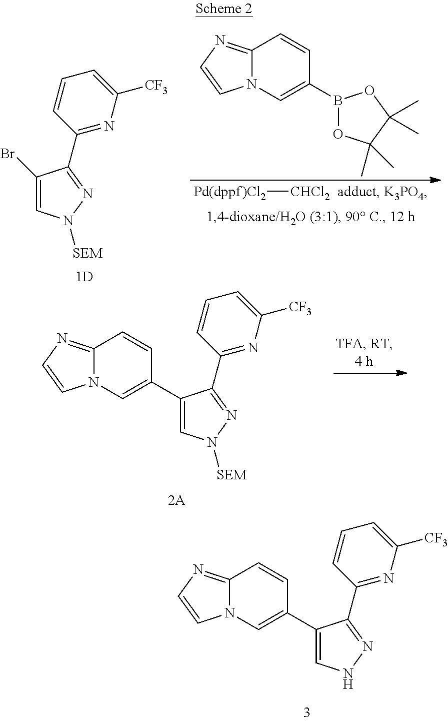

### Intermediate 2A: 6-(3-(6-(trifluoromethyl)pyridin-2-yl)-1-((2-(trimethylsilyl)ethoxy)methyl)-1H-pyrazol-4-yl)imidazo[1,2-a]pyridine

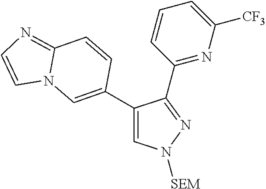 [SMILES: C[Si](C)(C)CCOCn1cc(-c2ccc3nccn3c2)c(-c2cccc(C(F)(F)F)n2)n1]

Intermediate 2A was synthesized analogous to intermediate 1E by coupling intermediate 1D and commercially available imidazo[1,2-a]pyridin-6-ylboronic acid (Prepared using procedure described in reference: CN 103275112 A The crude product was purified by silica gel chromatography (40 g RediSep® column, eluting with a gradient of 50-60% EtOAc in petroleum ether). Fractions containing the desired product were combined and evaporated to afford intermediate 2A. MS (ES): m/z=460.3 [M+H]⁺; HPLC Ret. Time 1.14 min. (HPLC Method B).

### Example 3: 6-(3-(6-(trifluoromethyl)pyridin-2-yl)-1H-pyrazol-4-yl)imidazo[1,2-a]pyridine

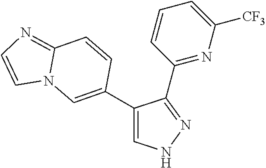 [SMILES: FC(F)(F)c1cccc(-c2n[nH]cc2-c2ccc3nccn3c2)n1]

Example 3 was synthesized from intermediate 2A and TFA by employing the experimental procedure described for Example 1 in Scheme 1. The crude product was dissolved in DMF and purified via preparative HPLC (Condition O). Fractions containing the desired product were combined and dried under vacuum to afford Example 3. MS (ES): m/z=330.1 [M+H]⁺; HPLC Ret. Time 1.38 min. (HPLC Method C). ¹H NMR (400 MHz, DMSO-d₆) δ ppm 8.75 (dd, J=1.2, 1.6 Hz, 1H), 8.11-8.14 (m, 3H), 7.81-7.83 (m, 2H), 7.55 (d, J=1.2 Hz, 1H), 7.47 (d, J=9.2 Hz, 1H), 7.27 (dd, J=2.0, 9.6 Hz, 1H).

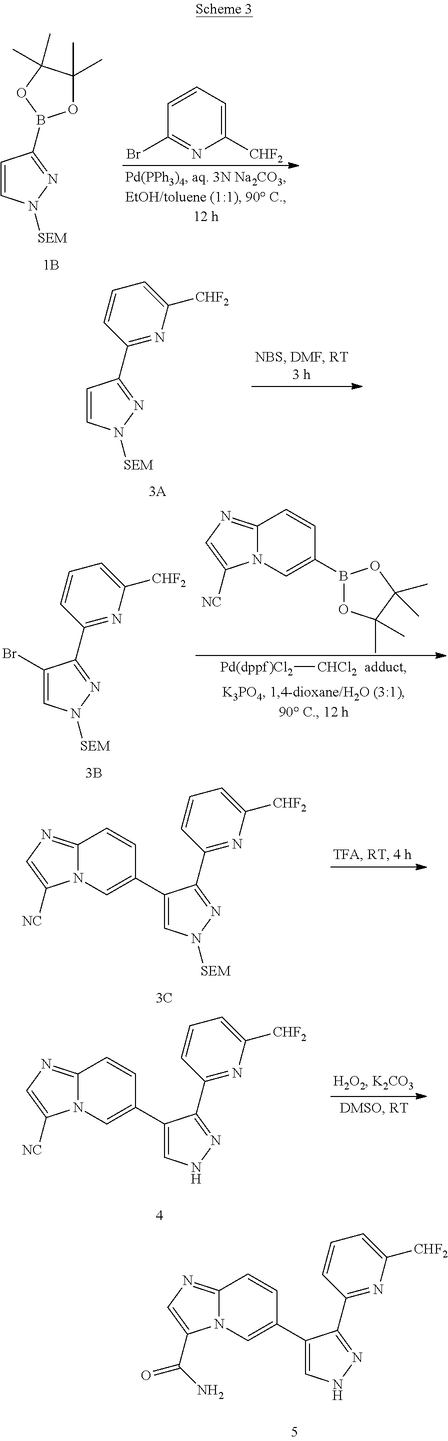

### Intermediate 3A: 2-(difluoromethyl)-6-(1-((2-(trimethylsilyl)ethoxy)methyl)-1H-pyrazol-3-yl)pyridine

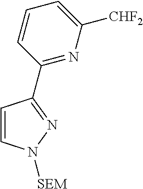 [SMILES: C[Si](C)(C)CCOCn1ccc(-c2cccc(C(F)F)n2)n1]

Intermediate 3A was synthesized analogous to intermediate 1C by coupling intermediate 1B and 2-bromo-6-(difluoromethyl)pyridine. The crude product was purified by silica gel chromatography (20 g RediSep® column, eluting with a gradient of 10-15% EtOAc in petroleum ether). Fractions containing the desired product were combined and evaporated under reduced pressure to afford intermediate 3A. MS (ES): m/z=326.2 [M+H]⁺; HPLC Ret. Time 3.38 min. (HPLC Method E).

### Intermediate 3B: 2-(4-bromo-1-((2-(trimethylsilyl)ethoxy)methyl)-1H-pyrazol-3-yl)-6-(difluoromethyl)pyridine

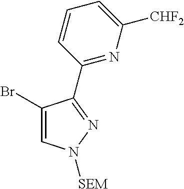 [SMILES: C[Si](C)(C)CCOCn1cc(Br)c(-c2cccc(C(F)F)n2)n1]

Intermediate 3B was synthesized by reacting intermediate 3A with NBS and by employing the experimental procedure described for intermediate 1D in Scheme 1. Intermediate 3B was obtained as a light yellow solid. MS (ES): m/z=406.1 [M+H]⁺; HPLC Ret. Time 1.27 min. (HPLC Method B).

### Intermediate 3C: 6-(3-(6-(difluoromethyl)pyridin-2-yl)-1-((2-(trimethylsilyl)ethoxy)methyl)-1H-pyrazol-4-yl)imidazo[1,2-a]pyridine-3-carbonitrile

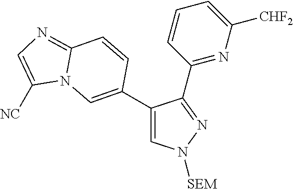 [SMILES: C[Si](C)(C)CCOCn1cc(-c2ccc3ncc(C#N)n3c2)c(-c2cccc(C(F)F)n2)n1]

Intermediate 3C was synthesized analogous to intermediate 1E by coupling intermediate 3B and 6-(4,4,5,5-tetramethyl-1,3,2-dioxaborolan-2-yl)imidazo[1,2-a]pyridine-3-carbonitrile. The crude product was purified by silica gel chromatography (24 g RediSep® column, eluting with a gradient of 30-50% EtOAc in petroleum ether). Fractions containing the desired product were combined and evaporated under reduced pressure to afford intermediate 3C. MS (ES): m/z=467.4 [M+H]⁺; HPLC Ret. Time 1.10 min. (HPLC Method B).

### Example 4: 6-(3-(6-(difluoromethyl)pyridin-2-yl)-1H-pyrazol-4-yl)imidazo[1,2-a]pyridine-3-carbonitrile

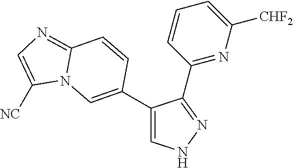 [SMILES: N#Cc1cnc2ccc(-c3c[nH]nc3-c3cccc(C(F)F)n3)cn12]

Example 4 was synthesized by reacting intermediate 3C with TFA and by employing the experimental procedure described for Example 1 in Scheme 1. The crude product was dissolved in DMF and purified via preparative HPLC (Condition K). Fractions containing the desired product were combined and dried under vacuum to afford Example 4. MS (ES): m/z=337.1 [M+H]⁺; HPLC Ret. Time 1.38 min. (HPLC Method C); ¹H NMR (400 MHz, DMSO-d₆) δ ppm 13.57 (bs, 1H), 8.77 (t, J=1.2 Hz, 1H), 8.44 (s, 1H), 8.06-8.31 (m, 3H), 7.77-7.80 (m, 1H), 7.64-7.70 (m, 2H), 6.80 (t, J=54.8 Hz, 1H).

### Example 5: 6-(3-(6-(difluoromethyl)pyridin-2-yl)-1H-pyrazol-4-yl)imidazo[1,2-a]pyridine-3-carboxamide

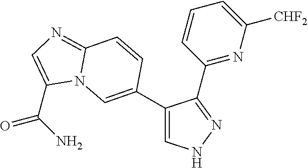 [SMILES: NC(=O)c1cnc2ccc(-c3c[nH]nc3-c3cccc(C(F)F)n3)cn12]

Example 5 was synthesized by reacting Example 4 with H₂O₂/K₂CO₃ and employing the experimental procedure described for Example 2 in Scheme 1. The crude product was dissolved in DMF and purified via preparative HPLC (Condition K). Fractions containing the desired product were combined and evaporated under reduced pressure to afford Example 5. MS (ES): m/z=355.1 [M+H]⁺; HPLC Ret. Time 1.07 min. (HPLC Method C). ¹H NMR (400 MHz, DMSO-d₆) δ ppm 13.52 (s, 1H), 9.46 (s, 1H), 8.32 (s, 1H), 8.11-8.19 (m, 3H), 7.79-7.92 (m, 2H), 7.62 (d, J=9.2 Hz, 1H), 7.51 (d, J=9.2 Hz, 1H), 7.31 (bs, 1H).

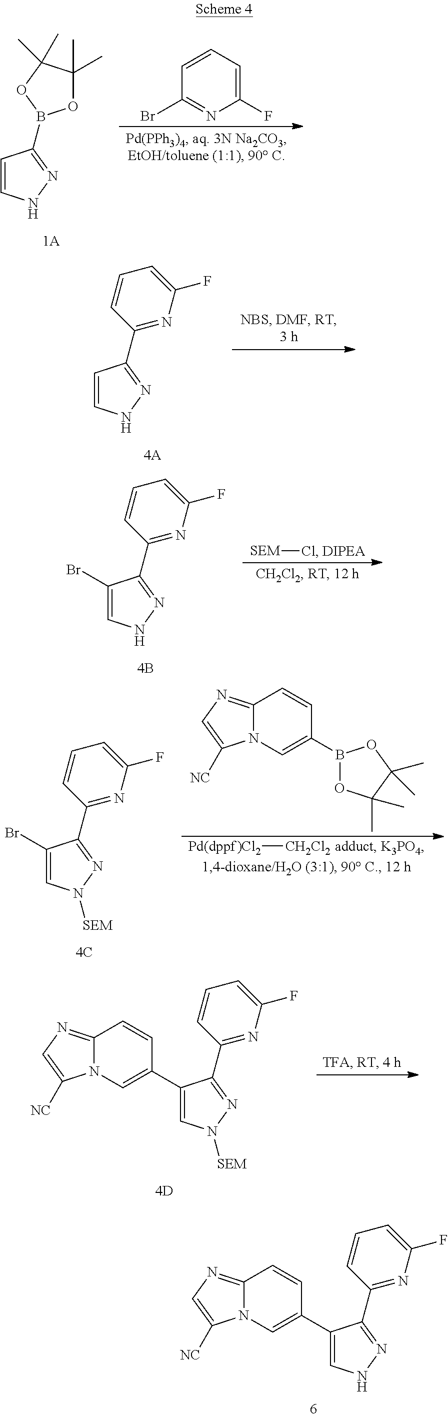

### Intermediate 4A: 2-fluoro-6-(1H-pyrazol-3-yl)pyridine

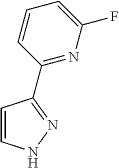 [SMILES: Fc1cccc(-c2cc[nH]n2)n1]

Intermediate 4A was synthesized analogous to intermediate 1E by coupling intermediate 1A and 2-bromo-6-fluoropyridine (Source: Alfa Aesar). The crude product was purified by silica gel chromatography (24 g RediSep® column, eluting with a gradient of 5-10% MeOH in chloroform). Fractions containing the desired product were combined and evaporated to afford intermediate 4A. MS (ES): m/z=164.1 [M+H]⁺; HPLC Ret. Time 0.41 min. (HPLC Method B).

### Intermediate 4B: 2-(4-bromo-1H-pyrazol-3-yl)-6-fluoropyridine

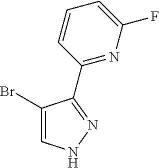 [SMILES: Fc1cccc(-c2n[nH]cc2Br)n1]

Intermediate 4B was synthesized by reacting intermediate 4A with NBS and by employing the experimental procedure described for intermediate 1D in Scheme 1. Intermediate 4B was obtained as light yellow solid. MS (ES): m/z=243.9 [M+H]⁺; HPLC Ret. Time 0.64 min. (HPLC Method A).

### Intermediate 4C: 2-(4-bromo-1-((2-(trimethylsilyl)ethoxy)methyl)-1H-pyrazol-3-yl)-6-fluoropyridine

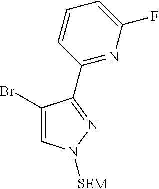 [SMILES: C[Si](C)(C)CCOCn1cc(Br)c(-c2cccc(F)n2)n1]

Intermediate 4C was synthesized by reacting intermediate 4B with SEM-Cl and by employing the experimental procedure described for intermediate 1B in Scheme 1.

The crude product was purified by silica gel chromatography (40 g RediSep® column, eluting with a gradient of 3-5% EtOAc in petroleum ether). Fractions containing the desired product were combined and evaporated to afford intermediate 4C. MS (ES): m/z=372.0 [M+H]⁺; HPLC Ret. Time 3.77 min. (HPLC Method E).

### Intermediate 4D: 6-(3-(6-fluoropyridin-2-yl)-1-((2-(trimethylsilyl)ethoxy)methyl)-1H-pyrazol-4-yl)imidazo[1,2-a]pyridine-3-carbonitrile

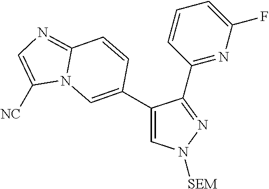 [SMILES: C[Si](C)(C)CCOCn1cc(-c2ccc3ncc(C#N)n3c2)c(-c2cccc(F)n2)n1]

Intermediate 4D was synthesized analogous to intermediate 1E by coupling intermediate 4B and 6-(4,4,5,5-tetramethyl-1,3,2-dioxaborolan-2-yl)imidazo [1,2-a]pyridine-3-carbonitrile. The crude product was purified by silica gel chromatography (24 g RediSep® column, eluting with a gradient of 30-50% EtOAc in petroleum ether). Fractions containing the desired product were combined and evaporated under reduced pressure to afford intermediate 4D. MS (ES): m/z=435.3 [M+H]⁺; HPLC Ret. Time 1.06 min. (HPLC Method B)

### Example 6: 6-(3-(6-fluoropyridin-2-yl)-1H-pyrazol-4-yl)imidazo[1,2-a]pyridine-3-carbonitrile

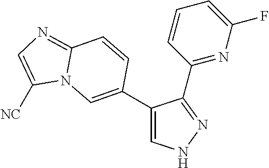 [SMILES: N#Cc1cnc2ccc(-c3c[nH]nc3-c3cccc(F)n3)cn12]

Example 6 was synthesized by reacting intermediate 4C with TFA and by employing the experimental procedure described for Example 1 in Scheme 1. The crude product was dissolved in DMF and purified via preparative HPLC (Condition I). Fractions containing the desired product were combined and dried under vacuum to afford Example 6. MS (ES): m/z=305.1 [M+H]⁺; HPLC Ret. Time 1.25 min. (HPLC Method C); ¹H NMR (400 MHz, DMSO-d₆) δ ppm 13.52 (s, 1H), 8.77-8.80 (m, 1H), 8.45-8.46 (m, 1H), 8.30 (s, 1H), 8.02-8.08 (m, 1H), 7.80-7.93 (m, 2H), 7.64 (dd, J=1.6, 9.6 Hz, 1H), 7.12 (dd, J=2.4, 8.0 Hz, 1H).

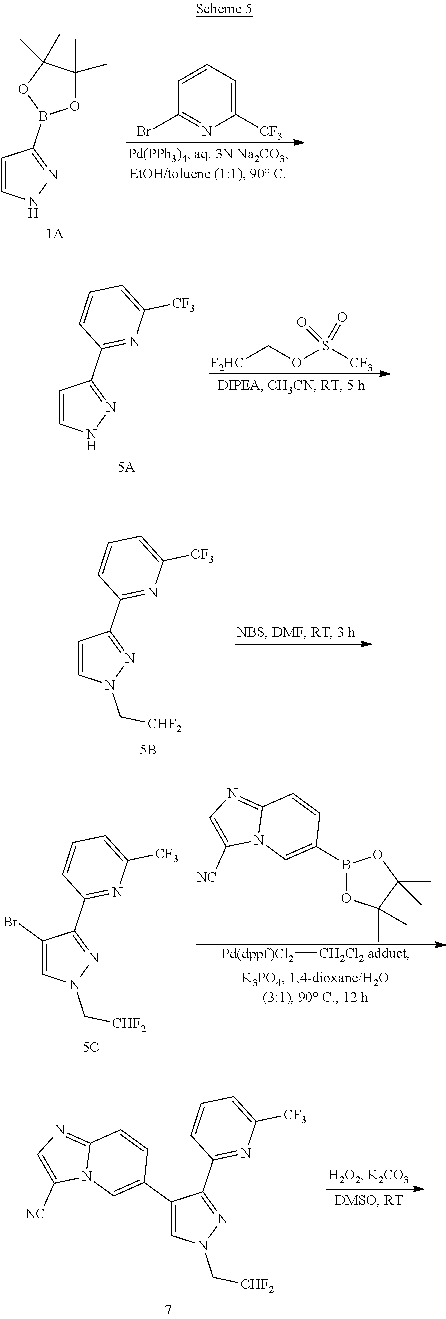

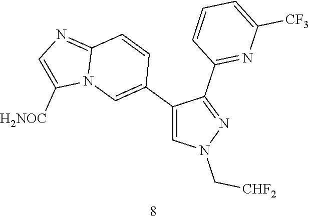 [SMILES: NC(=O)c1cnc2ccc(-c3cn(CC(F)F)nc3-c3cccc(C(F)(F)F)n3)cn12]

### Intermediate 5A: 2-(1H-pyrazol-3-yl)-6-(trifluoromethyl)pyridine

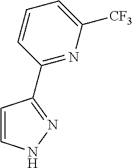 [SMILES: FC(F)(F)c1cccc(-c2cc[nH]n2)n1]

Intermediate 5A was synthesized analogous to intermediate 1E by coupling intermediate 1A and 2-bromo-6-(trifluoromethyl)pyridine (Source: COMBI-BLOCKS, CAS: 189278-27-1 The crude product was purified by silica gel chromatography (40 g RediSep® column, eluting with a gradient of 20-50% EtOAc in petroleum ether). Fractions containing the desired product were combined and evaporated under reduced pressure to afford intermediate 5A. MS (ES): m/z=214.1 [M+H]⁺; HPLC Ret. Time 0.98 min. (HPLC Method B).

### Intermediate 5B: 2-(1-(2,2-difluoroethyl)-1H-pyrazol-3-yl)-6-(trifluoromethyl)pyridine

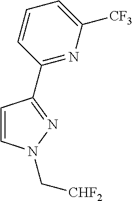 [SMILES: FC(F)Cn1ccc(-c2cccc(C(F)(F)F)n2)n1]

To a solution of 5A (450 mg, 2.111 mmol) in acetonitrile (9 mL) at 0° C. was added DIPEA (0.737 mL, 4.22 mmol) and the mixture was stirred for 10 min. Then 2,2-difluoroethyl trifluoromethanesulfonate (542 mg, 2.53 mmol) was added and the reaction stirred at RT for 5 h. The volatiles were removed under reduced pressure and the residue was dissolved in ethyl acetate (20 mL) and washed with water (15 mL). The aqueous layer was back-extracted with ethyl acetate (2×15 mL). The combined organic layers were washed with brine, dried over Na₂SO₄, filtered and the filtrate was concentrated under reduced pressure to get a light yellow liquid. The crude product was purified by silica gel chromatography (24 g RediSep® column, eluting with a gradient of 30-50% EtOAc in petroleum ether). Fractions containing the desired product were combined and evaporated to afford intermediate 5B (234 mg, 40.0% yield). MS (ES): m/z=278.1 [M+H]⁺; HPLC Ret. Time 1.23 min. (HPLC Method B).

### Intermediate 5C: 2-(4-bromo-1-(2,2-difluoroethyl)-1H-pyrazol-3-yl)-6-(trifluoromethyl)pyridine

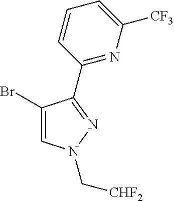 [SMILES: FC(F)Cn1cc(Br)c(-c2cccc(C(F)(F)F)n2)n1]

Intermediate 5C was synthesized by reacting intermediate 5B with NBS and by employing the experimental procedure described for intermediate 1D in Scheme 1. Intermediate 5C was obtained as light yellow solid. MS (ES): m/z=356.1 [M+H]⁺; HPLC Ret. Time 1.35 min. (HPLC Method B).

### Example 7: 6-(1-(2,2-difluoroethyl)-3-(6-(trifluoromethyl)pyridin-2-yl)-1H-pyrazol-4-yl)imidazo[1,2-a]pyridine-3-carbonitrile

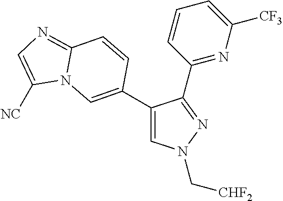 [SMILES: N#Cc1cnc2ccc(-c3cn(CC(F)F)nc3-c3cccc(C(F)(F)F)n3)cn12]

Example 7 was synthesized analogous to intermediate 1E by coupling intermediate 5C and 6-(4,4,5,5-tetramethyl-1,3,2-dioxaborolan-2-yl)imidazo[1,2-a]pyridine-3-carbonitrile. The crude product was dissolved in DMF and purified via preparative HPLC (Condition K). Fractions containing the desired product were combined and dried under vacuum to afford Example 7. MS (ES): m/z=419.1 [M+H]⁺; HPLC Ret. Time 1.95 min. (HPLC Method C). ¹H NMR (400 MHz, DMSO-d₆) δ ppm 8.64 (s, 1H), 8.45 (s, 1H), 8.34 (s, 1H), 8.15-8.20 (m, 2H), 7.85 (dd, J=1.6, 6.8 Hz, 1H), 7.79 (dd, J=0.8, 9.2 Hz, 1H), 7.63 (dd, J=1.6, 9.2 Hz, 1H), 6.53 (tt, J=54.4, 3.6 Hz, 1H), 4.82 (dt, J=3.6, 15.2 Hz, 2H).

### Example 8: 6-(1-(2,2-difluoroethyl)-3-(6-(trifluoromethyl)pyridin-2-yl)-1H-pyrazol-4-yl)imidazo[1,2-a]pyridine-3-carboxamide

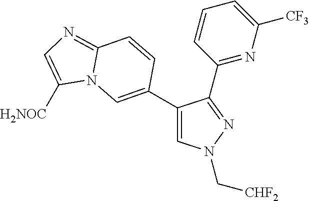 [SMILES: NC(=O)c1cnc2ccc(-c3cn(CC(F)F)nc3-c3cccc(C(F)(F)F)n3)cn12]

Example 8 was synthesized by reacting Example 7 with H₂O₂/K₂CO₃ and by employing the experimental procedure described for Example 2 in Scheme 1. The crude product was dissolved in DMF and purified via preparative HPLC (Condition L). Fractions containing the desired product were combined and evaporated under reduced pressure to afford Example 8. MS (ES): m/z=437.2 [M+H]⁺; HPLC Ret. Time 1.32 min. (HPLC Method C). ¹H NMR (400 MHz, DMSO-d₆) δ ppm 9.46 (s, 1H), 8.32 (s, 1H), 8.25 (s, 1H), 8.12-8.17 (m, 2H), 7.91 (s, 1H), 7.80-7.84 (m, 1H), 7.62 (dd, J=0.8, 9.2 Hz, 1H), 7.45 (dd, J=2, 9.2 Hz, 1H), 7.32 (s, 1H), 6.53 (tt, J=3.6, 54.4 Hz, 1H), 4.81 (dt, J=3.6, 15.2 Hz, 2H).

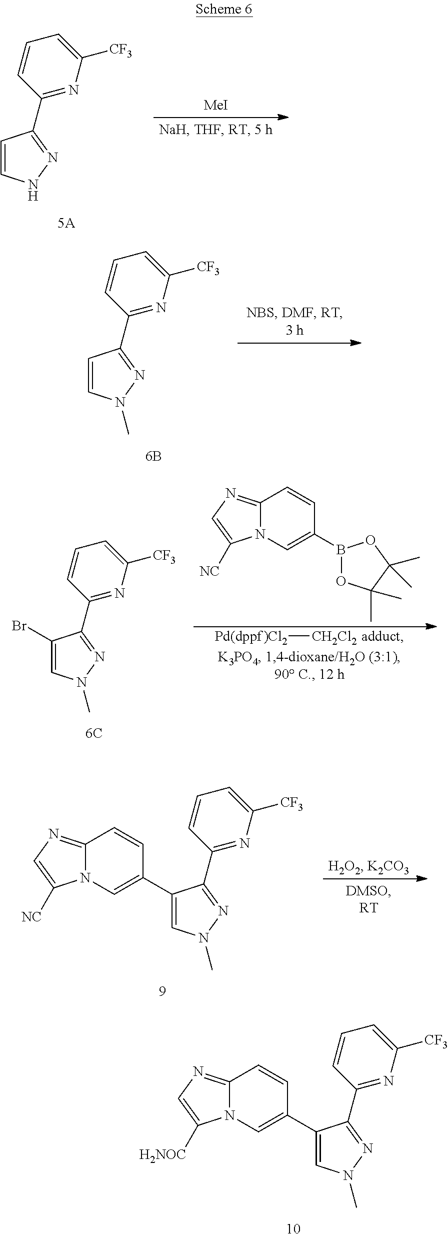

### Intermediate 6B: 2-(1-methyl-1H-pyrazol-3-yl)-6-(trifluoromethyl)pyridine

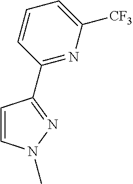 [SMILES: Cn1ccc(-c2cccc(C(F)(F)F)n2)n1]

To a solution of 5A (0.45 g, 2.111 mmol) in THF (25 mL) at 0° C. was added NaH (0.101 g, 2.53 mmol, 60% in mineral oil) and the mixture was stirred for 10 min. To the mixture was then added methyl iodide (0.264 mL, 4.22 mmol) and the resultant reaction mixture was stirred at RT for 2 h. The volatiles were removed under reduced pressure and the residue was dissolved in ethyl acetate (20 mL) and washed with water (10 mL). The aqueous layer was back-extracted with ethyl acetate (2×10 mL). The combined organic layers were washed with brine, dried over Na₂SO₄, filtered and the filtrate was concentrated under reduced pressure to afford the crude product. The crude product was purified by silica gel chromatography (24 g RediSep® column, eluting with a gradient of 20-50% EtOAc in petroleum ether). Fractions containing the desired product were combined and evaporated under reduced pressure to afford intermediate 6B (345 mg, 71.9% yield). MS (ES): m/z=228.2 [M+H]⁺; HPLC Ret. Time 2.22 min. (HPLC Method E).

### Intermediate 6C: 2-(4-bromo-1-methyl-1H-pyrazol-3-yl)-6-(trifluoromethyl)pyridine

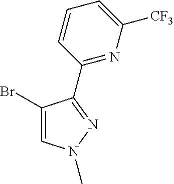 [SMILES: Cn1cc(Br)c(-c2cccc(C(F)(F)F)n2)n1]

Intermediate 6C was synthesized by reacting intermediate 6B with NBS and by employing the experimental procedure described for intermediate for intermediate 1D in Scheme 1. Intermediate 6C was obtained as a light yellow solid. MS (ES): m/z=308.0 [M+H]⁺; HPLC Ret. Time 2.53 min. (HPLC Method E).

### Example 9: 6-(1-methyl-3-(6-(trifluoromethyl)pyridin-2-yl)-1H-pyrazol-4-yl)imidazo[1,2-a]pyridine-3-carbonitrile

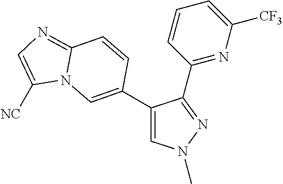 [SMILES: Cn1cc(-c2ccc3ncc(C#N)n3c2)c(-c2cccc(C(F)(F)F)n2)n1]

Example 9 was synthesized analogous to intermediate 1E by coupling intermediate 6C and 6-(4,4,5,5-tetramethyl-1,3,2-dioxaborolan-2-yl)imidazo[1,2-a]pyridine-3-carbonitrile. The crude product was dissolved in DMF and purified via preparative HPLC (Condition K). Fractions containing the desired product were combined and evaporated to afford Example 9. MS (ES): m/z=369.1 [M+H]⁺; HPLC Ret. Time 1.79 min. (HPLC Method C); ¹H NMR (400 MHz, DMSO-d₆): δ ppm 8.62 (s, 1H), 8.45 (s, 1H), 8.24 (s, 1H), 8.12-8.20 (m, 2H), 7.77-7.82 (m, 2H), 7.63-7.65 (m, 1H), 4.01 (s, 3H).

### Example 10: 6-(1-methyl-3-(6-(trifluoromethyl)pyridin-2-yl)-1H-pyrazol-4-yl)imidazo[1,2-a]pyridine-3-carboxamide

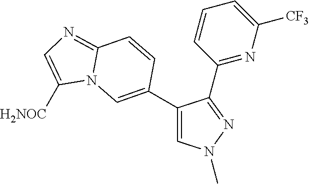 [SMILES: Cn1cc(-c2ccc3ncc(C(N)=O)n3c2)c(-c2cccc(C(F)(F)F)n2)n1]

Example 10 was synthesized by reacting Example 9 with H₂O₂/K₂CO₃ and by employing the experimental procedure described for Example 2 in Scheme 1. The crude product was dissolved in DMF and purified via preparative HPLC (Condition K). Fractions containing the desired product were combined and dried under vacuum to afford Example 10. MS (ES): m/z=387.2 [M+H]⁺; HPLC Ret. Time 1.16 min. (HPLC Method C); ¹H NMR (400 MHz, DMSO-d₆) δ ppm 9.47 (s, 1H), 8.35 (s, 1H), 8.09-8.16 (m, 3H), 7.91 (bs, 1H), 7.77 (dd, J=2.0, 6.8 Hz, 1H), 7.64 (d, J=9.2 Hz, 1H), 7.52 (dd, J=1.6, 9.2 Hz, 1H), 7.36 (bs, 1H), 4.01 (s, 3H).

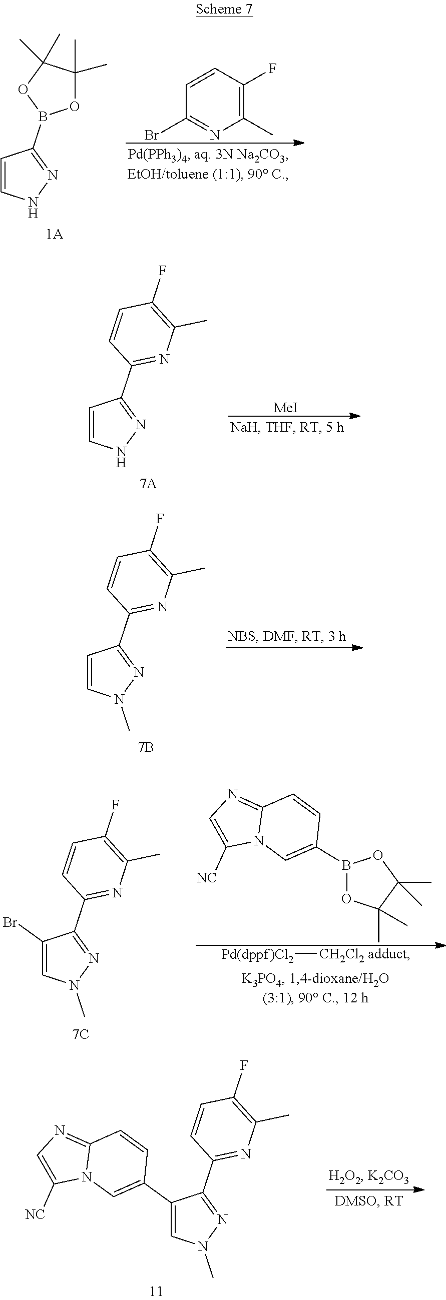

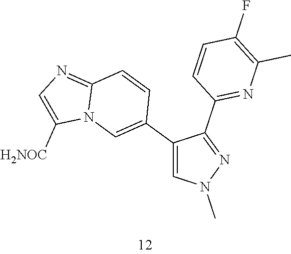 [SMILES: Cc1nc(-c2nn(C)cc2-c2ccc3ncc(C(N)=O)n3c2)ccc1F]

### Intermediate 7A: 3-fluoro-2-methyl-6-(1H-pyrazol-3-yl)pyridine

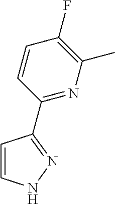

Intermediate 7A was synthesized analogous to intermediate 1E by coupling intermediate 1A and 6-bromo-3-fluoro-2-methylpyridine. The crude product was purified by silica gel chromatography (40 g RediSep® column, eluting with a gradient of 20-50% EtOAc in petroleum ether). Fractions containing the desired product were combined and evaporated under reduced pressure to afford intermediate 7A. MS (ES): m/z=178.2 [M+H]⁺; HPLC Ret. Time 1.97 min. (HPLC Method E).

### Intermediate 7B: 3-fluoro-2-methyl-6-(1-methyl-1H-pyrazol-3-yl)pyridine

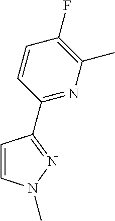

Intermediate 7B was synthesized analogous to intermediate 6B by reacting intermediate 7A and methyl iodide. The crude product was purified by silica gel chromatography (24 g RediSep® column, eluting with a gradient of 20-50% EtOAc in petroleum ether). Fractions containing the desired product were combined and evaporated under reduced pressure to afford intermediate 7B. MS (ES): m/z=192.2 [M+H]⁺; HPLC Ret. Time 1.61 min. (HPLC Method E).

### Intermediate 7C: 6-(4-bromo-1-methyl-1H-pyrazol-3-yl)-3-fluoro-2-methylpyridine

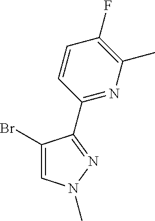

Intermediate 7C was synthesized by reacting intermediate 7B with NBS and by employing the experimental procedure described for intermediate 1D in Scheme 1. Intermediate 7C was obtained as a light yellow solid. MS (ES): m/z=270.7 [M+H]⁺; HPLC Ret. Time 3.17 min. (HPLC Method E).

### Example 11: 6-(3-(5-fluoro-6-methylpyridin-2-yl)-1-methyl-1H-pyrazol-4-yl)imidazo[1,2-a]pyridine-3-carbonitrile

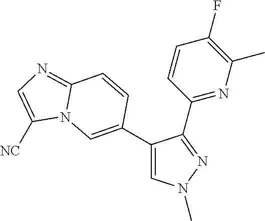 [SMILES: Cc1nc(-c2nn(C)cc2-c2ccc3ncc(C#N)n3c2)ccc1F]

Example 11 was synthesized analogous to intermediate 1E by coupling intermediate 7C and 6-(4,4,5,5-tetramethyl-1,3,2-dioxaborolan-2-yl)imidazo[1,2-a]pyridine-3-carbonitrile. The crude product was dissolved in DMF and purified via preparative HPLC (Condition L). Fractions containing the desired product were combined and evaporated under reduced pressure to afford Example 11. MS (ES): m/z=333.2 [M+H]⁺; HPLC Ret. Time 1.41 min. (HPLC Method C); ¹H NMR (400 MHz, DMSO-d₆) δ ppm 9.91 (s, 1H), 8.44 (s, 1H), 8.24 (s, 1H), 7.81 (d, J=9.2 Hz, 1H), 7.66-7.73 (m, 3H), 3.97 (s, 3H), 2.38 (d, J=2.8 Hz, 3H).

### Example 12: 6-(3-(5-fluoro-6-methylpyridin-2-yl)-1-methyl-1H-pyrazol-4-yl)imidazo[1,2-a]pyridine-3-carboxamide

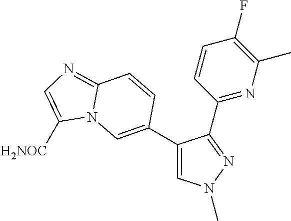 [SMILES: Cc1nc(-c2nn(C)cc2-c2ccc3ncc(C(N)=O)n3c2)ccc1F]

Example 12 was synthesized by reacting Example 11 with H₂O₂/K₂CO₃ and employing the experimental procedure described for Example 2 in Scheme 1. The crude product was dissolved in DMF and purified via preparative HPLC (Condition T). Fractions containing the desired product were combined and evaporated under reduced pressure to afford Example 12. MS (ES): m/z=351.0 [M+H]⁺; HPLC Ret. Time 1.19 min. (HPLC Method C); ¹H NMR (400 MHz, DMSO-d₆) δ ppm 9.61 (s, 1H), 8.32 (s, 1H), 8.10 (s, 1H), 7.89 (bs, 1H), 7.63-7.69 (m, 3H), 7.48 (dd, J=2.0, 9.2 Hz, 1H), 7.31 (s, 1H), 3.96 (s, 3H), 2.27 (d, J=2.8 Hz, 3H).

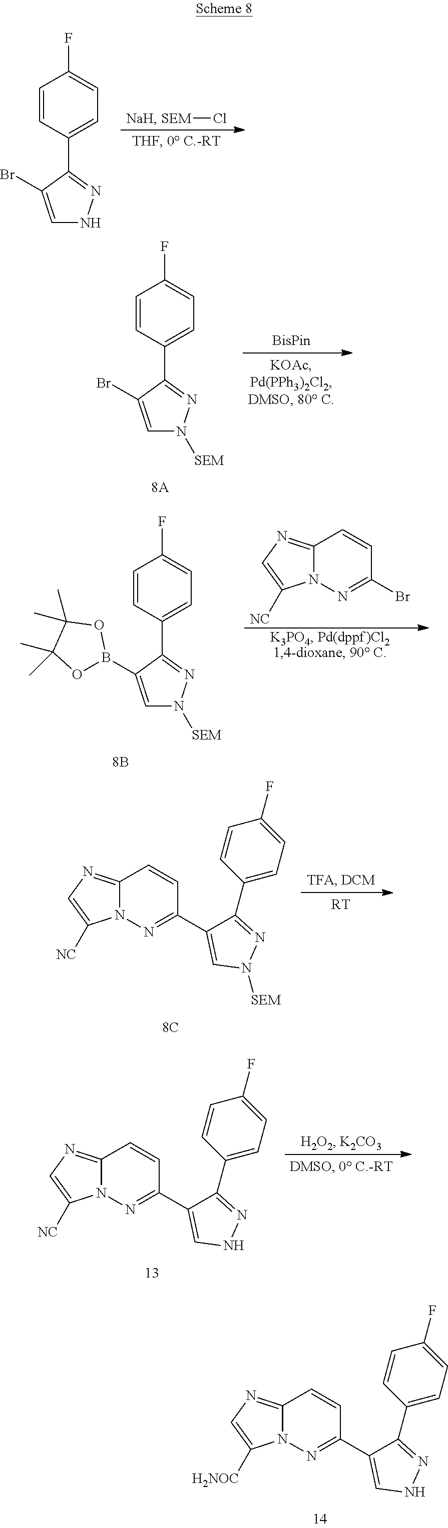

### Intermediate 8A: 4-bromo-3-(4-fluorophenyl)-1-((2-(trimethylsilyl)ethoxy)methyl)-1H-pyrazole

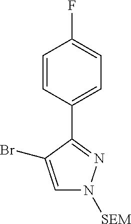 [SMILES: C[Si](C)(C)CCOCn1cc(Br)c(-c2ccc(F)cc2)n1]

To a solution of 4-bromo-3-(4-fluorophenyl)-1H-pyrazole (2.00 g, 8.30 mmol) in THF (10 mL) at 0° C. was added NaH (0.398 g, 9.96 mmol, 60% in mineral oil) and the mixture was stirred for 10 min. To the reaction was then added SEM-Cl (2.207 mL, 12.45 mmol) and the resultant reaction mixture was stirred at RT for additional 2 h. The reaction was quenched with water and extracted with ethyl acetate. The combined organic layers were washed with water, brine, dried over Na₂SO₄, filtered and the filtrate was concentrated under reduced pressure to give the crude compound. The crude product was purified by silica gel chromatography (24 g RediSep® column, eluting with a gradient of 10-100% EtOAc in petroleum ether). Fractions containing the desired product were combined and evaporated under reduced pressure to afford intermediate 8A (2.7 g, 88% yield). LCMS: m/z=371.2 [M+H]⁺; Ret. Time 3.90 min. (LCMS Method E).

### Intermediate 8B: 3-(4-fluorophenyl)-4-(4,4,5,5-tetramethyl-1,3,2-dioxaborolan-2-yl)-1-((2-(trimethylsilyl)ethoxy)methyl)-1H-pyrazole

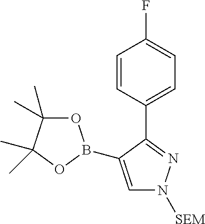 [SMILES: CC1(C)OB(c2cn(COCC[Si](C)(C)C)nc2-c2ccc(F)cc2)OC1(C)C]

To a solution of 8A (1.0 g, 2.69 mmol) in DMSO (2 mL) was added BisPin (1.368 g, 5.39 mmol), potassium acetate (0.793 g, 8.08 mmol), and bis(triphenylphosphine)palladium(II) chloride (0.095 g, 0.135 mmol). The reaction mixture was degassed for 10 min. with argon and heated overnight at 80° C. The reaction mixture was diluted with ethyl acetate and filtered through a Celite® pad. The filtrate was washed with water, brine, dried over Na₂SO₄, filtered and the filtrate was concentrated under reduced pressure to give the crude residue. The crude product was purified by silica gel chromatography (24 g RediSep® column, eluting with a gradient of 20-100% EtOAc in petroleum ether). Fractions containing the desired product were combined and evaporated under reduced pressure to afford intermediate 8B (0.5 g, 44.4% yield). LCMS: m/z=419.3 [M+H]⁺; Ret. Time 1.40 min. (LCMS Method B).

### Intermediate 8C: 6-(3-(4-fluorophenyl)-1-((2-(trimethylsilyl)ethoxy)methyl)-1H-pyrazol-4-yl)imidazo[1,2-b]pyridazine-3-carbonitrile

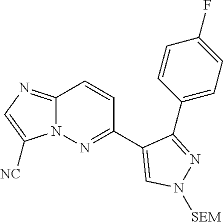 [SMILES: C[Si](C)(C)CCOCn1cc(-c2ccc3ncc(C#N)n3n2)c(-c2ccc(F)cc2)n1]

Intermediate 8C was synthesized by reacting intermediate 8B with 6-bromoimidazo[1,2-b]pyridazine-3-carbonitrile (Synthesized using procedure described in reference U.S. Pat. Appl. Publ., 20140134133) and by employing the experimental procedure described for Intermediate 1E in Scheme 1. The crude product was purified by silica gel chromatography (24 g RediSep® column, eluting with a gradient of 10-100% EtOAc in petroleum ether). Fractions containing the desired product were combined and evaporated under reduced pressure to afford intermediate 8C. LCMS: m/z=435.2 [M+H]⁺; Ret. Time 3.44 min. (LCMS Method E).

### Example 13: 6-(3-(4-fluorophenyl)-1H-pyrazol-4-yl)imidazo [1,2-b]pyridazine-3-carbonitrile

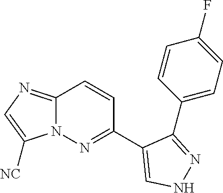 [SMILES: N#Cc1cnc2ccc(-c3c[nH]nc3-c3ccc(F)cc3)nn12]

Example 13 was synthesized by reacting Intermediate 8C with TFA and by employing the experimental procedure described for Example 1 in Scheme 1. The crude product was dissolved in DMF and purified via preparative HPLC (Condition N). Fractions containing the desired product were combined and dried under vacuum to afford Example 13. LCMS: m/z=305.1 [M+H]⁺; Ret. Time 1.505 min. (LCMS Method C). ¹H NMR (400 MHz, DMSO-d₆) δ ppm 7.50 (br. s. 2H), 7.97 (s, 1H), 7.82-7.90 (m, 1H), 7.87 (d, J=9.6 Hz, 1H), 7.97 (dd, J=8.7, 5.6 Hz, 2H), 8.56 (d, J=9.5 Hz, 1H), 8.76 (s, 1H).

### Example 14: 6-(3-(4-fluorophenyl)-1H-pyrazol-4-yl)imidazo[1,2-b]pyridazine-3-carboxamide

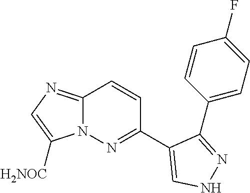 [SMILES: NC(=O)c1cnc2ccc(-c3c[nH]nc3-c3ccc(F)cc3)nn12]

Example 14 was synthesized by reacting Example 13 with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 14. LCMS: m/z=323.1 [M+H]⁺; Ret. Time 1.069 min. (LCMS Method C). ¹H NMR (400 MHz, DMSO-d₆) δ ppm 13.51 (s, 1H), 8.60 (d, J=1.7 Hz, 1H), 8.18-8.32 (m, 2H), 7.51-7.64 (m, 4H), 7.17-7.36 (m, 3H).

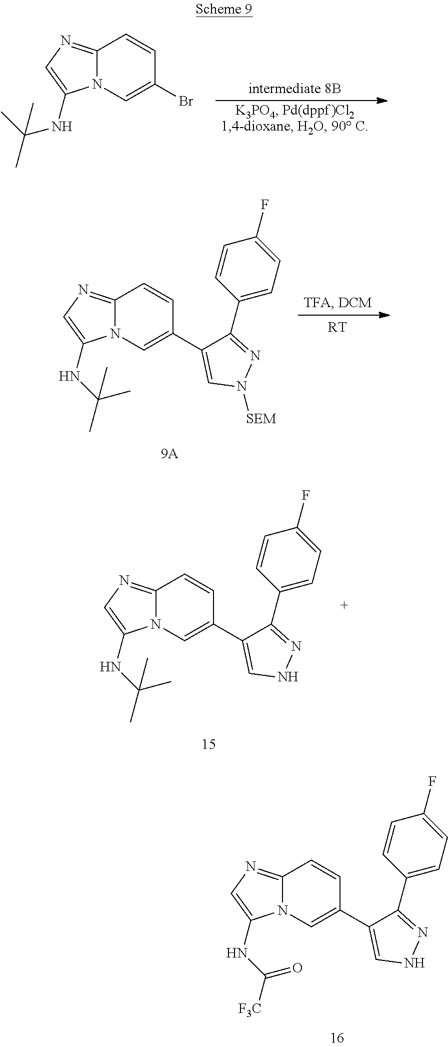 [SMILES: CC(C)(C)Nc1cnc2ccc(Br)cn12 ; "K3PO4, Pd(dppf)Cl21,4-dioxane, H2O, 90° C." ; CC(C)(C)Nc1cnc2ccc(-c3cn(COCC[Si](C)(C)C)nc3-c3ccc(F)cc3)cn12 ; "RT" ; CC(C)(C)Nc1cnc2ccc(-c3c[nH]nc3-c3ccc(F)cc3)cn12 ; O=C(Nc1cnc2ccc(-c3c[nH]nc3-c3ccc(F)cc3)cn12)C(F)(F)F]

### Intermediate 9A: N-(tert-butyl)-6-(3-(4-fluorophenyl)-1-((2-(trimethylsilyl)ethoxy)methyl)-1H-pyrazol-4-yl)imidazo[1,2-a]pyridin-3-amine

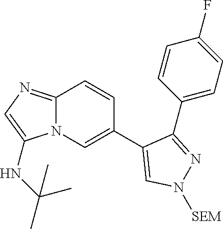 [SMILES: CC(C)(C)Nc1cnc2ccc(-c3cn(COCC[Si](C)(C)C)nc3-c3ccc(F)cc3)cn12]

Intermediate 9A was synthesized by reacting 6-bromo-N-(tert-butyl)imidazo[1,2-a]pyridin-3-amine (reference: WO 2013/064984 A1) with 8B and by employing the experimental procedure described for Intermediate 1E in Scheme 1. The crude product was purified by silica gel chromatography (24 g RediSep® column, eluting with a gradient of 40-100% EtOAc in petroleum ether). Fractions containing the desired product were combined and evaporated under reduced pressure to afford intermediate 9A. LCMS: m/z=480.2 [M+H]⁺; Ret. Time 1.26 min. (LCMS Method B).

### Example 15: N-(tert-butyl)-6-(3-(4-fluorophenyl)-1H-pyrazol-4-yl)imidazo[1,2-a]pyridin-3-amine

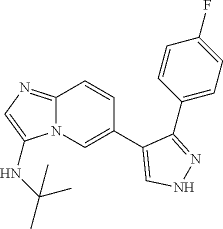 [SMILES: CC(C)(C)Nc1cnc2ccc(-c3c[nH]nc3-c3ccc(F)cc3)cn12]

Example 15 was synthesized by reacting Intermediate 9A with TFA and by employing the experimental procedure described for Example 1 in Scheme 1. The crude product was dissolved in DMF and purified via preparative HPLC (Condition L). Fractions containing the desired product were combined and evaporated under reduced pressure to afford Example 15. LCMS: m/z=350.2 [M+H]⁺; Ret. Time 1.69 min. (LCMS Method C). ¹H NMR (400 MHz, DMSO-d₆) δ ppm 8.07 (d, J=16.3 Hz, 2H), 7.76 (s, 1H), 7.43-7.54 (m, 2H), 7.41 (dd, J=9.2, 0.92 Hz, 1H), 7.24-7.33 (m, 1H), 7.09-7.22 (m, 2H), 6.97-7.06 (m, 1H), 4.32 (s, 1H), 1.04 (s, 9H).

### Example 16: 2,2,2-trifluoro-N-(6-(3-(4-fluorophenyl)-1H-pyrazol-4-yl)imidazo[1,2-a]pyridin-3-yl)acetamide

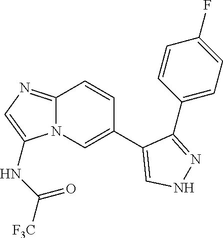 [SMILES: O=C(Nc1cnc2ccc(-c3c[nH]nc3-c3ccc(F)cc3)cn12)C(F)(F)F]

Example 16 was synthesized by reacting Intermediate 9A with TFA and by employing the experimental procedure described for Example 1 in Scheme 1. The crude product was dissolved in DMF and purified via preparative HPLC (Condition L). Fractions containing the desired product were combined and evaporated under reduced pressure to afford Example 16. LCMS: m/z=390.1 [M+H]⁺; Ret. Time 1.327 min. (LCMS Method C). ¹H NMR (400 MHz, DMSO-d₆) δ ppm 8.31-8.35 (m, 2H), 7.73-8.03 (m, 1H), 7.68 (d, J=8.7 Hz, 1H), 7.41-7.55 (m, 4H), 7.28 (t, J=8.5 Hz, 1H), 7.17 (t, J=8.9 Hz, 2H).

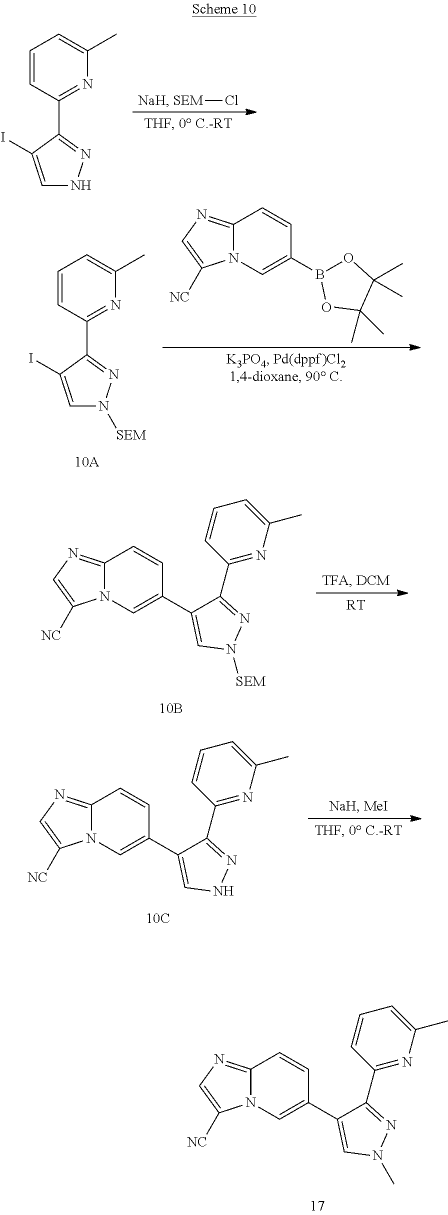

### Intermediate 10A: 2-(4-iodo-1-((2-(trimethylsilyl)ethoxy)methyl)-1H-pyrazol-3-yl)-6-methylpyridine

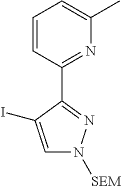 [SMILES: Cc1cccc(-c2nn(COCC[Si](C)(C)C)cc2I)n1]

Intermediate 10A was synthesized by reacting 2-(4-iodo-1H-pyrazol-3-yl)-6-methylpyridine (CAS: 1184917-36-9) with SEM-Cl and by employing the experimental procedure described for Intermediate 8A in Scheme 8. The crude intermediate 10A was used for the next reaction without purification. LCMS: m/z=416.0 [M+H]⁺; Ret. Time 3.694 min. (LCMS Method E).

### Intermediate 10B: 6-(3-(6-methylpyridin-2-yl)-1-((2-(trimethylsilyl)ethoxy)methyl)-1H-pyrazol-4-yl)imidazo[1,2-a]pyridine-3-carbonitrile

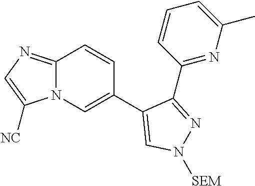 [SMILES: Cc1cccc(-c2nn(COCC[Si](C)(C)C)cc2-c2ccc3ncc(C#N)n3c2)n1]

Intermediate 10B was synthesized by reacting intermediate 10A with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude compound 10B (0.07 g, 45.0% yield) was used for the next step without purification. LCMS: m/z=431.2 [M+H]⁺; Ret. Time 3.18 min. (LCMS Method E).

### Intermediate 10C: 6-(3-(6-methylpyridin-2-yl)-1H-pyrazol-4-yl)imidazo[1,2-a]pyridine-3-carbonitrile

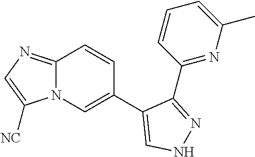 [SMILES: Cc1cccc(-c2n[nH]cc2-c2ccc3ncc(C#N)n3c2)n1]

Intermediate 10C was synthesized by reacting intermediate 10B with TFA and by employing the experimental procedure described for Example 1 in Scheme 1. Intermediate 10C was obtained as a pale yellow solid. LCMS: m/z=301.2 [M+H]⁺; Ret. Time 1.337 min. (LCMS Method E).

### Example 17: 6-(1-methyl-3-(6-methylpyridin-2-yl)-1H-pyrazol-4-yl)imidazo[1,2-a]pyridine-3-carbonitrile

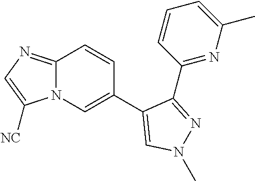 [SMILES: Cc1cccc(-c2nn(C)cc2-c2ccc3ncc(C#N)n3c2)n1]

Example 17 was synthesized by reacting intermediate 10C with MeI and by employing the experimental procedure described for Example 6B in Scheme 6. The crude product was dissolved in DMF and purified via preparative HPLC (Condition N). Fractions containing the desired product were combined and evaporated under reduced pressure to afford Example 17. LCMS: m/z=315.1 [M+H]⁺; Ret. Time 1.225 min. (LCMS Method C). ¹H NMR (400 MHz, DMSO-d₆) δ ppm 9.00 (s, 1H), 8.43 (s, 1H), 8.25 (s, 1H), 7.80 (s, 1H), 7.63-7.76 (m, 3H), 7.21 (d, J=7.6 Hz, 1H), 3.97 (s, 3H), 2.42 (s, 3H).

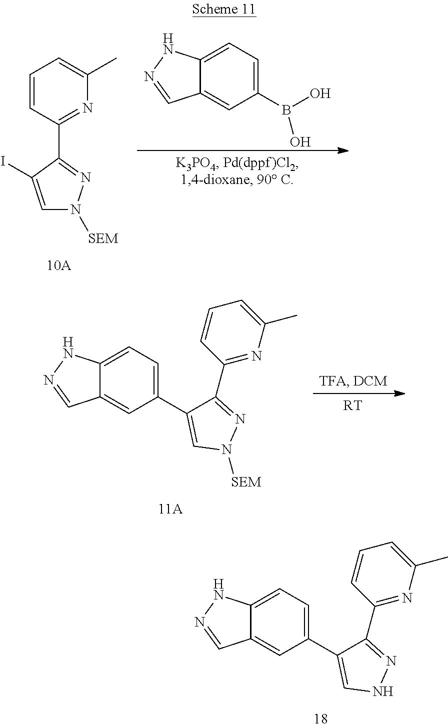

### Intermediate 11A: 5-(3-(6-methylpyridin-2-yl)-1-((2-(trimethylsilyl)ethoxy)methyl)-1H-pyrazol-4-yl)-1H-indazole

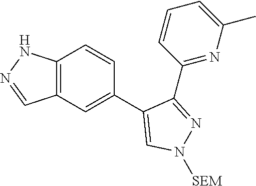 [SMILES: Cc1cccc(-c2nn(COCC[Si](C)(C)C)cc2-c2ccc3[nH]ncc3c2)n1]

Intermediate 11A was synthesized by reacting intermediate 10A with (1H-indazol-5-yl)boronic acid (CAS: 338454-14-1) and by employing the experimental procedure described for intermediate 1E in Scheme 1. The crude product was purified by silica gel chromatography (24 g RediSep® column, eluting with a gradient of 45-100% EtOAc in petroleum ether). Fractions containing the desired product were combined under reduced pressure and evaporated to afford intermediate 11A. LCMS: m/z=406.2 [M+H]⁺; Ret. Time 2.856 min. (LCMS Method E).

### Example 18: 5-(3-(6-methylpyridin-2-yl)-1H-pyrazol-4-yl)-1H-indazole

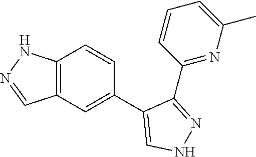 [SMILES: Cc1cccc(-c2n[nH]cc2-c2ccc3[nH]ncc3c2)n1]

Example 18 was synthesized by reacting intermediate 11A with TFA and by employing the experimental procedure described for Example 1 in Scheme 1. The crude product was dissolved in DMF and purified via preparative HPLC (Condition N). Fractions containing the desired product were combined and evaporated under reduced pressure to afford Example 11A. LCMS: m/z=276.1 [M+H]⁺; Ret. Time 1.222 min. (LCMS Method C). ¹H NMR (400 MHz, DMSO-d₆) δ ppm 13.03 (s, 1H) 8.02 (s, 1H), 7.78 (s, 2H), 7.78 (s, 1H), 7.61 (t, J=7.7 Hz, 1H), 7.47 (d, J=8.9 Hz, 1H), 7.33 (d, J 8.7 Hz, 2H), 7.17 (d, J=7.6 Hz, 1H), 2.44 (br, s, 3H).

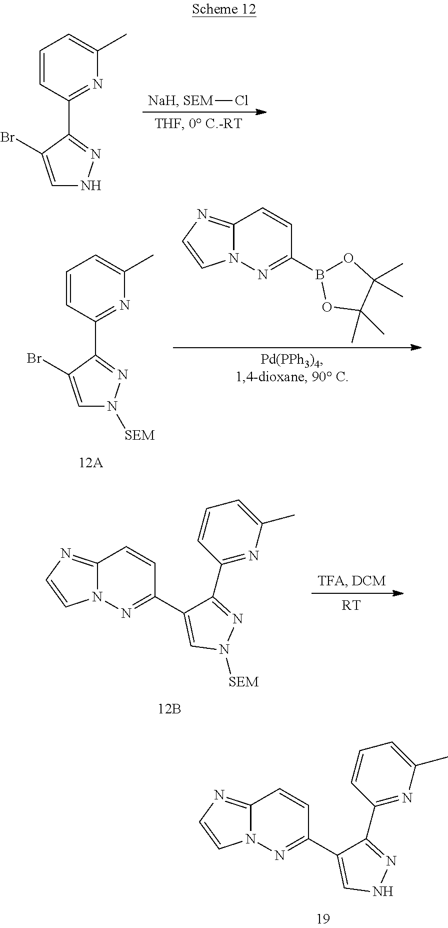

### Intermediate 12A: 2-(4-bromo-1-((2-(trimethylsilyl)ethoxy)methyl)-1H-pyrazol-3-yl)-6-methylpyridine

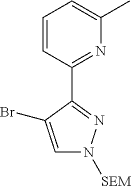 [SMILES: Cc1cccc(-c2nn(COCC[Si](C)(C)C)cc2Br)n1]

Intermediate 12A was synthesized by reacting commercially available 2-(4-bromo-1H-pyrazol-3-yl)-6-methylpyridine with SEM-Cl and by employing the experimental procedure described for intermediate 8A in Scheme 8 to provide intermediate 12A. The crude compound was used for the next reaction without further purification. LCMS: m/z=368.2 [M+H]⁺; Ret. Time 3.472 min. (LCMS Method E).

### Intermediate 12B: 6-(3-(6-methylpyridin-2-yl)-1-((2-(trimethylsilyl)ethoxy)methyl)-1H-pyrazol-4-yl)imidazo[1,2-b]pyridazine

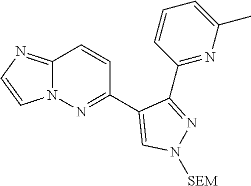 [SMILES: Cc1cccc(-c2nn(COCC[Si](C)(C)C)cc2-c2ccc3nccn3n2)n1]

To a solution of intermediate 12A (0.169 g, 0.459 mmol) in 1,4-dioxane (5 mL) was added hexamethylditin (0.095 mL, 0.459 mmol) and Pd(Ph₃P)₄ (0.027 g, 0.023 mmol). The reaction mixture was degassed for 10 min. and heated in a microwave instrument at 120° C. for 2 h. Then 6-(4,4,5,5-tetramethyl-1,3,2-dioxaborolan-2-yl)imidazo[1,2-b]pyridazine (reference: U.S. Pat. Appl. Publ., 20150038506, 5 Feb. 2015) (0.123 g, 0.505 mmol) and Pd(Ph₃P)₄ (0.027 g, 0.023 mmol) were added and the resultant reaction mixture was degassed once again and heating continued under microwave conditions at 120° C. for 2 h. The reaction was cooled to RT, diluted with ethyl acetate and filtered through Celite®. The filtrate was concentrated under reduced pressure to give the intermediate 12B (0.03 g, 15.0% yield). The crude compound was used for the next reaction without further purification. LCMS: m/z=407.4 [M+H]⁺; Ret. Time 1.39 min. (LCMS Method B).

### Example 19: 6-(3-(6-methylpyridin-2-yl)-1H-pyrazol-4-yl)imidazo[1,2-b]pyridazine

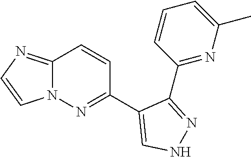 [SMILES: Cc1cccc(-c2n[nH]cc2-c2ccc3nccn3n2)n1]

Example 19 was synthesized by reacting intermediate 12B with TFA and by employing the experimental procedure described for Example 1 in Scheme 1. The crude product was dissolved in DMF and purified via preparative HPLC (Condition N). Fractions containing the desired product were combined and dried under vacuum to afford Example 19. LCMS: m/z=277.2 [M+H]⁺; Ret. Time 1.044 min. (LCMS Method C). ¹H NMR (400 MHz, DMSO-d₆) δ ppm 13.53 (s, 1H), 8.14-8.19 (m, 2H), 7.97-7.99 (m, 1H), 7.72-7.77 (m, 2H), 7.63-7.65 (m, 1H), 7.20-7.26 (m, 2H), 2.31-2.35 (m, 3H).

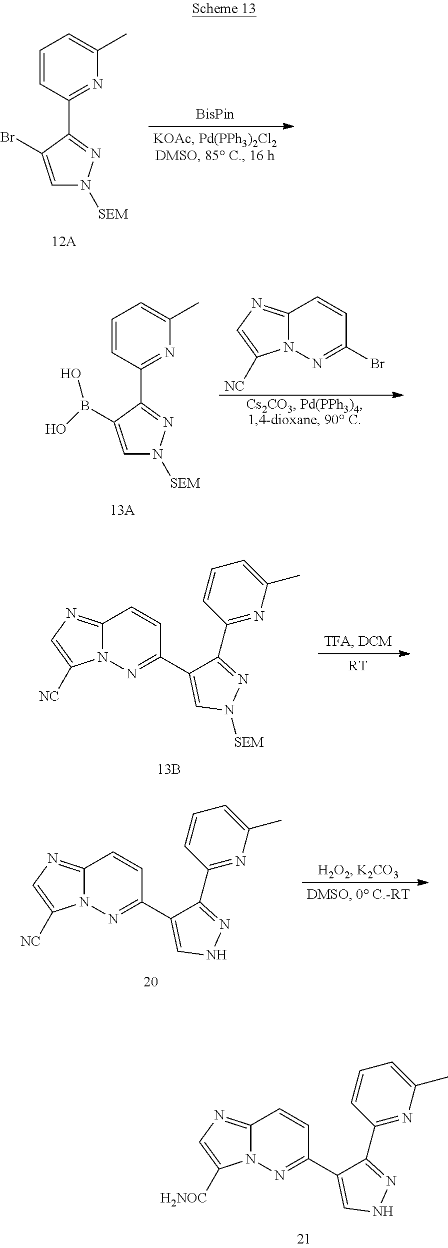

### Intermediate 13A: (3-(6-methylpyridin-2-yl)-1-((2-(trimethylsilyl)ethoxy)methyl)-1H-pyrazol-4-yl)boronic acid

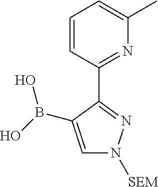 [SMILES: Cc1cccc(-c2nn(COCC[Si](C)(C)C)cc2B(O)O)n1]

Intermediate 13A was synthesized by reacting 2-(4-bromo-1-((2-(trimethylsilyl)ethoxy)methyl)-1H-pyrazol-3-yl)-6-methylpyridine with 4,4,4′,4′,5,5,5′,5′-octamethyl-2,2′-bi(1,3,2-dioxaborolane) and by employing the experimental procedure described for intermediate 8B in Scheme 8. The crude product was purified by silica gel chromatography (24 g RediSep® column, eluting with a gradient of 10-100% EtOAc in petroleum ether). Fractions containing the desired product were combined under reduced pressure and evaporated to afford intermediate 13A. LCMS: m/z=334.5 [M+H]⁺; Ret. Time 1.06 min. (LCMS Method B).

### Intermediate 13B: 6-(3-(6-methylpyridin-2-yl)-1-((2-(trimethylsilyl)ethoxy)methyl)-1H-pyrazol-4-yl)imidazo[1,2-b]pyridazine-3-carbonitrile

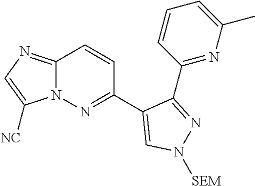 [SMILES: Cc1cccc(-c2nn(COCC[Si](C)(C)C)cc2-c2ccc3ncc(C#N)n3n2)n1]

To a solution of 6-bromoimidazo[1,2-b]pyridazine-3-carbonitrile (0.3 g, 1.345 mmol) in 1,4-dioxane (5 mL) was added intermediate 13A (0.672 g, 2.018 mmol), cesium carbonate (1.315 g, 4.04 mmol), Pd(Ph₃P)₄ (0.078 g, 0.067 mmol) and water (0.10 mL). The reaction mixture was degassed for 10 min. with argon and heated overnight at 90° C. The reaction mixture was diluted with ethyl acetate, filtered through a Celite® pad and the filtrate concentrated under reduced pressure to give the crude compound. The crude product was purified by silica gel chromatography (24 g RediSep® column, eluting with a gradient of 40-100% EtOAc in petroleum ether). Fractions containing the desired product were combined under reduced pressure and evaporated to afford intermediate 13B (0.22 g, 37.9% yield). LCMS: m/z=432.9 [M+H]⁺; Ret. Time 3.28 min. (LCMS Method E).

### Example 20: 6-(3-(6-methylpyridin-2-yl)-1H-pyrazol-4-yl)imidazo[1,2-b]pyridazine-3-carbonitrile

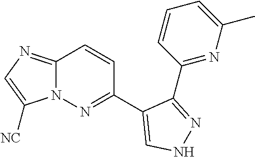 [SMILES: Cc1cccc(-c2n[nH]cc2-c2ccc3ncc(C#N)n3n2)n1]

Example 20 was synthesized by reacting intermediate 12B with TFA and by employing the experimental procedure described for Example 1 in Scheme 1. The crude product was dissolved in DMF and purified via preparative HPLC (Condition N). Fractions containing the desired product were combined and evaporated under reduced pressure to afford Example 20. LCMS: m/z=302.1 [M+H]⁺; Ret. Time 1.253 min. (LCMS Method C). ¹H NMR (400 MHz, DMSO-d₆) δ ppm 13.58 (s, 1H), 8.52 (s, 1H), 8.34 (br s, 1H), 8.25-8.28 (m, 1H), 7.66-7.85 (m, 3H), 7.24-7.32 (m, 1H), 2.29-2.43 (m, 3H).

### Example 21: 6-(3-(6-methylpyridin-2-yl)-1H-pyrazol-4-yl)imidazo[1,2-b]pyridazine-3-carboxamide

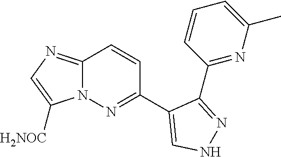 [SMILES: Cc1cccc(-c2n[nH]cc2-c2ccc3ncc(C(N)=O)n3n2)n1]

Example 21 was synthesized by reacting Example 20 with H₂O₂/K₂CO₃ and by employing the experimental procedure described for Example 2 in Scheme 1. The crude product was dissolved in DMF and purified via preparative HPLC (Condition N). Fractions containing the desired product were combined and evaporated under reduced pressure to afford Example 21. LCMS: m/z=320.1 [M+H]⁺; Ret. Time 0.817 min. (LCMS Method C). ¹H NMR (400 MHz, DMSO-d₆) δ ppm 13.6 (br s, 1H), 8.41 (br s, 1H), 8.21 (s, 2H), 7.71-7.84 (m, 1H), 7.46-7.68 (m, 4H), 7.15-7.33 (m, 1H), 2.25-2.42 (m, 3H).

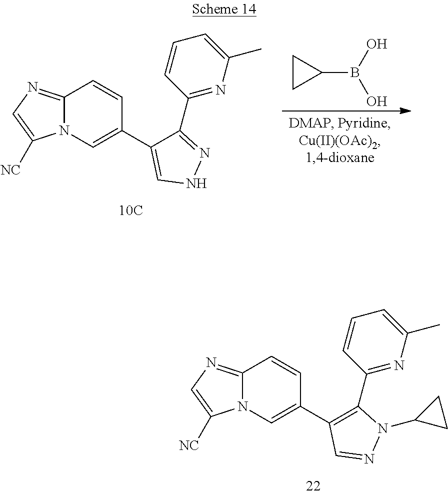

### Example 22: 6-(1-cyclopropyl-5-(6-methylpyridin-2-yl)-1H-pyrazol-4-yl)imidazo[1,2-a]pyridine-3-carbonitrile

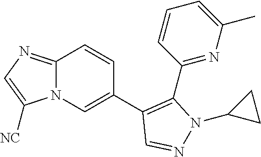 [SMILES: Cc1cccc(-c2c(-c3ccc4ncc(C#N)n4c3)cnn2C2CC2)n1]

To a solution of intermediate 10C (0.1 g, 0.333 mmol) in 1,4-dioxane (5 mL) at RT was added cyclopropylboronic acid (0.061 g, 0.706 mmol), DMAP (0.122 g, 0.999 mmol), copper(II) acetate (0.060 g, 0.333 mmol) and pyridine (0.027 mL, 0.333 mmol). The reaction mixture was heated overnight at 90° C. The reaction mixture was then diluted with ethyl acetate, filtered through Celite® pad and the filtrate concentrated under reduced pressure to give the crude compound. The crude product was dissolved in DMF and purified via preparative HPLC (Condition N). Fractions containing the desired product were combined and evaporated under reduced pressure to afford Example 22 (13 mg, 11.47% yield). LCMS: m/z=341.1 [M+H]⁺; Ret. Time 1.645 min. (LCMS Method C). ¹H NMR (400 MHz, DMSO-d₆) δ ppm 8.57 (d, J=1.4 Hz, 1H), 8.41 (br, s, 1H), 7.97 (s, 1H), 7.73-7.87 (m, 2H), 7.48 (dd, J=1.6, 9.2 Hz, 1H), 7.41 (dd, J=7.7, 12.6 Hz, 2H), 3.75 (td, J=3.6, 7.4 Hz, 1H), 2.61 (s, 3H), 0.97-1.04 (m, 2H), 0.82-0.90 (m, 2H).

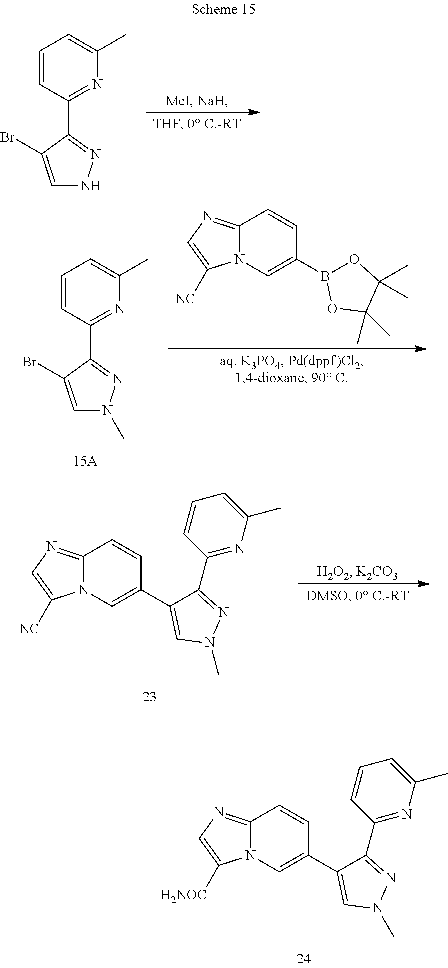

### Intermediate 15A: 2-(4-bromo-1-methyl-1H-pyrazol-3-yl)-6-methylpyridine

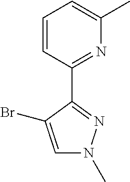

Intermediate 15A was synthesized by reacting 2-(4-bromo-1H-pyrazol-3-yl)-6-methylpyridine with methyl iodide and by employing the experimental procedure described for Example 6B in Scheme 6. The crude product was purified by silica gel chromatography (24 g RediSep® column, eluting with a gradient of 10-100% EtOAc in petroleum ether). Fractions containing the desired product were combined and evaporated under reduced pressure to afford intermediate 15A. LCMS: m/z=254.4 [M+H]⁺; Ret. Time 1.77 min. (LCMS Method E).

### Example 23: 6-(1-methyl-3-(6-methylpyridin-2-yl)-1H-pyrazol-4-yl)imidazo[1,2-a]pyridine-3-carbonitrile

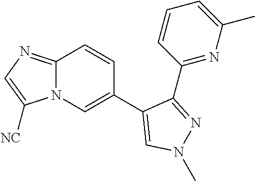 [SMILES: Cc1cccc(-c2nn(C)cc2-c2ccc3ncc(C#N)n3c2)n1]

Example 23 was synthesized by reacting intermediate 15A with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was dissolved in DMF and purified via preparative HPLC (Condition K). Fractions containing the desired product were combined and evaporated under reduced pressure to afford Example 23. LCMS: m/z=315.1 [M+H]⁺; Ret. Time 1.347 min. (LCMS Method C). ¹HNMR (400 MHz, DMSO-d₆) δ ppm 9.00 (t, J=1.3 Hz, 1H), 8.43 (s, 1H), 8.25 (s, 1H), 7.81 (dd, J=9.3, 1.0 Hz, 1H), 7.76-7.71 (m, 1H), 7.71-7.68 (m, 1H), 7.67-7.64 (m, 1H), 7.22-7.19 (m, 1H), 3.97 (s, 3H), 2.42 (s, 3H)

### Example 24: 6-(1-methyl-3-(6-methylpyridin-2-yl)-1H-pyrazol-4-yl)imidazo[1,2-a]pyridine-3-carboxamide

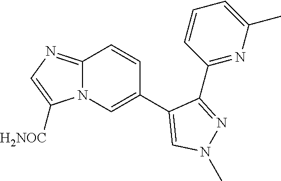 [SMILES: Cc1cccc(-c2nn(C)cc2-c2ccc3ncc(C(N)=O)n3c2)n1]

Example 24 was synthesized by reacting Example 23 with H₂O₂/K₂CO₃ and by employing the experimental procedure described for Example 2 in Scheme 1. The crude product was dissolved in DMF and purified via preparative HPLC (Condition F). Fractions containing the desired product were combined and evaporated under reduced pressure to afford Example 24. LCMS: m/z=333.2 [M+H]⁺; Ret. Time 0.945 min. (LCMS Method C). ¹H NMR (400 MHz, DMSO-d₆) δ ppm 9.69-9.78 (m, 1H), 8.53 (s, 1H), 8.20 (s, 1H), 7.74-7.92 (m, 4H), 7.65 (d, J=7.6 Hz, 2H), 7.23-7.32 (m, 1H), 3.89-4.07 (m, 3H), 2.42 (s, 3H).

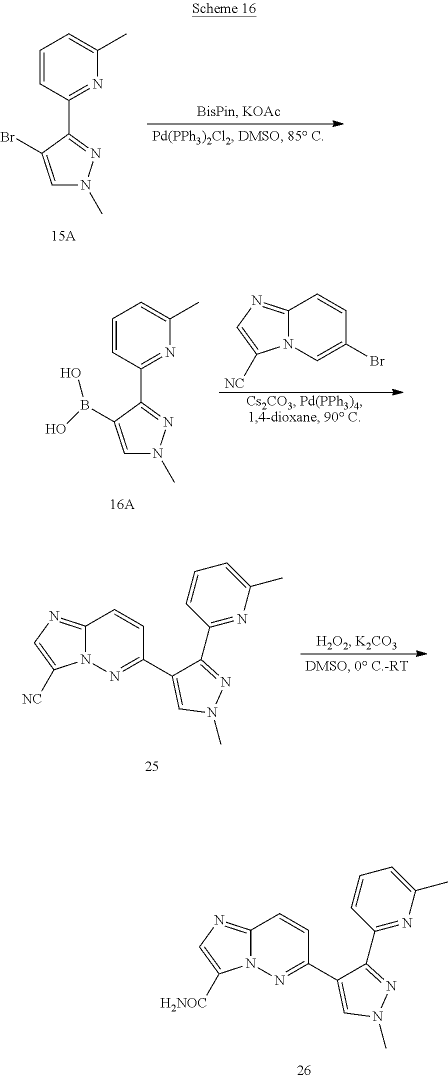

### Intermediate 16A: (1-methyl-3-(6-methylpyridin-2-yl)-1H-pyrazol-4-yl)boronic acid

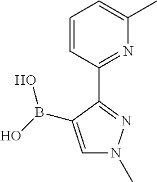 [SMILES: Cc1cccc(-c2nn(C)cc2B(O)O)n1]

Intermediate 16A was synthesized by reacting intermediate 15A with BisPin and by employing the experimental procedure described for intermediate 8B in Scheme 8 to afford intermediate 16A. LCMS: m/z=334.2 [M+H]⁺; Ret. Time 2.868 min. (LCMS Method E).

### Example 25: 6-(1-methyl-3-(6-methylpyridin-2-yl)-1H-pyrazol-4-yl)imidazo[1,2-b]pyridazine-3-carbonitrile

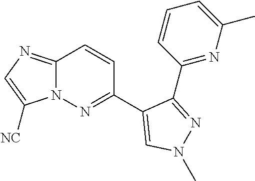 [SMILES: Cc1cccc(-c2nn(C)cc2-c2ccc3ncc(C#N)n3n2)n1]

Example 25 was synthesized by reacting intermediate 16A with 6-bromoimidazo[1,2-b]pyridazine-3-carbonitrile and by employing the experimental procedure described for intermediate 13B in Scheme 13. The crude product was dissolved in DMF and purified via preparative HPLC (Condition N). Fractions containing the desired product were combined and evaporated under reduced pressure to afford Example 25. LCMS: m/z=316.2 [M+H]⁺; Ret. Time 1.35 min. (LCMS Method C). ¹H NMR (400 MHz, DMSO-d₆) δ ppm 8.53 (s, 1H), 8.31 (s, 1H), 8.23 (d, J=9.6 Hz, 1H), 7.75 (d, J 7.6 Hz, 1H), 7.67-7.71 (m, 1H), 7.64 (d, J=9.6 Hz, 1H), 7.20 (d, J=7.4 Hz, 1H), 4.01 (s, 3H), 2.27 (s, 3H).

### Example 26: 6-(1-methyl-3-(6-methylpyridin-2-yl)-1H-pyrazol-4-yl)imidazo[1,2-b]pyridazine-3-carboxamide

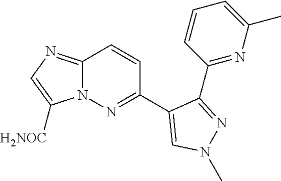 [SMILES: Cc1cccc(-c2nn(C)cc2-c2ccc3ncc(C(N)=O)n3n2)n1]

Example 26 was synthesized by reacting Example 25 with H₂O₂/K₂CO₃ and by employing the experimental procedure described for Example 2 in Scheme 1. The crude product was dissolved in DMF and purified via preparative HPLC (Condition N). Fractions containing the desired product were combined and evaporated under reduced pressure to afford Example 26. LCMS: m/z=334.1 [M+H]⁺; Ret. Time 1.137 min. (LCMS Method C). ¹H NMR (400 MHz, DMSO-d₆) δ ppm 8.47 (s, 1H), 8.15-8.29 (m, 2H), 7.69-7.81 (m, 2H), 7.63 (d, J=7.8 Hz, 2H), 7.54 (s, 1H), 7.22 (d, J=7.2 Hz, 1H), 4.01 (s, 3H), 2.29 (s, 3H).

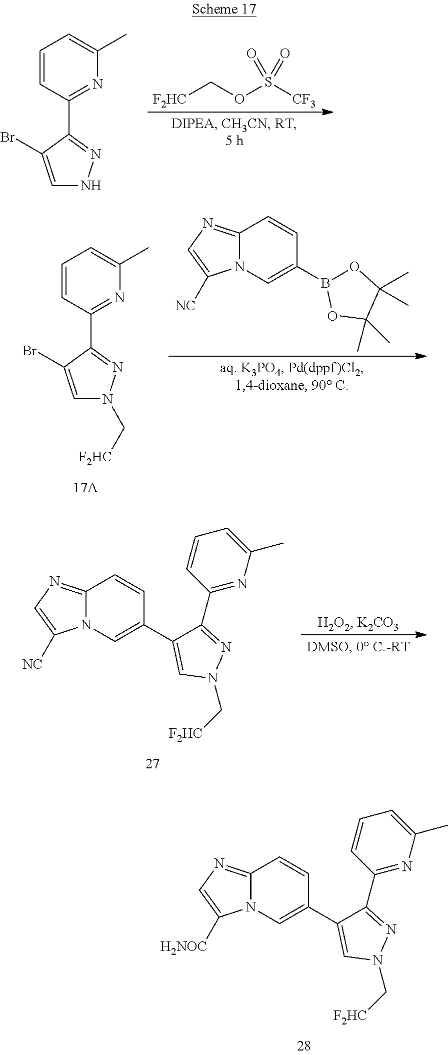

### Intermediate 17A: 2-(4-bromo-1-(2,2-difluoroethyl)-1H-pyrazol-3-yl)-6-methylpyridine

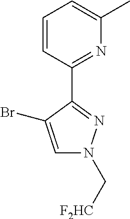 [SMILES: Cc1cccc(-c2nn(CC(F)F)cc2Br)n1]

Intermediate 17A was synthesized by reacting 2-(4-bromo-1H-pyrazol-3-yl)-6-methylpyridine with 2,2-difluoroethyl trifluoromethanesulfonate and by employing the experimental procedure described for Example 5B in Scheme 5. The crude product was purified by silica gel chromatography (24 g RediSep® column, eluting with a gradient of 30-100% EtOAc in petroleum ether). Fractions containing the desired product were combined and evaporated under reduced pressure to afford intermediate 17A. LCMS: m/z=304.0 [M+H]⁺; Ret. Time 2.013 min. (HPLC condition E).

### Example 27: 6-(1-(2,2-difluoroethyl)-3-(6-methylpyridin-2-yl)-1H-pyrazol-4-yl)imidazo[1,2-a]pyridine-3-carbonitrile

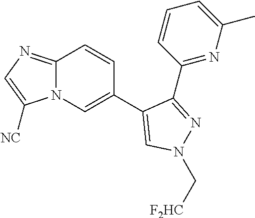 [SMILES: Cc1cccc(-c2nn(CC(F)F)cc2-c2ccc3ncc(C#N)n3c2)n1]

Example 27 was synthesized by reacting Intermediate 17A with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was dissolved in DMF and purified via preparative HPLC (Condition J). Fractions containing the desired product were combined and evaporated under reduced pressure to afford Example 27. LCMS: m/z=365.1 [M+H]⁺; Ret. Time 1.608 min. (LCMS Method C). ¹H NMR (400 MHz, DMSO-d₆) δ ppm 8.98 (s, 1H), 8.44 (s, 1H), 8.35 (s, 1H), 7.72-7.86 (m, 2H), 7.61-7.71 (m, 2H), 7.24 (d, J=7.5 Hz, 1H), 6.31-6.71 (m, 1H), 4.77 (td, J=15.1, 3.52 Hz, 2H), 2.42 (s, 3H).

### Example 28: 6-(1-(2,2-difluoroethyl)-3-(6-methylpyridin-2-yl)-1H-pyrazol-4-yl)imidazo[1,2-a]pyridine-3-carboxamide

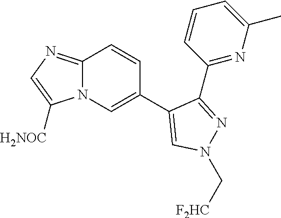 [SMILES: Cc1cccc(-c2nn(CC(F)F)cc2-c2ccc3ncc(C(N)=O)n3c2)n1]

Example 28 was synthesized by reacting Example 27 with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 28. LCMS: m/z=383.1 [M+H]⁺; Ret. Time 1.232 min. (LCMS Method C). ¹H NMR (400 MHz, DMSO-d₆) δ ppm 9.63 (dd, J=1.74, 0.95 Hz, 1H), 8.33 (s, 1H), 8.19 (s, 1H), 7.90 (br s, 1H) 7.72 (s, 1H), 7.65 (dd, J=9.3, 0.89 Hz, 1H), 7.60 (d, J 7.7 Hz, 1H), 7.51 (d, J=1.8 Hz, 1H), 7.36 (br s, 1H) 7.18 (d, J=7.6 Hz, 1H), 6.50 (s, 1H), 4.76 (td, J=15.1, 3.73 Hz, 2H), 2.30 (s, 3H).

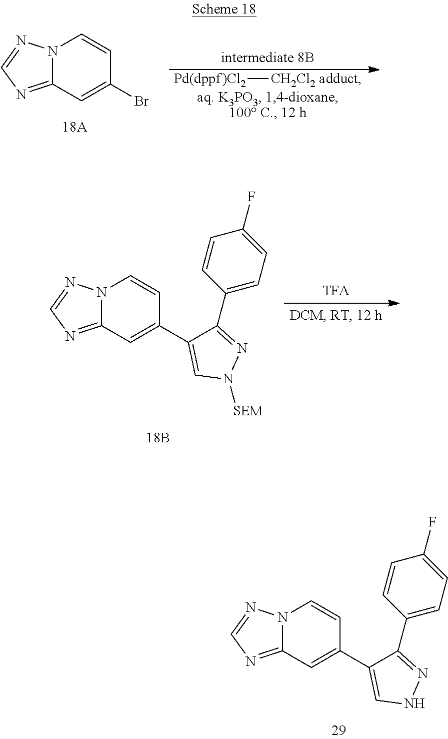

### Intermediate 18B: 7-(3-(4-fluorophenyl)-1-((2-(trimethylsilyl)ethoxy)methyl)-1H-pyrazol-4-yl)-[1,2,4]triazolo[1,5-a]pyridine

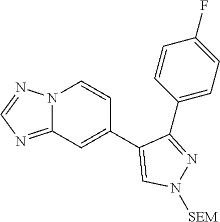 [SMILES: C[Si](C)(C)CCOCn1cc(-c2ccn3ncnc3c2)c(-c2ccc(F)cc2)n1]

Intermediate 18B was synthesized by reacting intermediate 8B with 7-bromo-[1,2,4]triazolo[1,5-a]pyridine (Source: ArkPharma, CAS-1053655-66-5) and by employing the experimental procedure described for Intermediate 1E in Scheme 1 to afford 18B as a gum. LCMS: m/z 410.4 [M+1]⁺; HPLC Ret. Time 1.09 (HPLC Method B).

### Example 29: 7-(3-(4-fluorophenyl)-1H-pyrazol-4-yl)-[1,2,4]triazolo[1,5-a]pyridine

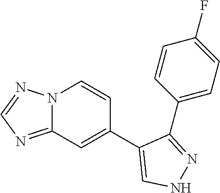 [SMILES: Fc1ccc(-c2n[nH]cc2-c2ccn3ncnc3c2)cc1]

Example 29 was synthesized by reacting intermediate 18A with TFA and by employing the experimental procedure described for Example 1 in Scheme 1. The crude product was dissolved in DMF and purified via preparative HPLC (Condition L). Fractions containing the desired product were combined and dried under vacuum to afford Example 29. LCMS: m/z 280.1 [M+1]⁺; HPLC Ret. Time 1.300 (HPLC Method C). ¹H NMR (400 MHz, DMSO-d₆) δ ppm 13.48-13.23 (m, 1H), 8.90-8.79 (m, 1H), 8.47-8.35 (m, 1H), 8.33-8.24 (m, 1H), 7.73-7.59 (m, 1H), 7.54-7.42 (m, 2H), 7.38-7.12 (m, 2H), 7.05-6.86 (m, 1H).

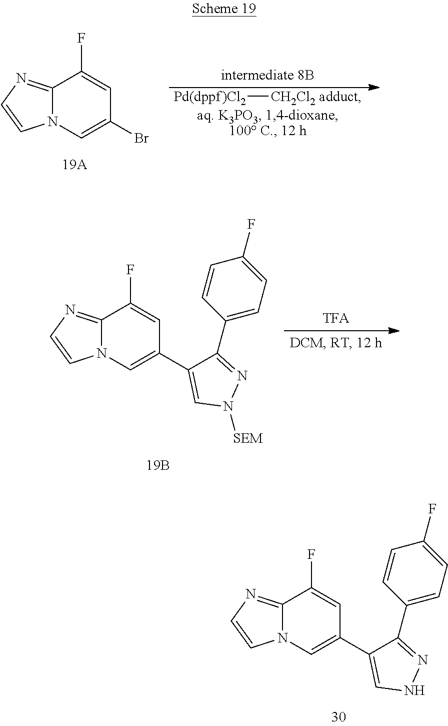

### Intermediate 19B: 8-fluoro-6-(3-(4-fluorophenyl)-1-((2-(trimethylsilyl)ethoxy)methyl)-1H-pyrazol-4-yl)imidazo[1,2-a]pyridine

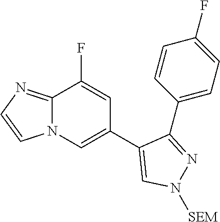 [SMILES: C[Si](C)(C)CCOCn1cc(-c2cc(F)c3nccn3c2)c(-c2ccc(F)cc2)n1]

Intermediate 19B was synthesized by reacting intermediate 8B with 6-bromo-8-fluoroimidazo[1,2-a]pyridine (Source: Arkharmi, CAS-474709-06-3) and by employing the experimental procedure described for intermediate 1E in Scheme 1 to afford 19B as a gum. LCMS: m/z 427.4 [M+1]⁺; HPLC Ret. Time 1.13 (HPLC Method B).

### Example 30: 8-fluoro-6-(3-(4-fluorophenyl)-1H-pyrazol-4-yl)imidazo[1,2-a]pyridine

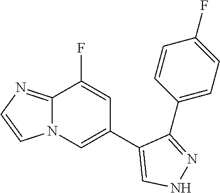 [SMILES: Fc1ccc(-c2n[nH]cc2-c2cc(F)c3nccn3c2)cc1]

Example 30 was synthesized by reacting Intermediate 19B with TFA and by employing the experimental procedure described for Example 1 in Scheme 1. The crude product was dissolved in DMF and purified via preparative HPLC (Condition N). Fractions containing the desired product were combined and dried under vacuum to afford Example 30. LCMS: m/z 297.1 [M+1]⁺; HPLC Ret. Time 1.287 (HPLC Method C). ¹H NMR (400 MHz, DMSO-d₆) δ ppm 13.42-13.11 (m, 1H), 8.34 (bs, 1H), 8.07 (bs, 1H), 7.78 (bs, 1H), 7.61 (s, 1H), 7.51 (dd, J=8.6, 5.6 Hz, 2H), 7.29 (bs, 2H), 6.96 (s, 1H).

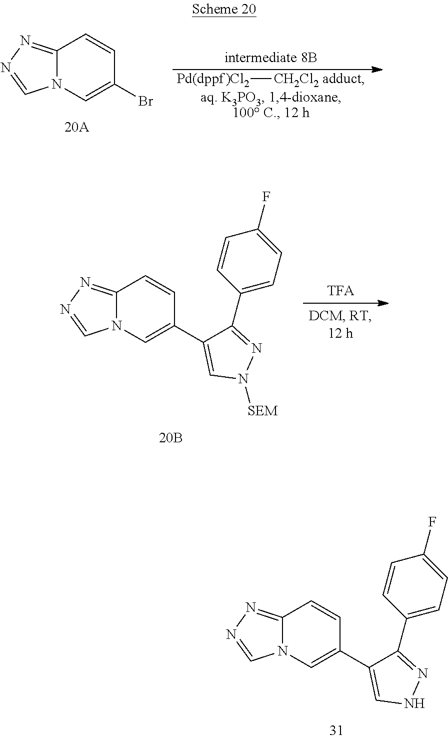

### Intermediate 20B: 6-(3-(4-fluorophenyl)-1-((2-(trimethylsilyl)ethoxy)methyl)-1H-pyrazol-4-yl)-[1,2,4]triazolo[4,3-a]pyridine

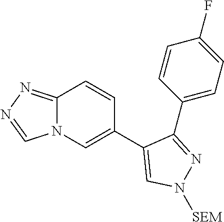 [SMILES: C[Si](C)(C)CCOCn1cc(-c2ccc3nncn3c2)c(-c2ccc(F)cc2)n1]

Intermediate 20B was synthesized by reacting 6-bromo-[1,2,4]triazolo[4,3-a]pyridine (Source: Aldrich, CAS-356560-80-0 with 8B and by employing the experimental procedure described for intermediate 1E in Scheme 1 to afford 20B as a gum. LCMS: m/z 410.4 [M+1]⁺; HPLC Ret. Time 0.97 (HPLC Method B).

### Example 31: 6-(3-(4-fluorophenyl)-1H-pyrazol-4-yl)-[1,2,4]triazolo[4,3-a]pyridine, TFA

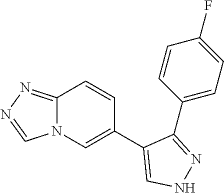 [SMILES: Fc1ccc(-c2n[nH]cc2-c2ccc3nncn3c2)cc1]

Example 31 was synthesized by reacting intermediate 20B with TFA and by employing the experimental procedure described for Example 1 in Scheme 1. The crude product was dissolved in DMF and purified via preparative HPLC (Condition L). Fractions containing the desired product were combined and dried under vacuum to afford Example 31. TFA. LCMS: m/z 280.1 [M+1]⁺; HPLC Ret. Time 1.106 (HPLC Method C). ¹H NMR (400 MHz, DMSO-d₆) δ ppm 13.47-13.09 (m, 1H), 9.25-9.09 (m, 1H), 8.55-8.40 (m, 1H), 8.12-7.87 (m, 1H), 7.77-7.62 (m, 1H), 7.54-7.39 (m, 2H), 7.17-6.80 (m, 3H).

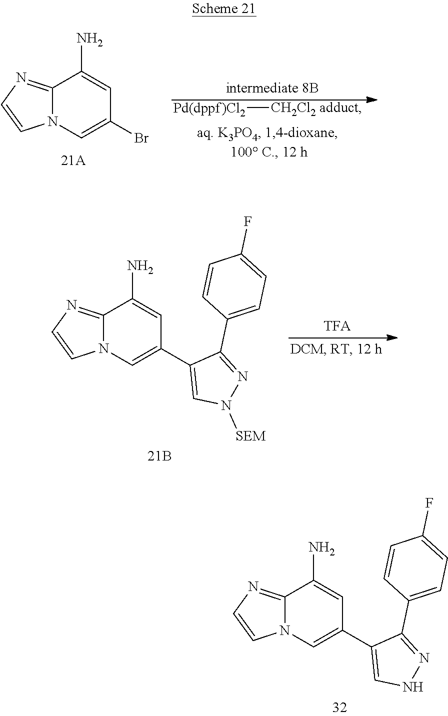

### Intermediate 21B: 6-(3-(4-fluorophenyl)-1-((2-(trimethylsilyl)ethoxy)methyl)-1H-pyrazol-4-yl)imidazo[1,2-a]pyridin-8-amine

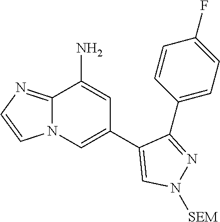 [SMILES: C[Si](C)(C)CCOCn1cc(-c2cc(N)c3nccn3c2)c(-c2ccc(F)cc2)n1]

Intermediate 21B was synthesized by reacting Intermediate 8B with 6-bromoimidazo[1,2-a]pyridin-8-amine (Source: Arkharmi 376371-00-9) and by employing the experimental procedure described for intermediate 1E in Scheme 1 to obtain 21B as a gum. LCMS: m/z 424.4 [M+1]⁺; HPLC Ret. Time 1.07 (HPLC Method B).

### Example 32: 6-(3-(4-fluorophenyl)-1H-pyrazol-4-yl)imidazo[1,2-a]pyridin-8-amine

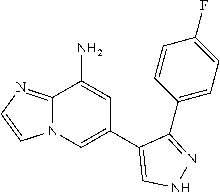 [SMILES: Nc1cc(-c2c[nH]nc2-c2ccc(F)cc2)cn2ccnc12]

Example 32 was synthesized by reacting intermediate 21B with TFA and by employing the experimental procedure described for Example 1 in Scheme 1. The crude product was dissolved in DMF and purified via preparative HPLC (Condition L). Fractions containing the desired product were combined and dried under vacuum to afford Example 32. LCMS: m/z 294.1 [M+1]⁺; HPLC Ret. Time 1.185 (HPLC Method C). ¹H NMR (400 MHz, DMSO-d₆) δ ppm 8.01 (dd, J=15.0, 1.3 Hz, 3H), 7.79 (s, 2H), 7.66 (d, J=1.2 Hz, 1H), 7.47 (s, 2H), 6.33 (d, J=1.5 Hz, 1H), 5.85 (s, 2H).

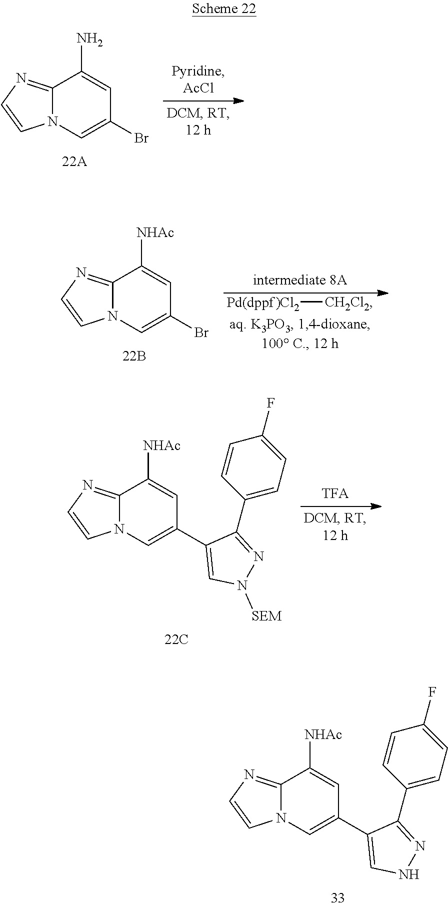

### Intermediate 22B: N-(6-bromoimidazo[1,2-a]pyridin-8-yl)acetamide

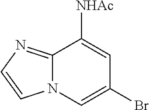 [SMILES: CC(=O)Nc1cc(Br)cn2ccnc12]

To a solution of 22A (0.3 g, 1.415 mmol) in dry DCM (8 mL) at 0° C. was added pyridine (0.172 mL, 2.122 mmol), followed by a dropwise addition of AcCl (0.121 mL, 1.698 mmol). The resulting mixture was stirred at RT for 12 h. The reaction mixture was diluted with DCM (50 mL) and washed with saturated aq. NaHCO₃ solution, dried over anhydrous Na₂SO₄, filtered and the filtrate evaporated under reduced pressure to get the crude residue. The crude product was purified by silica gel chromatography (40 g RediSep® column, eluting with a gradient of 30-50% EtOAc in petroleum ether). Fractions containing the desired product were combined and evaporated under reduced pressure to afford intermediate 22B (0.15 g, 41.7% yield). LCMS: m/z 256.0 [M+2]⁺; HPLC Ret. Time 1.423 (HPLC Method E).

### Intermediate 22C: N-(6-(3-(4-fluorophenyl)-1-((2-(trimethylsilyl)ethoxy)methyl)-1H-pyrazol-4-yl)imidazo[1,2-a]pyridin-8-yl)acetamide

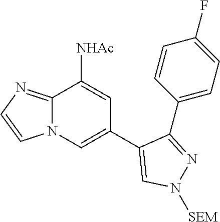 [SMILES: CC(=O)Nc1cc(-c2cn(COCC[Si](C)(C)C)nc2-c2ccc(F)cc2)cn2ccnc12]

Intermediate 22C was synthesized by reacting intermediate 8B with intermediate 22C and by employing the experimental procedure described for intermediate 1E in Scheme 1 to afford intermediate 22C. LCMS: m/z 466.4 [M+2]⁺; HPLC Ret. Time 1.09 (HPLC Method B).

### Example 33: N-(6-(3-(4-fluorophenyl)-1H-pyrazol-4-yl)imidazo[1,2-a]pyridin-8-yl)acetamide

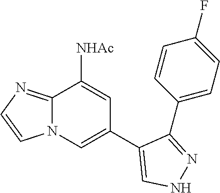 [SMILES: CC(=O)Nc1cc(-c2c[nH]nc2-c2ccc(F)cc2)cn2ccnc12]

Example 33 was synthesized by reacting intermediate 22C with TFA and by employing the experimental procedure described for Example 1 in Scheme 1. The crude product was dissolved in DMF and purified via preparative HPLC (Condition O). Fractions containing the desired product were combined and dried under vacuum to afford Example 33. LCMS: m/z 336.1 [M+1]⁺; HPLC Ret. Time 1.226 (HPLC Method C). ¹H NMR (400 MHz, DMSO-d₆) δ ppm 13.47 (bs, 1H), 10.21 (bs, 1H), 8.46 (d, J=1.5 Hz, 1H), 8.21-8.05 (m, 3H), 8.00-7.85 (m, 1H), 7.81-7.67 (m, 3H), 7.46 (t, J=8.8 Hz, 1H), 2.16 (s, 3H).

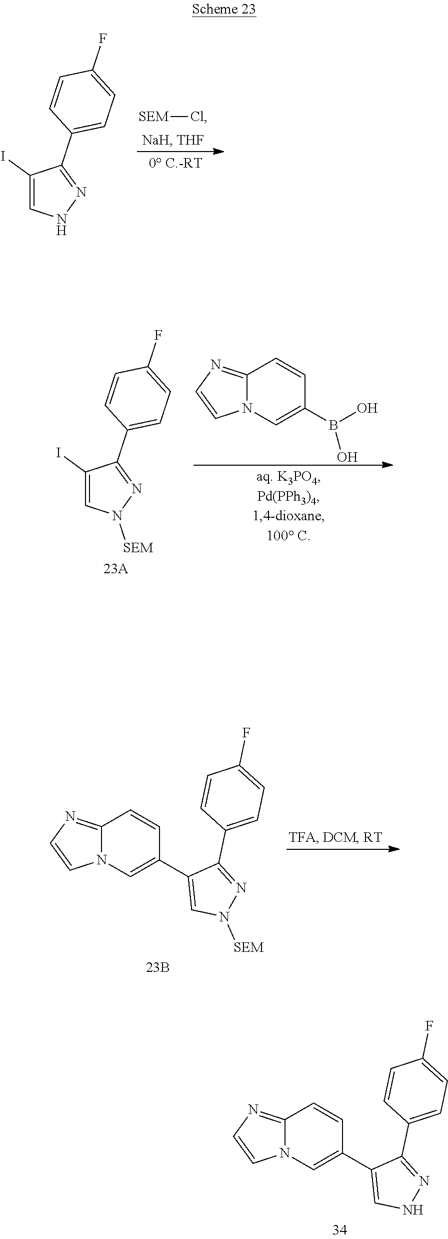

### Intermediate 23A: 3-(4-fluorophenyl)-4-iodo-1-((2-(trimethylsilyl)ethoxy)methyl)-1H-pyrazole

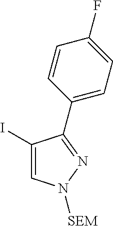 [SMILES: C[Si](C)(C)CCOCn1cc(I)c(-c2ccc(F)cc2)n1]

Intermediate 23A was synthesized by reacting intermediate 3-(4-fluorophenyl)-4-iodo-1H-pyrazole with SEM-Cl and by employing the experimental procedure described for intermediate 8A in Scheme 8 to afford intermediate 23A. MS (ES): m/z=419.3 [M+H]⁺; HPLC Ret. Time 1.79 min. (HPLC Method B).

### Intermediate 23B: 6-(3-(4-fluorophenyl)-1-((2-(trimethylsilyl)ethoxy)methyl)-1H-pyrazol-4-yl)imidazo[1,2-a]pyridine

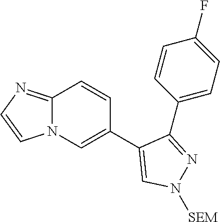 [SMILES: C[Si](C)(C)CCOCn1cc(-c2ccc3nccn3c2)c(-c2ccc(F)cc2)n1]

To a solution of intermediate 23A (100 mg, 0.239 mmol), imidazo[1,2-a]pyridin-6-ylboronic acid (58.1 mg, 0.359 mmol) in 1,4-dioxane was added 2M aq. solution of tripotassium phosphate (0.359 mL, 0.717 mmol). The solution was purged with argon for 2 min and Pd(Ph₃P)₄ (27.6 mg, 0.024 mmol) was added. The reaction mixture was heated at 100° C. for 16 h, cooled to RT and concentrated in vacuo. The residue was purified by silica gel chromatography (12 g RediSep® column, eluting with a gradient from 0-55% EtOAc in petroleum ether). Fractions containing the product were combined and evaporated under reduced pressure to afford intermediate 23B. MS (ES): m/z=409.5 [M+H]⁺; HPLC Ret. Time 1.45 min. (HPLC Method B).

### Example 34: 6-(3-(4-fluorophenyl)-1H-pyrazol-4-yl)imidazo[1,2-a]pyridine

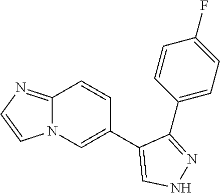 [SMILES: Fc1ccc(-c2n[nH]cc2-c2ccc3nccn3c2)cc1]

Example 34 was synthesized by reacting intermediate 23B with TFA and by employing the experimental procedure described for Example 1 in Scheme 1. The crude product was dissolved in a mixture of DMF and methanol and purified via preparative HPLC (Method F). Fractions containing the desired product were combined and dried under vacuum to afford Example 34. MS (ES): m/z=279.1 [M+H]⁺; HPLC Ret. Time 1.319 min. (HPLC Methods); ¹H NMR (400 MHz, DMSO-d₆) δ ppm 13.25 (br. s., 1H), 8.51 (s, 1H), 7.91 (s, 2H), 7.57 (d, J=1.0 Hz, 1H), 7.51 (dt, J=9.2, 6.0 Hz, 3H), 7.23 (t, J=8.8 Hz, 2H), 7.01 (dd, J=9.3, 1.8 Hz, 1H).

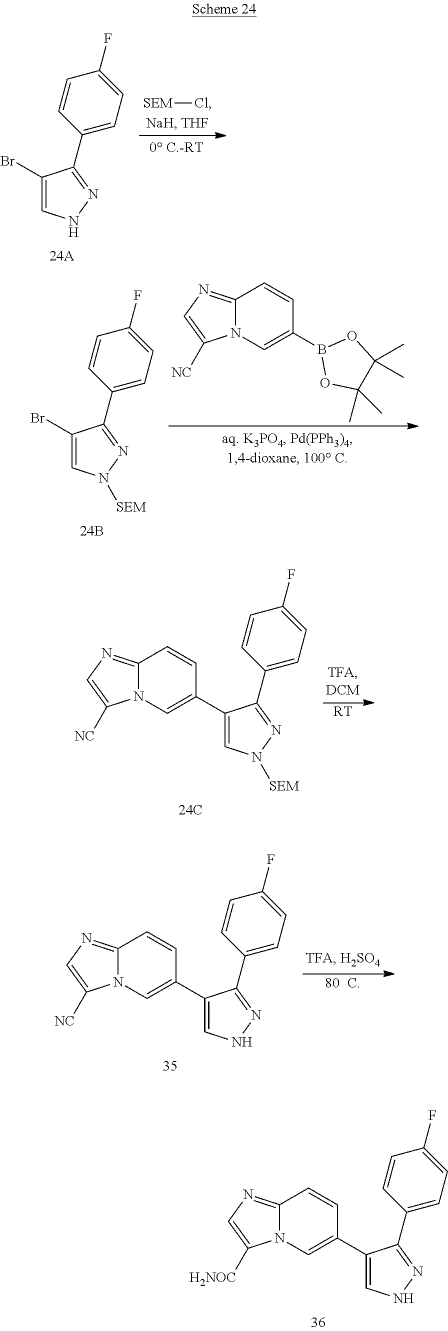

### Intermediate 24B: 4-bromo-3-(4-fluorophenyl)-1-((2-(trimethylsilyl)ethoxy)methyl)-1H-pyrazole

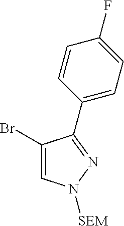 [SMILES: C[Si](C)(C)CCOCn1cc(Br)c(-c2ccc(F)cc2)n1]

Intermediate 24B was synthesized by reacting intermediate 24A (reference: WO2014/100 540 A1) with SEM-Cl and by employing the experimental procedure described for intermediate 8A in Scheme to afford intermediate 24B. MS (ES): m/z=373.1 [M+H]⁺; HPLC Ret. Time 1.79 min. (HPLC Method B).

### Intermediate 24C: 6-(3-(4-fluorophenyl)-1-((2-(trimethylsilyl)ethoxy)methyl)-1H-pyrazol-4-yl)imidazo[1,2-a]pyridine-3-carbonitrile

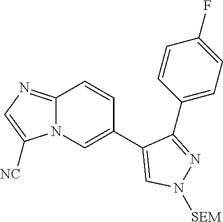 [SMILES: C[Si](C)(C)CCOCn1cc(-c2ccc3ncc(C#N)n3c2)c(-c2ccc(F)cc2)n1]

Intermediate 24C was synthesized by reacting intermediate 24B with 6-(4,4,5,5-tetramethyl-1,3,2-dioxaborolan-2-yl)imidazo[1,2-a]pyridine-3-carbonitrile and by employing the experimental procedure described for intermediate 23B in Scheme 23. The crude product was purified by silica gel chromatography (24 g RediSep® column, eluting with a gradient of 40-60% EtOAc in petroleum ether). Fractions containing the desired product were combined and evaporated to afford intermediate 24C. MS (ES): m/z=434.5 [M+H]⁺; HPLC Ret. Time 1.54 min. (HPLC Method B).

### Example 35: 6-(3-(4-fluorophenyl)-1H-pyrazol-4-yl)imidazo[1,2-a]pyridine-3-carbonitrile

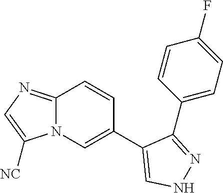 [SMILES: N#Cc1cnc2ccc(-c3c[nH]nc3-c3ccc(F)cc3)cn12]

Example 35 was synthesized by reacting intermediate 24C with TFA and by employing the experimental procedure described for Example 1 in Scheme 1. The crude product was purified via preparative HPLC (Method F). Fractions containing the product were combined and evaporated to afford Example 35. MS (ES): m/z=304.1 [M+H]⁺; HPLC Ret. Time 1.372 min. and 1.348 min. (HPLC Methods C and D, respectively); ¹H NMR (400 MHz, DMSO-d₆) δ ppm 8.47 (s, 1H), 8.43 (s, 1H), 8.13 (br. s., 1H), 7.79 (dd, J=9.3, 0.7 Hz, 1H), 7.56-7.45 (m, 2H), 7.36 (dd, J=9.3, 1.7 Hz, 1H), 7.23 (t, J=8.8 Hz, 2H).

### Example 36: 6-(3-(4-fluorophenyl)-1H-pyrazol-4-yl)imidazo[1,2-a]pyridine-3-carboxamide

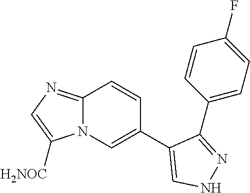 [SMILES: NC(=O)c1cnc2ccc(-c3c[nH]nc3-c3ccc(F)cc3)cn12]

To a solution of Example 35 (40 mg, 0.132 mmol)) in TFA (0.1 mL) was added H₂SO₄ (0.03 mL). The reaction mixture was heated at 80° C. for 2 h, cooled to RT and concentrated in vacuo. The residue was diluted with DCM, washed with 10% aq. NaHCO₃ solution, water, brine, dried over Na₂SO₄, filtered and the filtrate evaporated. The crude product was dissolved in a mixture of DMF and methanol and purified via preparative HPLC (Method F). Fractions containing the desired product were combined and dried under vacuum to afford Example 36 (19.7 mg, 46.5% yield). MS (ES): m/z=322.1 [M+H]⁺; HPLC Ret. Time 1.116 and 0.857 min. (HPLC Methods C and D, respectively); ¹H NMR (400 MHz, DMSO-d₆) δ ppm 13.29 (br. s., 1H), 9.49-9.41 (m, 1H), 8.36-8.27 (m, 1H), 7.92 (br. s., 2H), 7.72-7.61 (m, 1H), 7.53-7.42 (m, 2H), 7.39-7.13 (m, 4H).

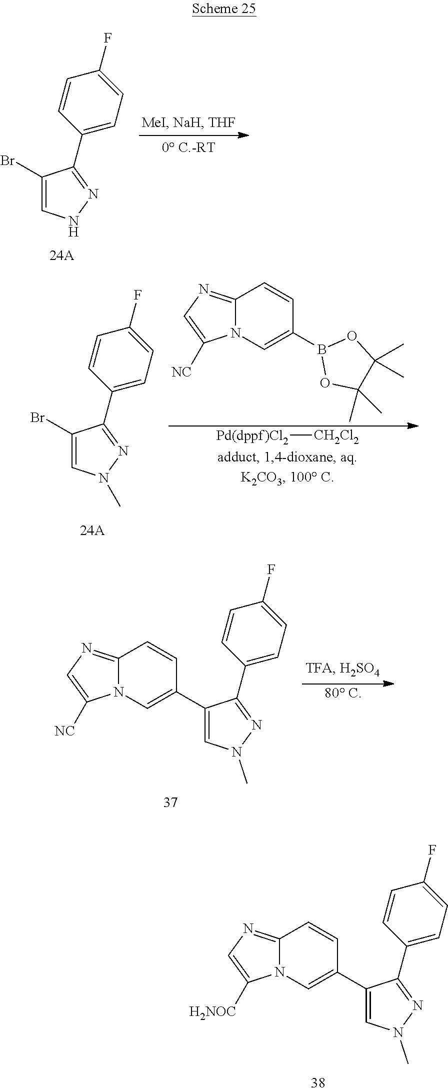

### Intermediate 25A: 4-bromo-3-(4-fluorophenyl)-1-methyl-1H-pyrazole

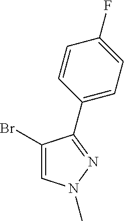

Intermediate 25A was synthesized by reacting intermediate 24A with SEM-Cl and by employing the experimental procedure described for intermediate 8A in Scheme 8. The crude product was purified using preparative HPLC (Method L) to afford the desired isomer as intermediate 25A. MS (ES): m/z=257.0 [M+2H]⁺; HPLC Ret. Time 0.82 min. (HPLC Method B).

### Example 37: 6-(3-(4-fluorophenyl)-1-methyl-1H-pyrazol-4-yl)imidazo[1,2-a]pyridine-3-carbonitrile

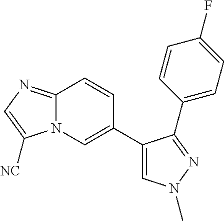 [SMILES: Cn1cc(-c2ccc3ncc(C#N)n3c2)c(-c2ccc(F)cc2)n1]

To a solution of intermediate 25A (150 mg, 0.588 mmol) in 1,4-dioxane (2 mL) was added 6-(4,4,5,5-tetramethyl-1,3,2-dioxaborolan-2-yl)imidazo[1,2-a]pyridine-3-carbonitrile (158 mg, 0.588 mmol) and 2M aq. K₂CO₃ solution (0.882 mL, 1.764 mmol).

The mixture was purged with argon for 2 min. and PdCl₂ (dppf)-CH₂Cl₂ adduct (48.0 mg, 0.059 mmol) was added. The reaction mixture was heated at 100° C. for 16 h, cooled to RT and concentrated in vacuo. The crude product was purified by preparative HPLC (Method F). Fractions containing the desired product were combined and evaporated under reduced pressure to afford Example 37 (120 mg, 64.3% yield). MS (ES): m/z=318.1 [M+H]⁺; HPLC Ret. Time 1.719 min. (HPLC Method C); ¹H NMR (400 MHz, DMSO-d₆) δ ppm 8.49-8.43 (m, 2H), 8.18 (s, 1H), 7.83-7.78 (m, 1H), 7.52-7.44 (m, 2H), 7.33 (dd, J=9.4, 1.8 Hz, 1H), 7.26-7.16 (m, 2H), 3.95 (s, 3H).

### Example 38: 6-(3-(4-fluorophenyl)-1-methyl-1H-pyrazol-4-yl)imidazo[1,2-a]pyridine-3-carboxamide

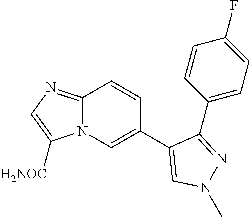 [SMILES: Cn1cc(-c2ccc3ncc(C(N)=O)n3c2)c(-c2ccc(F)cc2)n1]

Example 38 was synthesized by reacting Example 37 with TFA/H₂SO₄ and by employing the experimental procedure described for Example 36 in Scheme 24. The crude product was purified via preparative HPLC (Method F). Fractions containing the product were combined and evaporated under reduced pressure to afford Example 38. MS (ES): m/z=336.1 [M+H]⁺; HPLC Ret. Time 1.398 min. and 1.392 min. (HPLC Methods C and D, respectively); ¹H NMR (400 MHz, DMSO-d₆) δ ppm 9.46 (d, J=1.0 Hz, 1H), 8.34 (s, 1H), 8.07 (s, 1H), 7.94 (br. s., 1H), 7.73-7.65 (m, 2H), 7.49-7.42 (m, 2H), 7.37 (br. s., 1H), 7.26-7.10 (m, 3H), 3.94 (s, 3H).

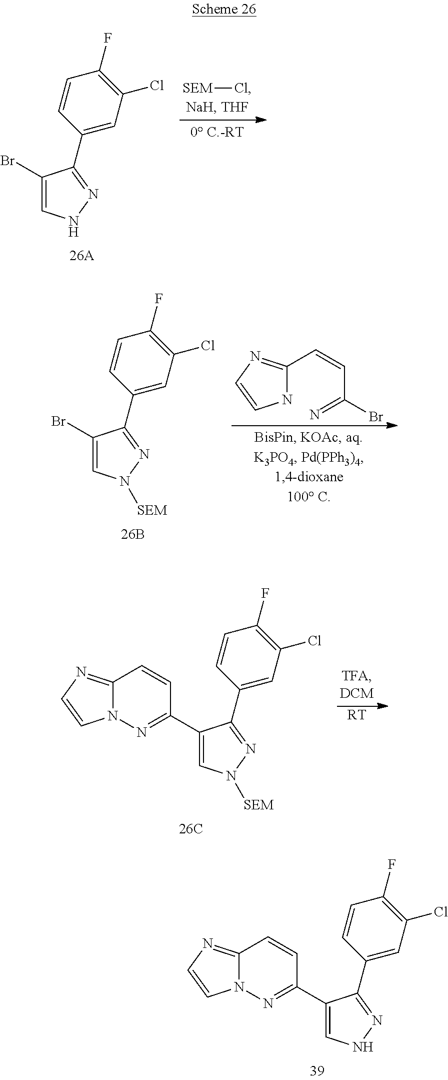

### Intermediate 26B: 4-bromo-3-(3-chloro-4-fluorophenyl)-1-((2-(trimethylsilyl)ethoxy)methyl)-1H-pyrazole

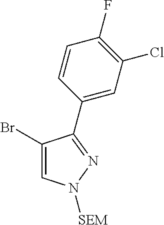 [SMILES: C[Si](C)(C)CCOCn1cc(Br)c(-c2ccc(F)c(Cl)c2)n1]

Intermediate 26B was synthesized by reacting intermediate 26A with SEM-Cl and by employing the experimental procedure described for intermediate 8A in Scheme 8. The crude product was triturated in petroleum ether and filtered to afford intermediate 26B. MS (ES): m/z=407.1 [M+H]⁺; HPLC Ret. Time 1.98 min. (HPLC Method B).

### Intermediate 26C: 6-(3-(3-chloro-4-fluorophenyl)-1-((2-(trimethylsilyl)ethoxy)methyl)-1H-pyrazol-4-yl)imidazo[1,2-b]pyridazine

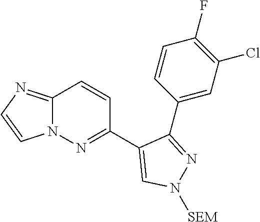 [SMILES: C[Si](C)(C)CCOCn1cc(-c2ccc3nccn3n2)c(-c2ccc(F)c(Cl)c2)n1]

To a solution of intermediate 26B (100 mg, 0.246 mmol) in 1,4-dioxane (3 mL) was added BisPin (94 mg, 0.370 mmol) and potassium acetate (72.6 mg, 0.739 mmol). The solution was purged with argon for 2 min. and PdCl₂(dppf)-CH₂Cl₂ adduct (10.06 mg, 0.012 mmol) was added. The reaction mixture was heated at 90° C. for 3 h and cooled to RT. 6-Bromoimidazo[1,2-b]pyridazine (48.8 mg, 0.246 mmol), aqueous 3M K₃PO₄ solution (0.370 mL, 0.739 mmol) and PdCl₂(dppf)-CH₂Cl₂ adduct (10.06 mg, 0.012 mmol) were added. The reaction mixture was heated at 90° C. for 16 h, cooled to RT and concentrated in vacuo. The crude product was purified by silica gel chromatography (12 g RediSep® column, eluting with a gradient of 40-100% EtOAc in petroleum ether). Fractions containing the desired product were combined and evaporated under reduced pressure to afford intermediate 26B (30 mg, 27.4% yield). MS (ES): m/z=444.5 [M+H]⁺; HPLC Ret. Time 1.25 min. (HPLC Methods B).

### Example 39: 6-(3-(3-chloro-4-fluorophenyl)-1H-pyrazol-4-yl)imidazo[1,2-b]pyridazine, TFA

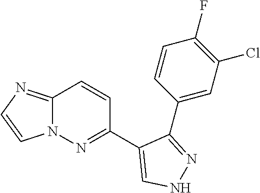 [SMILES: Fc1ccc(-c2n[nH]cc2-c2ccc3nccn3n2)cc1Cl]

Example 39 was synthesized by reacting intermediate 26C with TFA and by employing the experimental procedure described for Example 1 in Scheme 1. The crude product was purified via preparative HPLC (Method F). Fractions containing the product were combined and evaporated under reduced pressure to afford Example 39. MS (ES): m/z=314.0 [M+H]⁺; HPLC Ret. Time 1.554 min. and 0.927 min. (HPLC Methods C and D, respectively); ¹H NMR (400 MHz, DMSO-d₆) δ ppm 13.52 (br. s., 1H), 8.43 (br. s., 1H), 8.18-8.02 (m, 2H), 7.81 (br. s., 1H), 7.73 (d, J=1.0 Hz, 1H), 7.55 (br. s., 1H), 7.42 (br. s., 1H), 7.24 (d, J=8.8 Hz, 1H).

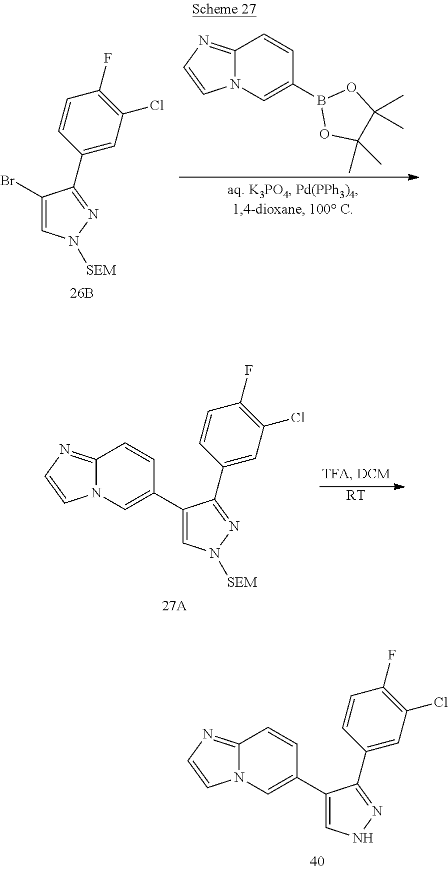 [SMILES: C[Si](C)(C)CCOCn1cc(Br)c(-c2ccc(F)c(Cl)c2)n1 ; CC1(C)OB(c2ccc3nccn3c2)OC1(C)C ; C[Si](C)(C)CCOCn1cc(-c2ccc3nccn3c2)c(-c2ccc(F)c(Cl)c2)n1 ; "RT" ; Fc1ccc(-c2n[nH]cc2-c2ccc3nccn3c2)cc1Cl]

### Intermediate 27A: 6-(3-(3-chloro-4-fluorophenyl)-1-((2-(trimethylsilyl)ethoxy)methyl)-1H-pyrazol-4-yl)imidazo[1,2-a]pyridine

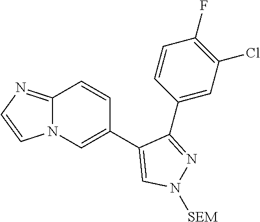 [SMILES: C[Si](C)(C)CCOCn1cc(-c2ccc3nccn3c2)c(-c2ccc(F)c(Cl)c2)n1]

Intermediate 27A was synthesized by reacting intermediate 26B with 6-(4,4,5,5-tetramethyl-1,3,2-dioxaborolan-2-yl)imidazo[1,2-a]pyridine and by employing the experimental procedure described for intermediate 23B in Scheme 23. The crude product was purified by silica gel chromatography (24 g RediSep® column, eluting with a gradient of 50-100% EtOAc in petroleum ether). Fractions containing the desired product were combined and evaporated under reduced pressure to afford intermediate 27A. MS (ES): m/z=443.5 [M+H]⁺; HPLC Ret. Time 1.22 min. (HPLC Method B).

### Example 40: 6-(3-(3-chloro-4-fluorophenyl)-1H-pyrazol-4-yl)imidazo[1,2-a]pyridine, TFA

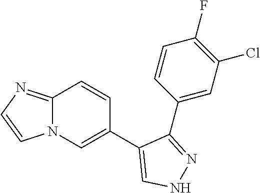 [SMILES: Fc1ccc(-c2n[nH]cc2-c2ccc3nccn3c2)cc1Cl]

Example 40 was synthesized by reacting Intermediate 27A with TFA and by employing the experimental procedure described for Example 1 in Scheme 1. The crude product was purified via preparative HPLC (Method F). Fractions containing the product were combined and evaporated under reduced pressure to afford Example 40. MS (ES): m/z=313.0 [M+H]⁺; HPLC Ret. Time 1.525 min. (HPLC Methods C); ¹H NMR (400 MHz, DMSO-d₆) δ ppm 13.40 (br. s., 1H), 8.53 (s, 1H), 7.98 (br. s., 1H), 7.92 (s, 1H), 7.66 (d, J=8.1 Hz, 1H), 7.60-7.50 (m, 2H), 7.45-7.36 (m, 2H), 7.03 (dd, J=9.3, 1.5 Hz, 1H).

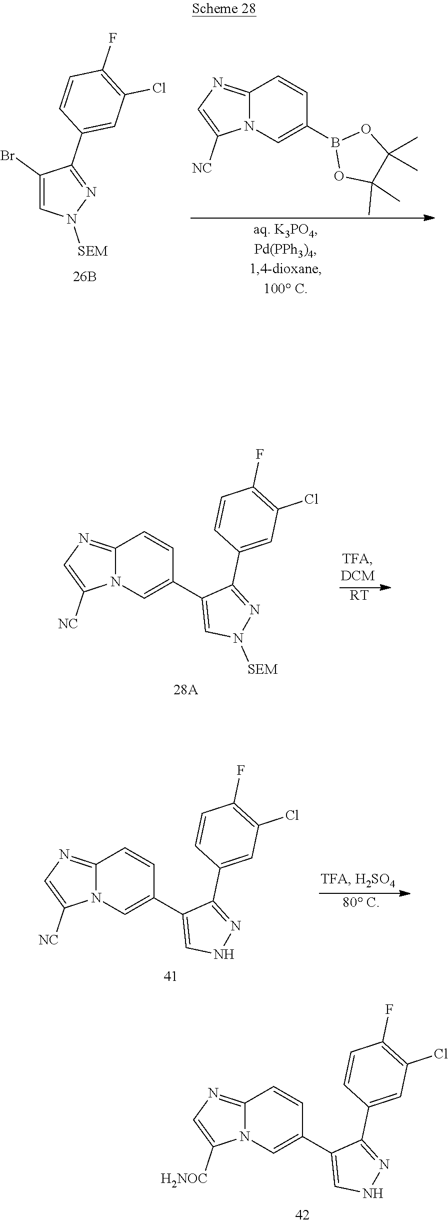

### Intermediate 28A: 6-(3-(3-chloro-4-fluorophenyl)-1-((2-(trimethylsilyl)ethoxy)methyl)-1H-pyrazol-4-yl)imidazo[1,2-a]pyridine-3-carbonitrile

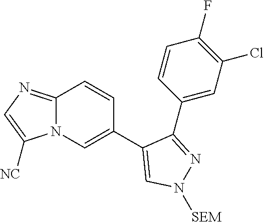 [SMILES: C[Si](C)(C)CCOCn1cc(-c2ccc3ncc(C#N)n3c2)c(-c2ccc(F)c(Cl)c2)n1]

Intermediate 28A was synthesized by reacting intermediate 26B with 6-(4,4,5,5-tetramethyl-1,3,2-dioxaborolan-2-yl)imidazo[1,2-a]pyridine-3-carbonitrile and by employing the experimental procedure described for intermediate 23B in Scheme 23. The crude product was purified by silica gel chromatography (24 g RediSep® column, eluting with a gradient of 50-100% EtOAc in petroleum ether). Fractions containing the desired product were combined and evaporated under reduced pressure to afford intermediate 28A. MS (ES): m/z=468.4 [M+H]⁺; HPLC Ret. Time 1.27 min. (HPLC Method B).

### Example 41: 6-(3-(3-chloro-4-fluorophenyl)-1H-pyrazol-4-yl)imidazo[1,2-a]pyridine-3-carbonitrile

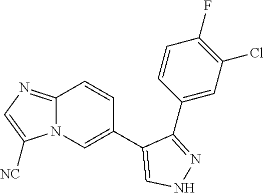 [SMILES: N#Cc1cnc2ccc(-c3c[nH]nc3-c3ccc(F)c(Cl)c3)cn12]

Example 41 was synthesized by reacting intermediate 28A with TFA and by employing the experimental procedure described for Example 1 in Scheme 1. The crude product was purified via preparative HPLC (Method F). Fractions containing the product were combined and evaporated under reduced pressure to afford Example 41. MS (ES): m/z=338.0 [M+H]⁺; HPLC Ret. Time 1.679 min. (HPLC Methods C); ¹H NMR (400 MHz, DMSO-d₆) δ ppm 13.38 (br. s., 1H), 8.52 (s, 1H), 8.45 (s, 1H), 8.25 (br. s., 1H), 7.80 (d, J=9.3 Hz, 2H), 7.69 (br. s., 2H), 7.45-7.33 (m, 3H).

### Example 42: 6-(3-(3-chloro-4-fluorophenyl)-1H-pyrazol-4-yl)imidazo[1,2-a]pyridine-3-carboxamide, TFA

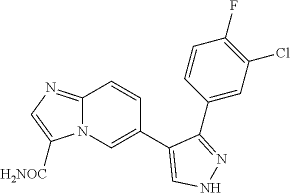 [SMILES: NC(=O)c1cnc2ccc(-c3c[nH]nc3-c3ccc(F)c(Cl)c3)cn12]

Example 42 was synthesized by reacting Example 41 with TFA/H₂SO₄ and by employing the experimental procedure described for Example 36 in Scheme 24. The crude product was purified via preparative HPLC (Method F). Fractions containing the product were combined and evaporated under reduced pressure to afford Example 42. MS (ES): m/z=356.0 [M+H]⁺; HPLC Ret. Time 1.271 min. (HPLC Methods C); ¹H NMR (400 MHz, DMSO-d₆) δ ppm 13.33 (br. s., 1H), 9.50-9.39 (m, 2H), 8.37-8.29 (m, 2H), 8.06 (br. s., 1H), 7.73-7.61 (m, 3H), 7.45-7.23 (m, 3H).

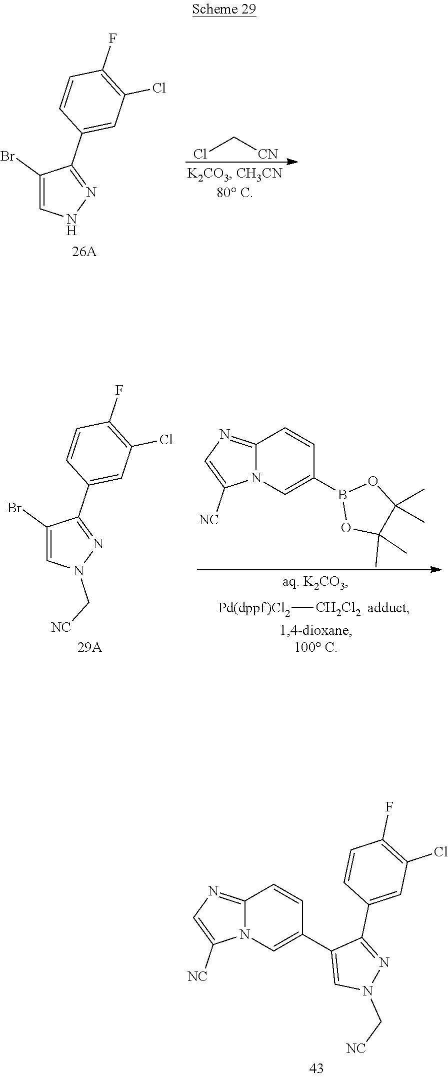

### Intermediate 29A: 2-(4-bromo-3-(3-chloro-4-fluorophenyl)-1H-pyrazol-1-yl)acetonitrile

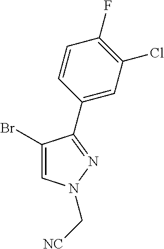 [SMILES: N#CCn1cc(Br)c(-c2ccc(F)c(Cl)c2)n1]

To a solution of intermediate 26A (1.0 g, 3.63 mmol) and K₂CO₃ (1.505 g, 10.89 mmol in CH₃CN (10 mL) was added 2-chloroacetonitrile (0.548 g, 7.26 mmol). The reaction mixture was heated at 80° C. for 12 h. The reaction was quenched with ice-water and extracted with EtOAc. The organic layer was washed with brine, dried over Na₂SO₄, filtered and the filtrate concentrated in vacuo. The crude product was purified by silica gel chromatography (24 g RediSep® column, eluting with a gradient of 50-100% EtOAc in petroleum ether). Fractions containing the desired product were combined and evaporated under reduced pressure to afford intermediate 29A (750 mg, 65.7% yield).

MS (ES): m/z=314.0 [M−H]⁺; HPLC Ret. Time 1.40 min. (HPLC Method B).

### Example 43: 6-(3-(3-chloro-4-fluorophenyl)-1-(cyanomethyl)-1H-pyrazol-4-yl)imidazo[1,2-a]pyridine-3-carbonitrile

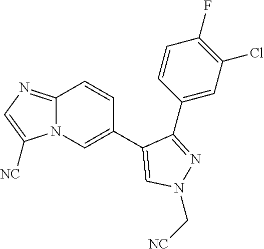 [SMILES: N#CCn1cc(-c2ccc3ncc(C#N)n3c2)c(-c2ccc(F)c(Cl)c2)n1]

Example 43 was synthesized by reacting intermediate 29A with 6-(4,4,5,5-tetramethyl-1,3,2-dioxaborolan-2-yl)imidazo[1,2-a]pyridine-3-carbonitrile and by employing the experimental procedure described for Example 37 in Scheme 25. The crude product was purified by preparative HPLC (Method L). Fractions containing the desired product were combined and evaporated under reduced pressure to afford Example 43. MS (ES): m/z=377.0 [M+H]⁺; HPLC Ret. Time 1.878 min. (HPLC Method C); ¹H NMR (400 MHz, DMSO-d₆) δ ppm 8.61 (s, 1H), 8.47 (s, 1H), 8.33 (s, 1H), 7.82 (d, J 9.0 Hz, 1H), 7.68 (d, J=7.3 Hz, 1H), 7.43 (d, J=7.8 Hz, 2H), 7.35 (dd, J=9.2, 1.6 Hz, 1H), 5.64 (s, 2H).

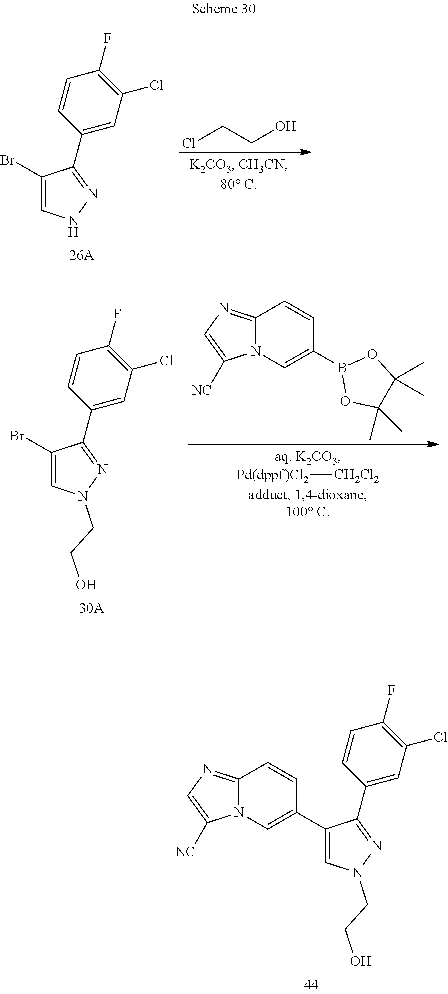

### Intermediate 30A: 2-(4-bromo-3-(3-chloro-4-fluorophenyl)-1H-pyrazol-1-yl)ethanol

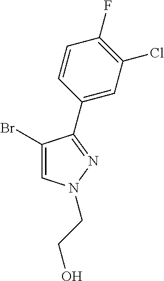 [SMILES: OCCn1cc(Br)c(-c2ccc(F)c(Cl)c2)n1]

Intermediate 30A was synthesized by reacting intermediate 26A with 2-bromoethanol and by employing the experimental procedure described for Example 29A in Scheme 29. The crude product was purified by silica gel chromatography (24 g RediSep® column, eluting with a gradient of 50-100% EtOAc in petroleum ether). Fractions containing the desired product were combined and evaporated under reduced pressure to afford intermediate 30A. MS (ES): m/z=320.9 [M−H]⁺; HPLC Ret. Time 1.16 min. (HPLC Method A).

### Example 44: 6-(3-(3-chloro-4-fluorophenyl)-1-(2-hydroxyethyl)-1H-pyrazol-4-yl)imidazo[1,2-a]pyridine-3-carbonitrile

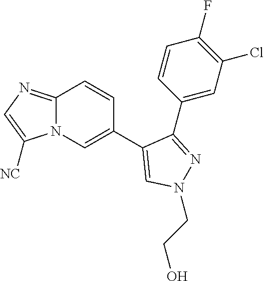 [SMILES: N#Cc1cnc2ccc(-c3cn(CCO)nc3-c3ccc(F)c(Cl)c3)cn12]

Example 44 was synthesized by reacting intermediate 30A with 6-(4,4,5,5-tetramethyl-1,3,2-dioxaborolan-2-yl)imidazo[1,2-a]pyridine-3-carbonitrile and by employing the experimental procedure described for Example 37 in Scheme 25. The crude product was purified by preparative HPLC (Method L). Fractions containing the desired product were combined and evaporated under reduced pressure to afford Example 44. MS (ES): m/z=382.0 [M+H]⁺; HPLC Ret. Time 1.642 min. and 1.570 min. (HPLC Method C and D, respectively); ¹H NMR (400 MHz, DMSO-d₆) δ ppm 8.52 (d, J=1.5 Hz, 1H), 8.46 (s, 1H), 8.22 (s, 1H), 7.81 (dd, J=9.3, 1.0 Hz, 1H), 7.69-7.64 (m, 1H), 7.43-7.38 (m, 2H), 7.35 (dd, J=9.3, 1.7 Hz, 1H), 5.01 (t, J=5.5 Hz, 1H), 4.25 (t, J=5.5 Hz, 2H), 3.85 (q, J=5.5 Hz, 2H).

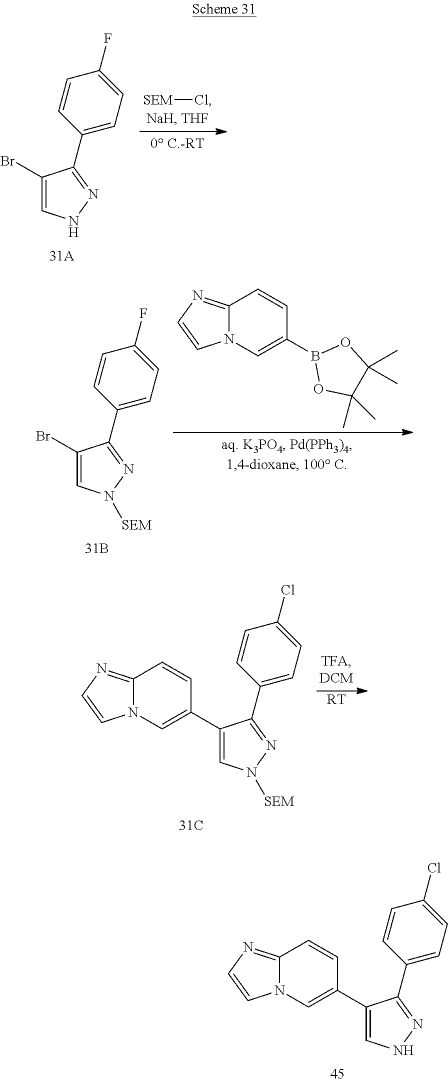

### Intermediate 31B: 4-bromo-3-(4-chlorophenyl)-1-((2-(trimethylsilyl)ethoxy)methyl)-1H-pyrazole

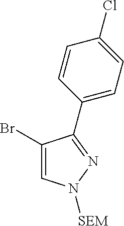 [SMILES: C[Si](C)(C)CCOCn1cc(Br)c(-c2ccc(Cl)cc2)n1]

Intermediate 31B was synthesized by reacting intermediate 31A with SEM-Cl and by employing the experimental procedure described for intermediate 8A in Scheme 8. The crude product was triturated with petroleum ether and filtered off to afford intermediate 31B. MS (ES): m/z=389.1 [M+2H]⁺; HPLC Ret. Time 1.94 min. (HPLC Method B).

### Intermediate 31C: 6-(3-(4-chlorophenyl)-1-((2-(trimethylsilyl)ethoxy)methyl)-1H-pyrazol-4-yl)imidazo[1,2-a]pyridine

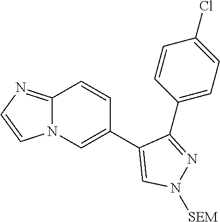 [SMILES: C[Si](C)(C)CCOCn1cc(-c2ccc3nccn3c2)c(-c2ccc(Cl)cc2)n1]

Intermediate 31C was synthesized by reacting intermediate 31B with 6-(4,4,5,5-tetramethyl-1,3,2-dioxaborolan-2-yl)imidazo[1,2-a]pyridine and by employing the experimental procedure described for intermediate 23B in Scheme 23. The crude product was purified by silica gel chromatography (24 g RediSep® column, eluting with a gradient of 50-100% EtOAc in petroleum ether). Fractions containing the desired product were combined and evaporated under reduced pressure to afford intermediate 31C. MS (ES): m/z=425.5 [M+H]⁺; HPLC Ret. Time 1.77 min. (HPLC Method B).

### Example 45: 6-(3-(4-chlorophenyl)-1H-pyrazol-4-yl)imidazo[1,2-a]pyridine

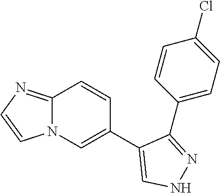 [SMILES: Clc1ccc(-c2n[nH]cc2-c2ccc3nccn3c2)cc1]

Example 45 was synthesized by reacting Intermediate 31C with TFA and by employing the experimental procedure described for Example 1 in Scheme 1. The crude product was purified via preparative HPLC (Method F). Fractions containing the product were combined and evaporated under reduced pressure to afford Example 45. MS (ES): m/z=295.0 [M+H]⁺; HPLC Ret. Time 1.269 min. and 1.059 min. (HPLC Methods C and D, respectively); ¹H NMR (400 MHz, DMSO-d₆) δ ppm 13.10-13.47 (m, 1.0H), 8.45-8.62 (m, 1.1H), 8.03 (s, 0.7H), 7.92 (s, 1.0H), 7.34-7.64 (m, 5.9H), 6.95-7.08 (m, 1.0H).

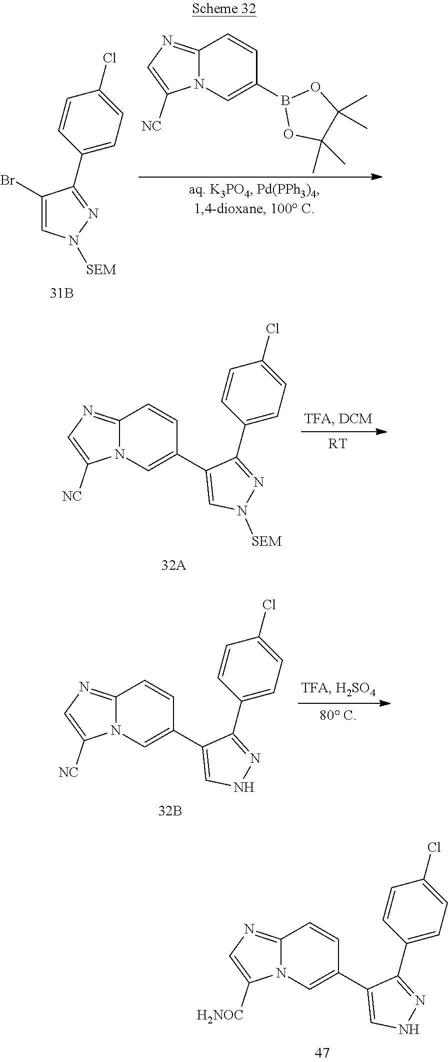

### Intermediate 32A: 6-(3-(4-chlorophenyl)-1-((2-(trimethylsilyl)ethoxy)methyl)-1H-pyrazol-4-yl)imidazo[1,2-a]pyridine-3-carbonitrile

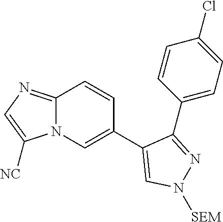 [SMILES: C[Si](C)(C)CCOCn1cc(-c2ccc3ncc(C#N)n3c2)c(-c2ccc(Cl)cc2)n1]

Intermediate 32A was synthesized by reacting intermediate 31B with 6-(4,4,5,5-tetramethyl-1,3,2-dioxaborolan-2-yl)imidazo[1,2-a]pyridine-3-carbonitrile and by employing the experimental procedure described for intermediate 23B in Scheme 23. The crude product was purified by silica gel chromatography (24 g RediSep® column, eluting with a gradient of 50-100% EtOAc in petroleum ether). Fractions containing the desired product were combined and evaporated to afford intermediate 32A. MS (ES): m/z=450.5 [M+H]⁺; HPLC Ret. Time 1.86 min. (HPLC Method B).

### Example 46: 6-(3-(4-chlorophenyl)-1H-pyrazol-4-yl)imidazo[1,2-a]pyridine-3-carbonitrile

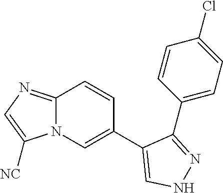 [SMILES: N#Cc1cnc2ccc(-c3c[nH]nc3-c3ccc(Cl)cc3)cn12]

Example 46 was synthesized by reacting intermediate 32A with TFA and by employing the experimental procedure described for Example 1 in Scheme 1. The crude product was purified via preparative HPLC (Method F). Fractions containing the product were combined and evaporated under reduced pressure to afford Example 46. MS (ES): m/z=320.0 [M+H]⁺; HPLC Ret. Time 1.448 min. and 1.466 min. (HPLC Methods C and D, respectively); ¹H NMR (400 MHz, DMSO-d₆): δ ppm 13.19-13.57 (m, 1.2H), 8.51 (s, 1.1H), 8.43 (s, 0.9H), 8.22 (br. s., 0.8H), 7.94 (br. s., 0.6H), 7.80 (d, J=9.5 Hz, 1.3H), 7.41-7.59 (m, 7.0H), 7.40-7.59 (m, 4.2H), 7.37 (dd, J=9.0, 1.5 Hz, 1.0H), 6.89-7.28 ppm (m, 2.0H).

### Example 47: 6-(3-(4-chlorophenyl)-1H-pyrazol-4-yl)imidazo[1,2-a]pyridine-3-carboxamide

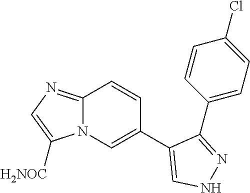 [SMILES: NC(=O)c1cnc2ccc(-c3c[nH]nc3-c3ccc(Cl)cc3)cn12]

Example 47 was synthesized by reacting Example 46 with TFA/H₂SO₄ and by employing the experimental procedure described for Example 36 in Scheme 24. The crude product was purified via preparative HPLC (Method F). Fractions containing the product were combined and evaporated under reduced pressure to afford Example 47. MS (ES): m/z=338.1 [M+H]⁺; HPLC Ret. Time 1.089 min. and 0.798 min. (HPLC Methods C and D, respectively); ¹H NMR (400 MHz, DMSO-d₆) δ ppm 9.47 (s, 1H), 8.33 (s, 1H), 8.11 (br. s., 1H), 8.01-7.75 (m, 2H), 7.72-7.63 (m, 1H), 7.52-7.34 (m, 5H), 7.30-7.20 (m, 1H).

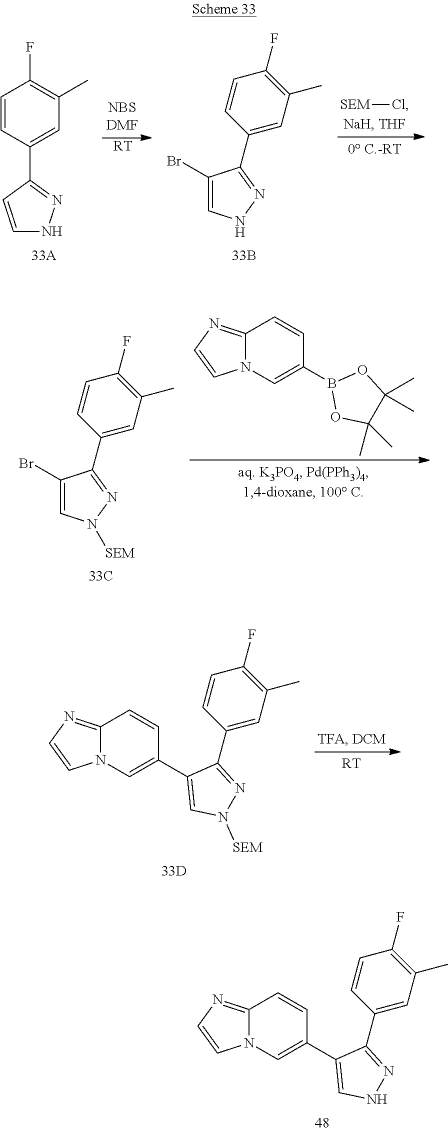

### Intermediate 33B: 4-bromo-3-(4-fluoro-3-methylphenyl)-1H-pyrazole

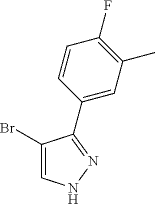

Intermediate 33B was synthesized by reacting intermediate 33A with NBS and by employing the experimental procedure described for intermediate 1D in Scheme 1. The crude product was triturated with petroleum ether and filtered to afford intermediate 33B. MS (ES): m/z=255.3 [M+H]⁺; HPLC Ret. Time 1.25 min. (HPLC Method B).

### Intermediate 33C: 4-bromo-3-(4-fluoro-3-methylphenyl)-1-((2-(trimethylsilyl)ethoxy)methyl)-1H-pyrazole

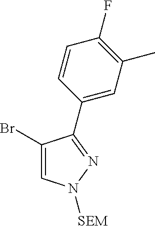 [SMILES: Cc1cc(-c2nn(COCC[Si](C)(C)C)cc2Br)ccc1F]

Intermediate 33C was synthesized by reacting intermediate 33B with SEM-Cl and by employing the experimental procedure described for intermediate 8A in Scheme 8. The crude product was triturated with petroleum ether and filtered off to afford intermediate 33C. MS (ES): m/z=387.1 [M+2H]⁺; HPLC Ret. Time 1.88 min. (HPLC Method B).

### Intermediate 33D: 6-(3-(4-fluoro-3-methylphenyl)-1-((2-(trimethylsilyl)ethoxy)methyl)-1H-pyrazol-4-yl)imidazo[1,2-a]pyridine

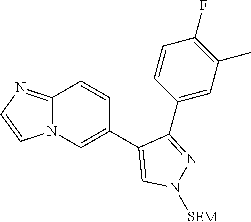 [SMILES: Cc1cc(-c2nn(COCC[Si](C)(C)C)cc2-c2ccc3nccn3c2)ccc1F]

Intermediate 33D was synthesized by reacting intermediate 33C with 6-(4,4,5,5-tetramethyl-1,3,2-dioxaborolan-2-yl)imidazo[1,2-a]pyridine and by employing the experimental procedure described for Intermediate 23B in Scheme 23. The crude product was purified by silica gel chromatography (24 g RediSep® column, eluting with a gradient of 50-100% EtOAc in petroleum ether). Fractions containing the desired product were combined and evaporated under reduced pressure to afford intermediate 33D. MS (ES): m/z=423.5 [M+H]⁺; HPLC Ret. Time 1.71 min. (HPLC Method B).

### Example 48: 6-(3-(4-fluoro-3-methylphenyl)-1H-pyrazol-4-yl)imidazo[1,2-a]pyridine

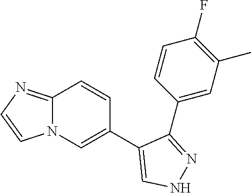 [SMILES: Cc1cc(-c2n[nH]cc2-c2ccc3nccn3c2)ccc1F]

Example 48 was synthesized by reacting intermediate 33D with TFA and by employing the experimental procedure described for Example 1 in Scheme 1. The crude product was purified via preparative HPLC (Method F). Fractions containing the product were combined and evaporated under reduced pressure to afford Example 48. MS (ES): m/z=293.1 [M+H]⁺; HPLC Ret. Time 1.254 min. and 1.051 min. (HPLC Methods C and D, respectively); ¹H NMR (400 MHz, DMSO-d₆): δ ppm 13.04-13.33 (m, 1H), 8.42-8.60 (m, 1H), 8.01 (br. s., 1H), 7.91 (s, 1H), 7.75 (br. s, 1H), 7.56 (d, J=1.0 Hz, 1H), 7.38-7.54 (m, 1H), 7.24 (br. s., 1H), 6.96-7.14 (m, 2H), 2.22 (br. s., 3H).

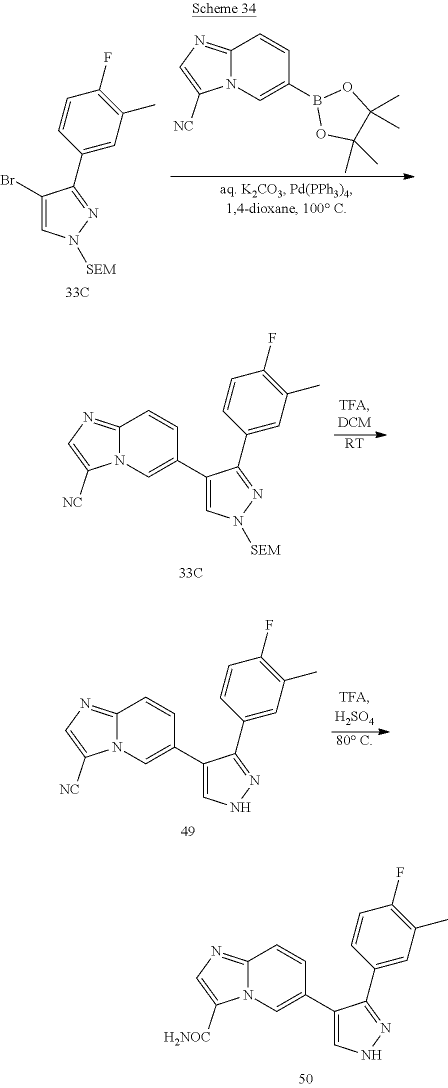

### Intermediate 34A: 6-(3-(4-fluoro-3-methylphenyl)-1-((2-(trimethylsilyl)ethoxy)methyl)-1H-pyrazol-4-yl)imidazo[1,2-a]pyridine-3-carbonitrile

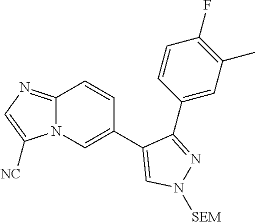 [SMILES: Cc1cc(-c2nn(COCC[Si](C)(C)C)cc2-c2ccc3ncc(C#N)n3c2)ccc1F]

Intermediate 34A was synthesized by reacting intermediate 33C with 6-(4,4,5,5-tetramethyl-1,3,2-dioxaborolan-2-yl)imidazo[1,2-a]pyridine-3-carbonitrile and by employing the experimental procedure described for Example 34A in Scheme 34. The crude product was purified by silica gel chromatography (24 g RediSep® column, eluting with a gradient of 50-100% EtOAc in petroleum ether). Fractions containing the desired product were combined and evaporated under reduced pressure to afford intermediate 34A. MS (ES): m/z=448.5 [M+H]⁺; HPLC Ret. Time 1.82 min. (HPLC Method B).

### Example 49: 6-(3-(4-fluoro-3-methylphenyl)-1H-pyrazol-4-yl)imidazo[1,2-a]pyridine-3-carbonitrile

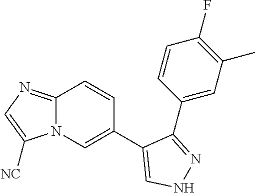 [SMILES: Cc1cc(-c2n[nH]cc2-c2ccc3ncc(C#N)n3c2)ccc1F]

Example 49 was synthesized by reacting Intermediate 34A with TFA and by employing the experimental procedure described for Example 1 in Scheme 1. The crude product was purified via preparative HPLC (Method F). Fractions containing the product were combined and evaporated under reduced pressure to afford Example 49. MS (ES): m/z=318.1 [M+H]⁺; HPLC Ret. Time 1.431 min. and 1.447 min. (HPLC Methods C and D, respectively); ¹H NMR (400 MHz, DMSO-d₆): δ ppm 13.14-13.42 (m, 1H), 8.37-8.53 (m, 2H), 8.24 (s, 1H), 7.72-7.84 (m, 1H), 7.35-7.50 (m, 2H), 7.19-7.33 (m, 1H), 7.05-7.16 (m, 1H), 2.18-2.30 (m, 3H).

### Example 50: 6-(3-(4-fluoro-3-methylphenyl)-1H-pyrazol-4-yl)imidazo[1,2-a]pyridine-3-carboxamide

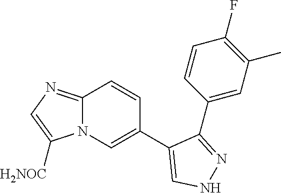 [SMILES: Cc1cc(-c2n[nH]cc2-c2ccc3ncc(C(N)=O)n3c2)ccc1F]

Example 50 was synthesized by reacting Example 49 with TFA/H₂SO₄ and by employing the experimental procedure described for Example 36 in Scheme 24. The crude product was purified via preparative HPLC (Method F). Fractions containing the product were combined and evaporated under reduced pressure to afford Example 50. MS (ES): m/z=336.1 [M+H]⁺; HPLC Ret. Time 1.071 min. and 0.782 min. (HPLC Methods C and D, respectively); ¹H NMR (400 MHz, DMSO-d₆) δ ppm 9.49 (d, J=1.0 Hz, 1H), 8.00 (br. s., 2H), 7.73-7.65 (m, 1H), 7.43 (dd, J=7.3, 1.8 Hz, 1H), 7.35 (dd, J=9.3, 1.8 Hz, 1H), 7.26-7.21 (m, 1H), 7.13 (d, J=9.5 Hz, 1H), 2.21 (d, J=1.5 Hz, 3H).

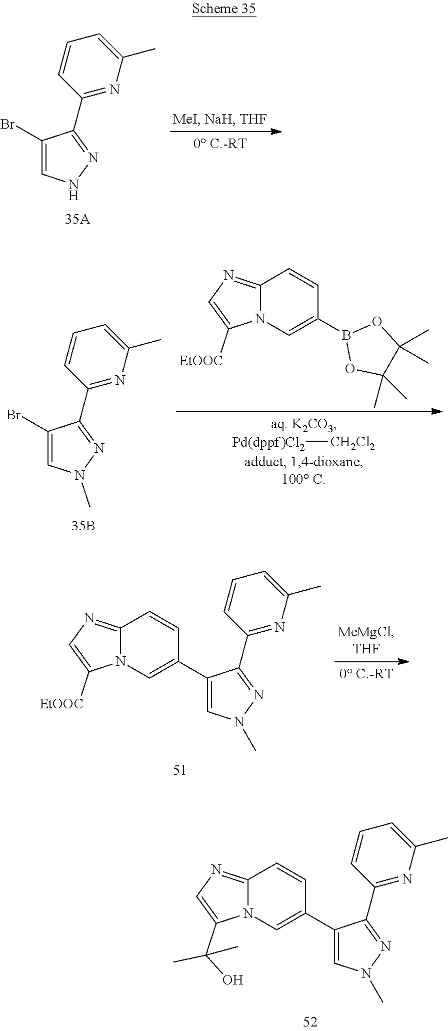

### Intermediate 35B: 2-(4-bromo-1-methyl-1H-pyrazol-3-yl)-6-methylpyridine

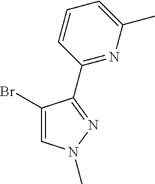

Intermediate 35B was synthesized by reacting intermediate 35A with methyl iodide and by employing the experimental procedure described for Example 6B in Scheme 6. The crude product was purified using preparative HPLC (Method L) to afford the desired isomer as intermediate 35B. MS (ES): m/z=252.3 [M+H]⁺; HPLC Ret. Time 1.06 min. (HPLC Method B).

### Example 51: ethyl 6-(1-methyl-3-(6-methylpyridin-2-yl)-1H-pyrazol-4-yl)imidazo[1,2-a]pyridine-3-carboxylate

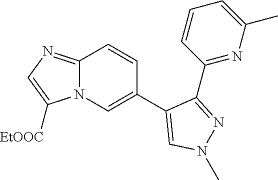 [SMILES: CCOC(=O)c1cnc2ccc(-c3cn(C)nc3-c3cccc(C)n3)cn12]

Example 51 was synthesized by reacting intermediate 35B with ethyl 6-(4,4,5,5-tetramethyl-1,3,2-dioxaborolan-2-yl)imidazo[1,2-a]pyridine-3-carboxylate and by employing the experimental procedure described for Example 37 in Scheme 25. The crude product was purified by preparative HPLC (Method H). Fractions containing the desired product were combined and evaporated under reduced pressure to afford Example 51. MS (ES): m/z=362.1 [M+H]⁺; HPLC Ret. Time 1.652 min. and 0959 min. (HPLC Method C and D respectively); ¹H NMR (400 MHz, DMSO-d₆) δ ppm 9.43 (s, 1H), 8.28 (s, 1H), 8.16 (s, 1H), 7.79-7.69 (m, 2H), 7.66-7.61 (m, 2H), 7.17 (d, J=7.6 Hz, 1H), 4.33 (q, J=7.1 Hz, 2H), 3.98 (s, 3H), 2.28 (s, 3H), 1.31 (t, J=7.1 Hz, 3H).

### Example 52: ethyl 6-(1-methyl-3-(6-methylpyridin-2-yl)-1H-pyrazol-4-yl)imidazo[1,2-a]pyridine-3-carboxylate

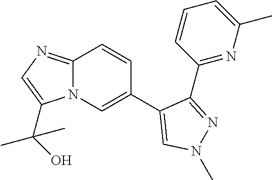 [SMILES: Cc1cccc(-c2nn(C)cc2-c2ccc3ncc(C(C)(C)O)n3c2)n1]

To a solution of Example 51 (30 mg, 0.083 mmol) in THF (2 mL) was added methylmagnesium chloride (0.138 mL, 0.415 mmol, 3M solution in THF) at 0° C. The reaction mixture was stirred at RT for 3 h, quenched with saturated aq. NH₄C₁ solution and extracted with EtOAc. The organic layer was washed with brine, dried over Na₂SO₄, filtered and the filtrate concentrated in vacuo. The crude product was purified using preparative HPLC (Method L) to afford Example 52 (6.7 mg, 22.30% yield). MS (ES): m/z=348.3 [M+H]⁺; HPLC Ret. Time 1.019 min. and 0.617 min. (HPLC Method C and D, respectively); ¹H NMR (400 MHz, DMSO-d₆) δ ppm 8.61 (s, 1H), 8.06 (s, 1H), 7.75-7.66 (m, 1H), 7.55-7.45 (m, 2H), 7.35 (s, 1H), 7.27 (dd, J=9.4, 1.6 Hz, 1H), 7.16 (d, J=7.8 Hz, 1H), 5.25 (d, J=14.4 Hz, 1H), 3.96 (s, 3H), 2.37-2.30 (m, 3H), 1.58-1.51 (m, 6H).

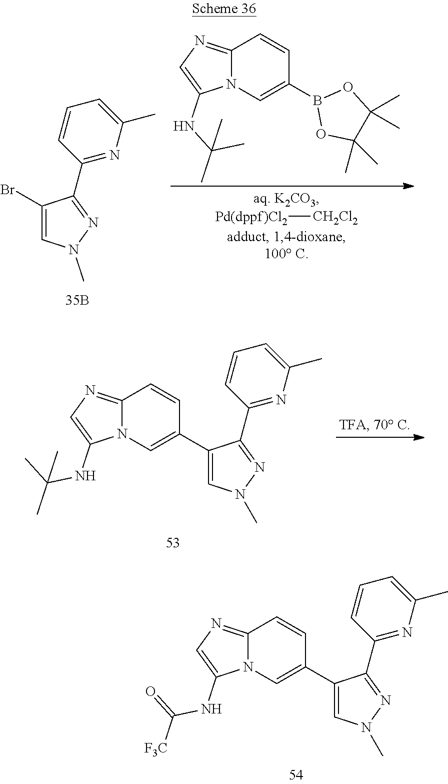

### Example 53: N-(tert-butyl)-6-(1-methyl-3-(6-methylpyridin-2-yl)-1H-pyrazol-4-yl)imidazo[1,2-a]pyridin-3-amine

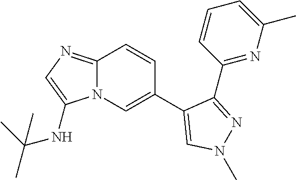 [SMILES: Cc1cccc(-c2nn(C)cc2-c2ccc3ncc(NC(C)(C)C)n3c2)n1]

Example 53 was synthesized by reacting intermediate 35B with N-(tert-butyl)-6-(4,4,5,5-tetramethyl-1,3,2-dioxaborolan-2-yl)imidazo[1,2-a]pyridin-3-amine (reference: J. Organic Chem., 2007, 72(3), 1013-1016) and by employing the experimental procedure described for Example 37 in Scheme 25. The crude product was purified by preparative HPLC (Method H). Fractions containing the desired product were combined and evaporated under reduced pressure to afford Example 53 (152 mg, 52.5% yield). MS (ES): m/z=361.2 [M+H]⁺; HPLC Ret. Time 1.581 min. and 0.974 min. (HPLC Method C and D, respectively); ¹H NMR (400 MHz, DMSO-d₆) δ ppm 8.39 (s, 1H), 8.06 (s, 1H), 7.73-7.66 (m, 1H), 7.50 (d, J=7.8 Hz, 1H), 7.36 (d, J=9.3 Hz, 1H), 7.20-7.14 (m, 2H), 7.12 (s, 1H), 3.96 (s, 3H), 2.39 (s, 3H), 1.08 (s, 9H).

### Example 54: Ethyl 6-(1-methyl-3-(6-methylpyridin-2-yl)-1H-pyrazol-4-yl)imidazo[1,2-a]pyridine-3-carboxylate

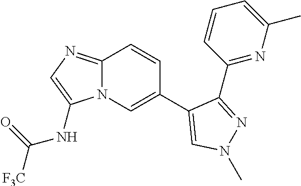 [SMILES: Cc1cccc(-c2nn(C)cc2-c2ccc3ncc(NC(=O)C(F)(F)F)n3c2)n1]

A solution of Example 53 (20 mg, 0.055 mmol) in TFA (1 mL) was heated at 70° C. for 1 h. The reaction mixture was cooled to RT and concentrated in vacuo. The crude product was purified via preparative HPLC (Method F) to afford Example 54. MS (ES): m/z=401.1 [M+H]⁺; HPLC Ret. Time 1.285 min. (HPLC Method C); ¹H NMR (400 MHz, DMSO-d₆) δ ppm 13.46 (s, 1H), 9.00 (s, 1H), 8.23 (br. s., 1H), 7.96-7.56 (m, 5H), 7.18 (d, J=7.6 Hz, 1H), 3.98 (s, 3H), 2.37 (s, 3H).

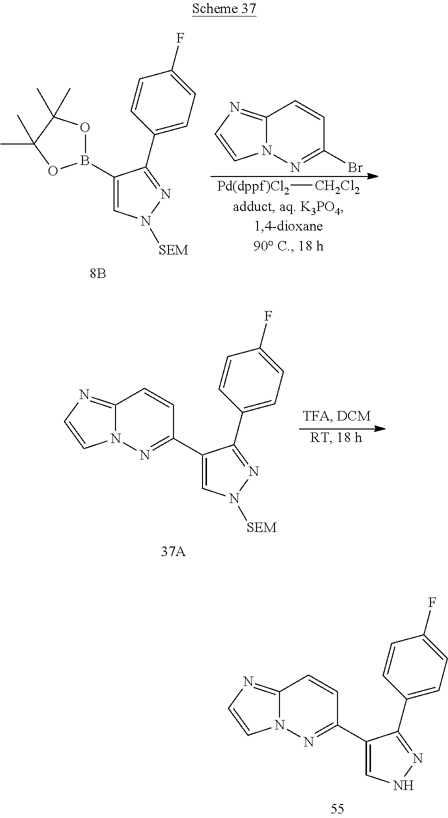

### Intermediate 37A: 6-(3-(4-fluorophenyl)-1-((2-(trimethylsilyl) ethoxy)methyl)-1H-pyrazol-4-yl)imidazo[1,2-b]pyridazine

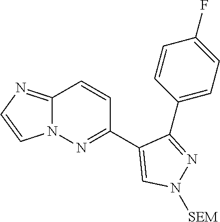 [SMILES: C[Si](C)(C)CCOCn1cc(-c2ccc3nccn3n2)c(-c2ccc(F)cc2)n1]

Intermediate 37A was synthesized by reacting intermediate 8B with 6-bromoimidazo[1,2-b]pyridazine and by employing the experimental procedure described for intermediate 1E in Scheme 1 to obtain intermediate 37A. LCMS: m/z=410.5 [M+H]⁺; HPLC Ret. Time 1.03 min. (HPLC Method D).

### Example 55: 6-(3-(4-fluorophenyl)-1H-pyrazol-4-yl)imidazo[1,2-b]pyridazine

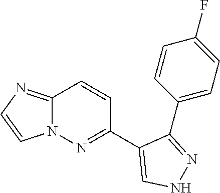 [SMILES: Fc1ccc(-c2n[nH]cc2-c2ccc3nccn3n2)cc1]

Example 55 was synthesized by reacting intermediate 37A with TFA and by employing the experimental procedure described for Example 1 in Scheme 1. The crude product was dissolved in DMF and purified via preparative HPLC (Condition N). Fractions containing the desired product were combined and dried under vacuum to afford Example 55. LCMS: m/z=280.1 [M+H]⁺; HPLC Ret. Time 1.064 min. (HPLC Method C); ¹H NMR (400 MHz, DMSO-d₆) δ ppm 8.50 (d, J=1.0 Hz, 1H), 8.41 (s, 2H), 8.27 (d, J=1.5 Hz, 1H), 7.75 (d, J=9.5 Hz, 1H), 7.70-7.60 (m, 2H), 7.32-7.21 (m, 2H).

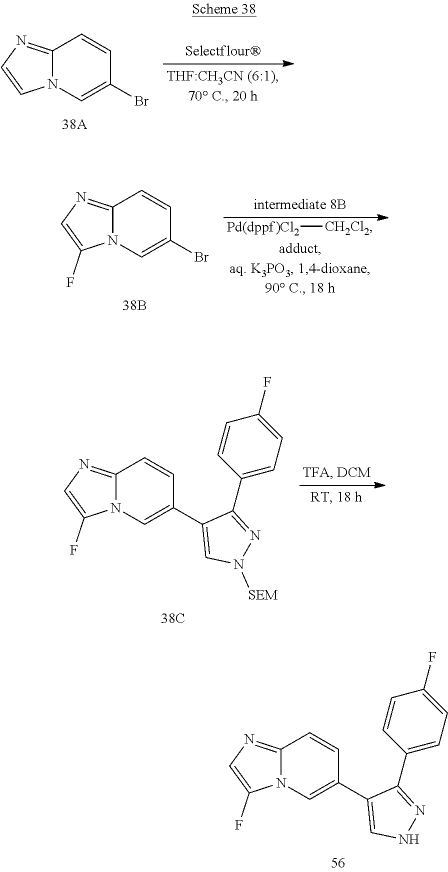

### Intermediate 38B: 6-bromo-3-fluoroimidazo[1,2-a]pyridine

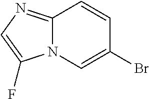 [SMILES: Fc1cnc2ccc(Br)cn12]

To a stirred solution of 38A (1.0 g, 5.08 mmol) in THF (60 mL) under argon condition was added NaH (0.122 g, 5.08 mmol, 60% in mineral oil). The reaction mixture was stirred at RT for 30 min. and to it was added Selectfluor® (3.87 g, 10.92 mmol) in acetonitrile (10 mL). The reaction mixture was heated at 70° C. for another 20 h. The reaction mixture was quenched with saturated aq. NH₄Cl solution (100 mL) and was extracted with ethyl acetate (3×100 mL). The combined organic layers were dried over anhydrous sodium sulfate, filtered and the filtrate evaporated under reduced pressure to get a brown gum. The crude product was purified by silica gel chromatography (24 g RediSep® column, eluting with a gradient of 17-19% EtOAc in petroleum ether). Fractions containing the desired product were combined and evaporated under reduced pressure to afford intermediate 38B (0.1 g, 9.16% yield). LCMS: m/z=215.3 [M+H]⁺; HPLC Ret. Time 0.37 min. (HPLC Method D).

### Intermediate 38C: 3-fluoro-6-(3-(4-fluorophenyl)-1-((2-(trimethylsilyl)ethoxy)methyl)-1H-pyrazol-4-yl)imidazo[1,2-a]pyridine

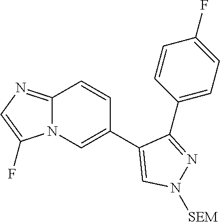 [SMILES: C[Si](C)(C)CCOCn1cc(-c2ccc3ncc(F)n3c2)c(-c2ccc(F)cc2)n1]

Intermediate 38C was synthesized by reacting intermediate 38B with 8B and by employing the experimental procedure described for intermediate 1E in Scheme 1 to afford 38C. LCMS: m/z=427.2 [M+H]⁺; HPLC Ret. Time 0.98 min. (HPLC Method D).

### Example 56: 3-fluoro-6-(3-(4-fluorophenyl)-1H-pyrazol-4-yl)imidazo[1,2-a]pyridine

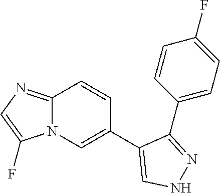 [SMILES: Fc1ccc(-c2n[nH]cc2-c2ccc3ncc(F)n3c2)cc1]

Example 56 was synthesized by reacting intermediate 38C with TFA and by employing the experimental procedure described for Example 1 in Scheme 1. The crude product was purified via preparative HPLC (Condition N). Fractions containing the desired product were combined and dried under vacuum to afford Example 56. LCMS: m/z=297.1 [M+H]⁺; HPLC Ret. Time 1.524 min. (HPLC Method C); ¹H NMR (400 MHz, DMSO-d₆) δ ppm 8.46 (s, 1H), 8.27 (s, 1H), 7.79-7.71 (m, 3H), 7.62 (d, J=7.3 Hz, 1H), 7.53-7.46 (m, 2H), 7.25 (dd, J=9.3, 1.7 Hz, 1H).

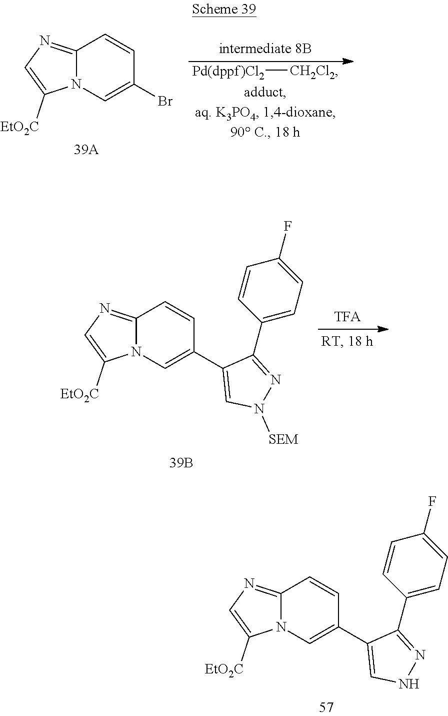

### Intermediate 39B: ethyl 6-(3-(4-fluorophenyl)-1-((2-(trimethylsilyl)ethoxy)methyl)-1H-pyrazol-4-yl)imidazo[1,2-a]pyridine-3-carboxylate

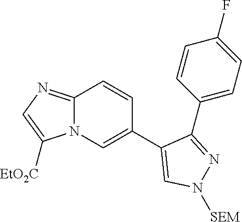 [SMILES: CCOC(=O)c1cnc2ccc(-c3cn(COCC[Si](C)(C)C)nc3-c3ccc(F)cc3)cn12]

Intermediate 39B was synthesized by reacting intermediate 39A with 8B and by employing the experimental procedure described for intermediate 1E in Scheme 1 to obtain intermediate 39B. LCMS: m/z=481.5 [M+H]⁺; HPLC Ret. Time 1.73 min. (HPLC Method B).

### Example 57: ethyl 6-(3-(4-fluorophenyl)-1H-pyrazol-4-yl)imidazo[1,2-a]pyridine-3-carboxylate

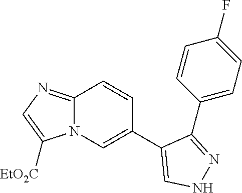 [SMILES: CCOC(=O)c1cnc2ccc(-c3c[nH]nc3-c3ccc(F)cc3)cn12]

Example 57 was synthesized by reacting intermediate 39B with TFA and by employing the experimental procedure described for Example 1 in Scheme 1. The crude product was dissolved in DMF and purified via preparative HPLC (Condition N). Fractions containing the desired product were combined and dried under vacuum to afford Example 57. LCMS: m/z=351.1 [M+H]⁺; HPLC Ret. Time 1.665 min. (HPLC Method C); ¹H NMR (400 MHz, DMSO-d₆) δ ppm 9.38 (s, 1H), 8.60-8.51 (m, 1H), 8.46-8.40 (m, 1H), 8.06 (d, J=1.0 Hz, 1H), 7.80-7.40 (m, 5H), 4.59 (m, 2H), 1.61-1.45 (m, 3H).

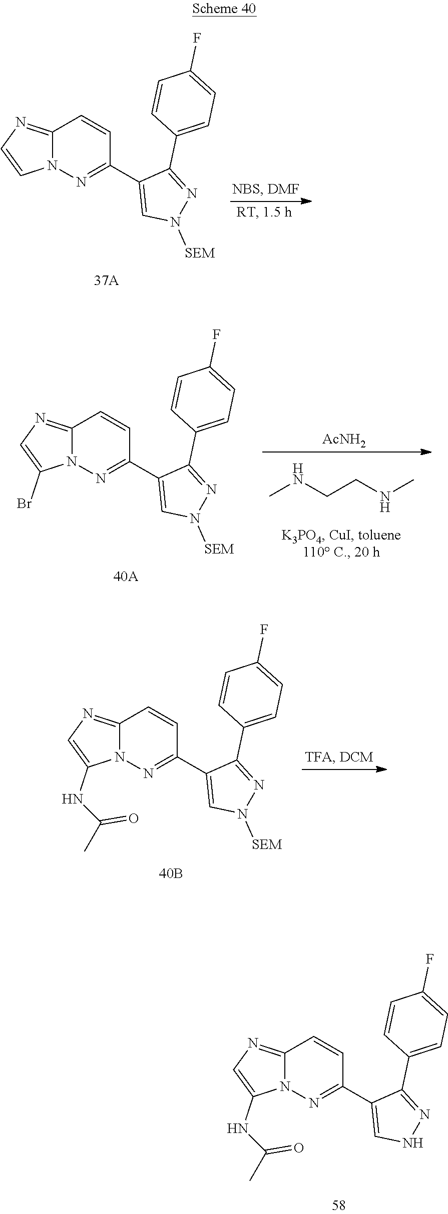

### Intermediate 40A: 3-bromo-6-(3-(4-fluorophenyl)-1-((2-(trimethylsilyl)ethoxy)methyl)-1H-pyrazol-4-yl)imidazo[1,2-b]pyridazine

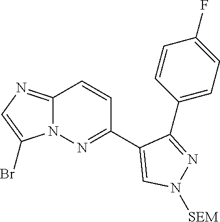 [SMILES: C[Si](C)(C)CCOCn1cc(-c2ccc3ncc(Br)n3n2)c(-c2ccc(F)cc2)n1]

Intermediate 40A was synthesized by reacting intermediate 37A with NBS and by employing the experimental procedure described for intermediate 1D in Scheme 1 to obtain intermediate 40A. LCMS: m/z=490.4 [M+H]⁺; HPLC Ret. Time 1.76 min. (HPLC Method B).

### Intermediate 40B: N-(6-(3-(4-fluorophenyl)-1-((2-(trimethylsilyl)ethoxy)methyl)-1H-pyrazol-4-yl)imidazo[1,2-b]pyridazin-3-yl)acetamide

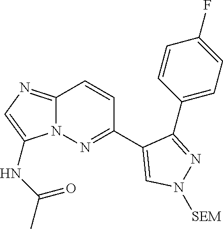 [SMILES: CC(=O)Nc1cnc2ccc(-c3cn(COCC[Si](C)(C)C)nc3-c3ccc(F)cc3)nn12]

To a stirred solution of 3-bromo-6-(3-(4-fluorophenyl)-1-((2-(trimethylsilyl)ethoxy)methyl)-1H-pyrazol-4-yl)imidazo[1,2-b]pyridazine (0.06 g, 0.123 mmol) in toluene (4 mL) was added acetamide (0.015 g, 0.246 mmol) and K₃PO₄ (0.052 g, 0.246 mmol). The reaction mixture was purged with argon for 10 min. and copper (I) iodide (2.34 mg, 0.012 mmol), followed by N,N-dimethylethylenediamine (3.25 mg, 0.037 mmol) were added. The resultant mixture was degassed for another 5 min. and then heated at 90° C. for 20 h. The reaction mixture was concentrated under high vacuum to get a brown gum that was washed with petroleum ether (2×5 mL) to get intermediate 40B (0.052 g, 55.3% yield) as a brown gum. LCMS: m/z=467.5 [M+H]⁺; HPLC Ret. Time 1.41 min. (HPLC Method B).

### Example 58: N-(6-(3-(4-fluorophenyl)-1H-pyrazol-4-yl)imidazo[1,2-b]pyridazin-3-yl)acetamide

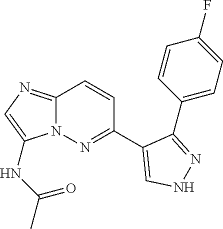 [SMILES: CC(=O)Nc1cnc2ccc(-c3c[nH]nc3-c3ccc(F)cc3)nn12]

Example 58 was synthesized by reacting intermediate 40B with TFA and by employing the experimental procedure described for Example 1 in Scheme 1. The crude product was dissolved in DMF and purified via preparative HPLC (Condition N). Fractions containing the desired product were combined and dried under vacuum to afford Example 58. LCMS: m/z=337.1 [M+H]⁺; HPLC Ret. Time 1.131 min. (HPLC

Method C); ¹H NMR (400 MHz, DMSO-d₆) δ ppm 10.23 (d, J=11.2 Hz, 1H), 8.70-8.64 (m, 1H), 8.39 (br. s., 1H), 8.30-8.23 (m, 1H), 8.02-7.96 (m, 2H), 7.90 (br. s., 1H), 7.58 (br. s., 1H), 7.50-7.37 (m, 1H), 2.35-2.28 (s, 3H).

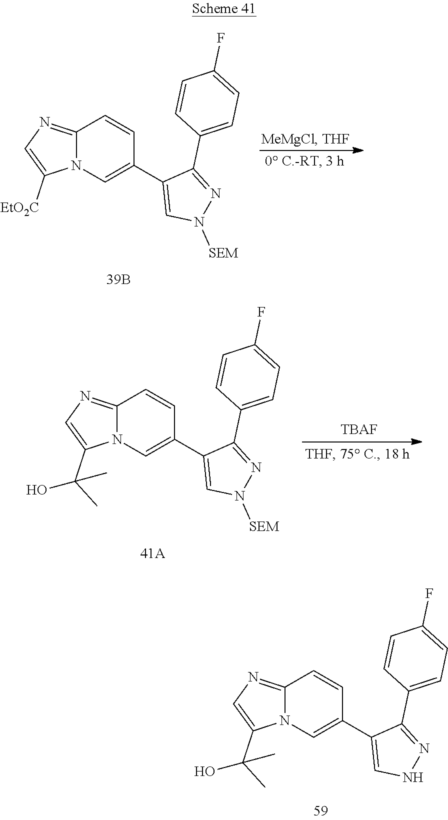

### Intermediate 41A: 2-(6-(3-(4-fluorophenyl)-1-((2-(trimethylsilyl)ethoxy)methyl)-1H-pyrazol-4-yl)imidazo[1,2-a]pyridin-3-yl)propan-2-ol

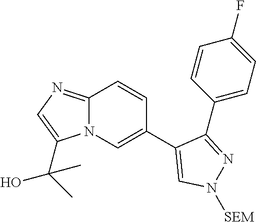 [SMILES: CC(C)(O)c1cnc2ccc(-c3cn(COCC[Si](C)(C)C)nc3-c3ccc(F)cc3)cn12]

Intermediate 41A was synthesized by reacting intermediate 39B with SEM-Cl and by employing the experimental procedure described for Example 52 in Scheme 34 to get intermediate 41A. LCMS: m/z=467.2 [M+H]⁺; HPLC Ret. Time 1.06 min. (HPLC Method A).

### Example 59: 2-(6-(3-(4-fluorophenyl)-1H-pyrazol-4-yl)imidazo[1,2-a]pyridin-3-yl)propan-2-ol

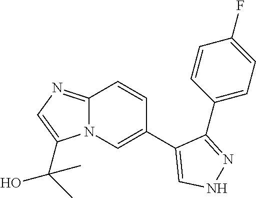 [SMILES: CC(C)(O)c1cnc2ccc(-c3c[nH]nc3-c3ccc(F)cc3)cn12]

To a solution of 41A (0.025 g, 0.054 mmol) was added TBAF (1.0 mL, 1.0 mmol, 1M in THF). The reaction mixture was heated at 75° C. for 18 h. The volatiles were then evaporated under reduced pressure to give a residue that was suspended in water (10 mL) and extracted with DCM (3×20 mL). The combined organic layers were dried over anhydrous sodium sulfate, filtered and the filtrate evaporated under reduced pressure to give the crude residue. The crude product was dissolved in DMF and purified via preparative HPLC (Condition N). Fractions containing the desired product were combined and dried under vacuum to afford Example 59 (3.9 mg, 21.64% yield). LCMS: m/z=337.1 [M+H]⁺; HPLC Ret. Time 1.352 min. (HPLC Method C); ¹H NMR (400 MHz, DMSO-d₆) δ ppm 13.40-13.14 (m, 1H), 8.59-8.48 (m, 1H), 8.08 (br. s., 1H), 7.58-7.45 (m, 3H), 7.37 (s, 1H), 7.15 (d, J=9.3 Hz, 3H), 5.16 (s, 1H), 1.49 (s, 6H).

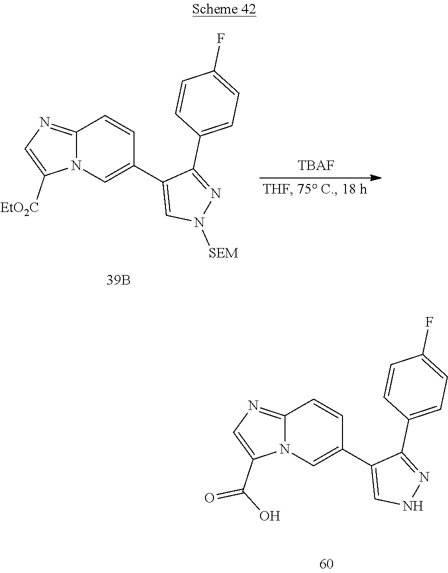

### Example 60: 6-(3-(4-fluorophenyl)-1H-pyrazol-4-yl)imidazo[1,2-a]pyridine-3-carboxylic acid

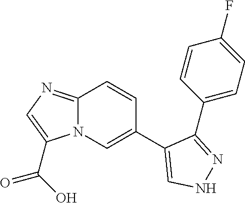 [SMILES: O=C(O)c1cnc2ccc(-c3c[nH]nc3-c3ccc(F)cc3)cn12]

A solution of 39B (0.1 g, 0.208 mmol) in TBAF in THF (1.5 mL, 1.5 mmol, 1.0 M solution in THF) was heated at 75° C. for 18 h. Then the reaction was quenched with ice cold water (10 mL) and extracted with DCM (3×20 mL). The combined organic layers were dried over anhydrous sodium sulfate, filtered and the filtrate was evaporated under reduced pressure to give the crude residue. The crude product was dissolved in DMF and purified via preparative HPLC (Condition N). Fractions containing the desired product were combined and dried under vacuum to afford Example 60 (2.3 mg, 11.50% yield). LCMS: m/z=323.1 [M+H]⁺; HPLC Ret. Time 0.817 min. (HPLC Method C); ¹H NMR (400 MHz, DMSO-d₆) δ ppm 13.22 (br, 2H), 9.18 (s, 1H), 8.21 (s, 1H), 8.14 (br. s., 1H), 7.77 (d, J=1.0 Hz, 1H), 7.54-7.45 (m, 2H), 7.41 (dd, J=9.0, 1.5 Hz, 1H), 7.20 (br. s., 2H).

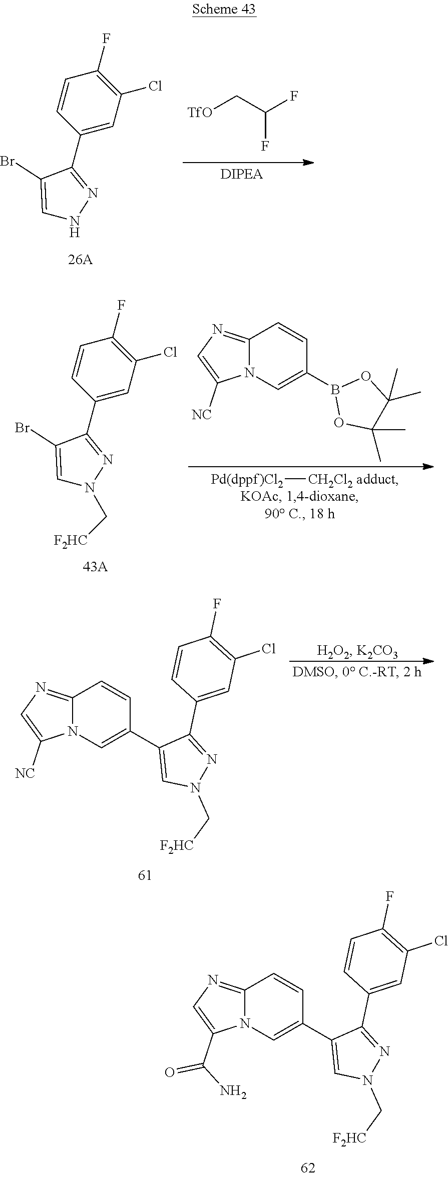

### Intermediate 43A: 4-bromo-3-(3-chloro-4-fluorophenyl)-1-(2,2-difluoroethyl)-1H-pyrazole

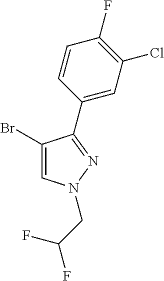 [SMILES: Fc1ccc(-c2nn(CC(F)F)cc2Br)cc1Cl]

Intermediate 43A was synthesized by reacting intermediate 26A with 2,2-difluoroethyl trifluoromethanesulfonate and by employing the experimental procedure described for Example 5B in Scheme 5. The crude product was dissolved in DMF and purified via preparative HPLC (Condition L). Fractions containing the desired product were combined and dried under vacuum to afford intermediate 43A. LCMS: m/z=340.7 [M+2]⁺; HPLC Ret. Time 3.846 min. (HPLC Method C).

### Example 61: 6-(3-(3-chloro-4-fluorophenyl)-1-(2,2-difluoroethyl)-1H-pyrazol-4-yl)imidazo[1,2-a]pyridine-3-carbonitrile

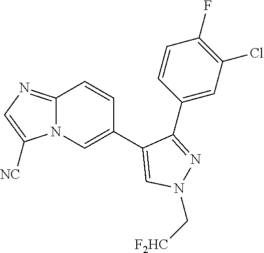 [SMILES: N#Cc1cnc2ccc(-c3cn(CC(F)F)nc3-c3ccc(F)c(Cl)c3)cn12]

Example 61 was synthesized by reacting intermediate 43A with 6-(4,4,5,5-tetramethyl-1,3,2-dioxaborolan-2-yl)imidazo[1,2-a]pyridine-3-carbonitrile and by employing the experimental procedure described for intermediate 26C in Scheme 26. The crude product was dissolved in DMF and purified via preparative HPLC (Condition N). Fractions containing the desired product were combined and dried under vacuum to afford Example 61. LCMS: m/z=402.0 [M+H]⁺; HPLC Ret. Time 2.068 min. (HPLC Method C); ¹H NMR (400 MHz, DMSO-d₆) δ ppm 8.57 (s, 1H), 8.47 (s, 1H), 8.30 (s, 1H), 7.82 (d, J=9.3 Hz, 1H), 7.68 (d, J=6.6 Hz, 1H), 7.44-7.39 (m, 2H), 7.34 (dd, J=9.3, 1.7 Hz, 1H), 6.67-6.35 (m, 1H), 4.76 (td, J=15.2, 3.7 Hz, 2H).

### Example 62: 6-(3-(3-chloro-4-fluorophenyl)-1-(2,2-difluoroethyl)-1H-pyrazol-4-yl)imidazo[1,2-a]pyridine-3-carboxamide

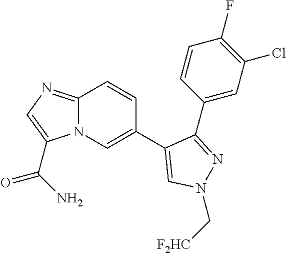 [SMILES: NC(=O)c1cnc2ccc(-c3cn(CC(F)F)nc3-c3ccc(F)c(Cl)c3)cn12]

Example 62 was synthesized by reacting Example 61 with H₂O₂/K₂CO₃ and by employing the experimental procedure described for Example 2 in Scheme 1. The crude product was purified via preparative HPLC (Condition N). Fractions containing the desired product were combined and dried under vacuum to afford Example 62. LCMS: m/z=419.9 [M+H]⁺; HPLC Ret. Time 1.720 min. (HPLC Method C); ¹H NMR (400 MHz, DMSO-d₆) δ ppm 9.50 (s, 1H), 8.38 (s, 1H), 8.22 (s, 1H), 8.00 (br. s., 1H), 7.76-7.64 (m, 2H), 7.51-7.36 (m, 3H), 7.31 (dd, J=9.2, 1.6 Hz, 1H), 6.67-6.34 (m, 1H), 4.76 (td, J=14.9, 3.7 Hz, 2H).

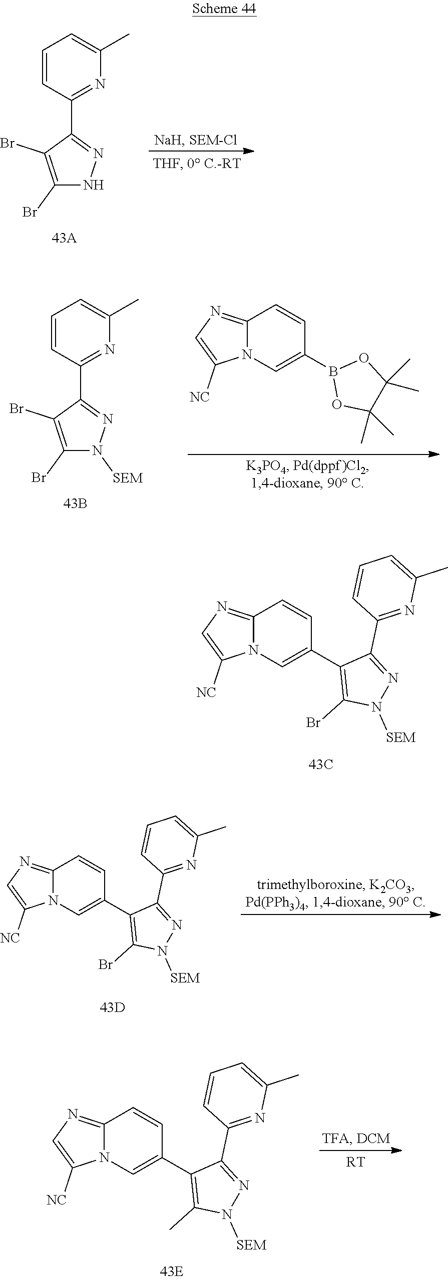

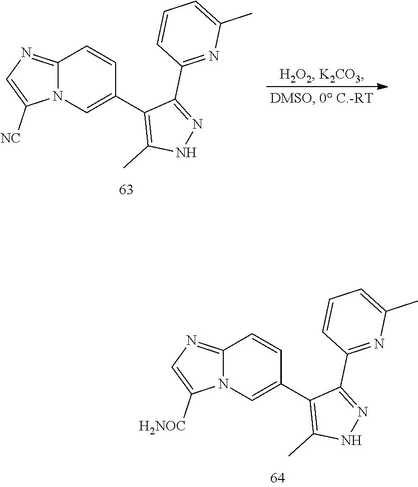 [SMILES: Cc1cccc(-c2n[nH]c(C)c2-c2ccc3ncc(C#N)n3c2)n1 ; Cc1cccc(-c2n[nH]c(C)c2-c2ccc3ncc(C(N)=O)n3c2)n1]

### Intermediate 43B: 2-(4,5-dibromo-1-((2-(trimethylsilyl)ethoxy)methyl)-1H-pyrazol-3-yl)-6-methylpyridine

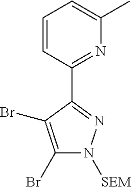 [SMILES: Cc1cccc(-c2nn(COCC[Si](C)(C)C)c(Br)c2Br)n1]

Intermediate 43B was synthesized by reacting intermediate 43A (Reference: WO 9429300 A1 19941222,) with SEM-Cl and by employing the experimental procedure described for intermediate 8A in Scheme 8 to afford intermediate 43B. LCMS: m/z=448.0 [M+H]⁺; Ret. Time 4.017 min. (LCMS Method E).

### Intermediate 43C: 6-(5-bromo-3-(6-methylpyridin-2-yl)-1-((2-(trimethylsilyl)ethoxy)methyl)-1H-pyrazol-4-yl)imidazo[1,2-a]pyridine-3-carbonitrile

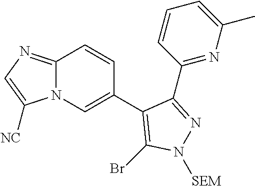 [SMILES: Cc1cccc(-c2nn(COCC[Si](C)(C)C)c(Br)c2-c2ccc3ncc(C#N)n3c2)n1]

Intermediate 43C was synthesized by reacting intermediate 43B with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (24 g RediSep® column, eluting with a gradient of 40-100% EtOAc in petroleum ether). Fractions containing the desired product were combined and evaporated under reduced pressure to afford intermediate 43C. LCMS: m/z=509.2 [M+H]⁺; Ret. Time 3.61 min. (LCMS Method E).

### Intermediate 43D: 6-(5-methyl-3-(6-methylpyridin-2-yl)-1-((2-(trimethylsilyl)ethoxy)methyl)-1H-pyrazol-4-yl)imidazo[1,2-a]pyridine-3-carbonitrile

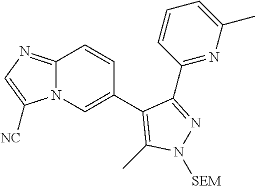 [SMILES: Cc1cccc(-c2nn(COCC[Si](C)(C)C)c(C)c2-c2ccc3ncc(C#N)n3c2)n1]

Intermediate 43D was synthesized by reacting intermediate 43C with trimethylboroxine and by employing the experimental procedure described for Example 34A in Scheme 34 to afford 43D. The crude compound was used for the next reaction without further purification. LCMS: m/z=445.6 [M+H]⁺; Ret. Time 1.53 min. (LCMS Method B).

### Example 63: 6-(5-methyl-3-(6-methylpyridin-2-yl)-1H-pyrazol-4-yl)imidazo[1,2-a]pyridine-3-carbonitrile

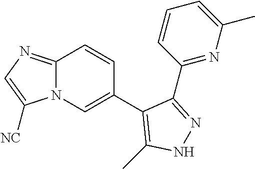 [SMILES: Cc1cccc(-c2n[nH]c(C)c2-c2ccc3ncc(C#N)n3c2)n1]

Example 63 was synthesized by reacting intermediate 43D with TFA and by employing the experimental procedure described for Example 1 in Scheme 1. The crude product was dissolved in DMF and purified via preparative HPLC (Condition N). Fractions containing the desired product were combined and dried under vacuum to afford Example 63. LCMS: m/z=315.1 [M+H]⁺; Ret. Time 1.361 min. (LCMS Method C). ¹HNMR (400 MHz, DMSO-d₆) δ ppm 8.70 (s, 1H), 8.45 (s, 1H), 7.80 (d, J=9.2 Hz, 1H), 7.68 (s, 1H), 7.53 (dd, J=9.3, 1.5 Hz, 1H), 7.14 (d, J=7.8 Hz, 1H), 3.17 (d, J=4.2 Hz, 2H), 2.26-2.38 (m, 6H).

### Example 64: 6-(5-methyl-3-(6-methylpyridin-2-yl)-1H-pyrazol-4-yl)imidazo[1,2-a]pyridine-3-carboxamide

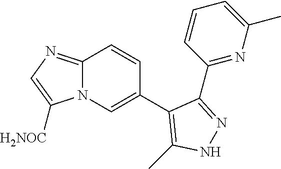 [SMILES: Cc1cccc(-c2n[nH]c(C)c2-c2ccc3ncc(C(N)=O)n3c2)n1]

Example 64 was synthesized by reacting Example 63 with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 64. LCMS: m/z=333.1 [M+H]⁺; Ret. Time 1.065 min. (LCMS Method C). ¹H NMR (400 MHz, DMSO-d₆) δ ppm 9.48 (s, 1H), 8.35 (bs, 1H), 7.90 (bs, 1H), 7.52-7.78 (m, 3H), 7.35 (d, J=9.0 Hz, 2H), 7.07 (d, J=7.4 Hz, 2H), 2.35, (s, 3H), 2.18 (s, 3H).

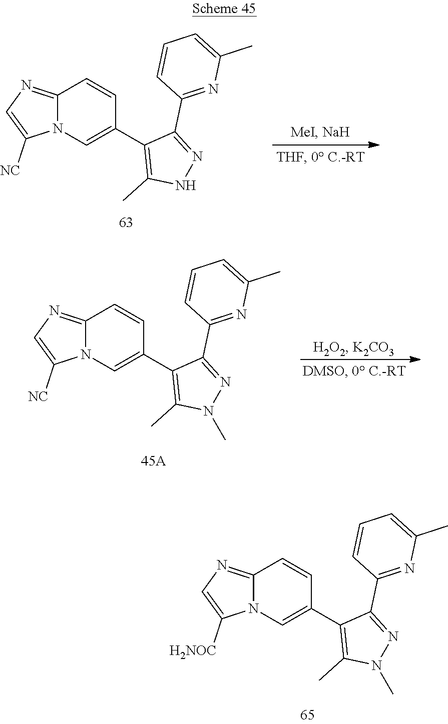 [SMILES: Cc1cccc(-c2n[nH]c(C)c2-c2ccc3ncc(C#N)n3c2)n1 ; Cc1cccc(-c2nn(C)c(C)c2-c2ccc3ncc(C#N)n3c2)n1 ; Cc1cccc(-c2nn(C)c(C)c2-c2ccc3ncc(C(N)=O)n3c2)n1]

### Intermediate 45A: 6-(1,5-dimethyl-3-(6-methylpyridin-2-yl)-1H-pyrazol-4-yl)imidazo[1,2-a]pyridine-3-carbonitrile

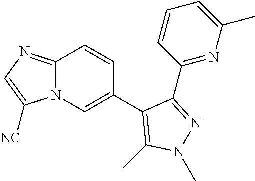 [SMILES: Cc1cccc(-c2nn(C)c(C)c2-c2ccc3ncc(C#N)n3c2)n1]

Intermediate 45A was synthesized by reacting Example 63 with methyl iodide and by employing the experimental procedure described for Example 6B in Scheme 6 to obtain intermediate 45A. LCMS: m/z=329.2 [M+H]⁺; Ret. Time 0.78 min. (LCMS Method B).

### Example 65: 6-(1,5-dimethyl-3-(6-methylpyridin-2-yl)-1H-pyrazol-4-yl)imidazo[1,2-a]pyridine-3-carboxamide

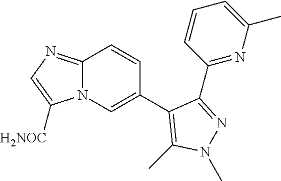 [SMILES: Cc1cccc(-c2nn(C)c(C)c2-c2ccc3ncc(C(N)=O)n3c2)n1]

Example 65 was synthesized by reacting intermediate 45A with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 65. LCMS: m/z=347.2 [M+H]⁺; Ret. Time 1.157 min. (LCMS Method C). ¹H NMR (400 MHz, DMSO-d₆) δ ppm 9.40-9.50 (m, 1H), 8.26-8.37 (m, 1H), 7.91 (bs, 1H), 7.52-7.70 (m, 3H), 7.27-7.44 (m, 2H), 6.98-7.14 (m, 1H), 3.90 (s, 3H), 2.28 (s, 3H), 2.18 (s, 3H).

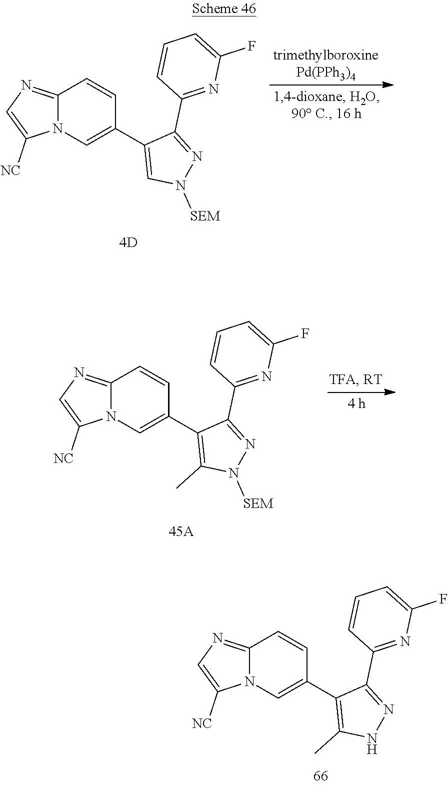 [SMILES: C[Si](C)(C)CCOCn1cc(-c2ccc3ncc(C#N)n3c2)c(-c2cccc(F)n2)n1 ; Cc1c(-c2ccc3ncc(C#N)n3c2)c(-c2cccc(F)n2)nn1COCC[Si](C)(C)C ; Cc1[nH]nc(-c2cccc(F)n2)c1-c1ccc2ncc(C#N)n2c1]

### Intermediate 45A: 6-(3-(6-fluoropyridin-2-yl)-5-methyl-1-((2-(trimethylsilyl)ethoxy)methyl)-1H-pyrazol-4-yl)imidazo [1,2-a]pyridine-3-carbonitrile

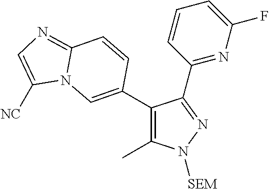 [SMILES: Cc1c(-c2ccc3ncc(C#N)n3c2)c(-c2cccc(F)n2)nn1COCC[Si](C)(C)C]

Intermediate 45A was synthesized by reacting intermediate 4D with trimethylboroxine and by employing the experimental procedure described for Example 34A in Scheme 34. The crude product was purified by silica gel chromatography (24 g RediSep column, eluting with a gradient of 30-50% EtOAc in petroleum ether). Fractions containing the desired product were combined and evaporated to afford intermediate 45A. MS (ES): m/z=449.1 [M+H]⁺; HPLC Ret. Time 3.54 min. (HPLC Method E).

### Example 66: 6-(3-(6-fluoropyridin-2-yl)-5-methyl-1H-pyrazol-4-yl)imidazo [1,2-a]pyridine-3-carbonitrile

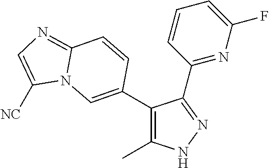 [SMILES: Cc1[nH]nc(-c2cccc(F)n2)c1-c1ccc2ncc(C#N)n2c1]

Example 66 was synthesized by reacting intermediate 45A with TFA and by employing the experimental procedure described for Example 1 in Scheme 1. The crude product was dissolved in DMF and purified via preparative HPLC (Condition N). Fractions containing the desired product were combined and dried under vacuum to afford Example 66. MS (ES): m/z=319.1 [M+H]⁺; HPLC Ret. Time 1.34 min. (HPLC Method C); ¹H NMR (400 MHz, DMSO-d₆) δ ppm 8.47-8.61 (m, 2H), 7.94-8.03 (m, 1H), 7.81-7.89 (m, 2H), 7.48 (d, J=9.2 Hz, 1H), 2.27 (s, 3H).

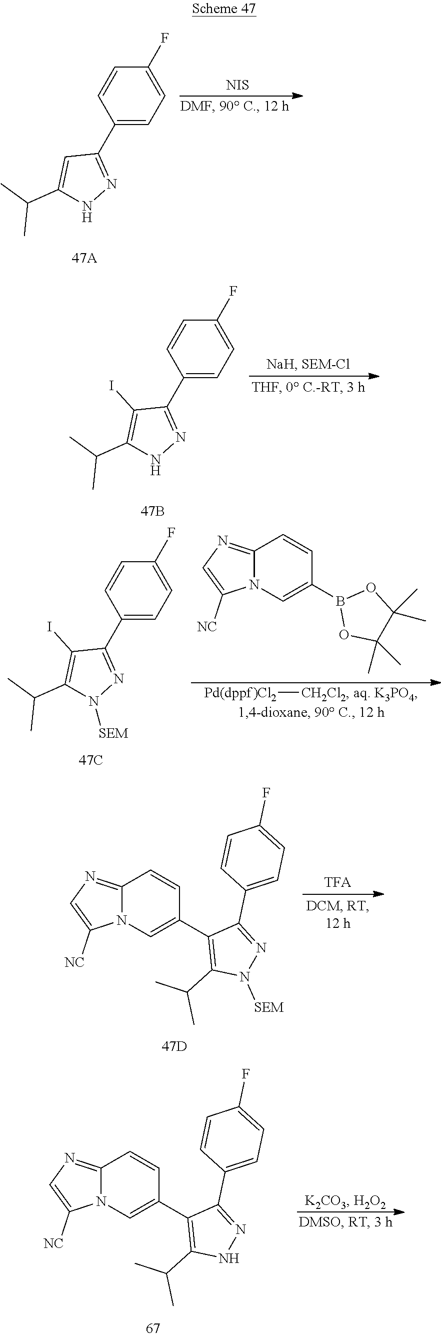

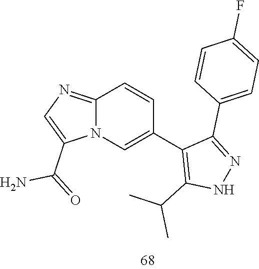 [SMILES: CC(C)c1[nH]nc(-c2ccc(F)cc2)c1-c1ccc2ncc(C(N)=O)n2c1]

### Intermediate 47B: 3-(4-fluorophenyl)-4-iodo-5-isopropyl-1H-pyrazole

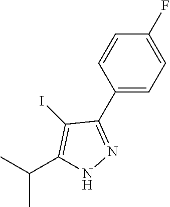 [SMILES: CC(C)c1[nH]nc(-c2ccc(F)cc2)c1I]

To an ice-cold solution of 47A (Reference: U.S. Pat. Appl. Publ. (2013), US 20130143870 A1 20130606; 1.8 g, 8.81 mmol) in dry DMF (15 mL) was added NIS (2.379 g, 10.58 mmol). The resulting orange solution was heated at 90° C. for 12 h. The solution was partitioned between DCM and saturated aq. NaHCO₃ solution. The organic layer was washed twice with saturated aq. NaHCO₃, water, brine, and dried over Na₂SO₄, filtered and the filtrate evaporated under reduced pressure to get intermediate 47B (2 g, 53.6% yield) as a liquid. The crude compound was used for the next reaction without further purification. LCMS: m/z=331.0 [M+H]⁺; HPLC Ret. Time 2.889 min. (HPLC Method E).

### Intermediate 47C: 3-(4-fluorophenyl)-4-iodo-5-isopropyl-1-((2-(trimethylsilyl)ethoxy)methyl)-1H-pyrazole

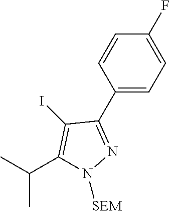 [SMILES: CC(C)c1c(I)c(-c2ccc(F)cc2)nn1COCC[Si](C)(C)C]

Intermediate 47C was synthesized by reacting intermediate 47B with SEM-Cl and by employing the experimental procedure described for intermediate 8A in Scheme 8. The crude product was purified by silica gel chromatography (40 g RediSep® column, eluting with a gradient of 5-8% EtOAc in petroleum ether). Fractions containing the desired product were combined and evaporated under reduced pressure to afford intermediate 47C. LCMS: m/z=461.0 [M+H]⁺; HPLC Ret. Time 4.513 min. (HPLC Method E).

### Intermediate 47D: 6-(3-(4-fluorophenyl)-5-isopropyl-1-((2-(trimethylsilyl)ethoxy)methyl)-1H-pyrazol-4-yl)imidazo[1,2-a]pyridine-3-carbonitrile

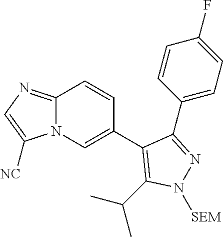 [SMILES: CC(C)c1c(-c2ccc3ncc(C#N)n3c2)c(-c2ccc(F)cc2)nn1COCC[Si](C)(C)C]

Intermediate 47D was synthesized by reacting intermediate 47C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated to afford intermediate 47D. LCMS: m/z=476.2 [M+H]⁺; HPLC Ret. Time 3.834 min. (HPLC Method E).

### Example 67: 6-(3-(4-fluorophenyl)-5-isopropyl-1H-pyrazol-4-yl)imidazo[1,2-a]pyridine-3-carbonitrile

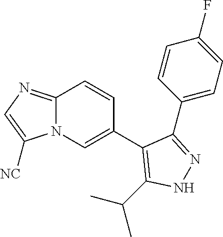 [SMILES: CC(C)c1[nH]nc(-c2ccc(F)cc2)c1-c1ccc2ncc(C#N)n2c1]

Example 67 was synthesized by reacting intermediate 47D with TFA and by employing the experimental procedure described for Example 1 in Scheme 1. The crude product was dissolved in DMF and purified via preparative HPLC (Condition L). Fractions containing the desired product were combined and dried under vacuum to afford Example 67. LCMS: m/z=346.1 [M+H]⁺; HPLC Ret. Time 1.505 min. (HPLC Method C); ¹H NMR (400 MHz, DMSO-d₆) δ ppm 13.05 (s, 1H), 8.46 (s, 1H), 8.41-8.35 (m, 1H), 7.83 (d, J=9.3 Hz, 1H), 7.41 (dd, J=8.6, 5.6 Hz, 2H), 7.29 (d, J=9.3 Hz, 1H), 7.23-7.16 (m, 1H), 7.11 (t, J=8.8 Hz, 1H), 3.07-2.97 (m, 1H), 1.24 (d, J=6.8 Hz, 6H).

### Example 68: 6-(3-(4-fluorophenyl)-5-isopropyl-1H-pyrazol-4-yl) imidazo [1,2-a]pyridine-3-carboxamide

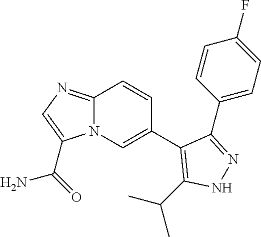 [SMILES: CC(C)c1[nH]nc(-c2ccc(F)cc2)c1-c1ccc2ncc(C(N)=O)n2c1]

Example 68 was synthesized by reacting Example 67 with H₂O₂/K₂CO₃ and by employing the experimental procedure described for Example 2 in Scheme 1. The crude product was dissolved in DMF and purified via preparative HPLC (Condition H). Fractions containing the desired product were combined and dried under vacuum to afford Example 68. LCMS: m/z=364.1 [M+H]⁺; HPLC Ret. Time 1.229 min. (HPLC Method C). ¹H NMR (400 MHz, DMSO-d₆) δ ppm 9.39 (s, 1H), 8.35 (s, 1H), 7.95 (bs, 1H), 7.70 (d, J=9.3 Hz, 1H), 7.43-7.33 (m, 3H), 7.19 (d, J=9.0 Hz, 2H), 7.10 (t, J=8.9 Hz, 2H), 2.99-2.89 (m, 1H), 1.28-1.15 (m, 6H).

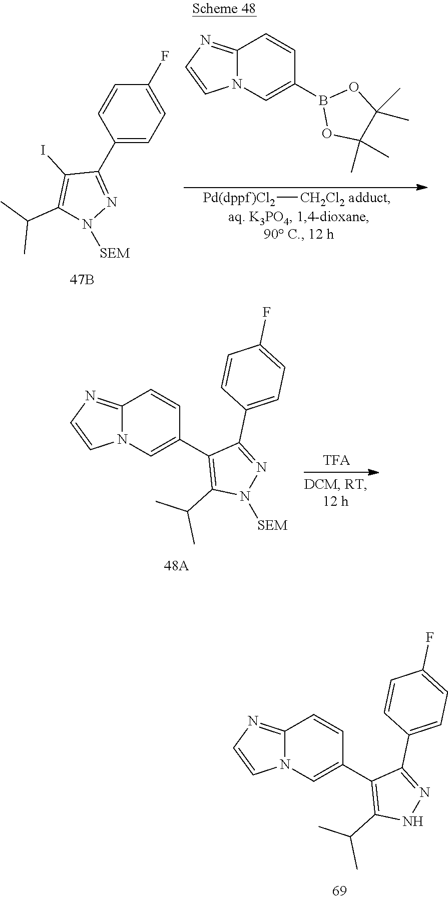

### Intermediate 48A: 6-(3-(4-fluorophenyl)-5-isopropyl-1-((2-(trimethylsilyl)ethoxy) methyl)-1H-pyrazol-4-yl)imidazo[1,2-a]pyridine

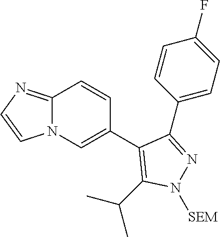 [SMILES: CC(C)c1c(-c2ccc3nccn3c2)c(-c2ccc(F)cc2)nn1COCC[Si](C)(C)C]

Intermediate 48A was synthesized by reacting intermediate 47B with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (24 g RediSep® column, eluting with a gradient of 20-30% EtOAc in petroleum ether). Fractions containing the desired product were combined and evaporated to afford intermediate 48A as a gum. LCMS: m/z=451.2 [M+H]⁺; HPLC Ret. Time 3.842 min. (HPLC Method E).

### Example 69: 6-(3-(4-fluorophenyl)-5-isopropyl-1H-pyrazol-4-yl)imidazo [1,2-a]pyridine

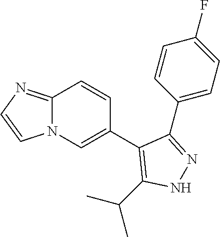 [SMILES: CC(C)c1[nH]nc(-c2ccc(F)cc2)c1-c1ccc2nccn2c1]

Example 69 was synthesized by reacting intermediate 48A with TFA and by employing the experimental procedure described for Example 1 in Scheme 1. The crude product was dissolved in DMF and purified via preparative HPLC (Condition H). Fractions containing the desired product were combined and dried under vacuum to afford Example 69. LCMS: m/z=321.1 [M+H]⁺; HPLC Ret. Time 1.427 min. (HPLC Method C), ¹H NMR (400 MHz, DMSO-d₆) δ ppm 12.98 (br. s., 1H), 8.49 (s, 1H), 7.98 (s, 1H), 7.66 (d, J=1.0 Hz, 1H), 7.60 (d, J=9.3 Hz, 1H), 7.41 (br. s., 2H), 7.21-7.02 (m, 3H), 3.00-2.91 (m, 1H), 1.23 (d, J=5.4 Hz, 6H).

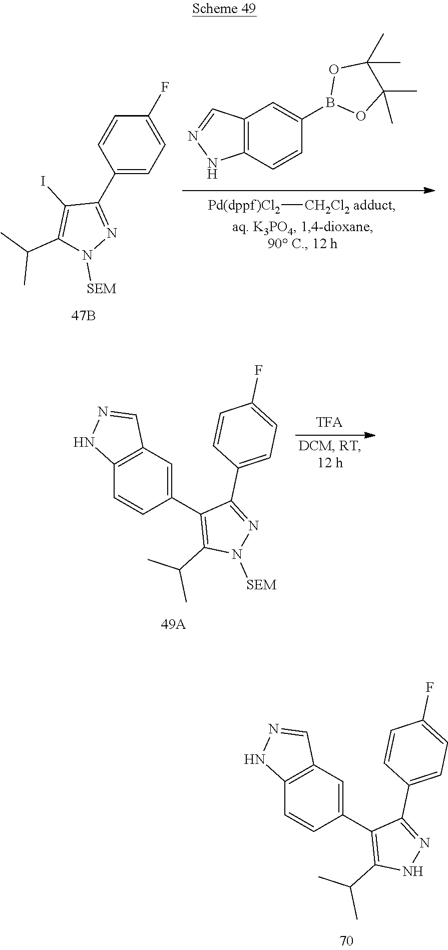

### Intermediate 49A: 5-(3-(4-fluorophenyl)-5-isopropyl-1-((2-(trimethylsilyl)ethoxy) methyl)-1H-pyrazol-4-yl)-1H-indazole

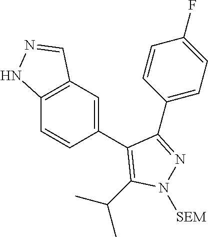 [SMILES: CC(C)c1c(-c2ccc3[nH]ncc3c2)c(-c2ccc(F)cc2)nn1COCC[Si](C)(C)C]

Intermediate 49A was synthesized by reacting intermediate 47B with 5-(4,4,5,5-tetramethyl-1,3,2-dioxaborolan-2-yl)-1H-indazole and by employing the experimental procedure described for intermediate 1E in Scheme 1 to afford intermediate 49A was obtained as semi solid. LCMS: m/z=451.6 [M+H]⁺; HPLC Ret. Time 1.26 min. (HPLC Method A).

### Example 70: 5-(3-(4-fluorophenyl)-5-isopropyl-1H-pyrazol-4-yl)-1H-indazole

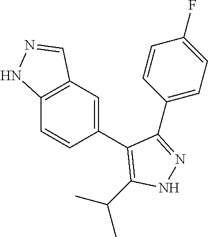 [SMILES: CC(C)c1[nH]nc(-c2ccc(F)cc2)c1-c1ccc2[nH]ncc2c1]

Example 70 was synthesized by reacting intermediate 49A with TFA and by employing the experimental procedure described for Example 1 in Scheme 1. The crude product was dissolved in DMF and purified via preparative HPLC (Condition L). Fractions containing the desired product were combined and dried under vacuum to afford Example 70. LCMS: m/z=321.2 [M+H]⁺; HPLC Ret. Time 1.617 min. (HPLC Method C); ¹H NMR (400 MHz, DMSO-d₆) δ ppm 13.11-13.02 (m, 1H), 12.88-12.76 (m, 1H), 8.07-7.99 (m, 1H), 7.59-7.48 (m, 2H), 7.37-7.26 (m, 2H), 7.14-6.98 (m, 3H), 2.97-2.84 (m, 1H), 1.25-1.07 (m, 6H).

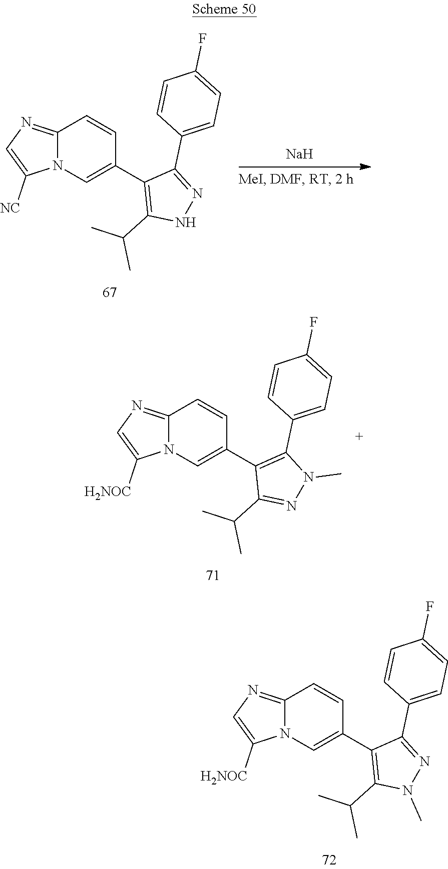

Example 71 and Example 72 were synthesized by reacting Example 67 with methyl iodide and by employing the experimental procedure described for Example 6B in Scheme 6. The crude product was dissolved in DMF and purified via preparative HPLC (Condition L). Fractions containing the desired product were combined and dried under vacuum to afford Example 71. LCMS: m/z=378.1 [M+H]⁺; HPLC Ret. Time 1.531 min. (HPLC Method C), ¹H NMR (400 MHz, DMSO-d₆) δ ppm 9.38 (s, 1H), 8.36 (s, 1H), 7.97 (br. s., 1H), 7.72 (d, J=9.0 Hz, 1H), 7.37-7.28 (m, 3H), 7.25 (d, J=9.0 Hz, 1H), 7.07 (t, J=8.8 Hz, 2H), 3.93 (s, 3H), 3.20-3.09 (m, 1H), 1.17 (d, J=7.1 Hz, 6H) and Example 72. LCMS: m/z=378.1 [M+H]⁺; HPLC Ret. Time 1.549 min. (HPLC Method C), ¹H NMR (400 MHz, DMSO-d₆) δ ppm 9.38 (s, 1H), 8.36 (s, 1H), 7.97 (bs, 1H), 7.72 (d, J=9.0 Hz, 1H), 7.37-7.28 (m, 3H), 7.25 (d, J=9.0 Hz, 1H), 7.07 (t, J=8.8 Hz, 2H), 3.93 (s, 3H), 3.20-3.09 (m, 1H), 1.17 (d, J=7.1 Hz, 6H).

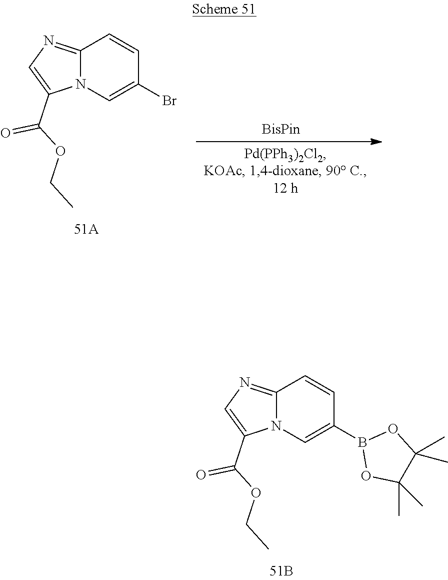 [SMILES: CCOC(=O)c1cnc2ccc(Br)cn12 ; CCOC(=O)c1cnc2ccc(B3OC(C)(C)C(C)(C)O3)cn12]

### Intermediate 51B: ethyl 6-(4,4,5,5-tetramethyl-1,3,2-dioxaborolan-2-yl)imidazo[1,2-a]pyridine-3-carboxylate

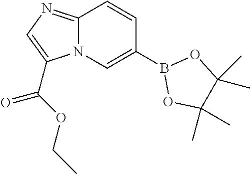 [SMILES: CCOC(=O)c1cnc2ccc(B3OC(C)(C)C(C)(C)O3)cn12]

To a solution of intermediate 51A (1.0 g, 3.72 mmol) in 1,4-dioxane (20 mL) was added 4,4,4′,4′,5,5,5′,5′-octamethyl-2,2′-bi(1,3,2-dioxaborolane) (1.416 g, 5.57 mmol). The reaction mixture was degassed with argon for 3 min., treated with Pd(PPh₃)₂Cl₂ (0.130 g, 0.186 mmol), and heated at 90° C. for 12 h. The mixture was cooled to RT and filtered through a Celite® pad. The filter-cake washed with ethyl acetate and the combined filtrate was evaporated under reduced pressure to afford intermediate 51B (1 g, 43.4% yield). LCMS: m/z=317.1 [M+1]⁺; HPLC Ret. Time 1.37 min. (HPLC Method B).

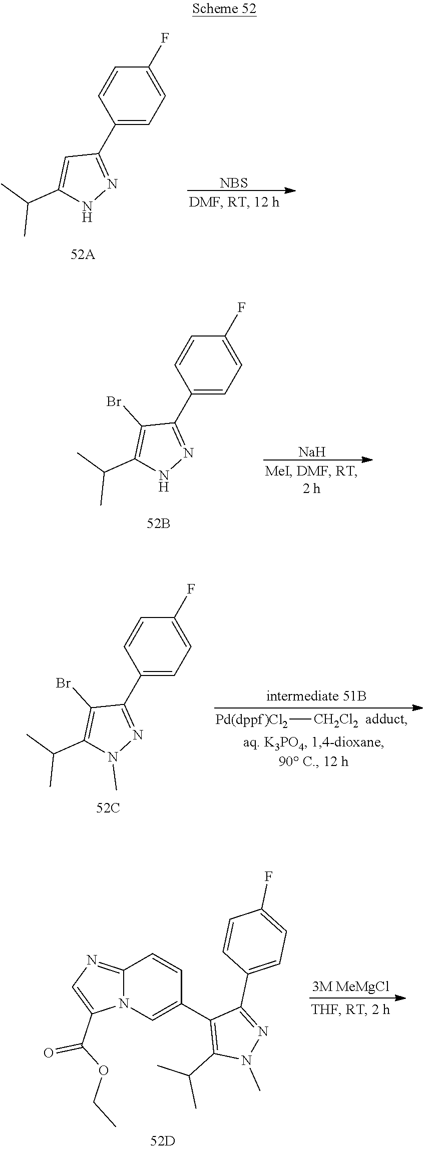

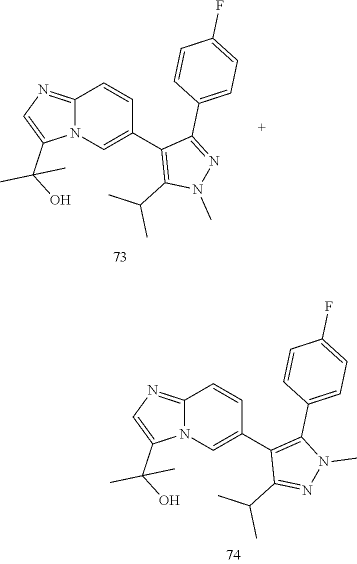 [SMILES: CC(C)c1c(-c2ccc3ncc(C(C)(C)O)n3c2)c(-c2ccc(F)cc2)nn1C ; CC(C)c1nn(C)c(-c2ccc(F)cc2)c1-c1ccc2ncc(C(C)(C)O)n2c1]

### Intermediate 52B: 4-bromo-3-(4-fluorophenyl)-5-isopropyl-1H-pyrazole

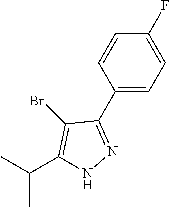 [SMILES: CC(C)c1[nH]nc(-c2ccc(F)cc2)c1Br]

Intermediate 52B was synthesized by reacting intermediate 52A with NBS and by employing the experimental procedure described for intermediate 1D in Scheme 1 to afford 52B as an off white solid. LCMS: m/z=285.0 [M+2]⁺; HPLC Ret. Time 2.808 min. (HPLC Method E).

### Intermediate 52C: 4-bromo-3-(4-fluorophenyl)-5-isopropyl-1-methyl-1H-pyrazole

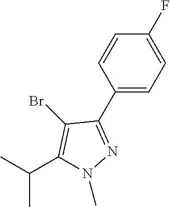 [SMILES: CC(C)c1c(Br)c(-c2ccc(F)cc2)nn1C]

Intermediate 52C was synthesized by reacting intermediate 52B with methyl iodide and by employing the experimental procedure described for Example 6B in Scheme 6 to obtain Intermediate 52C as a gum. The crude product was used in the next step without purification. LCMS: m/z=299.0 [M+2]⁺; HPLC Ret. Time 3.126 min. (HPLC Method E).

### Intermediate 52D: Ethyl 6-(3-(4-fluorophenyl)-5-isopropyl-1-methyl-1H-pyrazol-4-yl)imidazo[1,2-a]pyridine-3-carboxylate

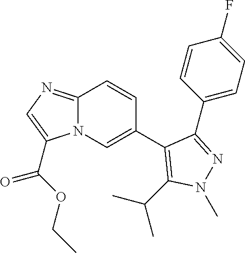 [SMILES: CCOC(=O)c1cnc2ccc(-c3c(-c4ccc(F)cc4)nn(C)c3C(C)C)cn12]

Intermediate 52D was synthesized by reacting intermediate 52C with intermediate 51B and by employing the experimental procedure described for Example 37 in Scheme 25. The crude product was purified by silica gel chromatography (40 g RediSep® column, eluting with a gradient of 20-30% EtOAc in petroleum ether). Fractions containing the desired product were combined and evaporated under reduced pressure to afford intermediate 52D. LCMS: m/z=407.3 [M+1]⁺; HPLC Ret. Time 1.35 min. (HPLC Method B).

### Example 73 and 74: 2-(6-(5-(4-fluorophenyl)-3-isopropyl-1-methyl-1H-pyrazol-4-yl)imidazo[1,2-a]pyridin-3-yl)propan-2-ol and 2-(6-(3-(4-fluorophenyl)-5-isopropyl-1-methyl-1H-pyrazol-4-yl)imidazo[1,2-a]pyridin-3-yl)propan-2-ol

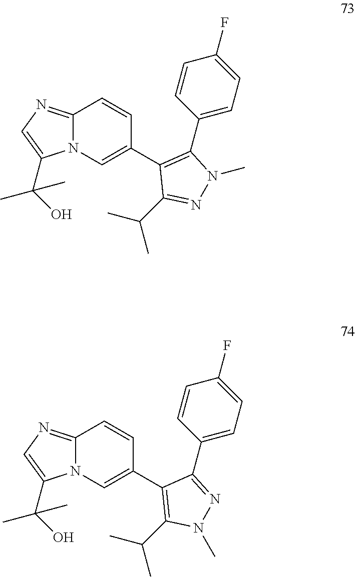

Example 73 and 74 were synthesized by reacting intermediate 52D with MeMgCl and by employing the experimental procedure described for Example 52 in Scheme 34. The crude product was dissolved in DMF and purified via preparative HPLC (Condition R). Fractions containing the desired product were combined and dried under vacuum to afford Example 73. (10 mg, 15.5%) LCMS: m/z=393.3 [M+1]⁺; HPLC Ret. Time 1.518 min. (HPLC Method C). ¹H NMR (400 MHz, DMSO-d₆) δ ppm 8.44 (s, 1H), 7.49 (d, J 9.3 Hz, 1H), 7.44-7.31 (m, 3H), 7.29-7.15 (m, 2H), 6.99 (dd, J=9.3, 1.5 Hz, 1H), 5.31 (s, 1H), 3.73 (s, 3H), 3.09-2.97 (m, 1H), 1.45 (s, 6H), 1.23 (d, J=6.8 Hz, 6H) and Example 74 (9.0 mg, 14.2%). LCMS: m/z=393.3 [M+1]⁺; HPLC Ret. Time 1.542 min. (HPLC Method C). ¹H NMR (400 MHz, DMSO-d₆) δ ppm 8.56 (s, 1H), 7.57 (d, J=9.0 Hz, 1H), 7.45-7.22 (m, 3H), 7.14-6.90 (m, 3H), 5.39 (bs, 1H), 3.93 (s, 3H), 3.18 (dt, J=14.2, 7.2 Hz, 1H), 1.54 (s, 6H), 1.21 (d, J=7.1 Hz, 6H).

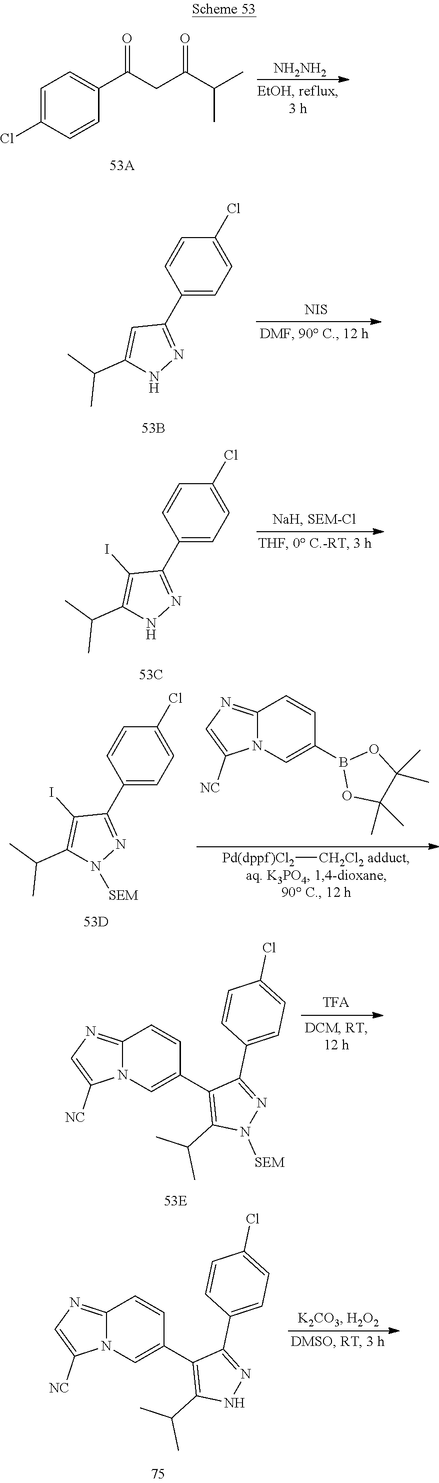

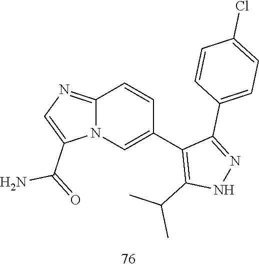 [SMILES: CC(C)c1[nH]nc(-c2ccc(Cl)cc2)c1-c1ccc2ncc(C(N)=O)n2c1]

### Intermediate 53B: 3-(4-chlorophenyl)-5-isopropyl-1H-pyrazole

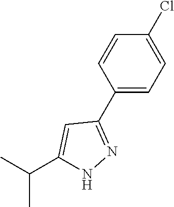

To a stirred solution of 53A (2.6 g, 11.57 mmol) in ethanol (10 mL) was added hydrazine hydrate (0.5 mL, 15.93 mmol) and the reaction mixture was reflux for 4 h. The mixture was cooled to RT and the volatiles were evaporated under reduced pressure to get a crude compound. The crude residue was dissolve in DCM and washed with water. The organic layer was dried over anhydrous Na₂SO₄, filtered and the filtrate was evaporated under reduced pressure to afford intermediate 53B (2.4 g, 82% yield) as a semi-solid. LCMS: m/z=221.2 [M+1]⁺; HPLC Ret. Time 2.71 min. (HPLC Method E).

### Intermediate 53C: 3-(4-chlorophenyl)-4-iodo-5-isopropyl-1H-pyrazole

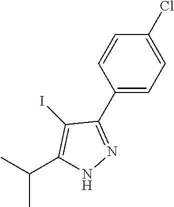 [SMILES: CC(C)c1[nH]nc(-c2ccc(Cl)cc2)c1I]

Intermediate 53C was synthesized by reacting intermediate 53B with NIS and by employing the experimental procedure described for intermediate 47B in Scheme to obtain intermediate 53C. LCMS: m/z=347.0 [M+1]⁺; HPLC Ret. Time 3.23 min. (HPLC Method E).

### Intermediate 53D: 3-(4-chlorophenyl)-4-iodo-5-isopropyl-1-((2-(trimethylsilyl)ethoxy)methyl)-1H-pyrazole

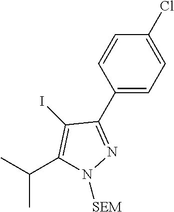 [SMILES: CC(C)c1c(I)c(-c2ccc(Cl)cc2)nn1COCC[Si](C)(C)C]

Intermediate 53D was synthesized by reacting intermediate 53C with SEM-Cl and by employing the experimental procedure described for intermediate 8A in Scheme 8. The crude product was purified by silica gel chromatography (40 g RediSep® column, eluting with a gradient of 5-8% EtOAc in petroleum ether). Fractions containing the desired product were combined and evaporated to afford intermediate 53D as a liquid. LCMS: m/z=477.2 [M+1]⁺; HPLC Ret. Time 4.59 min. (HPLC Method E).

### Intermediate 53E: 6-(3-(4-chlorophenyl)-5-isopropyl-1-((2-(trimethylsilyl)ethoxy)methyl)-1H-pyrazol-4-yl)imidazo[1,2-a]pyridine-3-carbonitrile

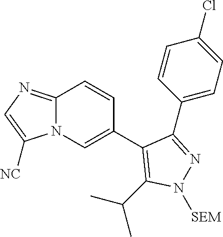 [SMILES: CC(C)c1c(-c2ccc3ncc(C#N)n3c2)c(-c2ccc(Cl)cc2)nn1COCC[Si](C)(C)C]

Intermediate 53E was synthesized by reacting intermediate 53D with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 53E as a gum. LCMS: m/z=492.2 [M+1]⁺; HPLC Ret. Time 4.006 min. (HPLC Method E).

### Example 75: 6-(3-(4-chlorophenyl)-5-isopropyl-1H-pyrazol-4-yl)imidazo[1,2-a]pyridine-3-carbonitrile

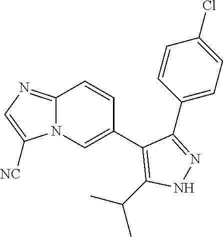 [SMILES: CC(C)c1[nH]nc(-c2ccc(Cl)cc2)c1-c1ccc2ncc(C#N)n2c1]

Example 75 was synthesized by reacting intermediate 53E with TFA and by employing the experimental procedure described for Example 1 in Scheme 1. The crude product was dissolved in DMF and purified via preparative HPLC (Condition L). Fractions containing the desired product were combined and dried under vacuum to afford Example 75. LCMS: m/z=362.1 [M+1]⁺; HPLC Ret. Time 1.863 min. (HPLC Method C). ¹H NMR (400 MHz, DMSO-d₆) δ ppm 13.11 (br. s., 1H), 8.46 (s, 1H), 8.40 (s, 1H), 7.84 (s, 1H), 7.42-7.26 (m, 5H), 3.06-2.98 (m, 1H), 1.23 (d, J=6.1 Hz, 6H).

### Example 76: 6-(3-(4-chlorophenyl)-5-isopropyl-1H-pyrazol-4-yl)imidazo[1,2-a]pyridine-3-carboxamide

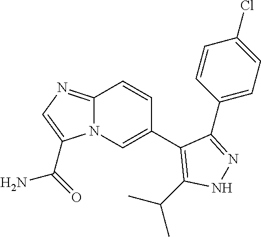 [SMILES: CC(C)c1[nH]nc(-c2ccc(Cl)cc2)c1-c1ccc2ncc(C(N)=O)n2c1]

Example 76 was synthesized by reacting Example 75 with H₂O₂/K₂CO₃ and by employing the experimental procedure described for Example 2 in Scheme 1. The crude product was dissolved in DMF and purified via preparative HPLC (Condition H). Fractions containing the desired product were combined and dried under vacuum to afford Example 76. LCMS: m/z=380.1 [M+1]⁺; HPLC Ret. Time 1.516 min. (HPLC Method C). ¹H NMR (400 MHz, DMSO-d₆) δ ppm 13.08 (br. s., 1H), 9.40 (s, 1H), 8.35 (s, 1H), 7.95 (bs, 1H), 7.72 (s, 1H), 7.41-7.26 (m, 5H), 7.20 (s, 1H), 3.00-2.85 (m, 1H), 1.23 (d, J=6.4 Hz, 6H).

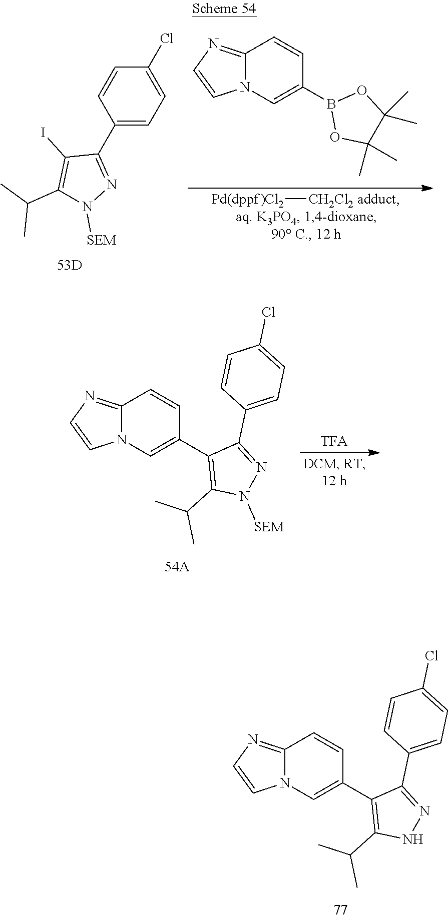

### Intermediate 54A: 6-(3-(4-chlorophenyl)-5-isopropyl-1-((2-(trimethylsilyl)ethoxy) methyl)-1H-pyrazol-4-yl)imidazo[1,2-a]pyridine

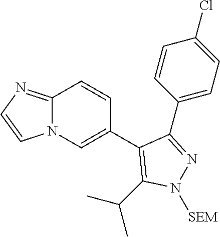 [SMILES: CC(C)c1c(-c2ccc3nccn3c2)c(-c2ccc(Cl)cc2)nn1COCC[Si](C)(C)C]

Intermediate 54A was synthesized by reacting intermediate 53D with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 54A as a gum. LCMS: m/z=467.2 [M+1]⁺; HPLC Ret. Time 4.192 min. (HPLC Method E).

### Example 77: 6-(3-(4-chlorophenyl)-5-isopropyl-1H-pyrazol-4-yl)imidazo[1,2-a]pyridine

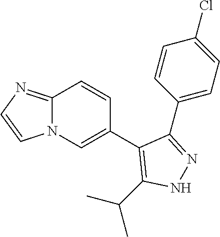 [SMILES: CC(C)c1[nH]nc(-c2ccc(Cl)cc2)c1-c1ccc2nccn2c1]

Example 77 was synthesized by reacting intermediate 54A with TFA and by employing the experimental procedure described for Example 1 in Scheme 1. The crude product was dissolved in DMF and purified via preparative HPLC (Condition H). Fractions containing the desired product were combined and dried under vacuum to afford Example 77. LCMS: m/z=337.1 [M+1]⁺; HPLC Ret. Time 1.711 min. (HPLC Method C). ¹H NMR (400 MHz, DMSO-d₆) δ ppm 13.02 (bs, 1H), 8.44 (s, 1H), 7.93 (s, 1H), 7.58 (s, 2H), 7.44-7.28 (m, 4H), 6.95 (bs, 1H), 2.98 (bs, 1H), 1.27-1.13 (m, 6H).

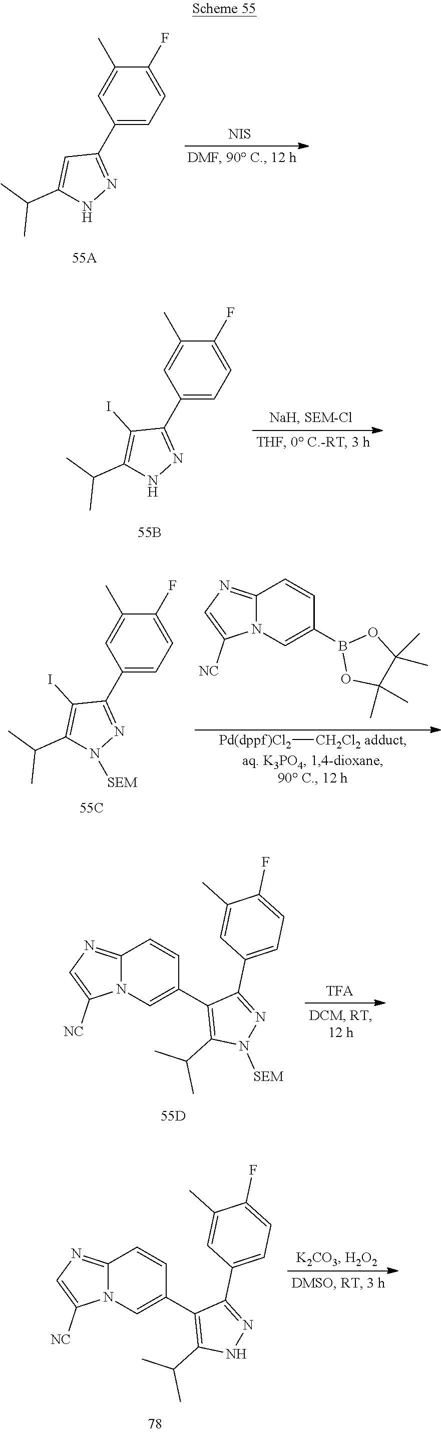

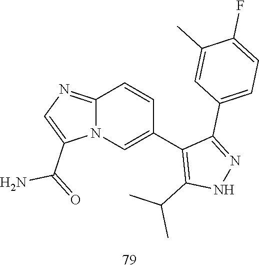 [SMILES: Cc1cc(-c2n[nH]c(C(C)C)c2-c2ccc3ncc(C(N)=O)n3c2)ccc1F]

### Intermediate 55B: 3-(4-fluoro-3-methylphenyl)-4-iodo-5-isopropyl-1H-pyrazole

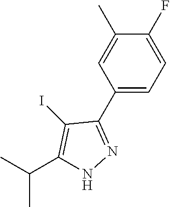 [SMILES: Cc1cc(-c2n[nH]c(C(C)C)c2I)ccc1F]

Intermediate 55B was synthesized by reacting intermediate 55A with NIS and by employing the experimental procedure described for intermediate 47B in Scheme 47. The crude product was purified by silica gel chromatography (40 g RediSep® column, eluting with a gradient of 5-10% EtOAc in petroleum ether). Fractions containing the desired product were combined and evaporated under reduced pressure to afford 55B. LCMS: m/z=345.3 [M+1]⁺; HPLC Ret. Time 1.50 min. (HPLC Method B).

### Intermediate 55C: 3-(4-fluoro-3-methylphenyl)-4-iodo-5-isopropyl-1-((2-(trimethylsilyl) ethoxy)methyl)-1H-pyrazole

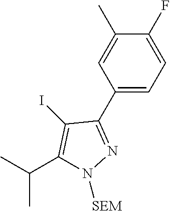 [SMILES: Cc1cc(-c2nn(COCC[Si](C)(C)C)c(C(C)C)c2I)ccc1F]

Intermediate 55C was synthesized by reacting intermediate 55B with SEM-Cl and by employing the experimental procedure described for intermediate 8A in Scheme 8 to afford 55C as a gum. LCMS: m/z=475.2 [M+1]⁺; HPLC Ret. Time 4.638 min. (HPLC Method E).

### Intermediate 55D: 6-(3-(4-fluoro-3-methylphenyl)-5-isopropyl-1-((2-(trimethylsilyl)ethoxy)methyl)-1H-pyrazol-4-yl)imidazo[1,2-a]pyridine-3-carbonitrile

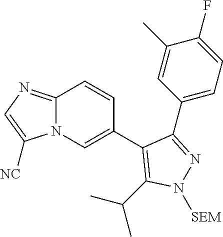 [SMILES: Cc1cc(-c2nn(COCC[Si](C)(C)C)c(C(C)C)c2-c2ccc3ncc(C#N)n3c2)ccc1F]

Intermediate 55D was synthesized by reacting intermediate 55C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1 to obtain intermediate 55D as a gum. LCMS: m/z=490.2 [M+1]⁺; HPLC Ret. Time 4.090 min. (HPLC Method E).

### Example 78: 6-(3-(4-fluoro-3-methylphenyl)-5-isopropyl-1H-pyrazol-4-yl)imidazo[1,2-a]pyridine-3-carbonitrile

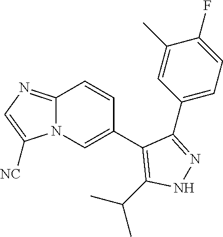 [SMILES: Cc1cc(-c2n[nH]c(C(C)C)c2-c2ccc3ncc(C#N)n3c2)ccc1F]

Example 78 was synthesized by reacting intermediate 55D with TFA and by employing the experimental procedure described for Example 1 in Scheme 1. The crude product was dissolved in DMF and purified via preparative HPLC (Condition L). Fractions containing the desired product were combined and dried under vacuum to afford Example 78. LCMS: m/z=360.1 [M+1]⁺; HPLC Ret. Time 1.790 min. (HPLC Method C). ¹H NMR (400 MHz, DMSO-d₆) δ ppm 13.02 (bs, H), 8.46 (s, 1H), 8.36 (s, 1H), 7.83 (s, 1H), 7.43-7.24 (m, 2H), 7.13-6.95 (m, 2H), 3.07-2.98 (m, 1H), 2.17 (s, 3H), 1.28-1.14 (m, 6H).

### Example 79: 6-(3-(4-fluoro-3-methylphenyl)-5-isopropyl-1H-pyrazol-4-yl)imidazo[1,2-a]pyridine-3-carboxamide

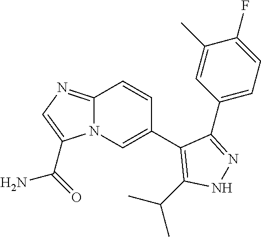 [SMILES: Cc1cc(-c2n[nH]c(C(C)C)c2-c2ccc3ncc(C(N)=O)n3c2)ccc1F]

Example 79 was synthesized by reacting Example 78 with H₂O₂/K₂CO₃ and by employing the experimental procedure described for Example 2 in Scheme 1. The crude product was dissolved in DMF and purified via preparative HPLC (Condition H). Fractions containing the desired product were combined and dried under vacuum to afford Example 79. LCMS: m/z=378.1 [M+1]⁺; HPLC Ret. Time 1.460 min. (HPLC Method C). ¹H NMR (400 MHz, DMSO-d₆) δ ppm 12.98 (bs, 1H), 9.39 (s, 1H), 8.35 (s, 1H), 7.95 (bs, 1H), 7.70 (s, 1H), 7.38 (bs, 2H), 7.18 (d, J=9.0 Hz, 1H), 7.12-6.91 (m, 2H), 3.01-2.84 (m, 1H), 2.15 (s, 3H), 1.23 (bs, 6H).

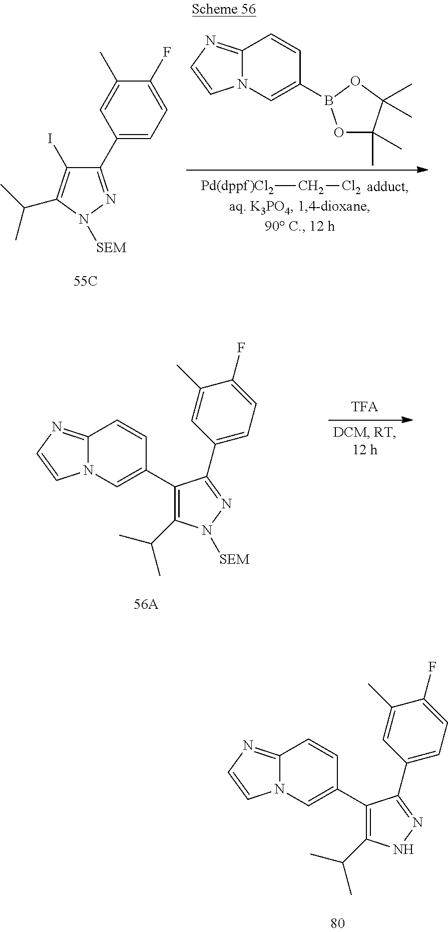

### Intermediate 56A: 6-(3-(4-fluoro-3-methylphenyl)-5-isopropyl-1-((2-(trimethylsilyl) ethoxy)methyl)-1H-pyrazol-4-yl)imidazo[1,2-a]pyridine

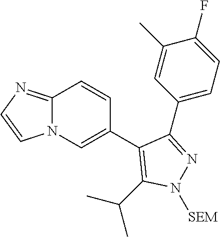 [SMILES: Cc1cc(-c2nn(COCC[Si](C)(C)C)c(C(C)C)c2-c2ccc3nccn3c2)ccc1F]

Intermediate 56A was synthesized by reacting intermediate 55C with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 56A as a gum. LCMS: m/z=465.4 [M+1]⁺; HPLC Ret. Time 3.852 min. (HPLC Method E).

### Example 80: 6-(3-(4-fluoro-3-methylphenyl)-5-isopropyl-1H-pyrazol-4-yl)imidazo[1,2-a]pyridine

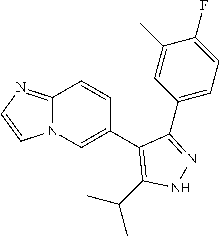 [SMILES: Cc1cc(-c2n[nH]c(C(C)C)c2-c2ccc3nccn3c2)ccc1F]

Example 80 was synthesized by reacting intermediate 56A with TFA and by employing the experimental procedure described for Example 1 in Scheme 1. The crude product was dissolved in DMF and purified via preparative HPLC (Condition H). Fractions containing the desired product were combined and dried under vacuum to afford Example 80. LCMS: m/z=335.2 [M+1]⁺; HPLC Ret. Time 1.629 min. (HPLC Method C). ¹H NMR (400 MHz, DMSO-d₆) δ ppm 12.93 (s, 1H), 8.42 (s, 1H), 7.93 (s, 1H), 7.59-7.48 (m, 2H), 7.41 (d, J=7.3 Hz, 1H), 7.12-7.04 (m, 1H), 7.03-6.89 (m, 2H), 3.01-2.88 (m, 1H), 2.16 (s, 3H), 1.28-1.09 (m, 6H).

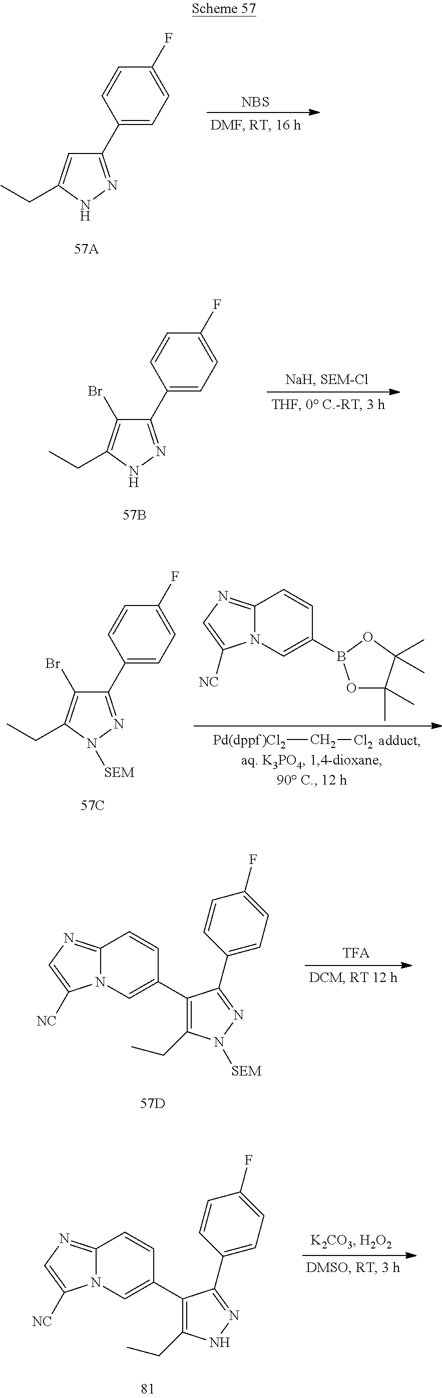

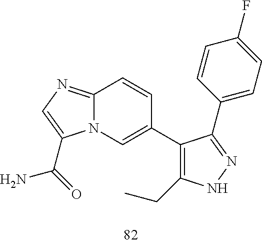 [SMILES: CCc1[nH]nc(-c2ccc(F)cc2)c1-c1ccc2ncc(C(N)=O)n2c1]

### Intermediate 57B: 4-bromo-5-ethyl-3-(4-fluorophenyl)-1H-pyrazole

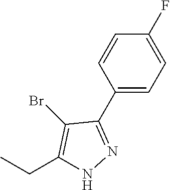

Intermediate 57B was synthesized by reacting intermediate 57A with NBS and by employing the experimental procedure described for Intermediate 1D in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 57B. LCMS: m/z=271.0 [M+2]⁺; HPLC Ret. Time 2.526 min. (HPLC Method E).

### Intermediate 57C: 4-bromo-5-ethyl-3-(4-fluorophenyl)-1-((2-(trimethylsilyl)ethoxy)methyl)-1H-pyrazole

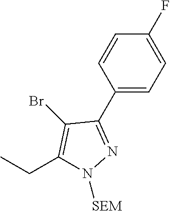 [SMILES: CCc1c(Br)c(-c2ccc(F)cc2)nn1COCC[Si](C)(C)C]

Intermediate 57C was synthesized by reacting intermediate 57B with SEM-Cl and by employing the experimental procedure described for intermediate 8A in Scheme 8. The crude product was purified by silica gel chromatography (40 g RediSep® column, eluting with a gradient of 5-10% EtOAc in petroleum ether). Fractions containing the desired product were combined and evaporated under reduced pressure to afford intermediate 57C. ¹H NMR (300 MHz, DMSO-d₆) δ ppm 7.83 (dd, J=8.9, 5.5 Hz, 1H), 7.57 (d, J=8.7 Hz, 1H), 7.45-7.16 (m, 2H), 5.49 (s, 2H), 5.26 (s, 2H), 3.67-3.43 (m, 3H), 1.27-1.06 (m, 2H), 0.93-0.64 (m, 2H), −0.03-−0.08 (m, 9H).

### Intermediate 57D: 6-(5-ethyl-3-(4-fluorophenyl)-1-((2-(trimethylsilyl)ethoxy)methyl)-1H-pyrazol-4-yl)imidazo[1,2-a]pyridine-3-carbonitrile

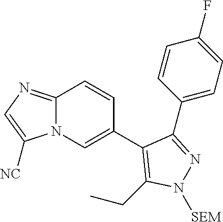 [SMILES: CCc1c(-c2ccc3ncc(C#N)n3c2)c(-c2ccc(F)cc2)nn1COCC[Si](C)(C)C]

Intermediate 57D was synthesized by reacting intermediate 57C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated to afford intermediate 57D. LCMS: m/z=462.2 [M+1]⁺; HPLC Ret. Time 3.543 min. (HPLC Method E).

### Example 81: 6-(5-ethyl-3-(4-fluorophenyl)-1H-pyrazol-4-yl)imidazo[1,2-a]pyridine-3-carbonitrile

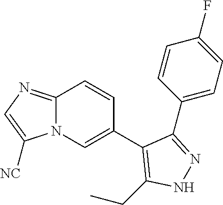 [SMILES: CCc1[nH]nc(-c2ccc(F)cc2)c1-c1ccc2ncc(C#N)n2c1]

Example 81 was synthesized by reacting intermediate 57D with TFA and by employing the experimental procedure described for Example 1 in Scheme 1. The crude product was dissolved in DMF and purified via preparative HPLC (Condition L). Fractions containing the desired product were combined and dried under vacuum to afford Example 81. LCMS: m/z=332.1 [M+1]⁺; HPLC Ret. Time 1.615 min. (HPLC Method C). ¹H NMR (400 MHz, DMSO-d₆) δ ppm 13.04 (bs., 1H), 8.46 (s, 1H), 8.40 (s, 1H), 7.83 (d, J=9.0 Hz, 1H), 7.45-7.40 (m, 2H), 7.31-7.09 (m, 3H), 2.68 (dd, J=3.5, 2.0 Hz, 2H), 1.17 (t, J=7.3 Hz, 3H).

### Example 82: 6-(5-ethyl-3-(4-fluorophenyl)-1H-pyrazol-4-yl)imidazo[1,2-a]pyridine-3-carboxamide

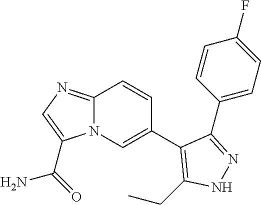 [SMILES: CCc1[nH]nc(-c2ccc(F)cc2)c1-c1ccc2ncc(C(N)=O)n2c1]

Example 82 was synthesized by reacting Example 81 with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 82. LCMS: m/z=350.1 [M+1]⁺; HPLC Ret. Time 1.283 min. (HPLC Method C). ¹H NMR (400 MHz, DMSO-d₆) δ ppm 13.01 (s, 1H), 9.40 (s, 1H), 8.28 (s, 1H), 7.95 (bs, 1H), 7.71 (d, J=1.0 Hz, 1H), 7.41 (d, J=5.5 Hz, 3H), 7.26-7.01 (m, 3H), 2.77-2.54 (m, 2H), 1.26-1.02 (m, 3H).

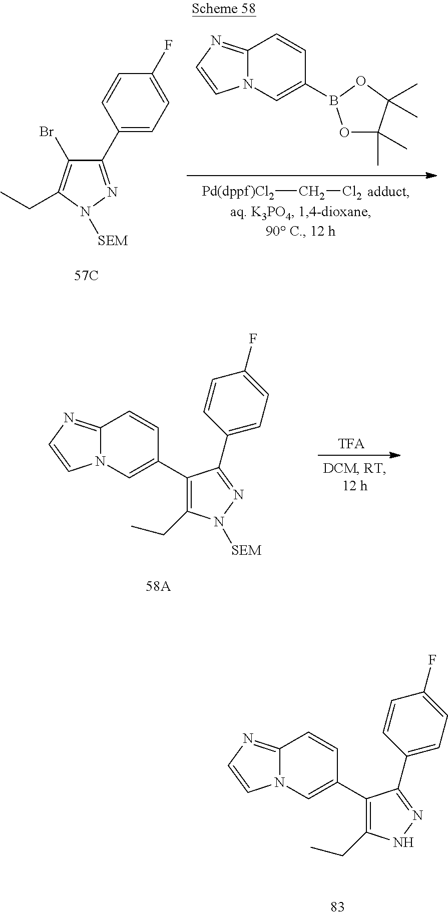

### Intermediate 58A: 6-(5-ethyl-3-(4-fluorophenyl)-1-((2-(trimethylsilyl)ethoxy)methyl)-1H-pyrazol-4-yl)imidazo[1,2-a]pyridine

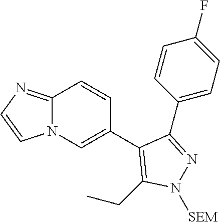 [SMILES: CCc1c(-c2ccc3nccn3c2)c(-c2ccc(F)cc2)nn1COCC[Si](C)(C)C]

Intermediate 58A was synthesized by reacting intermediate 57C with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 58A as a gum. LCMS: m/z=437.6 [M+1]⁺; HPLC Ret. Time 1.55 min. (HPLC Method B).

### Example 83: 6-(5-ethyl-3-(4-fluorophenyl)-1H-pyrazol-4-yl)imidazo[1,2-a]pyridine

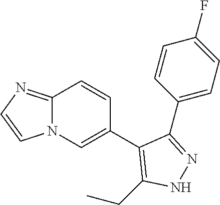 [SMILES: CCc1[nH]nc(-c2ccc(F)cc2)c1-c1ccc2nccn2c1]

Example 83 was synthesized by reacting intermediate 58A with TFA and by employing the experimental procedure described for Example 1 in Scheme 1. The crude product was dissolved in DMF and purified via preparative HPLC (Condition L). Fractions containing the desired product were combined and dried under vacuum to afford Example 83. LCMS: m/z=307.1 [M+1]⁺; HPLC Ret. Time 1.444 min. (HPLC Method C). ¹H NMR (400 MHz, DMSO-d₆) δ ppm 8.46 (d, J=1.0 Hz, 1H), 7.93 (s, 1H), 7.62-7.50 (m, 2H), 7.46-7.35 (m, 2H), 7.15 (t, J=8.8 Hz, 2H), 6.96 (dd, J=9.3, 1.8 Hz, 1H), 2.66-2.54 (m, 2H), 1.20-1.08 (m, 3H).

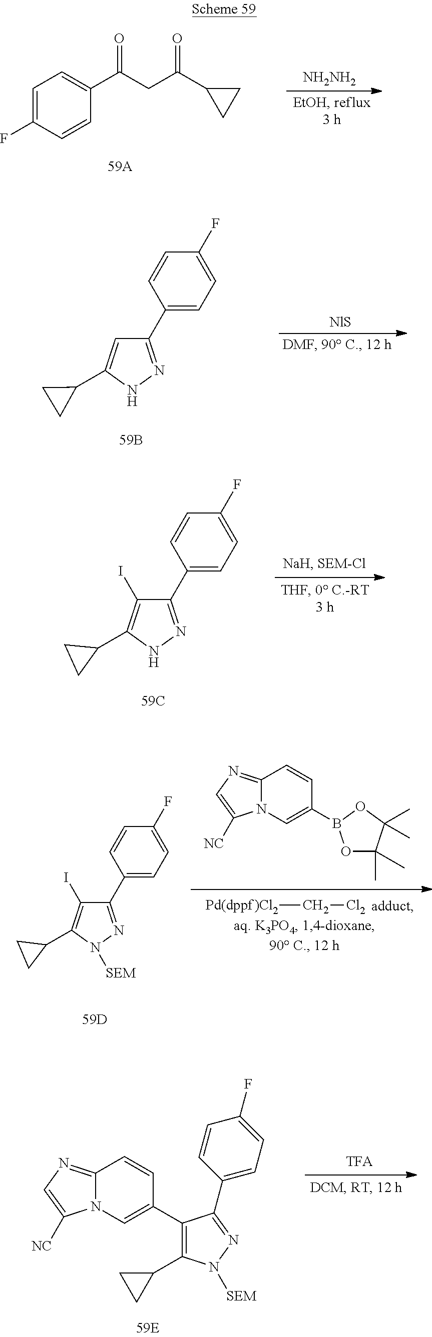

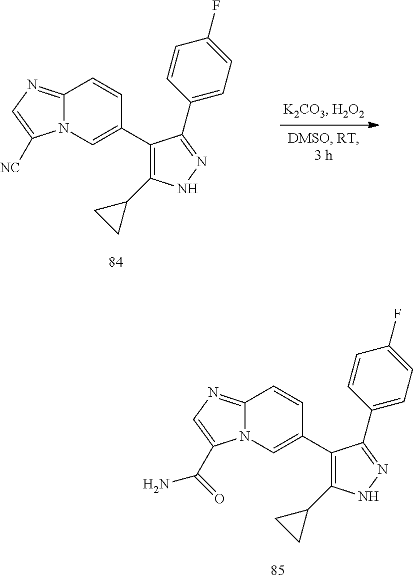 [SMILES: N#Cc1cnc2ccc(-c3c(-c4ccc(F)cc4)n[nH]c3C3CC3)cn12 ; NC(=O)c1cnc2ccc(-c3c(-c4ccc(F)cc4)n[nH]c3C3CC3)cn12]

### Intermediate 59B: 5-cyclopropyl-3-(4-fluorophenyl)-1H-pyrazole

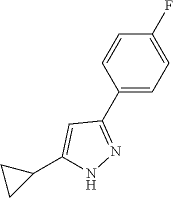 [SMILES: Fc1ccc(-c2cc(C3CC3)[nH]n2)cc1]

Intermediate 59B was synthesized by reacting intermediate 59A with methyl iodide and by employing the experimental procedure described for intermediate 53B in Scheme 53 to afford 59B. LCMS: m/z=203.2 [M-1]⁺; HPLC Ret. Time 2.19 min. (HPLC Method E).

### Intermediate 59C: 5-cyclopropyl-3-(4-fluorophenyl)-4-iodo-1H-pyrazole

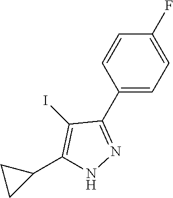 [SMILES: Fc1ccc(-c2n[nH]c(C3CC3)c2I)cc1]

Intermediate 59C was synthesized by reacting intermediate 59B with NIS and by employing the experimental procedure described for intermediate 47B in Scheme 47. The crude product was purified by silica gel chromatography (40 g RediSep® column, eluting with a gradient of 5-10% EtOAc in petroleum ether). Fractions containing the desired product were combined and evaporated under reduced pressure to afford intermediate 59C. LCMS: m/z=329.0 [M+1]⁺; HPLC Ret. Time 2.559 min. (HPLC Method E).

### Intermediate 59D: 5-cyclopropyl-3-(4-fluorophenyl)-4-iodo-1-((2-(trimethylsilyl)ethoxy) methyl)-1H-pyrazole

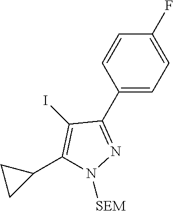 [SMILES: C[Si](C)(C)CCOCn1nc(-c2ccc(F)cc2)c(I)c1C1CC1]

Intermediate 59D was synthesized by reacting intermediate 59C with SEM-Cl and by employing the experimental procedure described for intermediate 8A in Scheme 8. The crude product was purified by silica gel chromatography (40 g RediSep® column, eluting with a gradient of 5-8% EtOAc in petroleum ether). Fractions containing the desired product were combined and evaporated under reduced pressure to afford intermediate 59D as a liquid. LCMS: m/z=459.0 [M+1]⁺; HPLC Ret. Time 4.367 min. (HPLC Method E).

### Intermediate 59E: 6-(5-cyclopropyl-3-(4-fluorophenyl)-1-((2-(trimethylsilyl)ethoxy)methyl)-1H-pyrazol-4-yl)imidazo[1,2-a]pyridine-3-carbonitrile

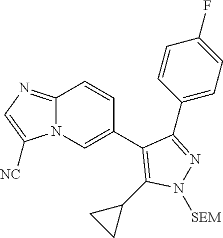 [SMILES: C[Si](C)(C)CCOCn1nc(-c2ccc(F)cc2)c(-c2ccc3ncc(C#N)n3c2)c1C1CC1]

Intermediate 59E was synthesized by reacting intermediate 59D with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 59E as a gum. LCMS: m/z=474.2 [M+1]⁺; HPLC Ret. Time 3.784 min. (HPLC Method E).

### Example 84: 6-(5-cyclopropyl-3-(4-fluorophenyl)-1H-pyrazol-4-yl)imidazo[1,2-a]pyridine-3-carbonitrile

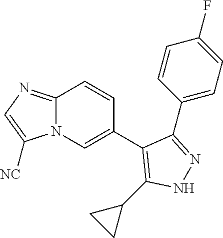 [SMILES: N#Cc1cnc2ccc(-c3c(-c4ccc(F)cc4)n[nH]c3C3CC3)cn12]

Example 84 was synthesized by reacting intermediate 59E with TFA and by employing the experimental procedure described for Example 1 in Scheme 1. The crude product was dissolved in DMF and purified via preparative HPLC (Condition L). Fractions containing the desired product were combined and dried under vacuum to afford Example 84. LCMS: m/z=344.1 [M+1]⁺; HPLC Ret. Time 1.843 min. (HPLC Method C). ¹H NMR (400 MHz, DMSO-d₆) δ ppm 8.45 (s, 2H), 7.82 (d, J=9.3 Hz, 1H), 7.47-7.38 (m, 2H), 7.32 (d, J=8.8 Hz, 1H), 7.26-7.05 (m, 2H), 2.01-1.84 (m, 1H), 0.95-0.78 (m, 4H).

### Example 85: 6-(5-cyclopropyl-3-(4-fluorophenyl)-1H-pyrazol-4-yl)imidazo[1,2-a]pyridine-3-carboxamide

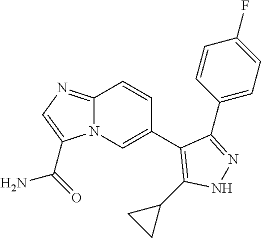 [SMILES: NC(=O)c1cnc2ccc(-c3c(-c4ccc(F)cc4)n[nH]c3C3CC3)cn12]

Example 85 was synthesized by reacting Example 84 with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 85. LCMS: m/z=362.1 [M+1]⁺; HPLC Ret. Time 1.346 min. (HPLC Method C). ¹H NMR (400 MHz, DMSO-d₆) δ ppm 12.79 (bs, 1H), 9.51 (s, 1H), 8.34 (s, 1H), 7.92 (bs, 1H), 7.70 (s, 1H), 7.40 (dd, J=7.9, 5.7 Hz, 2H), 7.24-7.06 (m, 4H), 1.81 (bs, 1H), 0.88 (bs, 4H).

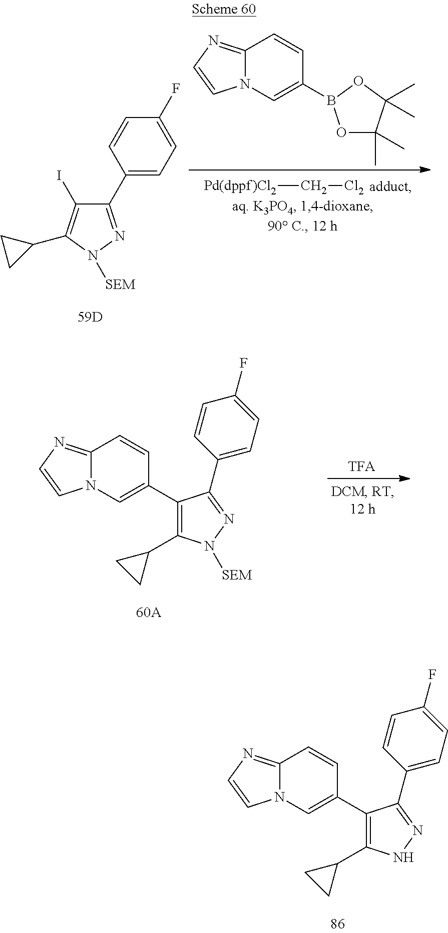

### Intermediate 60A: 6-(5-cyclopropyl-3-(4-fluorophenyl)-1-((2-(trimethylsilyl)ethoxy)methyl)-1H-pyrazol-4-yl)imidazo [1,2-a]pyridine

Intermediate 60A was synthesized by reacting intermediate 59D with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 60A as a gum. LCMS: m/z=449.2 [M+1]⁺; HPLC Ret. Time 3.76 min. (HPLC Method E).

### Example 86: 6-(5-cyclopropyl-3-(4-fluorophenyl)-1H-pyrazol-4-yl)imidazo [1,2-a]pyridine

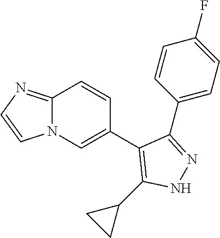 [SMILES: Fc1ccc(-c2n[nH]c(C3CC3)c2-c2ccc3nccn3c2)cc1]

Example 86 was synthesized by reacting intermediate 60A with TFA and by employing the experimental procedure described for Example 1 in Scheme 1. The crude product was dissolved in DMF and purified via preparative HPLC (Condition H). Fractions containing the desired product were combined and dried under vacuum to afford Example 86. LCMS: m/z=319.1 [M+1]⁺; HPLC Ret. Time 1.55 min. (HPLC Method C). ¹H NMR (400 MHz, DMSO-d₆) δ ppm 8.50 (s, 1H), 7.94 (s, 1H), 7.61-7.49 (m, 2H), 7.41 (bs, 2H), 7.26-7.07 (m, 2H), 6.98 (bs, 1H), 1.91-1.73 (m, 1H), 0.90-0.72 (m, 4H).

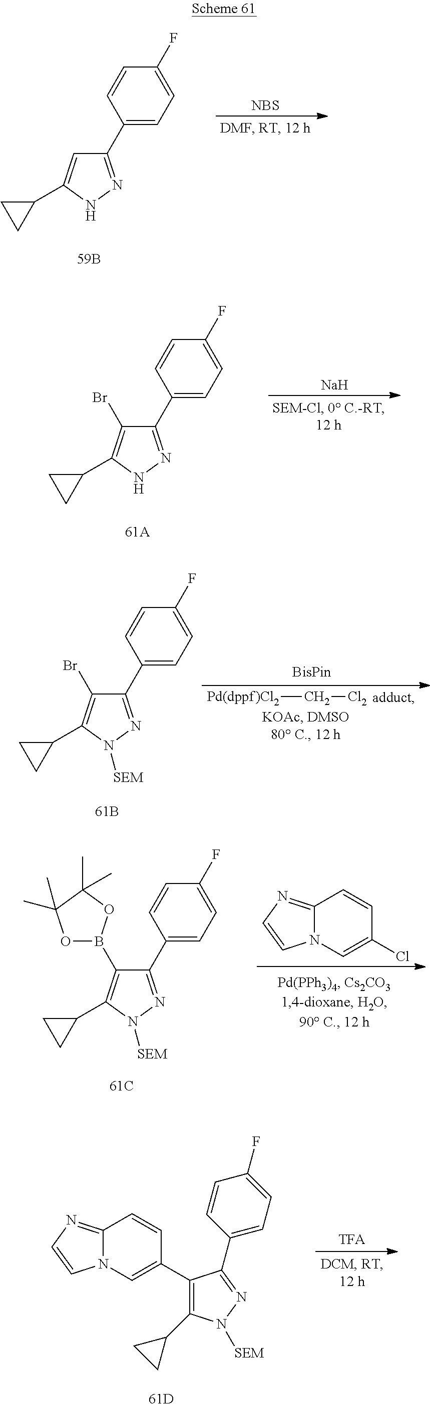

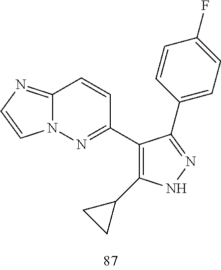 [SMILES: Fc1ccc(-c2n[nH]c(C3CC3)c2-c2ccc3nccn3n2)cc1]

### Intermediate 61A: 4-bromo-5-cyclopropyl-3-(4-fluorophenyl)-1H-pyrazole

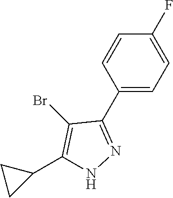 [SMILES: Fc1ccc(-c2n[nH]c(C3CC3)c2Br)cc1]

Intermediate 61A was synthesized by reacting intermediate 59B with NBS and by employing the experimental procedure described for intermediate 1D in Scheme 1 to afford 61A as a pale yellow solid. LCMS: m/z=281.1 [M+1]⁺; HPLC Ret. Time 2.677 min. (HPLC Method E).

### Intermediate 61B: 4-bromo-5-cyclopropyl-3-(4-fluorophenyl)-1-((2-(trimethylsilyl)ethoxy) methyl)-1H-pyrazole

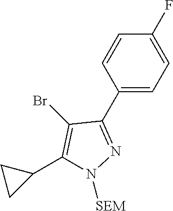 [SMILES: C[Si](C)(C)CCOCn1nc(-c2ccc(F)cc2)c(Br)c1C1CC1]

Intermediate 61B was synthesized by reacting intermediate 61A with SEM-Cl and by employing the experimental procedure described for intermediate 8A in Scheme 8 to get 61B. LCMS: m/z 411.1 [M+1]⁺; HPLC Ret. Time 4.288 min. (HPLC Method E).

### Intermediate 61C: 5-cyclopropyl-3-(4-fluorophenyl)-4-(4,4,5,5-tetramethyl-1,3,2-dioxaborolan-2-yl)-1-((2-(trimethylsilyl)ethoxy)methyl)-1H-pyrazole

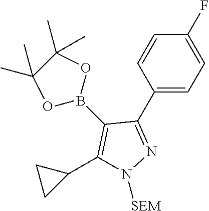 [SMILES: CC1(C)OB(c2c(-c3ccc(F)cc3)nn(COCC[Si](C)(C)C)c2C2CC2)OC1(C)C]

Intermediate 61C was synthesized by reacting intermediate 61B with BisPin and by employing the experimental procedure described for intermediate 8B in Scheme 8. The crude product was purified by silica gel chromatography (40 g RediSep® column, eluting with a gradient of 2-3% EtOAc in petroleum ether). Fractions containing the desired product were combined and evaporated to afford intermediate 61C. LCMS: m/z 459.4 [M+1]⁺; HPLC Ret. Time 1.44 min. (HPLC Method B).

### Intermediate 61D: 6-(5-cyclopropyl-3-(4-fluorophenyl)-1-((2-(trimethylsilyl)ethoxy)methyl)-1H-pyrazol-4-yl)imidazo[1,2-b]pyridazine

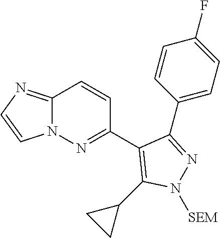 [SMILES: C[Si](C)(C)CCOCn1nc(-c2ccc(F)cc2)c(-c2ccc3nccn3n2)c1C1CC1]

Intermediate 61D was synthesized by reacting intermediate 61C with 6-chloroimidazo[1,2-b]pyridazine and by employing the experimental procedure described for intermediate 13B in Scheme 13. The crude product was purified by silica gel chromatography (40 g RediSep® column, eluting with a gradient of 20-30% EtOAc in petroleum ether). Fractions containing the desired product were combined and evaporated to afford intermediate 61D. LCMS: m/z 450.2 [M+1]⁺; HPLC Ret. Time 3.468 min. (HPLC Method E).

### Example 87: 6-(5-cyclopropyl-3-(4-fluorophenyl)-1H-pyrazol-4-yl)imidazo [1,2-b]pyridazine

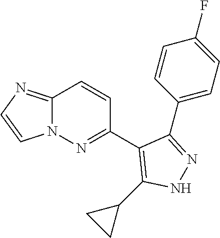 [SMILES: Fc1ccc(-c2n[nH]c(C3CC3)c2-c2ccc3nccn3n2)cc1]

Example 87 was synthesized by reacting intermediate 61D with TFA and by employing the experimental procedure described for Example 1 in Scheme 1. The crude product was dissolved in DMF and purified via preparative HPLC (Condition L). Fractions containing the desired product were combined and dried under vacuum to afford Example 87. LCMS: m/z 320.1 [M+1]⁺; HPLC Ret. Time 1.339 min. (HPLC Method C). ¹H NMR (400 MHz, CD3OD) δ ppm 8.12 (s, 1H), 7.90 (d, J=9.5 Hz, 1H), 7.75 (d, J=1.0 Hz, 1H), 7.54-7.33 (m, 2H), 7.19-6.94 (m, 3H), 2.25 (bs, 1H), 1.05-0.79 (m, 4H).

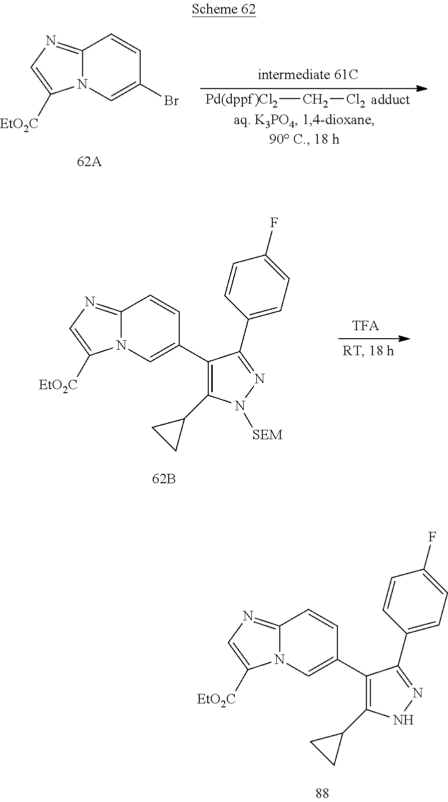

### Intermediate 62B: ethyl 6-(5-cyclopropyl-3-(4-fluorophenyl)-1-((2-(trimethylsilyl)ethoxy)methyl)-1H-pyrazol-4-yl)imidazo[1,2-a]pyridine-3-carboxylate

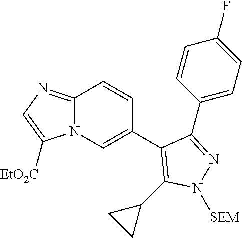 [SMILES: CCOC(=O)c1cnc2ccc(-c3c(-c4ccc(F)cc4)nn(COCC[Si](C)(C)C)c3C3CC3)cn12]

Intermediate 62B was synthesized by reacting intermediate 62A with 61C and by employing the experimental procedure described for intermediate 1E in Scheme 1 to afford 62B. LCMS: m/z=521.5 [M+H]⁺; HPLC Ret. Time 1.84 min. (HPLC Method B).

### Example 88: ethyl 6-(5-cyclopropyl-3-(4-fluorophenyl)-1H-pyrazol-4-yl)imidazo[1,2-a]pyridine-3-carboxylate

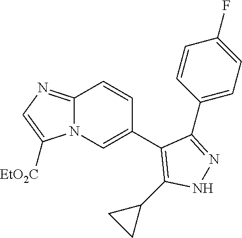 [SMILES: CCOC(=O)c1cnc2ccc(-c3c(-c4ccc(F)cc4)n[nH]c3C3CC3)cn12]

Example 88 was synthesized by reacting intermediate 62B with TFA and by employing the experimental procedure described for Example 1 in Scheme 1. The crude product was dissolved in DMF and purified via preparative HPLC (Condition N). Fractions containing the desired product were combined and dried under vacuum to afford Example 88. LCMS: m/z=391.1 [M+H]⁺; HPLC Ret. Time 1.930 min. (HPLC Method C); ¹H NMR (400 MHz, DMSO-d₆) δ ppm 9.47 (d, J=0.7 Hz, 1H), 8.58 (s, 1H), 8.06 (dd, J=9.3, 1.0 Hz, 1H), 7.72-7.57 (m, 2H), 7.51-7.20 (m, 3H), 4.60 (q, J 7.1 Hz, 2H), 2.17-2.08 (m, 1H), 1.62-1.52 (m, 3H), 1.19-1.09 (m, 4H).

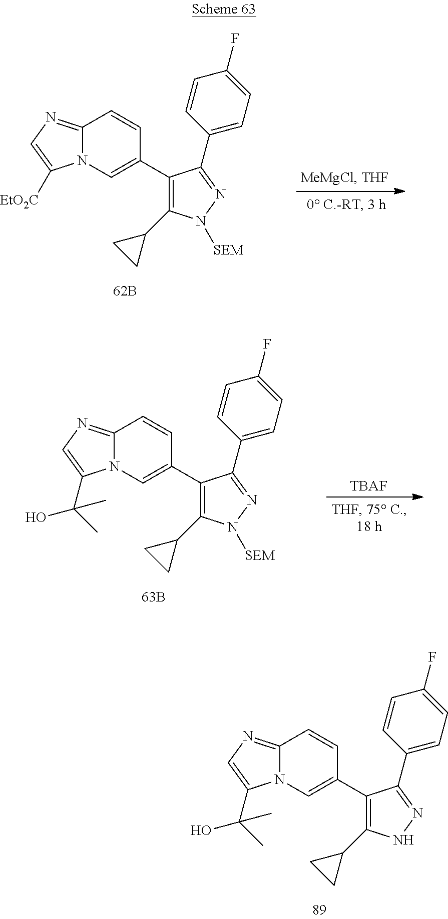 [SMILES: CCOC(=O)c1cnc2ccc(-c3c(-c4ccc(F)cc4)nn(COCC[Si](C)(C)C)c3C3CC3)cn12 ; CC(C)(O)c1cnc2ccc(-c3c(-c4ccc(F)cc4)nn(COCC[Si](C)(C)C)c3C3CC3)cn12 ; "TBAF" ; CC(C)(O)c1cnc2ccc(-c3c(-c4ccc(F)cc4)n[nH]c3C3CC3)cn12]

### Intermediate 63A: 2-(6-(5-cyclopropyl-3-(4-fluorophenyl)-1-((2-(trimethylsilyl)ethoxy)methyl)-1H-pyrazol-4-yl)imidazo[1,2-a]pyridin-3-yl)propan-2-ol

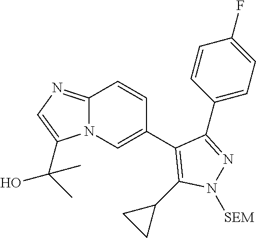 [SMILES: CC(C)(O)c1cnc2ccc(-c3c(-c4ccc(F)cc4)nn(COCC[Si](C)(C)C)c3C3CC3)cn12]

Intermediate 63A was synthesized by reacting intermediate 62B with MeMgCl and by employing the experimental procedure described for Example 52 in Scheme 34. LCMS: m/z=507.3 [M+H]⁺; HPLC Ret. Time 1.60 min. (HPLC Method B).

### Example 89: 2-(6-(5-cyclopropyl-3-(4-fluorophenyl)-1H-pyrazol-4-yl)imidazo[1,2-a]pyridin-3-yl)propan-2-ol

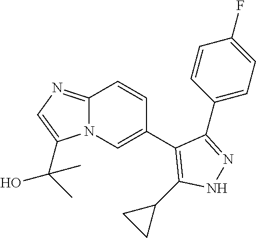 [SMILES: CC(C)(O)c1cnc2ccc(-c3c(-c4ccc(F)cc4)n[nH]c3C3CC3)cn12]

Example 89 was synthesized by reacting intermediate 63A with TFA and by employing the experimental procedure described for Example 59 in Scheme 41. The crude product was dissolved in DMF and purified via preparative HPLC (Condition N). Fractions containing the desired product were combined and dried under vacuum to afford Example 89. LCMS: m/z=377.2 [M+H]⁺; HPLC Ret. Time 1.470 min. (HPLC Method C); ¹H NMR (400 MHz, DMSO-d₆) δ ppm 13.25-12.98 (m, 1H), 8.98-8.85 (m, 1H), 7.84 (s, 1H), 7.70 (s, 3H), 7.53-7.29 (m, 3H), 5.70-5.51 (s, 1H), 2.16 (d, J 13.9 Hz, 1H), 1.79 (s, 6H), 1.24-1.04 (m, 4H).

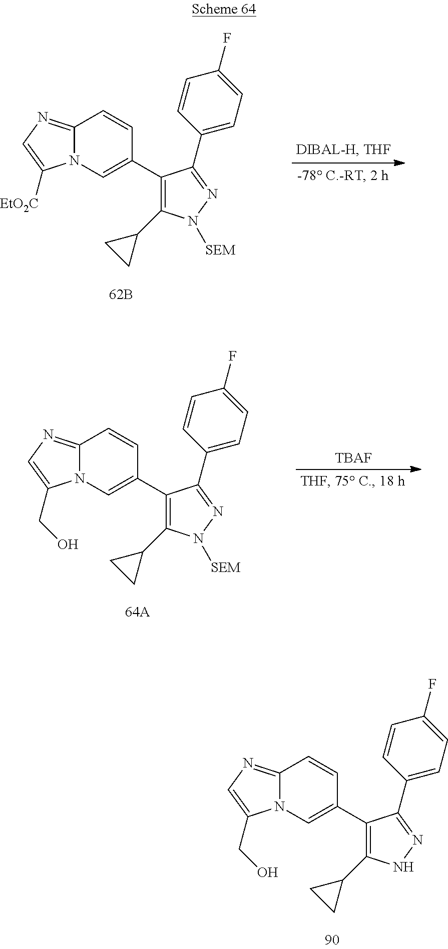 [SMILES: CCOC(=O)c1cnc2ccc(-c3c(-c4ccc(F)cc4)nn(COCC[Si](C)(C)C)c3C3CC3)cn12 ; C[Si](C)(C)CCOCn1nc(-c2ccc(F)cc2)c(-c2ccc3ncc(CO)n3c2)c1C1CC1 ; "TBAF" ; OCc1cnc2ccc(-c3c(-c4ccc(F)cc4)n[nH]c3C3CC3)cn12]

### Intermediate 64A: (6-(5-cyclopropyl-3-(4-fluorophenyl)-1-((2-(trimethylsilyl)ethoxy)methyl)-1H-pyrazol-4-yl)imidazo[1,2-a]pyridin-3-yl)methanol

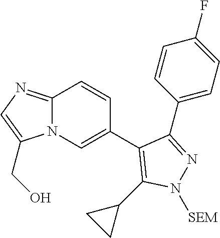 [SMILES: C[Si](C)(C)CCOCn1nc(-c2ccc(F)cc2)c(-c2ccc3ncc(CO)n3c2)c1C1CC1]

To a stirred solution of intermediate 62B (0.2 g, 0.384 mmol) in THF (8 mL) was added DIBAL-H (0.768 mL, 0.768 mmol, 1M in toluene) at −78° C. The reaction mixture was slowly warmed to RT and was stirred at the same temperature for 2 h. The reaction mixture was quenched with a 10% saturated aq. NaHCO₃ solution (15.0 mL) and was diluted with ethyl acetate (100 mL), filtered through Celite®. The aq. layer was extracted with ethyl acetate (3×50 mL). The combined organic layer was dried over anhydrous sodium sulfate, filtered and the filtrate evaporated under reduced pressure to afford 64A (0.18 g, 98% yield) as a brown oil. The crude product was used in the next step without purification. LCMS: m/z=479.4 [M+H]⁺; HPLC Ret. Time 3.106 min. (HPLC Method E).

### Example 90: (6-(5-cyclopropyl-3-(4-fluorophenyl)-1H-pyrazol-4-yl)imidazo[1,2-a]pyridin-3-yl)methanol

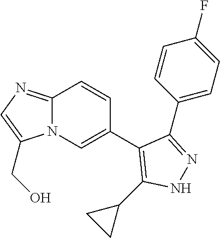 [SMILES: OCc1cnc2ccc(-c3c(-c4ccc(F)cc4)n[nH]c3C3CC3)cn12]

Example 90 was synthesized by reacting intermediate 64A with TBAF and by employing the experimental procedure described for Example 59 in Scheme 41. The crude product was dissolved in DMF and purified via preparative HPLC (Condition N). Fractions containing the desired product were combined and dried under vacuum to afford Example 90. LCMS: m/z=349.2 [M+H]⁺; HPLC Ret. Time 1.273 min. (HPLC Method C); ¹H NMR (400 MHz, DMSO-d₆) δ ppm 13.25-12.98 (m, 1H), 8.66-8.57 (m, 1H), 7.83 (s, 2H), 7.73-7.64 (m, 2H), 7.40 (br. s., 2H), 7.28 (d, J=8.6 Hz, 1H), 5.45 (s, 1H), 5.04 (s, 2H), 2.15 (br. s., 1H), 1.09 (dd, J=5.0, 2.1 Hz, 4H).

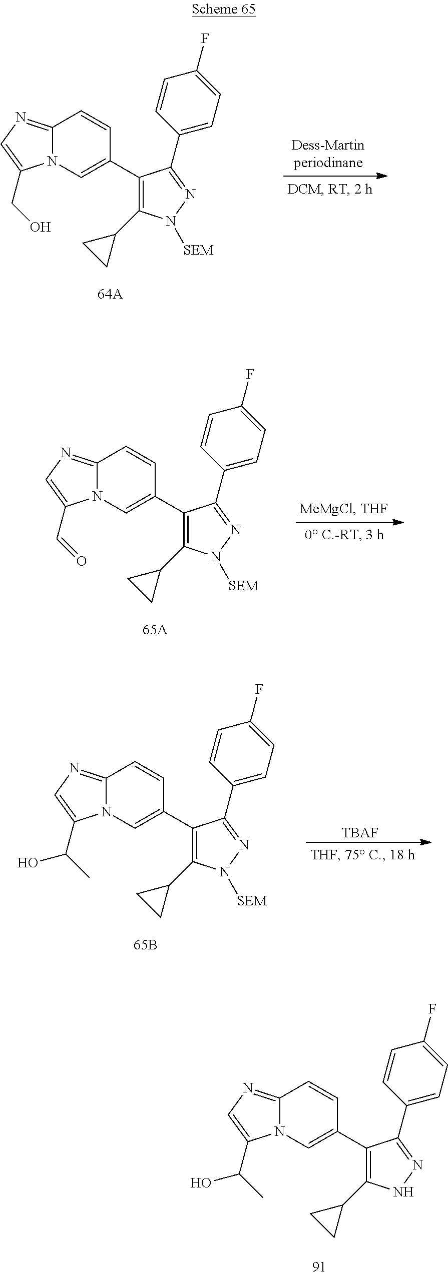 [SMILES: C[Si](C)(C)CCOCn1nc(-c2ccc(F)cc2)c(-c2ccc3ncc(CO)n3c2)c1C1CC1 ; C[Si](C)(C)CCOCn1nc(-c2ccc(F)cc2)c(-c2ccc3ncc(C=O)n3c2)c1C1CC1 ; CC(O)c1cnc2ccc(-c3c(-c4ccc(F)cc4)nn(COCC[Si](C)(C)C)c3C3CC3)cn12 ; "TBAF" ; CC(O)c1cnc2ccc(-c3c(-c4ccc(F)cc4)n[nH]c3C3CC3)cn12]

### Intermediate 65A: 6-(5-cyclopropyl-3-(4-fluorophenyl)-1-((2-(trimethylsilyl)ethoxy)methyl)-1H-pyrazol-4-yl)imidazo[1,2-a]pyridine-3-carbaldehyde

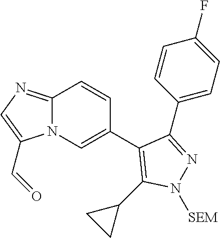 [SMILES: C[Si](C)(C)CCOCn1nc(-c2ccc(F)cc2)c(-c2ccc3ncc(C=O)n3c2)c1C1CC1]

To a stirred solution of intermediate 64A (0.15 g, 0.313 mmol) in DCM (6 mL) was added Dess-Martin periodinane (0.199 g, 0.470 mmol). The reaction mixture was stirred at RT for 2 h. The reaction was quenched with saturated aq. NaHCO₃ solution (100 mL) and was extracted with DCM (3×200 mL). The combined organic layer was dried over anhydrous sodium sulfate, filtered and the filtrate evaporated under reduced pressure to provide 65A (0.128 g, 86% yield) as a brown gum. LCMS: m/z=477.2 [M+H]⁺; HPLC Ret. Time 3.547 min. (HPLC Method E).

### Intermediate 65B: 1-(6-(5-cyclopropyl-3-(4-fluorophenyl)-1-((2-(trimethylsilyl)ethoxy)methyl)-1H-pyrazol-4-yl)imidazo[1,2-a]pyridin-3-yl)ethanol

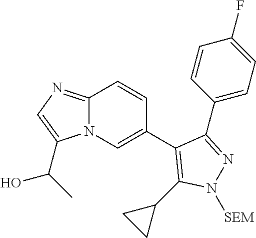 [SMILES: CC(O)c1cnc2ccc(-c3c(-c4ccc(F)cc4)nn(COCC[Si](C)(C)C)c3C3CC3)cn12]

Intermediate 65B was synthesized by reacting intermediate 65A with MeMgCl and by employing the experimental procedure described for Example 52 in Scheme 34 to afford intermediate 65B as a brown gum. The crude product was used in the next step without further purification. LCMS: m/z=493.2 [M+H]⁺; HPLC Ret. Time 3.512 min. (HPLC Method E).

### Example 91: 1-(6-(5-cyclopropyl-3-(4-fluorophenyl)-1H-pyrazol-4-yl)imidazo[1,2-a]pyridin-3-yl)ethanol

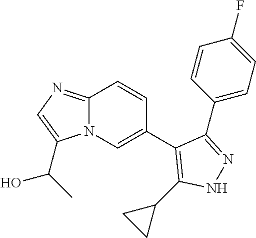 [SMILES: CC(O)c1cnc2ccc(-c3c(-c4ccc(F)cc4)n[nH]c3C3CC3)cn12]

Example 91 was synthesized by reacting intermediate 65B with TBAF and by employing the experimental procedure described for Example 59 in Scheme 41. The crude product was dissolved in DMF and purified via preparative HPLC (Condition N). Fractions containing the desired product were combined and dried under vacuum to afford Example 91. LCMS: m/z=363.2 [M+H]⁺; HPLC Ret. Time 1.395 min. (HPLC Method C); ¹H NMR (400 MHz, DMSO-d₆) δ ppm 12.98-12.71 (m, 1H), 8.39 (br. s., 1H), 7.54 (d, J=9.5 Hz, 1H), 7.48-7.36 (m, 3H), 7.26-6.96 (m, 3H), 5.29 (d, J=6.0 Hz, 1H), 5.06 (quin, J=6.3 Hz, 1H), 1.89 (br. s., 1H), 1.54 (d, J=6.5 Hz, 3H), 0.96-0.73 (m, 4H).

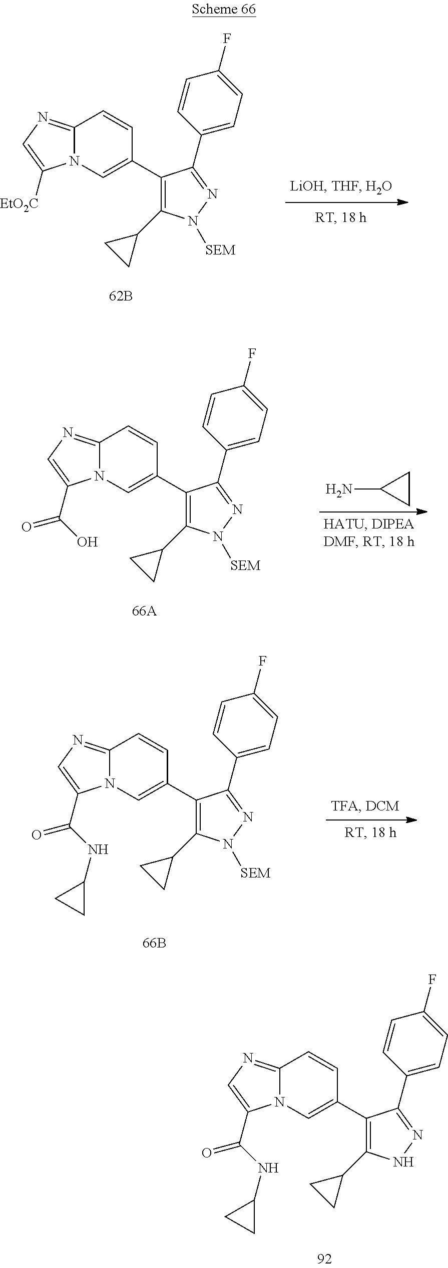

### Intermediate 66A: 6-(5-cyclopropyl-3-(4-fluorophenyl)-1-((2-(trimethylsilyl)ethoxy)methyl)-1H-pyrazol-4-yl)imidazo[1,2-a]pyridine-3-carboxylic acid

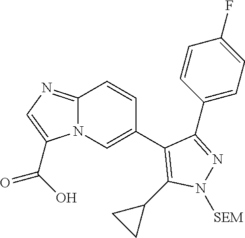 [SMILES: C[Si](C)(C)CCOCn1nc(-c2ccc(F)cc2)c(-c2ccc3ncc(C(=O)O)n3c2)c1C1CC1]

To a stirred solution of intermediate 62B (0.22 g, 0.423 mmol) in THF (4 mL) was added LiOH (0.025 g, 1.056 mmol) in water (0.5 mL). The reaction mixture was stirred at RT for 18 h. Then the mixture was acidified to pH 6 with 1.5N aq. HCl solution. The precipitate was filtered off and the filter-cake was dried to afford 66A (0.11 g, 52.8% yield) as off white solid. LCMS: m/z=493.2 [M+H]⁺; HPLC Ret. Time 2.735 min. (HPLC Method E).

### Intermediate 66B: N-cyclopropyl-6-(5-cyclopropyl-3-(4-fluorophenyl)-1-((2-(trimethylsilyl)ethoxy)methyl)-1H-pyrazol-4-yl)imidazo[1,2-a]pyridine-3-carboxamide

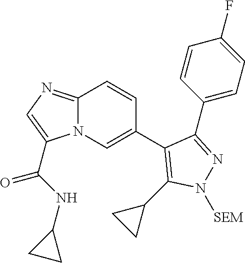 [SMILES: C[Si](C)(C)CCOCn1nc(-c2ccc(F)cc2)c(-c2ccc3ncc(C(=O)NC4CC4)n3c2)c1C1CC1]

To a stirred solution of intermediate 66A (0.045 g, 0.091 mmol) in DMF (5 mL) was added HATU (0.035 g, 0.091 mmol) and DIPEA (0.048 mL, 0.274 mmol), followed by cyclopropanamine (5.22 mg, 0.091 mmol). The reaction mixture was stirred at RT for 18 h. After that the reaction mixture was quenched with water (30 mL) and extracted with ethyl acetate (3×50 mL). The combined organic layers were dried over anhydrous sodium sulfate, filtered and the filtrate evaporated under reduced pressure to get intermediate 66B (0.031 g, 47.9% yield) as a brown gum. The crude product was used in the next step without purification. LCMS: m/z=532.4 [M+H]⁺; HPLC Ret. Time 3.480 min. (HPLC Method E).

### Example 92: N-cyclopropyl-6-(5-cyclopropyl-3-(4-fluorophenyl)-1H-pyrazol-4-yl)imidazo[1,2-a]pyridine-3-carboxamide

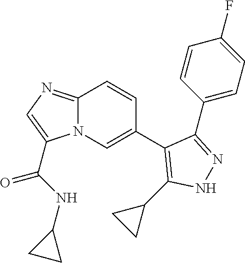 [SMILES: O=C(NC1CC1)c1cnc2ccc(-c3c(-c4ccc(F)cc4)n[nH]c3C3CC3)cn12]

Example 92 was synthesized by reacting intermediate 66B with TFA and by employing the experimental procedure described for Example 1 in Scheme 1. The crude product was dissolved in DMF and purified via preparative HPLC (Condition N). Fractions containing the desired product were combined and dried under vacuum to afford Example 92. LCMS: m/z=402.2 [M+H]⁺; HPLC Ret. Time 1.618 min. (HPLC Method C); ¹H NMR (400 MHz, DMSO-d₆) δ ppm 13.02-12.72 (m, 1H), 9.50 (br. s., 1H), 8.44 (d, J=3.4 Hz, 1H), 8.29 (s, 1H), 7.67 (d, J=9.0 Hz, 1H), 7.39 (dd, J=8.3, 5.6 Hz, 2H), 7.27-7.05 (m, 2H), 2.80 (ddt, J=11.0, 7.3, 3.9 Hz, 1H), 1.91-1.72 (m, 1H), 0.96-0.78 (m, 4H), 0.75-0.65 (m, 2H), 0.59-0.46 (m, 2H).

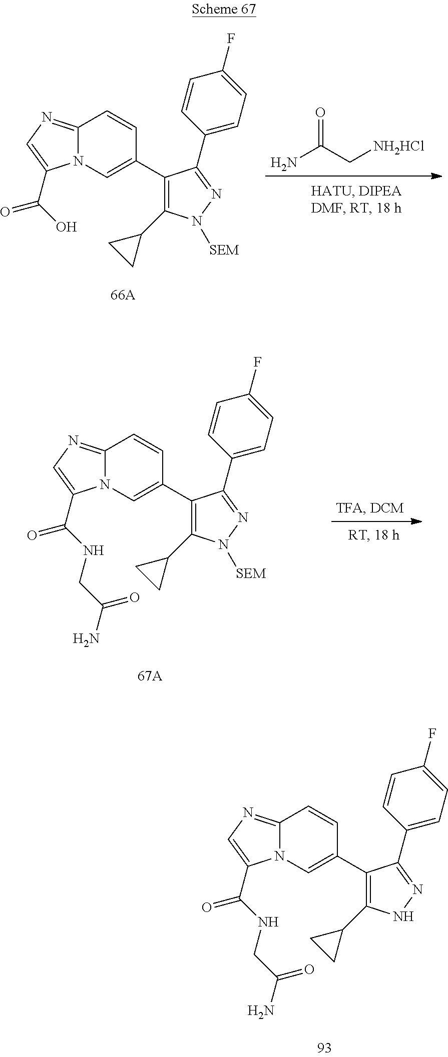

### Intermediate 67A: N-(2-amino-2-oxoethyl)-6-(5-cyclopropyl-3-(4-fluorophenyl)-1-((2-(trimethylsilyl)ethoxy)methyl)-1H-pyrazol-4-yl)imidazo[1,2-a]pyridine-3-carboxamide

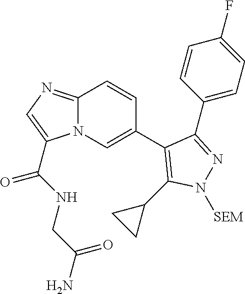 [SMILES: C[Si](C)(C)CCOCn1nc(-c2ccc(F)cc2)c(-c2ccc3ncc(C(=O)NCC(N)=O)n3c2)c1C1CC1]

Intermediate 67A was synthesized by reacting intermediate 66A with 2-aminoacetamide hydrochloride and by employing the experimental procedure described for Intermediate 66B in Scheme 66 to afford intermediate 67A as a brown gum. The crude product was used in the next step without further purification. LCMS: m/z=549.4 [M+H]⁺; HPLC Ret. Time 2.813 min. (HPLC Method E).

### Example 93: N-(2-amino-2-oxoethyl)-6-(5-cyclopropyl-3-(4-fluorophenyl)-1H-pyrazol-4-yl)imidazo[1,2-a]pyridine-3-carboxamide

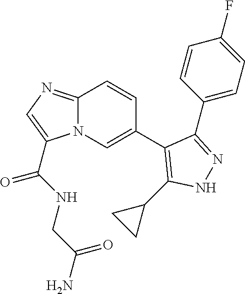 [SMILES: NC(=O)CNC(=O)c1cnc2ccc(-c3c(-c4ccc(F)cc4)n[nH]c3C3CC3)cn12]

Example 93 was synthesized by reacting intermediate 67A with TFA and by employing the experimental procedure described for Example 1 in Scheme 1. The crude product was dissolved in DMF and purified via preparative HPLC (Condition N). Fractions containing the desired product were combined and dried under vacuum to afford Example 93. LCMS: m/z=419.2 [M+H]⁺; HPLC Ret. Time 1.039 min. (HPLC Method C); ¹H NMR (400 MHz, DMSO-d₆) δ ppm 13.05-12.75 (m, 1H), 9.56-9.40 (m, 1H), 8.80-8.70 (m, 1H), 8.40 (s, 1H), 7.71 (d, J=9.3 Hz, 1H), 7.41 (d, J=12.7 Hz, 3H), 7.29-7.17 (m, 2H), 7.14-7.02 (m, 1H), 3.84 (s, 2H), 1.89-1.71 (m, 1H), 0.95-0.79 (m, 4H).

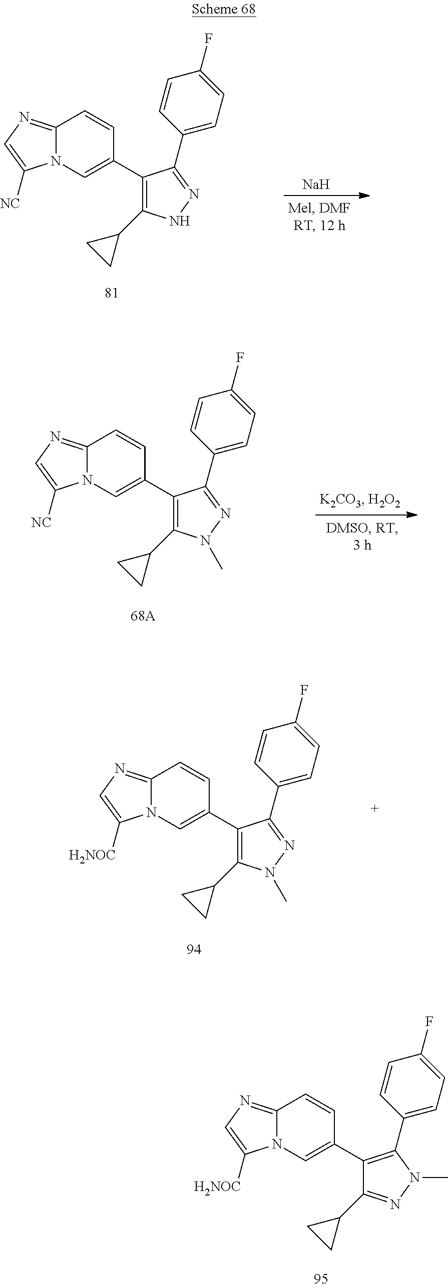

### Intermediate 68A: 6-(5-cyclopropyl-3-(4-fluorophenyl)-1-methyl-1H-pyrazol-4-yl)imidazo[1,2-a]pyridine-3-carbonitrile

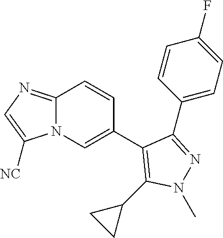 [SMILES: Cn1nc(-c2ccc(F)cc2)c(-c2ccc3ncc(C#N)n3c2)c1C1CC1]

Intermediate 68A was synthesized by reacting Example 81 with SEM-Cl and by employing the experimental procedure described for intermediate 8A in Scheme 8 to provide intermediate 68A as a gum. The regioisomeric mixture was used in the next step without purification. LCMS: m/z 358.1 [M+1]⁺; HPLC Ret. Time 1.01 min. (HPLC Method A).

### Example 94 and Example 95: 6-(5-cyclopropyl-3-(4-fluorophenyl)-1-methyl-1H-pyrazol-4-yl)imidazo[1,2-a]pyridine-3-carboxamide and 6-(3-cyclopropyl-5-(4-fluorophenyl)-1-methyl-1H-pyrazol-4-yl)imidazo[1,2-a]pyridine-3-carboxamide

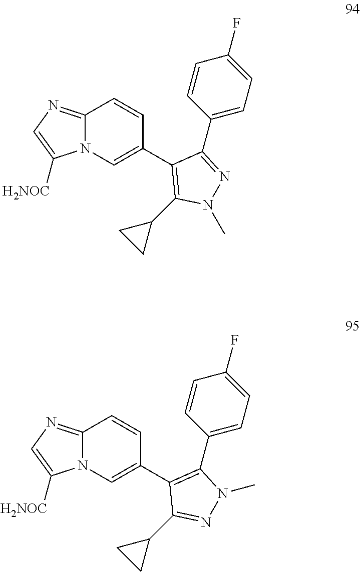 [SMILES: Cn1nc(-c2ccc(F)cc2)c(-c2ccc3ncc(C(N)=O)n3c2)c1C1CC1 ; Cn1nc(C2CC2)c(-c2ccc3ncc(C(N)=O)n3c2)c1-c1ccc(F)cc1]

Examples 94 and Example 95 were synthesized by reacting intermediate 68A with H₂O₂/K₂CO₃ and by employing the experimental procedure described for Example 2 in Scheme 1. The crude product was dissolved in DMF and purified via preparative HPLC (Condition S). Fractions containing the desired product were combined and dried under vacuum to afford Example 94. LCMS: m/z=376.1 [M+1]⁺; HPLC Ret. Time 1.484 min. (HPLC Method C). ¹H NMR (400 MHz, DMSO-d₆) δ ppm 9.40 (s, 1H), 8.36 (s, 1H), 7.97 (bs, 1H), 7.70 (d, J=9.3 Hz, 1H), 7.36 (d, J=5.4 Hz, 3H), 7.26-7.17 (m, 1H), 7.14-6.99 (m, 2H), 3.95 (s, 3H), 1.96-1.85 (m, 1H), 0.85-0.73 (m, 2H), 0.35-0.26 (m, 2H) and Example 95. LCMS: m/z=376.2 [M+1]⁺; HPLC Ret. Time 1.485 min. (HPLC Method C). ¹H NMR (400 MHz, DMSO-d₆) δ ppm 9.50 (s, 1H), 8.29 (s, 1H), 7.89 (bs, 1H), 7.60 (d, J=9.3 Hz, 1H), 7.44-7.34 (m, 2H), 7.31-7.18 (m, 3H), 7.10 (d, J=9.5 Hz, 1H), 3.66 (s, 3H), 1.86-1.71 (m, 1H), 0.91-0.78 (m, 4H).

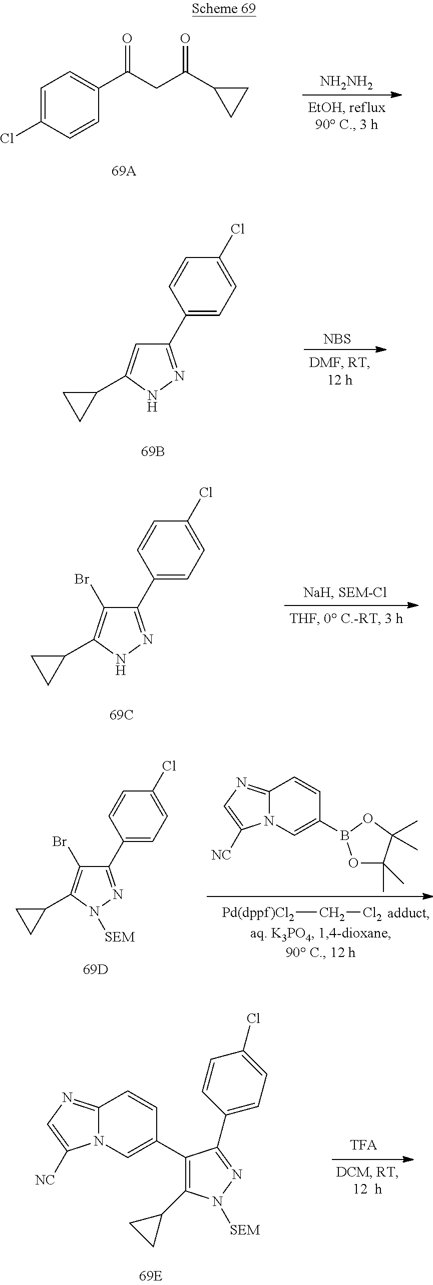

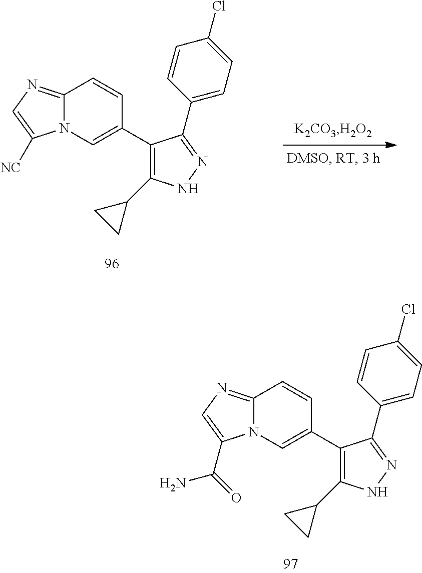 [SMILES: N#Cc1cnc2ccc(-c3c(-c4ccc(Cl)cc4)n[nH]c3C3CC3)cn12 ; NC(=O)c1cnc2ccc(-c3c(-c4ccc(Cl)cc4)n[nH]c3C3CC3)cn12]

### Intermediate 69B: 3-(4-chlorophenyl)-5-cyclopropyl-1H-pyrazole

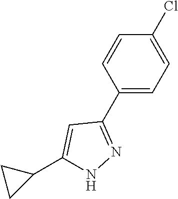 [SMILES: Clc1ccc(-c2cc(C3CC3)[nH]n2)cc1]

Intermediate 69B was synthesized by reacting intermediate 69A with hydrazine and by employing the experimental procedure described for intermediate 53B in Scheme 53. The crude product was purified by silica gel chromatography (40 g RediSep® column, eluting with a gradient of 5-10% EtOAc in petroleum ether). Fractions containing the desired product were combined and evaporated to afford 69B as an off white solid. LCMS: m/z=219.0 [M+1]⁺; HPLC Ret. Time 2.502 min. (HPLC Method E).

### Intermediate 69C: 4-bromo-3-(4-chlorophenyl)-5-cyclopropyl-1H-pyrazole

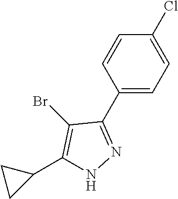 [SMILES: Clc1ccc(-c2n[nH]c(C3CC3)c2Br)cc1]

Intermediate 69C was synthesized by reacting intermediate 69B with NBS and by employing the experimental procedure described for intermediate 1D in Scheme 1. Intermediate 69C was obtained as an off white solid. LCMS: m/z=297 [M+1]⁺; HPLC Ret. Time 2.881 min. (HPLC Method E).

### Intermediate 69D: 4-bromo-3-(4-chlorophenyl)-5-cyclopropyl-1-((2-(trimethylsilyl) ethoxy)methyl)-1H-pyrazole

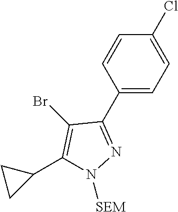 [SMILES: C[Si](C)(C)CCOCn1nc(-c2ccc(Cl)cc2)c(Br)c1C1CC1]

Intermediate 69D was synthesized by reacting intermediate 69C with SEM-Cl and by employing the experimental procedure described for intermediate 8A in Scheme 8 to afford intermediate 69D as a liquid. LCMS: m/z=427 [M+1]⁺; HPLC Ret. Time 2.253 min. (HPLC Method E).

### Intermediate 69E: 6-(3-(4-chlorophenyl)-5-cyclopropyl-1-((2-(trimethylsilyl)ethoxy)methyl)-1H-pyrazol-4-yl)imidazo[1,2-a]pyridine-3-carbonitrile

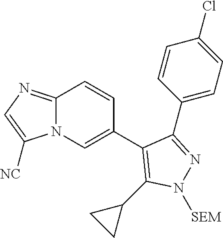 [SMILES: C[Si](C)(C)CCOCn1nc(-c2ccc(Cl)cc2)c(-c2ccc3ncc(C#N)n3c2)c1C1CC1]

Intermediate 69E was synthesized by reacting intermediate 69D with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 69E as a gum. LCMS: m/z 490.4 [M+1]⁺; HPLC Ret. Time 1.31 min. (HPLC Method B).

### Example 96: 6-(3-(4-chlorophenyl)-5-cyclopropyl-1H-pyrazol-4-yl)imidazo[1,2-a]pyridine-3-carbonitrile

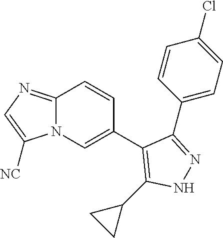 [SMILES: N#Cc1cnc2ccc(-c3c(-c4ccc(Cl)cc4)n[nH]c3C3CC3)cn12]

Example 96 was synthesized by reacting intermediate 69E with TFA and by employing the experimental procedure described for Example 1 in Scheme 1. The crude product was dissolved in DMF and purified via preparative HPLC (Condition L). Fractions containing the desired product were combined and dried under vacuum to afford Example 96. LCMS: m/z=360.1 [M+1]⁺; HPLC Ret. Time 1.874 min. (HPLC Method C). ¹H NMR (400 MHz, DMSO-d₆) δ ppm 13.12-12.85 (m, 1H), 8.49 (s, 2H), 7.84 (s, 1H), 7.40 (br. s., 5H), 1.97 (br. s., 1H), 0.95-0.75 (m, 4H).

### Example 97: 6-(3-(4-chlorophenyl)-5-cyclopropyl-1H-pyrazol-4-yl)imidazo[1,2-a]pyridine-3-carboxamide

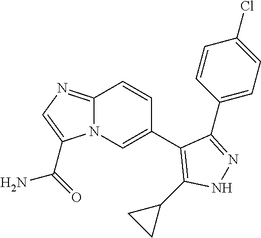 [SMILES: NC(=O)c1cnc2ccc(-c3c(-c4ccc(Cl)cc4)n[nH]c3C3CC3)cn12]

Example 97 was synthesized by reacting Example 96 with H₂O₂/K₂CO₃ and by employing the experimental procedure described for Example 2 in Scheme 1. The crude product was dissolved in DMF and purified via preparative HPLC (Condition H). Fractions containing the desired product were combined and dried under vacuum to afford Example 97. LCMS: m/z=378.1 [M+1]⁺; HPLC Ret. Time 1.504 min. (HPLC Method C). ¹H NMR (400 MHz, DMSO-d₆) δ ppm 13.10-12.74 (m, 1H), 9.51 (bs, 1H), 8.34 (s, 1H), 7.94 (bs, 1H), 7.70 (s, 1H), 7.36 (d, J=10.0 Hz, 5H), 7.21 (d, J=9.5 Hz, 1H), 1.83 (bs, 1H), 0.94-0.76 (m, 4H).

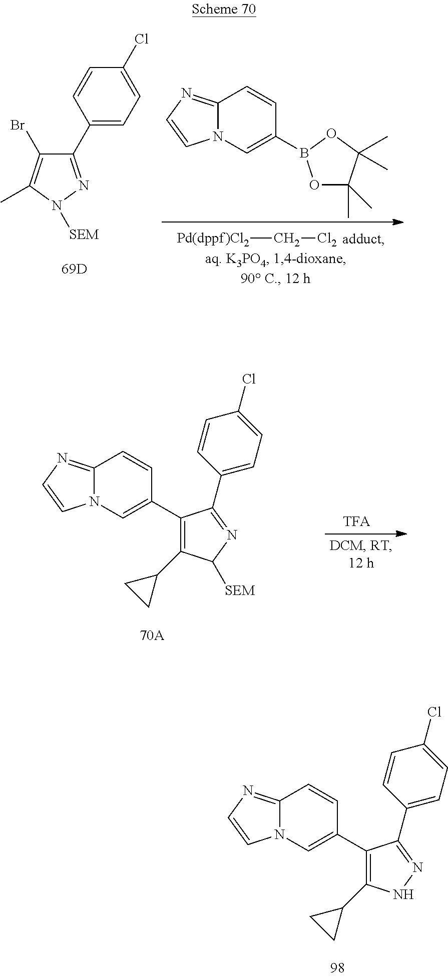

### Intermediate 70A: 6-(3-(4-chlorophenyl)-5-cyclopropyl-1-((2-(trimethylsilyl)ethoxy)methyl)-1H-pyrazol-4-yl)imidazo[1,2-a]pyridine

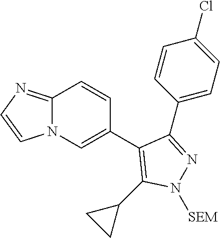 [SMILES: C[Si](C)(C)CCOCn1nc(-c2ccc(Cl)cc2)c(-c2ccc3nccn3c2)c1C1CC1]

Intermediate 70A was synthesized by reacting intermediate 69D with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 70A as a gum. LCMS: m/z=465.5 [M+1]⁺; HPLC Ret. Time 1.26 min. (HPLC Method B).

### Example 98: 6-(3-(4-chlorophenyl)-5-cyclopropyl-1H-pyrazol-4-yl)imidazo[1,2-a]pyridine

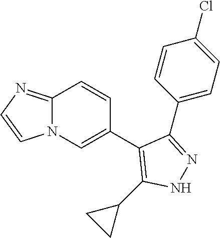 [SMILES: Clc1ccc(-c2n[nH]c(C3CC3)c2-c2ccc3nccn3c2)cc1]

Example 98 was synthesized by reacting intermediate 70A with TFA and by employing the experimental procedure described for Example 1 in Scheme 1. The crude product was dissolved in DMF and purified via preparative HPLC (Condition P). Fractions containing the desired product were combined and dried under vacuum to afford Example 98. LCMS: m/z=335.1 [M+1]⁺; HPLC Ret. Time 1.701 min. (HPLC Method C). ¹H NMR (400 MHz, DMSO-d₆) δ ppm 12.22-11.91 (m, 1H), 7.72-7.65 (m, 1H), 7.13 (s, 1H), 6.78-6.70 (m, 2H), 6.57 (bs, 4H), 6.19 (s, 1H), 1.09 (s, 1H), 0.11-0.09 (m, 4H).

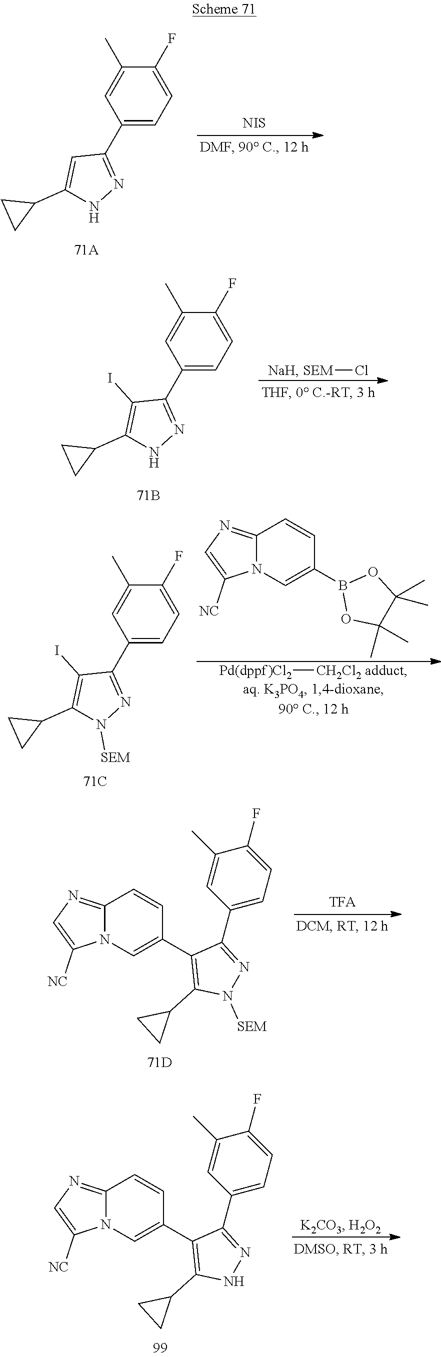

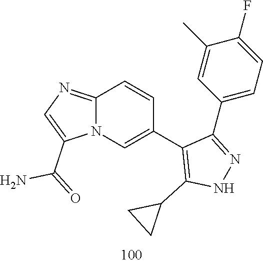 [SMILES: Cc1cc(-c2n[nH]c(C3CC3)c2-c2ccc3ncc(C(N)=O)n3c2)ccc1F]

### Intermediate 71B: 5-cyclopropyl-3-(4-fluoro-3-methylphenyl)-4-iodo-1H-pyrazole

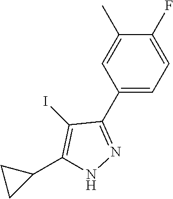 [SMILES: Cc1cc(-c2n[nH]c(C3CC3)c2I)ccc1F]

Intermediate 71B was synthesized by reacting intermediate 71A with NIS and by employing the experimental procedure described for intermediate 47B in Scheme 47. The crude product was purified by silica gel chromatography (40 g RediSep® column, eluting with a gradient of 5-10% EtOAc in petroleum ether). Fractions containing the desired product were combined and evaporated under reduced pressure to afford intermediate 71B as an off white solid. LCMS: m/z=343.0 [M+1]⁺; HPLC Ret. Time 2.924 min. (HPLC Method E).

### Intermediate 71C: 5-cyclopropyl-3-(4-fluoro-3-methylphenyl)-4-iodo-1-((2-(trimethylsilyl)ethoxy)methyl)-1H-pyrazole

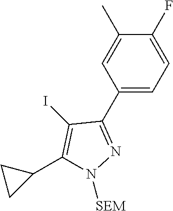 [SMILES: Cc1cc(-c2nn(COCC[Si](C)(C)C)c(C3CC3)c2I)ccc1F]

Intermediate 71C was synthesized by reacting intermediate 71B with SEM-Cl and by employing the experimental procedure described for intermediate 8A in Scheme 8 to afford intermediate 71C as a gum. LCMS: m/z=473.2 [M+1]⁺; HPLC Ret. Time 4.531 min. (HPLC Method E).

### Intermediate 71D: 6-(5-cyclopropyl-3-(4-fluoro-3-methylphenyl)-1-((2-(trimethylsilyl) ethoxy)methyl)-1H-pyrazol-4-yl)imidazo[1,2-a]pyridine-3-carbonitrile

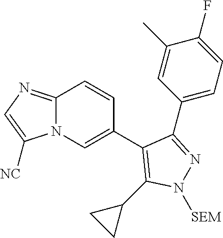 [SMILES: Cc1cc(-c2nn(COCC[Si](C)(C)C)c(C3CC3)c2-c2ccc3ncc(C#N)n3c2)ccc1F]

Intermediate 71D was synthesized by reacting intermediate 71C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 71D as a gum. LCMS: m/z=488.2 [M+1]⁺; HPLC Ret. Time 4.531 min. (HPLC Method E).

### Example 99: 6-(5-cyclopropyl-3-(4-fluoro-3-methylphenyl)-1H-pyrazol-4-yl)imidazo[1,2-a]pyridine-3-carbonitrile

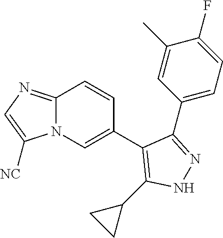 [SMILES: Cc1cc(-c2n[nH]c(C3CC3)c2-c2ccc3ncc(C#N)n3c2)ccc1F]

Example 99 was synthesized by reacting intermediate 71D with TFA and by employing the experimental procedure described for Example 1 in Scheme 1. The crude product was dissolved in DMF and purified via preparative HPLC (Condition L). Fractions containing the desired product were combined and dried under vacuum to afford Example 99 (139 mg, 81% yield). LCMS: m/z=358.1 [M+1]⁺; HPLC Ret. Time 1.750 min. (HPLC Method C). ¹H NMR (400 MHz, DMSO-d₆) δ ppm 13.01-12.77 (m, 1H), 8.50-8.35 (m, 2H), 7.86-7.75 (m, 1H), 7.38 (bs, 2H), 7.17-6.97 (m, 2H), 2.18 (bs, 3H), 2.02-1.81 (m, 1H), 0.95-0.79 (m, 4H).

### Example 100: 6-(5-cyclopropyl-3-(4-fluoro-3-methylphenyl)-1H-pyrazol-4-yl)imidazo[1,2-a]pyridine-3-carboxamide

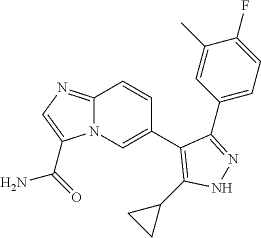 [SMILES: Cc1cc(-c2n[nH]c(C3CC3)c2-c2ccc3ncc(C(N)=O)n3c2)ccc1F]

Example 100 was synthesized by reacting Example 99 with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 100. LCMS: m/z=376.2 [M+1]⁺; HPLC Ret. Time 1.425 min. (HPLC Method C). ¹H NMR (400 MHz, DMSO-d₆) δ ppm 12.95-12.71 (m, 1H), 9.59-9.49 (m, 1H), 8.34 (s, 1H), 7.93 (bs, 1H), 7.68 (d, J=9.3 Hz, 1H), 7.38 (d, J=6.4 Hz, 1H), 7.20 (d, J=8.8 Hz, 2H), 7.10 (bs, 1H), 7.02 (d, J=9.0 Hz, 1H), 2.17 (bs, 3H), 1.87-1.72 (m, 1H), 0.90 (d, J=6.4 Hz, 4H).

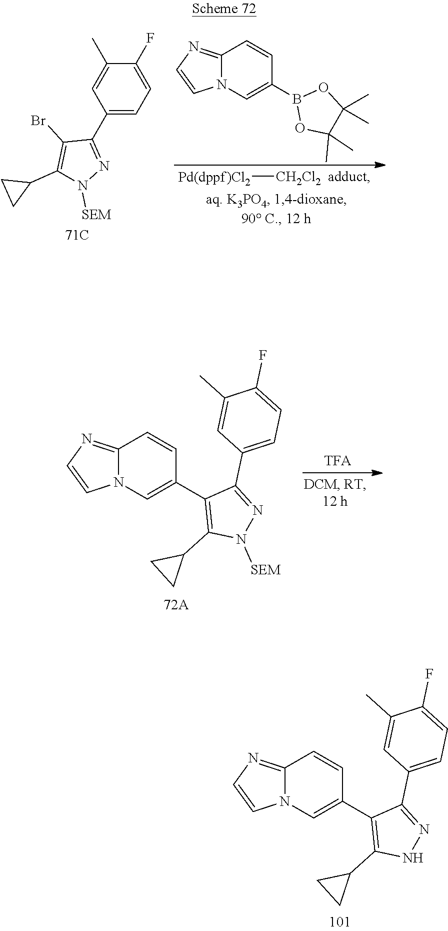

### Intermediate 72A: 6-(5-cyclopropyl-3-(4-fluoro-3-methylphenyl)-1-((2-(trimethylsilyl)ethoxy)methyl)-1H-pyrazol-4-yl)imidazo[1,2-a]pyridine

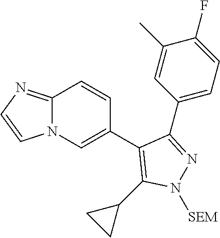 [SMILES: Cc1cc(-c2nn(COCC[Si](C)(C)C)c(C3CC3)c2-c2ccc3nccn3c2)ccc1F]

Intermediate 72A was synthesized by reacting intermediate 71C with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 72A as a gum. LCMS: m/z=463.6 [M+1]⁺; HPLC Ret. Time 1.425 min. (HPLC Method B).

### Example 101: 6-(5-cyclopropyl-3-(4-fluoro-3-methylphenyl)-1H-pyrazol-4-yl)imidazo[1,2-a]pyridine

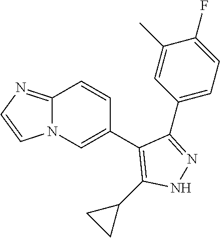 [SMILES: Cc1cc(-c2n[nH]c(C3CC3)c2-c2ccc3nccn3c2)ccc1F]

Example 101 was synthesized by reacting intermediate 72A with TFA and by employing the experimental procedure described for Example 1 in Scheme 1. The crude product was dissolved in DMF and purified via preparative HPLC (Condition L). Fractions containing the desired product were combined and dried under vacuum to afford Example 101. LCMS: m/z=333.2 [M+1]⁺; HPLC Ret. Time 1.649 min. (HPLC Method C). ¹H NMR (400 MHz, DMSO-d₆) δ ppm 12.95 (br. s., 1H), 8.82-8.61 (m, 1H), 8.26-8.10 (m, 1H), 7.89-7.65 (m, 2H), 7.67-7.56 (m, 1H), 7.41-7.18 (m, 3H), 2.43 (s, 3H), 2.17-1.98 (m, 1H), 1.20-0.93 (m, 4H).

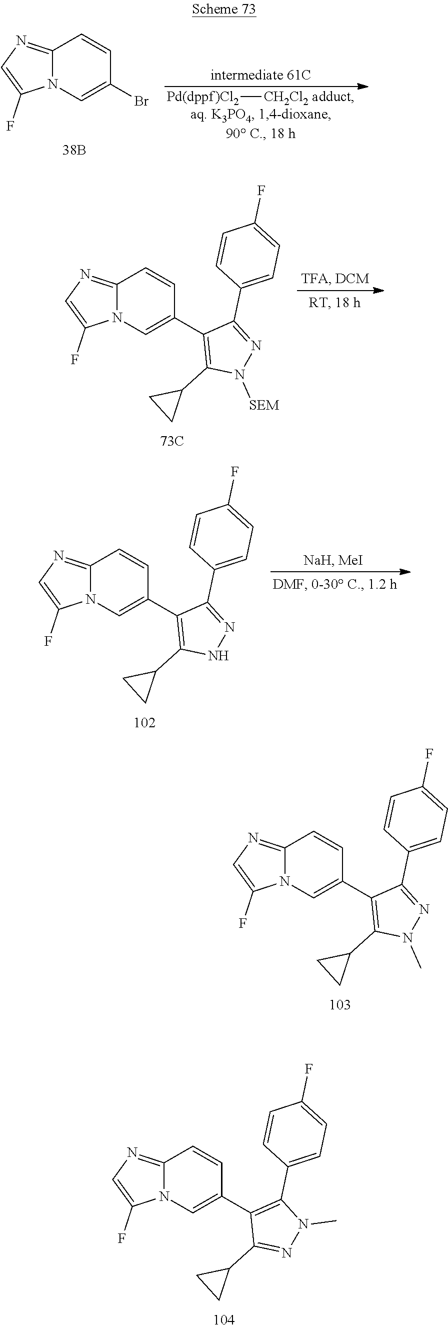

### Intermediate 73C: 6-(5-cyclopropyl-3-(4-fluorophenyl)-1-((2-(trimethylsilyl)ethoxy)methyl)-1H-pyrazol-4-yl)-3-fluoroimidazo[1,2-a]pyridine

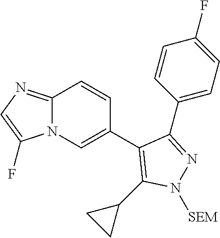 [SMILES: C[Si](C)(C)CCOCn1nc(-c2ccc(F)cc2)c(-c2ccc3ncc(F)n3c2)c1C1CC1]

Intermediate 73C was synthesized by coupling intermediate 61C with intermediate 38B and by employing the experimental procedure described for intermediate 1E in Scheme 1. The crude product was purified by silica gel chromatography (24 g RediSep® column, eluting with a gradient of 49-51% EtOAc in petroleum ether). Fractions containing the desired product were combined and evaporated under reduced pressure to afford intermediate 73C. LCMS: m/z=467.2 [M+H]⁺; HPLC Ret. Time 3.66 min. (HPLC Method E).

### Example 102: 6-(5-cyclopropyl-3-(4-fluorophenyl)-1H-pyrazol-4-yl)-3-fluoroimidazo[1,2-a]pyridine

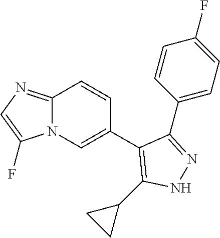 [SMILES: Fc1ccc(-c2n[nH]c(C3CC3)c2-c2ccc3ncc(F)n3c2)cc1]

Example 102 was synthesized by reacting intermediate 73C with TFA and by employing the experimental procedure described for Example 1 in Scheme 1. The crude product was dissolved in DMF and purified via preparative HPLC (Condition N). Fractions containing the desired product were combined and dried under vacuum to afford Example 102. LCMS: m/z=337.1 [M+H]⁺; HPLC Ret. Time 1.612 min. (HPLC Method C); ¹H NMR (400 MHz, DMSO-d₆) δ ppm 13.02 (br. s. 1H), 8.48-8.38 (m, 1H), 7.63 (s, 4H), 7.53-7.33 (m, 2H), 7.23 (d, J=8.6 Hz, 1H), 2.24-2.03 (m, 1H), 1.21-1.02 (m, 4H).

### Example 103 and 104: 6-(5-cyclopropyl-3-(4-fluorophenyl)-1-methyl-1H-pyrazol-4-yl)-3-fluoroimidazo[1,2-a]pyridine and 6-(3-cyclopropyl-5-(4-fluorophenyl)-1-methyl-1H-pyrazol-4-yl)-3-fluoroimidazo[1,2-a]pyridine

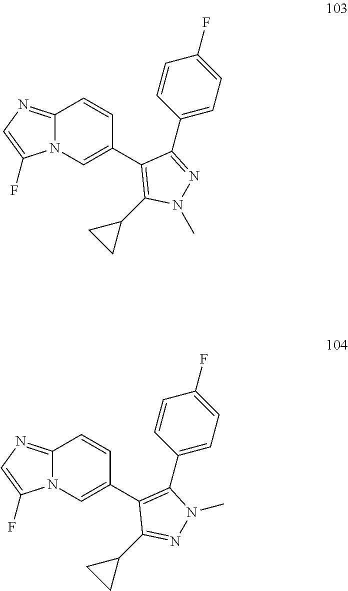 [SMILES: Cn1nc(-c2ccc(F)cc2)c(-c2ccc3ncc(F)n3c2)c1C1CC1 ; Cn1nc(C2CC2)c(-c2ccc3ncc(F)n3c2)c1-c1ccc(F)cc1]

Examples 103 and 104 were synthesized by reacting intermediate 102 with methyl iodide and by employing the experimental procedure described for Example 6B in Scheme 6. The crude product was dissolved in DMF and purified via preparative HPLC (Condition N). Fractions containing the desired product were combined and dried under vacuum to afford Example 103. LCMS: m/z=351.1 [M+H]⁺; HPLC Ret. Time 1.883 min. (HPLC Method C); ¹H NMR (400 MHz, DMSO-d₆) δ ppm 8.19-8.17 (m, 1H), 7.55-7.50 (m, 1H), 7.45-7.37 (m, 2H), 7.23-7.20 (m, 2H), 7.16-7.08 (m, 1H), 7.01-6.95 (m, 1H), 3.96-3.94 (s, 3H), 2.00-1.95 (m, 1H), 0.88-0.82 (m, 2H), 0.38-0.32 (m, 2H) and Example 104. LCMS: m/z=351.1 [M+H]⁺; HPLC Ret. Time 1.903 min. (HPLC Method C); ¹H NMR (400 MHz, DMSO-d₆) δ ppm 8.04 (s, 1H), 7.46-7.39 (m, 2H), 7.34 (d, J=7.5 Hz, 1H), 7.31-7.24 (m, 2H), 7.22-6.96 (m, 1H), 6.90 (dd, J=9.3, 1.8 Hz, 1H), 3.66 (s, 3H), 1.93-1.83 (m, 1H), 0.88-0.80 (m, 4H).

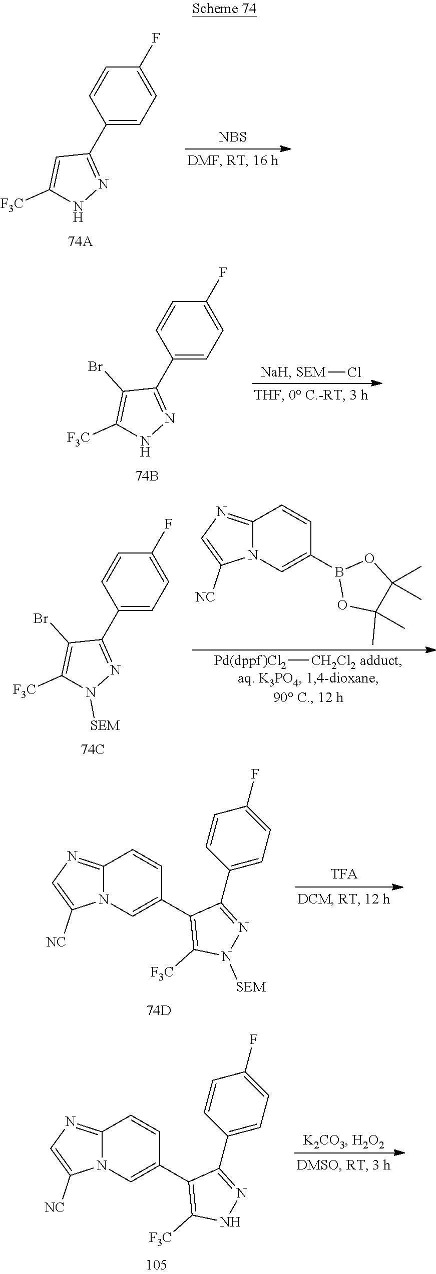

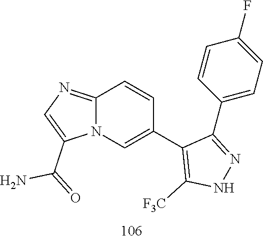 [SMILES: NC(=O)c1cnc2ccc(-c3c(-c4ccc(F)cc4)n[nH]c3C(F)(F)F)cn12]

### Intermediate 74B: 4-bromo-3-(4-fluorophenyl)-5-(trifluoromethyl)-1H-pyrazole

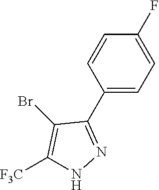 [SMILES: Fc1ccc(-c2n[nH]c(C(F)(F)F)c2Br)cc1]

Intermediate 74B was synthesized by reacting intermediate 74A with NBS and by employing the experimental procedure described for intermediate 1D in Scheme 1 to afford 74B as an off white solid. LCMS: m/z=309.0 [M+1]⁺; HPLC Ret. Time 2.807 min (HPLC Method E).

### Intermediate 74C: 4-bromo-3-(4-fluorophenyl)-5-(trifluoromethyl)-1-((2-(trimethylsilyl)ethoxy)methyl)-1H-pyrazole

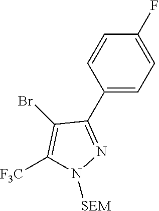 [SMILES: C[Si](C)(C)CCOCn1nc(-c2ccc(F)cc2)c(Br)c1C(F)(F)F]

Intermediate 74C was synthesized by reacting intermediate 74B with SEM-Cl and by employing the experimental procedure described for intermediate 8A in Scheme 8 to afford intermediate 74C as a liquid. LCMS: m/z=439.0 [M+1]⁺; HPLC Ret. Time 1.441 (HPLC Method E).

### Intermediate 74D: 6-(3-(4-fluorophenyl)-5-(trifluoromethyl)-1-((2-(trimethylsilyl)ethoxy)methyl)-1H-pyrazol-4-yl)imidazo[1,2-a]pyridine-3-carbonitrile

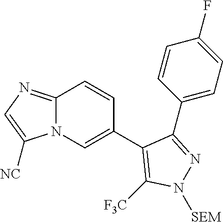 [SMILES: C[Si](C)(C)CCOCn1nc(-c2ccc(F)cc2)c(-c2ccc3ncc(C#N)n3c2)c1C(F)(F)F]

Intermediate 74D was synthesized by reacting intermediate 74C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 74D as a gum. LCMS: m/z=502.4 [M+1]⁺; HPLC Ret. Time 1.29 (HPLC Method B).

### Example 105: 6-(3-(4-fluorophenyl)-5-(trifluoromethyl)-1H-pyrazol-4-yl)imidazo[1,2-a]pyridine-3-carbonitrile

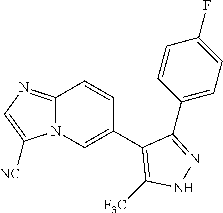 [SMILES: N#Cc1cnc2ccc(-c3c(-c4ccc(F)cc4)n[nH]c3C(F)(F)F)cn12]

Example 105 was synthesized by reacting intermediate 74D with TFA and by employing the experimental procedure described for Example 1 in Scheme 1. The crude product was dissolved in DMF and purified via preparative HPLC (Condition T). Fractions containing the desired product were combined and dried under vacuum to afford Example 105. LCMS: m/z=372.1 [M+1]⁺; HPLC Ret. Time 1.818 (HPLC Method C). ¹H NMR (400 MHz, DMSO-d₆) δ ppm 14.28 (bs, 1H), 8.57 (s, 1H), 8.50 (s, 1H), 7.87 (d, J=9.3 Hz, 1H), 7.49-7.43 (m, 2H), 7.39 (dd, J=9.2, 1.6 Hz, 1H), 7.25 (t, J=8.8 Hz, 2H).

### Example 106: 6-(3-(4-fluorophenyl)-5-(trifluoromethyl)-1H-pyrazol-4-yl)imidazo[1,2-a]pyridine-3-carboxamide

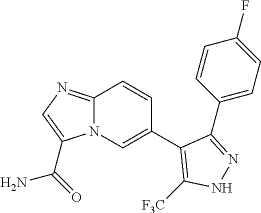 [SMILES: NC(=O)c1cnc2ccc(-c3c(-c4ccc(F)cc4)n[nH]c3C(F)(F)F)cn12]

Example 106 was synthesized by reacting Example 105 with H₂O₂/K₂CO₃ and by employing the experimental procedure described for Example 2 in Scheme 1. The crude product was dissolved in DMF and purified via preparative HPLC (Condition H). Fractions containing the desired product were combined and dried under vacuum to afford Example 106. LCMS: m/z=390.1 [M+1]⁺; HPLC Ret. Time 1.435 (HPLC Method C). ¹H NMR (400 MHz, DMSO-d₆) δ ppm 4.24 (bs, 1H), 9.47 (s, 1H), 8.37 (s, 1H), 7.99 (bs, 1H), 7.75 (s, 1H), 7.43 (dd, J=8.6, 5.4 Hz, 2H), 7.39-7.31 (m, 1H), 7.29-7.22 (m, 3H).

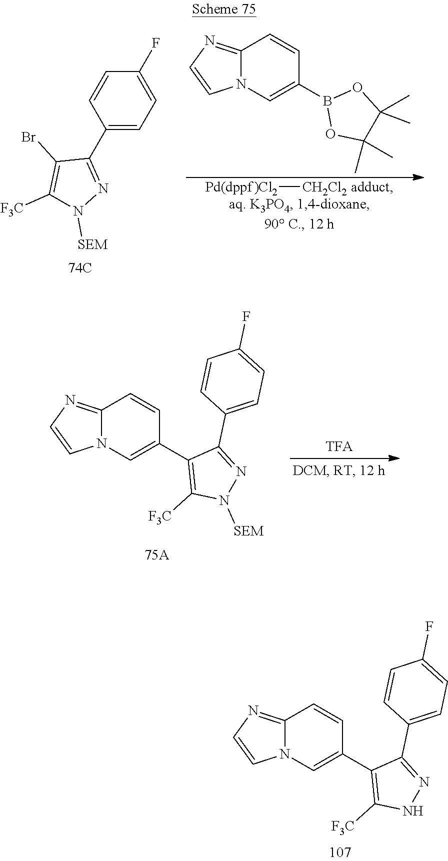

### Intermediate 75A: 6-(3-(4-fluorophenyl)-5-(trifluoromethyl)-1-((2-(trimethylsilyl)ethoxy)methyl)-1H-pyrazol-4-yl)imidazo[1,2-a]pyridine

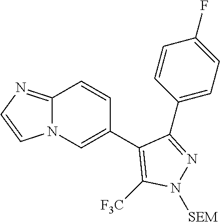 [SMILES: C[Si](C)(C)CCOCn1nc(-c2ccc(F)cc2)c(-c2ccc3nccn3c2)c1C(F)(F)F]

Intermediate 75A was synthesized by reacting intermediate 74C with 6-(4,4,5,5-tetramethyl-1,3,2-dioxaborolan-2-yl)imidazo[1,2-a]pyridine and by employing the experimental procedure described for Intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 75A as a gum. LCMS: m/z=477.5 [M+1]⁺; HPLC Ret. Time 1.25 (HPLC Method B).

### Example 107: 6-(3-(4-fluorophenyl)-5-(trifluoromethyl)-1H-pyrazol-4-yl)imidazo[1,2-a]pyridine

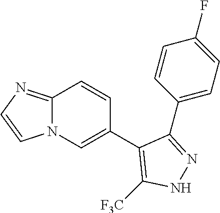 [SMILES: Fc1ccc(-c2n[nH]c(C(F)(F)F)c2-c2ccc3nccn3c2)cc1]

Example 107 was synthesized by reacting intermediate 75A with TFA and by employing the experimental procedure described for Example 1 in Scheme 1. The crude product was dissolved in DMF and purified via preparative HPLC (Condition H). Fractions containing the desired product were combined and dried under vacuum to afford Example 107. LCMS: m/z=347.1 [M+1]⁺; HPLC Ret. Time 1.645 (HPLC Method C). ¹H NMR (400 MHz, DMSO-d₆) δ ppm 14.21 (bs, 1H), 8.53 (s, 1H), 7.96 (s, 1H), 7.64-7.52 (m, 2H), 7.45 (s, 2H), 7.31-7.17 (m, 2H), 7.05 (dd, J=9.4, 1.6 Hz, 1H).

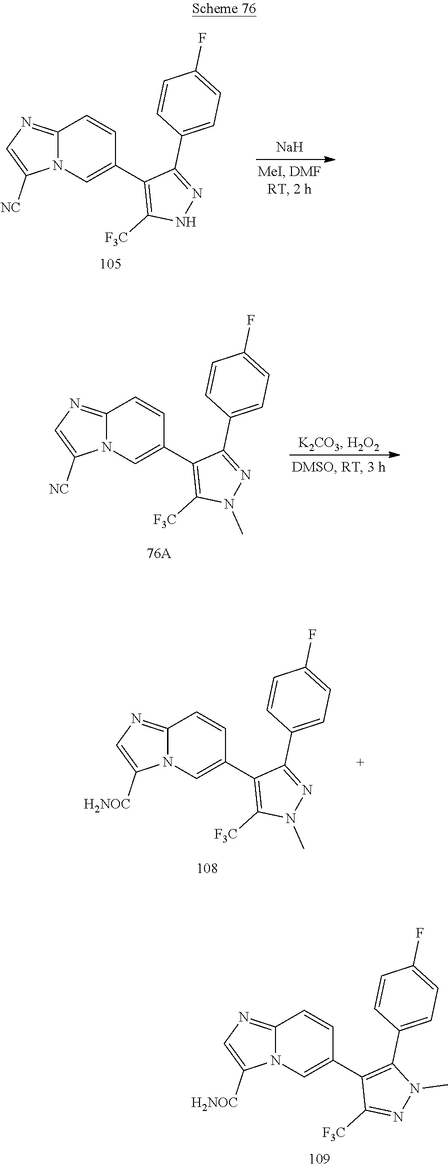

### Intermediate 76A: 6-(3-(4-fluorophenyl)-1-methyl-5-(trifluoromethyl)-1H-pyrazol-4-yl)imidazo[1,2-a]pyridine-3-carbonitrile

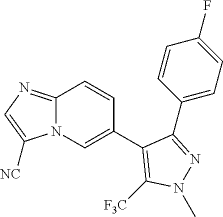 [SMILES: Cn1nc(-c2ccc(F)cc2)c(-c2ccc3ncc(C#N)n3c2)c1C(F)(F)F]

Example 76A was synthesized by reacting Example 105 with methyl iodide and by employing the experimental procedure described for Example 6B in Scheme 6 to afford Intermediate 76A as a gum. The crude product was used in the next step without purification. LCMS: m/z=386.5 [M+1]⁺; HPLC Ret. Time 0.85 min. (HPLC Method A).

### Example 108 and 109: 6-(3-(4-fluorophenyl)-1-methyl-5-(trifluoromethyl)-1H-pyrazol-4-yl)imidazo[1,2-a]pyridine-3-carboxamide and 6-(5-(4-fluorophenyl)-1-methyl-3-(trifluoromethyl)-1H-pyrazol-4-yl)imidazo[1,2-a]pyridine-3-carboxamide

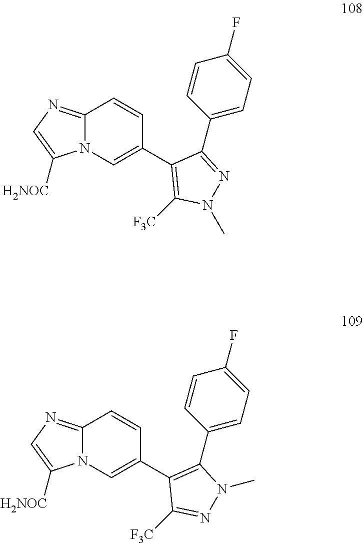 [SMILES: Cn1nc(-c2ccc(F)cc2)c(-c2ccc3ncc(C(N)=O)n3c2)c1C(F)(F)F ; Cn1nc(C(F)(F)F)c(-c2ccc3ncc(C(N)=O)n3c2)c1-c1ccc(F)cc1]

Examples 108 and 109 were synthesized by reacting intermediate 76A with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 108. LCMS: m/z=404.1 [M+1]⁺; HPLC Ret. Time 1.676 (HPLC Method C). ¹H NMR (400 MHz, DMSO-d₆) δ ppm 9.43 (d, J=2.0 Hz, 1H), 8.39 (s, 1H), 8.00 (bs, 1H), 7.77 (dd, J=9.0, 1.0 Hz, 1H), 7.42-7.29 (m, 4H), 7.18-7.11 (m, 2H), 4.12 (d, J=1.0 Hz, 3H) and Example 109. LCMS: m/z=404.1 [M+1]⁺; HPLC Ret. Time 1.585 (HPLC Method C). ¹H NMR (400 MHz, DMSO-d₆) δ ppm 9.42 (s, 1H), 8.33 (s, 1H), 7.93 (bs, 1H), 7.67 (d, J=1.0 Hz, 1H), 7.57-7.42 (m, 2H), 7.39-7.21 (m, 3H), 7.20 (d, J=2.0 Hz, 1H), 3.17 (s, 3H).

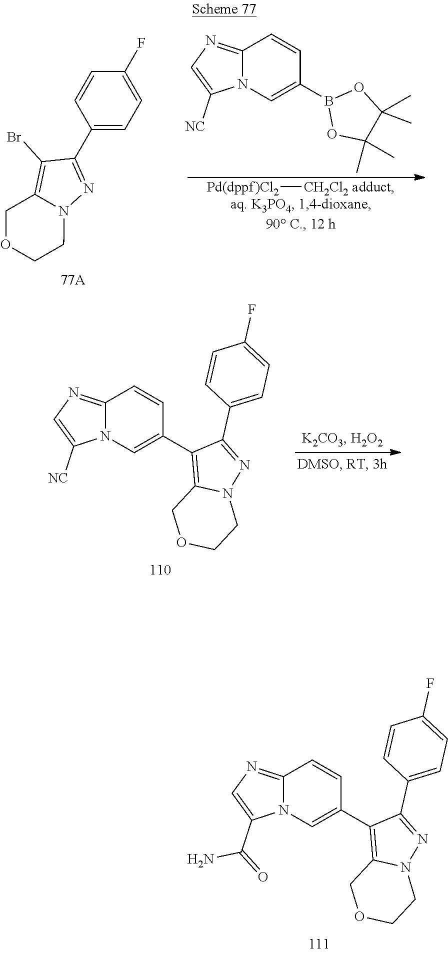

### Example 110: 6-(2-(4-fluorophenyl)-6,7-dihydro-4H-pyrazolo[5,1-c][1,4]oxazin-3-yl)imidazo[1,2-a]pyridine-3-carbonitrile

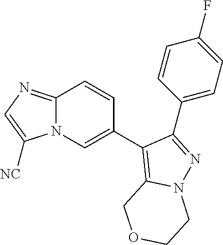 [SMILES: N#Cc1cnc2ccc(-c3c(-c4ccc(F)cc4)nn4c3COCC4)cn12]

Example 110 was synthesized by reacting intermediate 77A (reference: WO 2011/124539 A1) with 6-(4,4,5,5-tetramethyl-1,3,2-dioxaborolan-2-yl)imidazo[1,2-a]pyridine-3-carbonitrile and by employing the experimental procedure described for Intermediate 1E in Scheme 1. The crude product was dissolved in DMF and purified via preparative HPLC (Condition L). Fractions containing the desired product were combined and dried under vacuum to afford Example 110. LCMS: m/z=360.1 [M+1]⁺; HPLC Ret. Time 1.371 (HPLC Method C). ¹H NMR (400 MHz, DMSO-d₆) δ ppm 8.46-8.42 (m, 2H), 7.80 (dd, J=9.3, 1.0 Hz, 1H), 7.46 (dd, J=9.0, 5.6 Hz, 2H), 7.23 (dd, J=9.3, 1.7 Hz, 1H), 7.16 (t, J=8.9 Hz, 2H), 4.93 (s, 2H), 4.26-4.12 (m, 4H).

### Example 111: 6-(2-(4-fluorophenyl)-6,7-dihydro-4H-pyrazolo[5,1-c][1,4]oxazin-3-yl)imidazo[1,2-a]pyridine-3-carboxamide

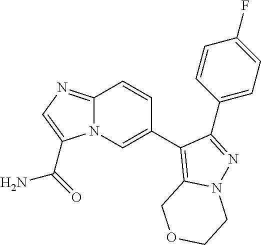 [SMILES: NC(=O)c1cnc2ccc(-c3c(-c4ccc(F)cc4)nn4c3COCC4)cn12]

Example 111 was synthesized by reacting Example 110 with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 111. LCMS: m/z=378.1 [M+1]⁺; HPLC Ret. Time 1.313 (HPLC Method C). ¹H NMR (400 MHz, DMSO-d₆) δ ppm 9.31 (dd, J=1.7, 1.0 Hz, 1H), 8.32 (s, 1H), 7.91 (bs, 1H), 7.66 (dd, J=9.2, 0.9 Hz, 1H), 7.46-7.37 (m, 2H), 7.34-7.23 (m, 1H), 7.19-7.01 (m, 3H), 4.82 (s, 2H), 4.27-4.09 (m, 4H).

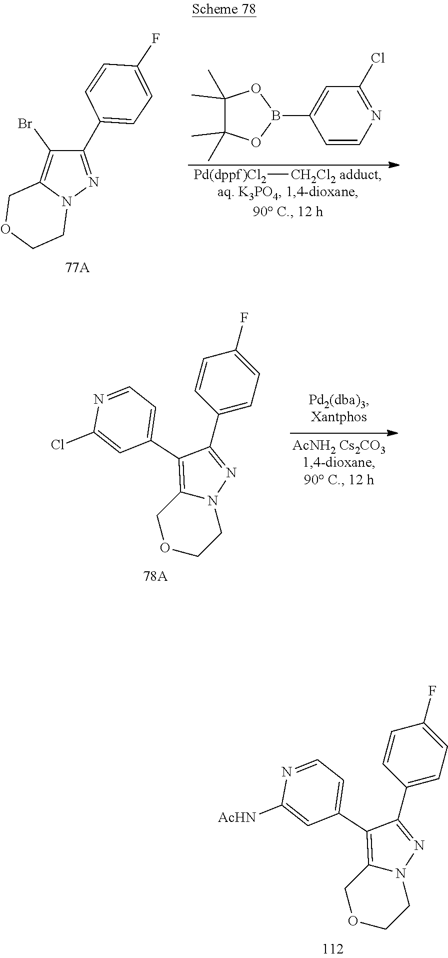

### Intermediate 78A: 3-(2-chloropyridin-4-yl)-2-(4-fluorophenyl)-6,7-dihydro-4H-pyrazolo[5,1-c][1,4]oxazine

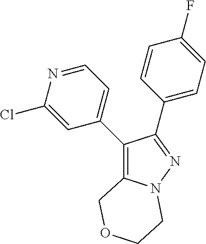 [SMILES: Fc1ccc(-c2nn3c(c2-c2ccnc(Cl)c2)COCC3)cc1]

Intermediate 78A was synthesized by reacting intermediate 77A with 2-chloropyridine-4-boronic acid pinacoleter and by employing the experimental procedure described for Example 37 in Scheme 25. The crude product was purified by silica gel chromatography (40 g RediSep® column, eluting with a gradient of 20-30% EtOAc in petroleum ether). Fractions containing the desired product were combined and evaporated under reduced pressure to afford intermediate 78A. LCMS: m/z=330.0 [M+1]⁺; HPLC Ret. Time 1.17 (HPLC Method B).

### Example 112: N-(4-(2-(4-fluorophenyl)-6,7-dihydro-4H-pyrazolo[5,1-c][1,4]oxazin-3-yl)pyridin-2-yl)acetamide

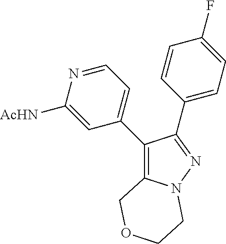 [SMILES: CC(=O)Nc1cc(-c2c(-c3ccc(F)cc3)nn3c2COCC3)ccn1]

To a stirred solution of 78A (60 mg, 0.182 mmol) in 1,4-dioxane (8 mL) was added 4,5-bis(diphenylphosphino)-9,9-dimethylxanthene (21.06 mg, 0.036 mmol) acetamide (21.49 mg, 0.364 mmol) and Cs₂CO₃ (119 mg, 0.364 mmol). The reaction mixture was degassed for 1 min., and treated with Pd₂(dba)₃ (16.66 mg, 0.018 mmol). The resultant mixture was heated to 100° C. for 16 h. The mixture was cooled to RT, filtered through a Celite® pad and the filter-cake was washed with ethyl acetate. The combined filtrate was evaporated under reduced pressure. The crude product was dissolved in DMF and purified via preparative HPLC (Condition H). Fractions containing the desired product were combined and dried under vacuum to afford Example 112 (13.6 mg, 21.21% yield). LCMS: m/z=353.1 [M+1]⁺; HPLC Ret. Time 1.437 (HPLC Method C). ¹H NMR (400 MHz, DMSO-d₆) δ ppm 10.47 (s, 1H), 8.20 (dd, J=5.1, 0.7 Hz, 1H), 7.88 (s, 1H), 7.42 (dd, J=8.9, 5.5 Hz, 2H), 7.19 (t, J=9.0 Hz, 2H), 6.76 (dd, J=5.1, 1.7 Hz, 1H), 4.87 (s, 2H), 4.28-3.93 (m, 4H), 2.06 (s, 3H).

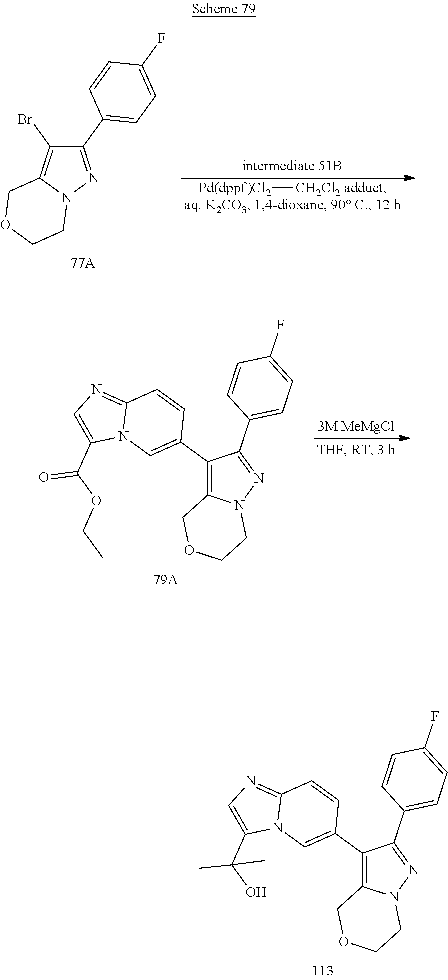

### Intermediate 79A: ethyl 6-(2-(4-fluorophenyl)-6,7-dihydro-4H-pyrazolo[5,1-c][1,4]oxazin-3-yl)imidazo[1,2-a]pyridine-3-carboxylate

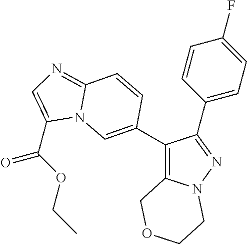 [SMILES: CCOC(=O)c1cnc2ccc(-c3c(-c4ccc(F)cc4)nn4c3COCC4)cn12]

Intermediate 79A was synthesized by reacting intermediate 77A with intermediate 51B and by employing the experimental procedure described for Example 37 in Scheme 25. The crude product was purified by silica gel chromatography (40 g RediSep® column, eluting with a gradient of 20-30% EtOAc in petroleum ether). Fractions containing the desired product were combined and evaporated to afford intermediate 79A as an off white solid. LCMS: m/z=407.3 [M+1]⁺; HPLC Ret. Time 2.589 (HPLC Method E).

### Example 113: 2-(6-(2-(4-fluorophenyl)-6,7-dihydro-4H-pyrazolo[5,1-c][1,4]oxazin-3-yl)imidazo[1,2-a]pyridin-3-yl)propan-2-ol

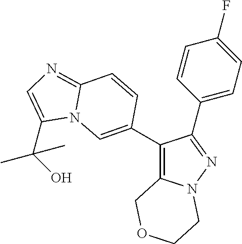 [SMILES: CC(C)(O)c1cnc2ccc(-c3c(-c4ccc(F)cc4)nn4c3COCC4)cn12]

Example 113 was synthesized by reacting intermediate 79A with MeMgCl and by employing the experimental procedure described for Example 52 in Scheme 34. The crude product was dissolved in DMF and purified via preparative HPLC (Condition H). Fractions containing the desired product were combined and dried under vacuum to afford Example 113. LCMS: m/z 393 [M+1]⁺; HPLC Ret. Time 1.437 (HPLC Method C). ¹H NMR (400 MHz, DMSO-d₆) δ ppm 8.50 (s, 1H), 7.54 (d, J=9.3 Hz, 1H), 7.50-7.44 (m, 2H), 7.40 (s, 1H), 7.21-7.13 (m, 2H), 6.99 (dd, J=9.3, 1.7 Hz, 1H), 5.33-5.28 (m, 1H), 4.88 (s, 2H), 4.26-4.14 (m, 4H), 1.53 (s, 6H).

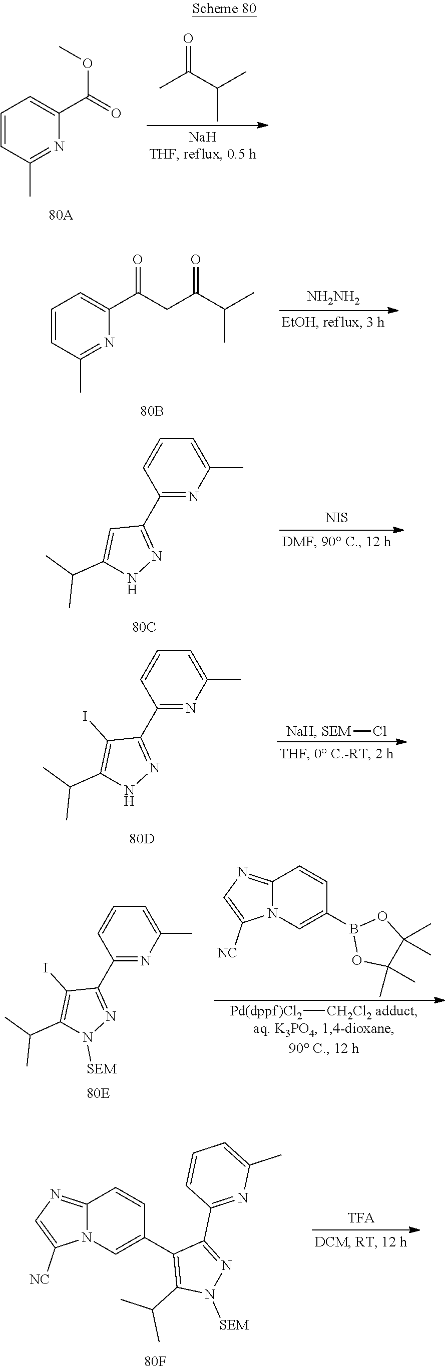

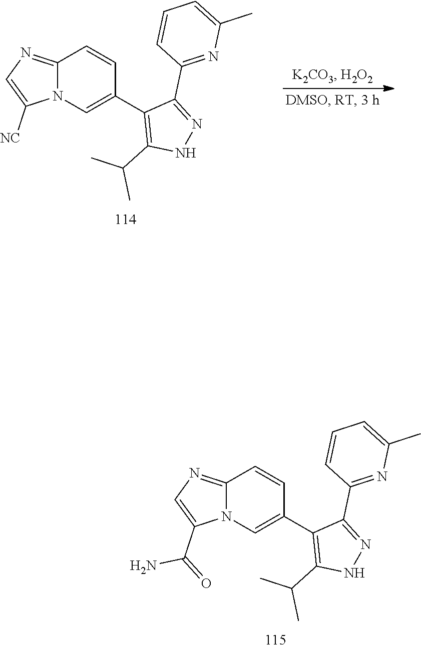 [SMILES: Cc1cccc(-c2n[nH]c(C(C)C)c2-c2ccc3ncc(C#N)n3c2)n1 ; Cc1cccc(-c2n[nH]c(C(C)C)c2-c2ccc3ncc(C(N)=O)n3c2)n1]

### Intermediate 80B: 4-methyl-1-(6-methylpyridin-2-yl)pentane-1,3-dione

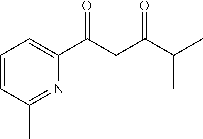

To a suspension of NaH (0.661 g, 16.54 mmol, 60% in mineral oil) in THF (15 mL) was added 3-methylbutan-2-one (1.559 mL, 14.55 mmol). The reaction mixture was stirred at RT for 30 min, heated to reflux, followed by the addition of a solution of 80A (1.0 g, 6.62 mmol) in THF (3 mL). The resultant mixture was maintained at reflux for an additional 30 min., cooled to RT, poured into 1N HCl solution (pH 7-8) and extracted with ethyl acetate. The organic layer was dried over anhydrous Na₂SO₄, filtered and the filtrate was evaporated under reduced pressure. The crude product was purified by silica gel chromatography (80 g RediSep® column, eluting with a gradient of 5-10% EtOAc in petroleum ether). Fractions containing the desired product were combined and evaporated under reduced pressure to afford intermediate 80B (840 mg, 50.1% yield). LCMS: m/z=206.2 [M+H]⁺; HPLC Ret. Time 3.039 min. (HPLC Method E).

### Intermediate 80C: 2-(5-isopropyl-1H-pyrazol-3-yl)-6-methylpyridine

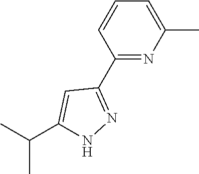

Intermediate 80C was synthesized by reacting intermediate 80B with methyl iodide and by employing the experimental procedure described for intermediate 53B in Scheme 53 to afford intermediate 80C as a semi-solid. LCMS: m/z=202.2 [M+H]⁺; HPLC Ret. Time 1.734 min. (HPLC Method E).

### Intermediate 80D: 2-(4-iodo-5-isopropyl-1H-pyrazol-3-yl)-6-methylpyridine

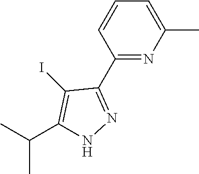 [SMILES: Cc1cccc(-c2n[nH]c(C(C)C)c2I)n1]

Intermediate 80D was synthesized by reacting intermediate 80C with NIS and by employing the experimental procedure described for intermediate 47B in Scheme 47 to afford intermediate 80D as a gum. LCMS: m/z=328.3 [M+H]⁺; HPLC Ret. Time 1.35 min. (HPLC Method B).

### Intermediate 80E: 2-(4-iodo-5-isopropyl-1-((2-(trimethylsilyl)ethoxy)methyl)-1H-pyrazol-3-yl)-6-methylpyridine

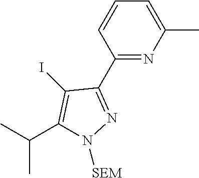 [SMILES: Cc1cccc(-c2nn(COCC[Si](C)(C)C)c(C(C)C)c2I)n1]

Intermediate 80E was synthesized by reacting intermediate 80D with SEM-Cl and by employing the experimental procedure described for intermediate 8A in Scheme 8. The crude product was purified by silica gel chromatography (40 g RediSep® column, eluting with a gradient of 5-8% EtOAc in petroleum ether). Fractions containing the desired product were combined and evaporated under reduced pressure to afford intermediate 80E. LCMS: m/z=458.0 [M+H]⁺; HPLC Ret. Time 4.274 min. (HPLC Method E).

### Intermediate 80F: 6-(5-isopropyl-3-(6-methylpyridin-2-yl)-1-((2-(trimethylsilyl) ethoxy)methyl)-1H-pyrazol-4-yl)imidazo [1,2-a]pyridine-3-carbonitrile

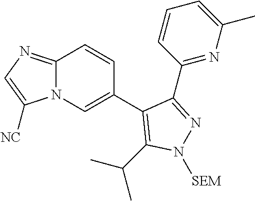 [SMILES: Cc1cccc(-c2nn(COCC[Si](C)(C)C)c(C(C)C)c2-c2ccc3ncc(C#N)n3c2)n1]

Intermediate 80F was synthesized by reacting intermediate 80E with 6-(4,4,5,5-tetramethyl-1,3,2-dioxaborolan-2-yl)imidazo[1,2-a]pyridine-3-carbonitrile and by employing the experimental procedure described for Intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 80F. LCMS: m/z=473.2 [M+H]⁺; HPLC Ret. Time 4.274 min. (HPLC Method E).

### Example 114: 6-(5-isopropyl-3-(6-methylpyridin-2-yl)-1H-pyrazol-4-yl) imidazo [1,2-a]pyridine-3-carbonitrile

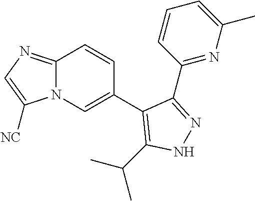 [SMILES: Cc1cccc(-c2n[nH]c(C(C)C)c2-c2ccc3ncc(C#N)n3c2)n1]

Example 114 was synthesized by reacting intermediate 80F with TFA and by employing the experimental procedure described for Example 1 in Scheme 1. The crude product was dissolved in DMF and purified via preparative HPLC (Condition N). Fractions containing the desired product were combined and dried under vacuum to afford Example 114. LCMS: m/z=343.1 [M+H]⁺; HPLC Ret. Time 1.659 min. (HPLC Method C); ¹H NMR (400 MHz, DMSO-d₆) δ ppm 13.11 (bs, 1H), 8.60 (s, 1H), 8.46 (s, 1H), 7.81 (d, J=8.6 Hz, 1H), 7.65 (bs, 2H), 7.47 (s, 1H), 7.11 (bs, 1H), 3.00 (bs, 1H), 2.24 (bs, 3H), 1.24 (bs, 6H).

### Example 115: 6-(5-isopropyl-3-(6-methylpyridin-2-yl)-1H-pyrazol-4-yl)imidazo[1,2-a]pyridine-3-carboxamide

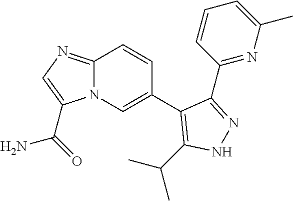 [SMILES: Cc1cccc(-c2n[nH]c(C(C)C)c2-c2ccc3ncc(C(N)=O)n3c2)n1]

Example 115 was synthesized by reacting Example 114 with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 115. LCMS: m/z=361.1 [M+H]⁺; HPLC Ret. Time 1.335 min. (HPLC Method C); ¹H NMR (400 MHz, DMSO-d₆) δ ppm 13.04 (bs, 1H), 9.46 (s, 1H), 8.34 (bs, 1H), 7.87 (bs, 1H), 7.61 (br. s., 2H), 7.33 (dd, J=9.2, 1.6 Hz, 3H), 7.05 (bs, 1H), 2.93 (bs, 1H), 2.13 (bs, 3H), 1.26 (bs, 6H).

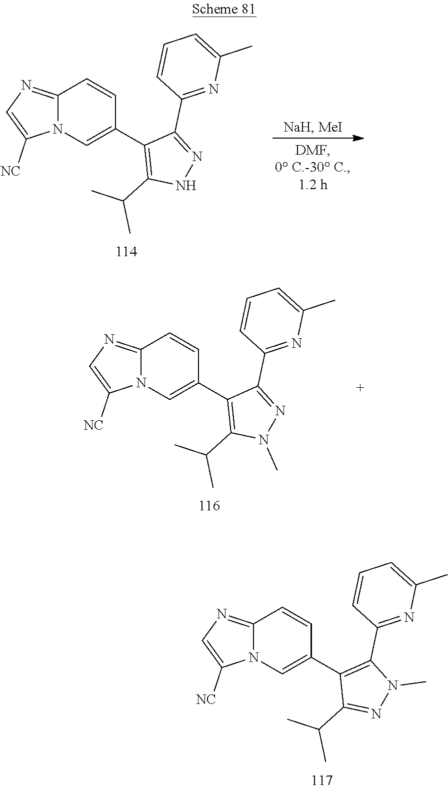 [SMILES: Cc1cccc(-c2n[nH]c(C(C)C)c2-c2ccc3ncc(C#N)n3c2)n1 ; Cc1cccc(-c2nn(C)c(C(C)C)c2-c2ccc3ncc(C#N)n3c2)n1 ; Cc1cccc(-c2c(-c3ccc4ncc(C#N)n4c3)c(C(C)C)nn2C)n1]

### Example 116 and 117: 6-(5-isopropyl-1-methyl-3-(6-methylpyridin-2-yl)-1H-pyrazol-4-yl)imidazo[1,2-a]pyridine-3-carbonitrile and BMT-268875-01-001: 6-(3-isopropyl-1-methyl-5-(6-methylpyridin-2-yl)-1H-pyrazol-4-yl)imidazo[1,2-a]pyridine-3-carbonitrile

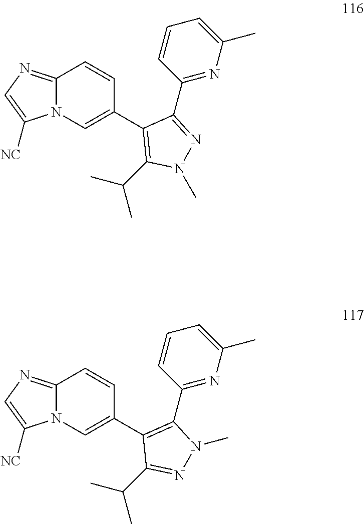 [SMILES: Cc1cccc(-c2nn(C)c(C(C)C)c2-c2ccc3ncc(C#N)n3c2)n1 ; Cc1cccc(-c2c(-c3ccc4ncc(C#N)n4c3)c(C(C)C)nn2C)n1]

Example 116 and 117 were synthesized by reacting Example 114 with methyl iodide and by employing the experimental procedure described for Example 6B in Scheme 6. The crude product was dissolved in DMF and purified via preparative HPLC (Condition N). Fractions containing the desired product were combined and dried under vacuum to afford Example 116. LCMS: m/z=357.3 [M+H]⁺; HPLC Ret. Time 1.565 min. (HPLC Method C); ¹H NMR (400 MHz, DMSO-d₆) δ ppm 8.52 (s, 1H), 8.48 (s, 1H), 7.81 (d, J=9.0 Hz, 1H), 7.66-7.61 (m, 1H), 7.59-7.54 (m, 1H), 7.48 (d, J=9.3 Hz, 1H), 7.05 (d, J=7.6 Hz, 1H), 3.98 (s, 3H), 3.24-3.16 (m, 1H), 2.10 (s, 3H), 1.24 (d, J=7.1 Hz, 6H) and Example 117. LCMS: m/z=357.2 [M+H]⁺; HPLC Ret. Time 1.723 min. (HPLC Method C); ¹H NMR (400 MHz, DMSO-d₆) δ ppm 8.49-8.45 (m, 2H), 7.82-7.77 (m, 1H), 7.67 (t, J=7.8 Hz, 1H), 7.35-7.26 (m, 2H), 7.18-7.11 (m, 1H), 3.90-3.86 (m, 3H), 3.03 (dt, J=13.7, 7.0 Hz, 1H), 2.55 (s, 3H), 1.24-1.18 (m, 6H). MS (ES): m/z=325.4 [M+H]⁺; HPLC Ret. Time 3.53 min. (HPLC Method E).

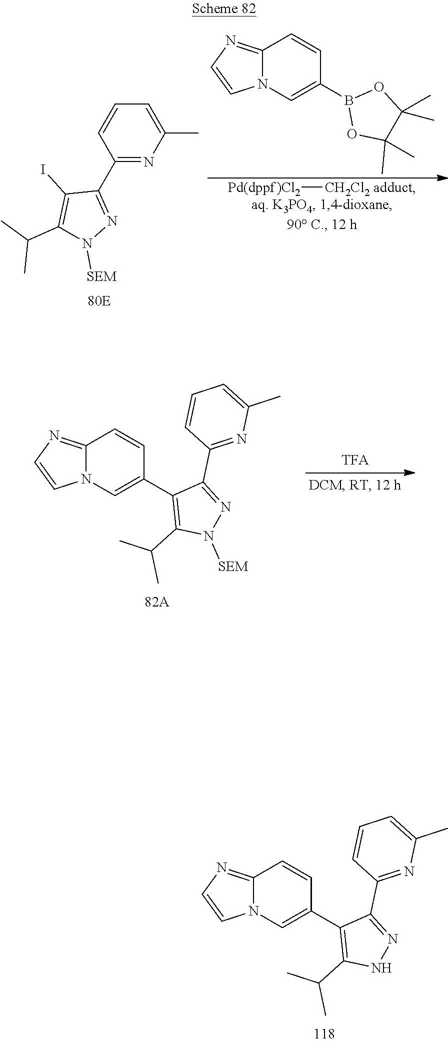

### Intermediate 82A: 6-(5-isopropyl-3-(6-methylpyridin-2-yl)-1-((2-(trimethylsilyl) ethoxy)methyl)-1H-pyrazol-4-yl)imidazo [1,2-a]pyridine

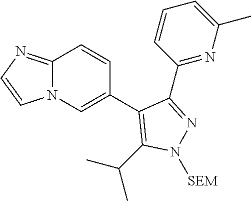 [SMILES: Cc1cccc(-c2nn(COCC[Si](C)(C)C)c(C(C)C)c2-c2ccc3nccn3c2)n1]

Intermediate 82A was synthesized by reacting intermediate 80E with 6-(4,4,5,5-tetramethyl-1,3,2-dioxaborolan-2-yl)imidazo[1,2-a]pyridine and by employing the experimental procedure described for intermediate 1E in Scheme 1 to afford Intermediate 82A as a gum. LCMS: m/z=448.2 [M+H]⁺; HPLC Ret. Time 3.506 min. (HPLC Method E).

### Example 118: 6-(5-isopropyl-3-(6-methylpyridin-2-yl)-1H-pyrazol-4-yl)imidazo[1,2-a]pyridine

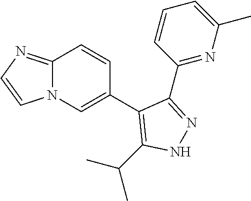 [SMILES: Cc1cccc(-c2n[nH]c(C(C)C)c2-c2ccc3nccn3c2)n1]

Example 118 was synthesized by reacting intermediate 82A with TFA and by employing the experimental procedure described for Example 1 in Scheme 1. The crude product was dissolved in DMF and purified via preparative HPLC (Condition M). Fractions containing the desired product were combined and dried under vacuum to afford Example 118. LCMS: m/z=318.1 [M+H]⁺; HPLC Ret. Time 1.492 min. (HPLC Method C); ¹HNMR (400 MHz, DMSO-d₆) δ ppm 13.23 (bs, 1H), 8.83 (s, 1H), 8.30 (d, J=2.0 Hz, 1H), 8.14 (s, 1H), 7.94-7.85 (m, 1H), 7.80 (s, 1H), 7.70-7.47 (m, 2H), 7.27-6.88 (m, 1H), 3.03 (d, J=7.1 Hz, 1H), 2.19 (bs, 3H), 1.25 (d, J=6.8 Hz, 6H).

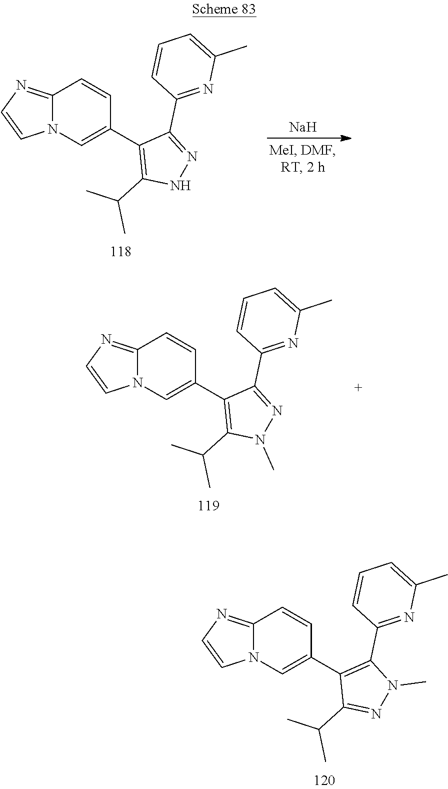

### Example 119 and 120: 6-(5-isopropyl-1-methyl-3-(6-methylpyridin-2-yl)-1H-pyrazol-4-yl)imidazo[1,2-a]pyridine and 6-(3-isopropyl-1-methyl-5-(6-methylpyridin-2-yl)-1H-pyrazol-4-yl)imidazo[1,2-a]pyridine

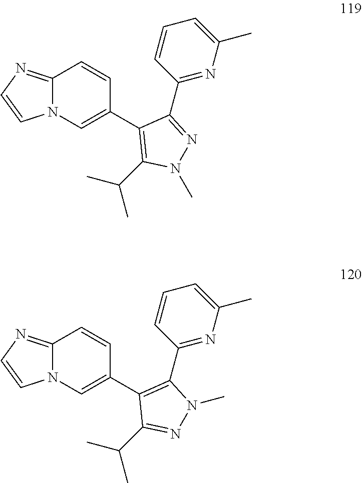 [SMILES: Cc1cccc(-c2nn(C)c(C(C)C)c2-c2ccc3nccn3c2)n1 ; Cc1cccc(-c2c(-c3ccc4nccn4c3)c(C(C)C)nn2C)n1]

Examples 119 and 120 were synthesized by reacting Example 118 with methyl iodide and by employing the experimental procedure described for Example 6B in Scheme 6. The crude product was dissolved in DMF and purified via preparative HPLC (Condition J). Fractions containing the desired product were combined and dried under vacuum to afford Example 119. LCMS: m/z=332.2 [M+H]⁺; HPLC Ret. Time 1.577 min. (HPLC Method C); ¹H NMR (400 MHz, DMSO-d₆) δ ppm 8.82 (d, J=1.0 Hz, 1H), 8.34 (s, 1H), 8.21 (s, 1H), 8.00-7.89 (m, 1H), 7.87-7.74 (m, 1H), 7.72-7.63 (m, 1H), 7.62-7.49 (m, 1H), 7.32-6.93 (m, 1H), 3.99 (s, 3H), 3.27-3.05 (m, 1H), 2.080 (s, 3H), 1.22 (d, J=7.0 Hz, 6H) and Example 120. LCMS: m/z=332.2 [M+H]⁺; HPLC Ret. Time 1.621 min. (HPLC Method C); ¹H NMR (400 MHz, DMSO-d₆) δ ppm 8.82 (d, J=1.0 Hz, 1H), 8.31 (d, J=1.5 Hz, 1H), 8.16 (s, 1H), 7.91 (d, J=9.5 Hz, 1H), 7.66-7.53 (m, 2H), 7.32-7.10 (m, 1H), 7.06-6.96 (m, 1H), 3.85 (bs, 3H), 3.01 (dt, J=13.7, 7.0 Hz, 1H), 2.529 (s, 3H), 1.23-1.14 (m, 6H).

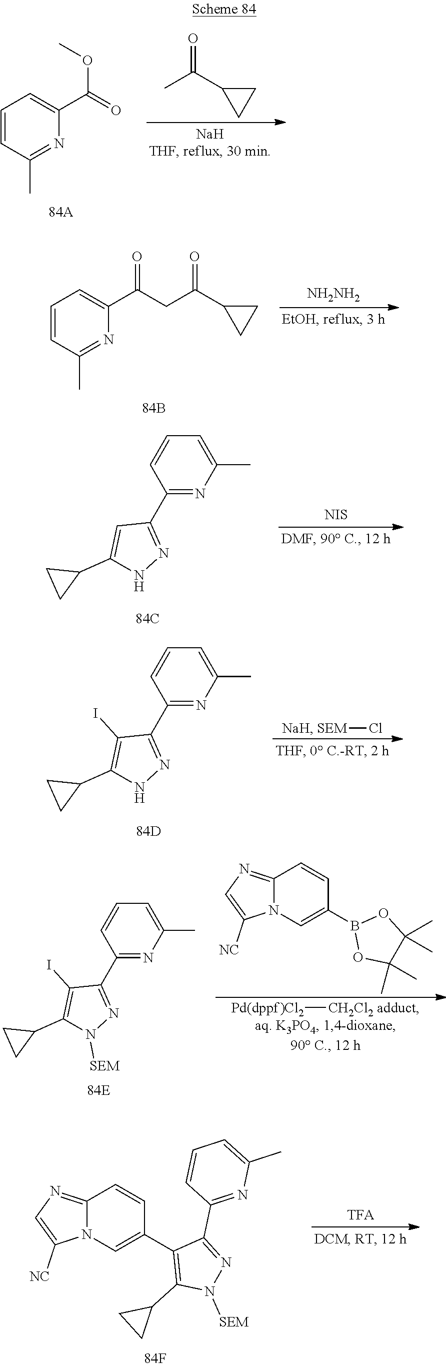

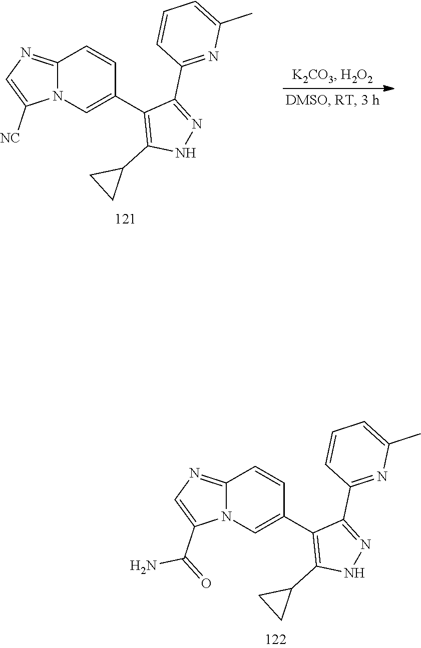 [SMILES: Cc1cccc(-c2n[nH]c(C3CC3)c2-c2ccc3ncc(C#N)n3c2)n1 ; Cc1cccc(-c2n[nH]c(C3CC3)c2-c2ccc3ncc(C(N)=O)n3c2)n1]

### Intermediate 84B: 1-cyclopropyl-3-(6-methylpyridin-2-yl)propane-1,3-dione

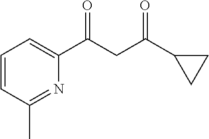

Intermediate 84B was synthesized by reacting intermediate 84A with 1-cyclopropylethanone and by employing the experimental procedure described for intermediate 80B in Scheme 80. The crude product was purified by silica gel chromatography (40 g RediSep® column, eluting with a gradient of 5-10% EtOAc in petroleum ether). Fractions containing the desired product were combined and evaporated to afford intermediate 84B. LCMS: m/z=204.2 [M+H]⁺; HPLC Ret. Time 1.577 min. (HPLC Method E).

### Intermediate 84C: 2-(5-cyclopropyl-1H-pyrazol-3-yl)-6-methylpyridine

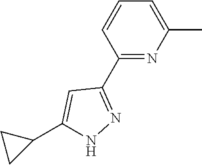

Intermediate 84C was synthesized by reacting intermediate 84B with hydrazine hydrate and by employing the experimental procedure described for intermediate 53B in Scheme 53 to get intermediate 84C as a semi-solid. LCMS: m/z=200.2 [M+H]⁺; HPLC Ret. Time 1.652 min. (HPLC Method E).

### Intermediate 84D: 2-(5-cyclopropyl-4-iodo-1H-pyrazol-3-yl)-6-methylpyridine

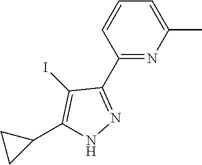 [SMILES: Cc1cccc(-c2n[nH]c(C3CC3)c2I)n1]

Intermediate 84D was synthesized by reacting intermediate 84C with NIS and by employing the experimental procedure described for intermediate 47B in Scheme 47. The crude product was purified by silica gel chromatography (40 g RediSep® column, eluting with a gradient of 10-60% EtOAc in petroleum ether). Fractions containing the desired product were combined and evaporated to afford intermediate 84D. LCMS: m/z=326.2 [M+H]⁺; HPLC Ret. Time 2.262 min. (HPLC Method E).

### Intermediate 84E: 2-(5-cyclopropyl-4-iodo-1-((2-(trimethylsilyl)ethoxy)methyl)-1H-pyrazol-3-yl)-6-methylpyridine

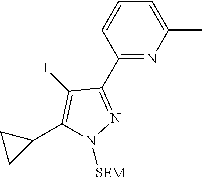 [SMILES: Cc1cccc(-c2nn(COCC[Si](C)(C)C)c(C3CC3)c2I)n1]

Intermediate 84E was synthesized by reacting intermediate 84D with SEM-Cl and by employing the experimental procedure described for intermediate 8A in Scheme 8 to get intermediate 84E as a gum. LCMS: m/z=456.2 [M+H]⁺; HPLC Ret. Time 4.202 min. (HPLC Method E).

### Intermediate 84F: 6-(5-cyclopropyl-3-(6-methylpyridin-2-yl)-1-((2-(trimethylsilyl)ethoxy)methyl)-1H-pyrazol-4-yl)imidazo[1,2-a]pyridine-3-carbonitrile

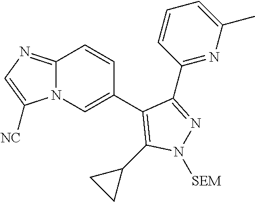 [SMILES: Cc1cccc(-c2nn(COCC[Si](C)(C)C)c(C3CC3)c2-c2ccc3ncc(C#N)n3c2)n1]

Intermediate 84F was synthesized by reacting intermediate 84E with -(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated to afford intermediate 84F. LCMS: m/z=471.2 [M+H]⁺; HPLC Ret. Time 3.735 min. (HPLC Method E).

### Example 121: 6-(5-cyclopropyl-3-(6-methylpyridin-2-yl)-1H-pyrazol-4-yl)imidazo[1,2-a]pyridine-3-carbonitrile

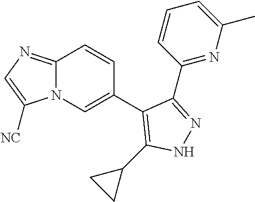 [SMILES: Cc1cccc(-c2n[nH]c(C3CC3)c2-c2ccc3ncc(C#N)n3c2)n1]

Example 121 was synthesized by reacting intermediate 84F with TFA and by employing the experimental procedure described for Example 1 in Scheme 1. The crude product was dissolved in DMF and purified via preparative HPLC (Condition H). Fractions containing the desired product were combined and dried under vacuum to afford Example 121. LCMS: m/z=341.1 [M+H]⁺; HPLC Ret. Time 1.487 min. (HPLC Method C); ¹H NMR (400 MHz, DMSO-d₆) δ ppm 13.21-12.87 (m, 1H), 8.78-8.66 (m, 1H), 8.45 (s, 1H), 7.91-7.76 (m, 1H), 7.71-7.50 (m, 3H), 7.27-7.05 (m, 1H), 2.45-2.18 (m, 3H), 1.98 (bs, 1H), 0.96-0.79 (m, 4H).

### Example 122: 6-(5-cyclopropyl-3-(6-methylpyridin-2-yl)-1H-pyrazol-4-yl)imidazo[1,2-a]pyridine-3-carboxamide

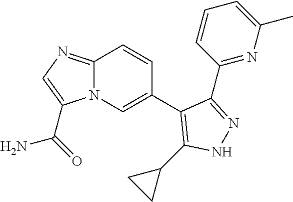 [SMILES: Cc1cccc(-c2n[nH]c(C3CC3)c2-c2ccc3ncc(C(N)=O)n3c2)n1]

Example 122 was synthesized by reacting Example 121 with H₂O₂/K₂CO₃ and by employing the experimental procedure described for Example 2 in Scheme 1. The crude product was dissolved in DMF and purified via preparative HPLC (Condition L). Fractions containing the desired product were combined and dried under vacuum to afford Example 122. LCMS: m/z=359.1 [M+H]⁺; HPLC Ret. Time 1.153 min. (HPLC Method C); ¹H NMR (400 MHz, DMSO-d₆) δ ppm 13.14-12.77 (m, 1H), 9.64-9.52 (m, 1H), 8.35 (s, 1H), 7.93 (bs, 1H), 7.75-7.67 (m, 1H), 7.58 (bs, 2H), 7.39 (t, J=8.8 Hz, 1H), 7.17-6.95 (m, 2H), 2.18 (s, 3H), 1.93-1.69 (m, 1H), 0.92 (d, J=7.8 Hz, 4H).

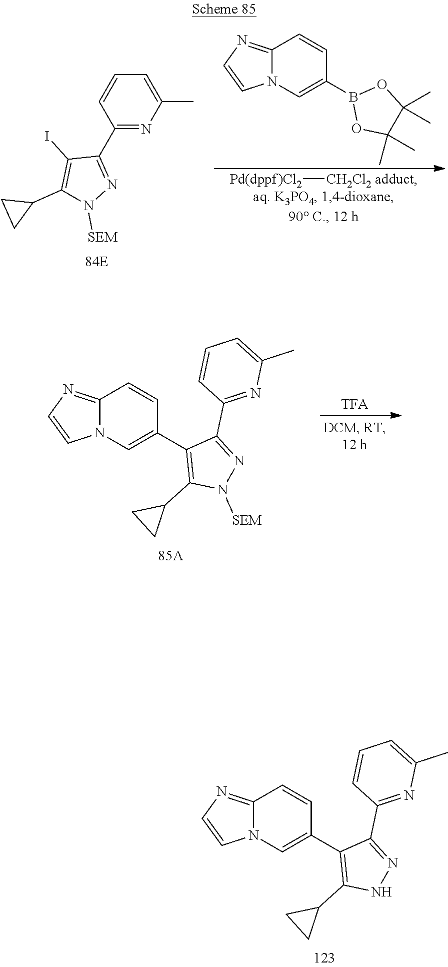

### Intermediate 85A: 6-(5-cyclopropyl-3-(6-methylpyridin-2-yl)-1-((2-(trimethylsilyl)ethoxy) methyl)-1H-pyrazol-4-yl) imidazo [1,2-a]pyridine

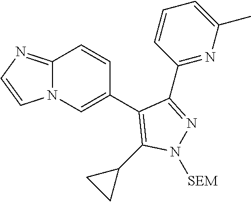 [SMILES: Cc1cccc(-c2nn(COCC[Si](C)(C)C)c(C3CC3)c2-c2ccc3nccn3c2)n1]

Intermediate 85A was synthesized by reacting intermediate 84E with 6-(4,4,5,5-tetramethyl-1,3,2-dioxaborolan-2-yl)imidazo[1,2-a]pyridine and by employing the experimental procedure described for intermediate 1E in Scheme 1. The crude product was purified by silica gel chromatography (40 g RediSep® column, eluting with a gradient of 20-30% EtOAc in petroleum ether). Fractions containing the desired product were combined and evaporated under reduced pressure to afford intermediate 85A. LCMS: m/z=446.2 [M+H]⁺; HPLC Ret. Time 3.385 min. (HPLC Method E).

### Example 123: 6-(5-cyclopropyl-3-(6-methylpyridin-2-yl)-1H-pyrazol-4-yl)imidazo[1,2-a]pyridine

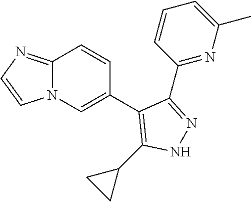 [SMILES: Cc1cccc(-c2n[nH]c(C3CC3)c2-c2ccc3nccn3c2)n1]

Example 123 was synthesized by reacting intermediate 85A with TFA and by employing the experimental procedure described for Example 1 in Scheme 1. The crude product was dissolved in DMF and purified via preparative HPLC (Condition H). Fractions containing the desired product were combined and dried under vacuum to afford Example 123. LCMS: m/z=316.1 [M+H]⁺; HPLC Ret. Time 1.130 min. (HPLC Method C); ¹H NMR (400 MHz, DMSO-d₆) δ ppm 13.08-12.72 (m, 1H), 8.60 (bs, 1H), 7.96 (bs, 1H), 7.68-7.44 (m, 3H), 7.20-6.95 (m, 3H), 2.22 (bs, 3H), 1.95-1.68 (m, 1H), 0.907-0.816 (m, 4H).

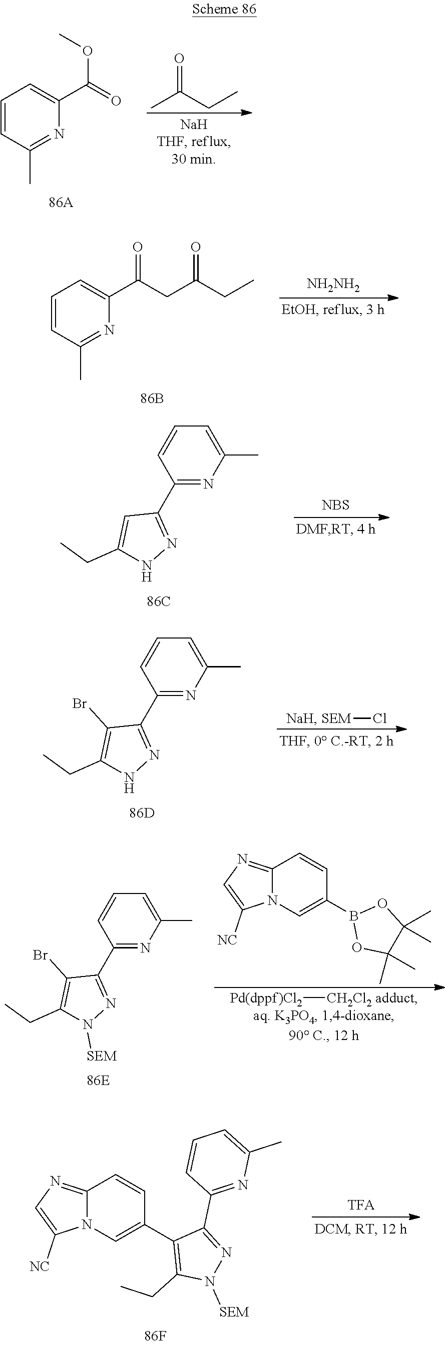

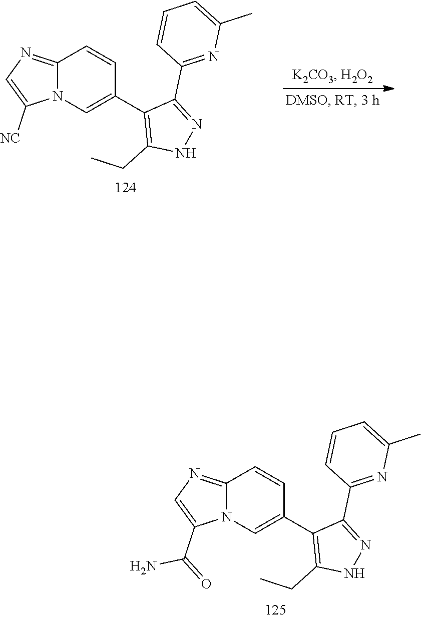 [SMILES: CCc1[nH]nc(-c2cccc(C)n2)c1-c1ccc2ncc(C#N)n2c1 ; CCc1[nH]nc(-c2cccc(C)n2)c1-c1ccc2ncc(C(N)=O)n2c1]

### Intermediate 86B: 1-(6-methylpyridin-2-yl)pentane-1,3-dione

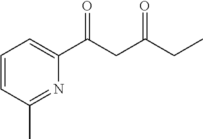

Intermediate 86B was synthesized by reacting intermediate 86A with butan-2-one and by employing the experimental procedure described for intermediate 80B in Scheme 80. The crude product was purified by silica gel chromatography (80 g RediSep® column, eluting with a gradient of 10-60% EtOAc in petroleum ether). Fractions containing the desired product were combined and evaporated under reduced pressure to afford intermediate 86B. LCMS: m/z=192.2 [M+H]⁺; HPLC Ret. Time 2.551 min. (HPLC Method E).

### Intermediate 86C: 2-(5-ethyl-1H-pyrazol-3-yl)-6-methylpyridine

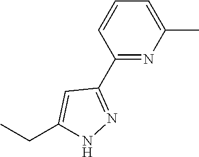

Intermediate 86C was synthesized by reacting intermediate 86B with hydrazine hydrate and by employing the experimental procedure described for intermediate 53B in Scheme 53. The crude product was purified by silica gel chromatography (40 g RediSep® column, eluting with a gradient of 10-60% EtOAc in petroleum ether). Fractions containing the desired product were combined and evaporated to afford intermediate 86C. LCMS: m/z=188.2 [M+H]⁺; HPLC Ret. Time 1.492 min. (HPLC Method E).

### Intermediate 86D: 2-(4-bromo-5-ethyl-1H-pyrazol-3-yl)-6-methylpyridine

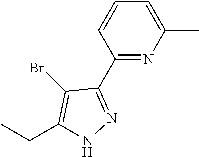

Intermediate 86D was synthesized by reacting intermediate 86C with NBS and by employing the experimental procedure described for intermediate 1D in Scheme 1 to afford Example 86D as a gum. LCMS: m/z=268.0 [M+2]⁺; HPLC Ret. Time 2.377 min. (HPLC Method E).

### Intermediate 86E: 2-(4-bromo-5-ethyl-1-((2-(trimethylsilyl)ethoxy)methyl)-1H-pyrazol-3-yl)-6-methylpyridine

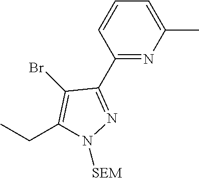 [SMILES: CCc1c(Br)c(-c2cccc(C)n2)nn1COCC[Si](C)(C)C]

Intermediate 86E was synthesized by reacting intermediate 86D with SEM-Cl and by employing the experimental procedure described for intermediate 8A in Scheme 8 to afford intermediate 86E as a liquid. LCMS: m/z=398.2 [M+2]⁺; HPLC Ret. Time 1.34 min. (HPLC Method B).

### Intermediate 86F: 6-(5-ethyl-3-(6-methylpyridin-2-yl)-1-((2-(trimethylsilyl)ethoxy)methyl)-1H-pyrazol-4-yl)imidazo[1,2-a]pyridine-3-carbonitrile

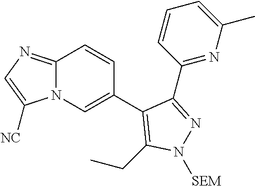 [SMILES: CCc1c(-c2ccc3ncc(C#N)n3c2)c(-c2cccc(C)n2)nn1COCC[Si](C)(C)C]

Intermediate 86F was synthesized by reacting intermediate 86E with 6-(4,4,5,5-tetramethyl-1,3,2-dioxaborolan-2-yl)imidazo[1,2-a]pyridine-3-carbonitrile and by employing the experimental procedure described for Intermediate 1E in Scheme 1 to provide intermediate 86F as a gum. The crude product was used in the next step without purification. LCMS: m/z=459.5 [M+1]⁺; HPLC Ret. Time 1.67 min. (HPLC Method B).

### Example 124: 6-(5-ethyl-3-(6-methylpyridin-2-yl)-1H-pyrazol-4-yl)imidazo[1,2-a]pyridine-3-carbonitrile

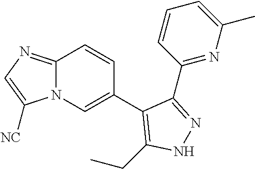 [SMILES: CCc1[nH]nc(-c2cccc(C)n2)c1-c1ccc2ncc(C#N)n2c1]

Example 124 was synthesized by reacting intermediate 86F with TFA and by employing the experimental procedure described for Example 1 in Scheme 1. The crude product was dissolved in DMF and purified via preparative HPLC (Condition K). Fractions containing the desired product were combined and dried under vacuum to afford Example 124. LCMS: m/z=329.2 [M+1]⁺; HPLC Ret. Time 1.469 min. (HPLC Method C). ¹H NMR (400 MHz, DMSO-d₆) δ ppm 13.38 (bs, 1H), 8.91 (s, 1H), 8.72 (s, 1H), 8.06 (d, J=9.0 Hz, 1H), 7.98-7.85 (m, 1H), 7.81-7.66 (m, 1H), 7.38 (bs, 2H), 3.03-2.86 (m, 3H), 2.55-2.36 (m, 2H), 1.53-1.29 (m, 3H).

### Example 125: 6-(5-ethyl-3-(6-methylpyridin-2-yl)-1H-pyrazol-4-yl)imidazo[1,2-a]pyridine-3-carboxamide

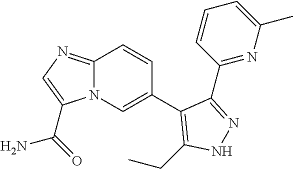 [SMILES: CCc1[nH]nc(-c2cccc(C)n2)c1-c1ccc2ncc(C(N)=O)n2c1]

Example 125 was synthesized by reacting Example 124 with H₂O₂/K₂CO₃ and by employing the experimental procedure described for Example 2 in Scheme 1. The crude product was dissolved in DMF and purified via preparative HPLC (Condition K). Fractions containing the desired product were combined and dried under vacuum to afford Example 124. LCMS: m/z=347.2 [M+1]⁺; HPLC Ret. Time 1.227 min. (HPLC Method C). ¹H NMR (400 MHz, DMSO-d₆) δ ppm 13.50-13.24 (m, 1H), 9.73 (dd, J 1.7, 1.0 Hz, 1H), 8.59 (bs, 1H), 7.88 (bs, 4H), 7.64-7.23 (m, 3H), 3.05-2.85 (m, 2H), 2.43 (s, 3H), 1.51-1.17 (m, 3H).

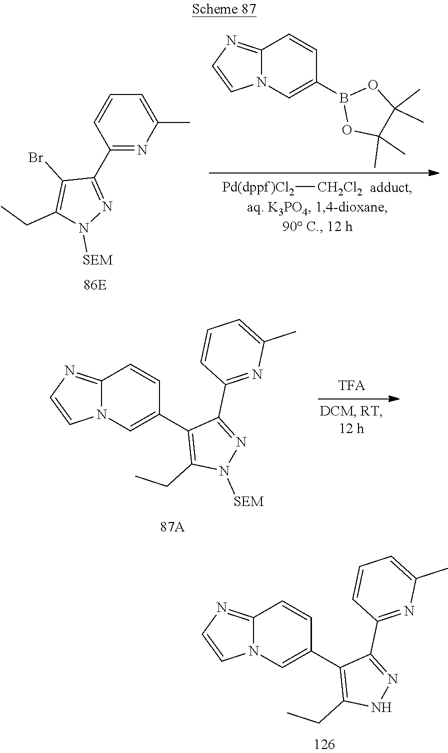

### Intermediate 87A: 6-(5-ethyl-3-(6-methylpyridin-2-yl)-1-((2-(trimethylsilyl)ethoxy)methyl)-1H-pyrazol-4-yl)imidazo[1,2-a]pyridine

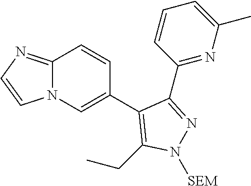 [SMILES: CCc1c(-c2ccc3nccn3c2)c(-c2cccc(C)n2)nn1COCC[Si](C)(C)C]

Intermediate 87A was synthesized by reacting intermediate 86E with 6-(4,4,5,5-tetramethyl-1,3,2-dioxaborolan-2-yl)imidazo[1,2-a]pyridine and by employing the experimental procedure described for intermediate 1E in Scheme 1 to afford intermediate 87A as a gum. The crude product was used in the next step without purification. LCMS: m/z=434.5 [M+1]⁺; HPLC Ret. Time 1.53 min. (HPLC Method B).

### Example 126: 6-(5-ethyl-3-(6-methylpyridin-2-yl)-1H-pyrazol-4-yl)imidazo[1,2-a]pyridine

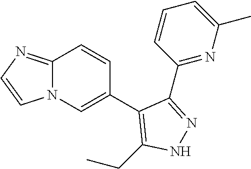 [SMILES: CCc1[nH]nc(-c2cccc(C)n2)c1-c1ccc2nccn2c1]

Example 126 was synthesized by reacting intermediate 87A with TFA and by employing the experimental procedure described for Example 1 in Scheme 1. The crude product was dissolved in DMF and purified via preparative HPLC (Condition K). Fractions containing the desired product were combined and dried under vacuum to afford Example 126. LCMS: m/z=304.2 [M+1]⁺; HPLC Ret. Time 1.303 min. (HPLC Method C). ¹H NMR (400 MHz, DMSO-d₆) δ ppm 13.43-13.04 (m, 1H), 8.84-8.59 (m, 1H), 8.58 (s, 1H), 8.26-8.06 (m, 1H), 7.95-7.52 (m, 3H), 7.35 (dd, J=9.3, 1.2 Hz, 2H), 3.07-2.83 (m, 3H), 2.47 (bs, 2H), 1.43 (bs, 3H).

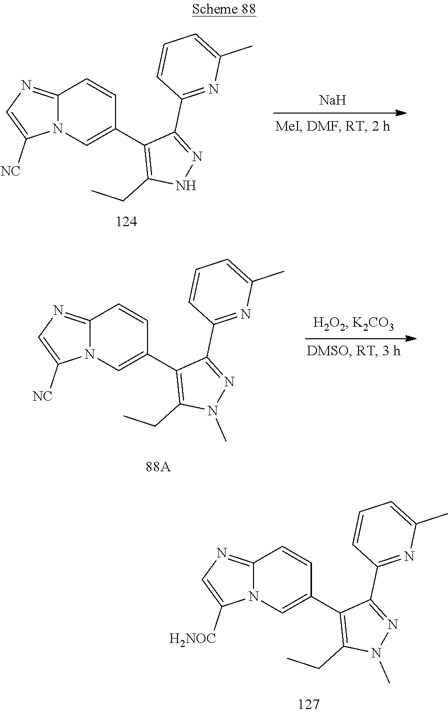

### Intermediate 88A: 6-(5-ethyl-1-methyl-3-(6-methylpyridin-2-yl)-1H-pyrazol-4-yl)imidazo[1,2-a]pyridine-3-carbonitrile

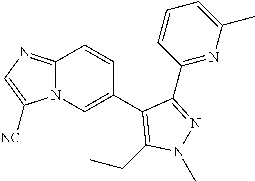 [SMILES: CCc1c(-c2ccc3ncc(C#N)n3c2)c(-c2cccc(C)n2)nn1C]

Intermediate 88A was synthesized by reacting Example 124 with methyl iodide and by employing the experimental procedure described for Example 6B in Scheme 6 to get intermediate 88A as a gum. The crude product was used in the next step without purification. LCMS: m/z=343.2 [M+1]⁺; HPLC Ret. Time 0.60 min. (HPLC Method B).

### Example 127: 6-(5-ethyl-1-methyl-3-(6-methylpyridin-2-yl)-1H-pyrazol-4-yl)imidazo[1,2-a]pyridine-3-carboxamide

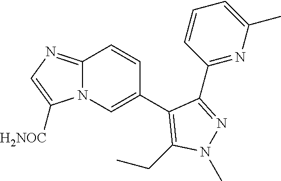 [SMILES: CCc1c(-c2ccc3ncc(C(N)=O)n3c2)c(-c2cccc(C)n2)nn1C]

Example 127 was synthesized by reacting intermediate 88A with H₂O₂/K₂CO₃ and by employing the experimental procedure described for Example 2 in Scheme 1. The crude product was dissolved in DMF and purified via preparative HPLC (Condition J). Fractions containing the desired product were combined and dried under vacuum to afford Example 127. LCMS: m/z=361.3 [M+1]⁺; HPLC Ret. Time 1.055 min. (HPLC Method C); ¹H NMR (400 MHz, DMSO-d₆) δ ppm 9.60 (dd, J=1.6, 0.9 Hz, 1H), 8.59 (s, 1H), 8.20 (bs, 1H), 7.87 (dd, J=9.2, 0.9 Hz, 1H), 7.79 (t, J=7.8 Hz, 1H), 7.68 (dd, J=9.2, 1.6 Hz, 2H), 7.57 (d, J=7.8 Hz, 1H), 7.27-7.15 (m, 1H), 3.97 (s, 3H), 2.74-2.64 (m, 2H), 2.33-2.21 (m, 3H), 1.21-1.00 (m, 3H).

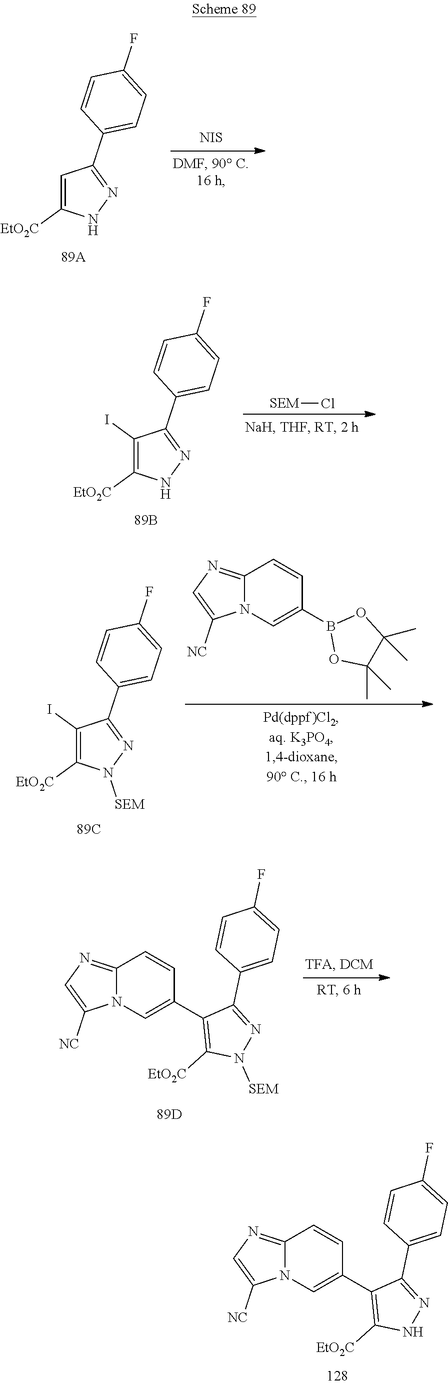

### Intermediate 89B: ethyl 3-(4-fluorophenyl)-4-iodo-1-((2(trimethylsilyl)ethoxy)methyl)-1H-pyrazole-5-carboxylate

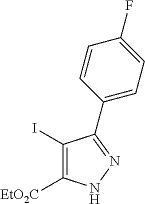 [SMILES: CCOC(=O)c1[nH]nc(-c2ccc(F)cc2)c1I]

Intermediate 89B was synthesized by reacting intermediate 89A with NIS and by employing the experimental procedure described for intermediate 47B in Scheme 47 to afford intermediate 89B. LCMS: m/z=361.0 [M+H]⁺; HPLC Ret. Time 2.34 min. (HPLC Method E).

### Intermediate 89C: ethyl-3-(4-fluorophenyl)-4-iodo-1-((2-(trimethylsilyl)ethoxy)methyl)-1H-pyrazole-5-carboxylate

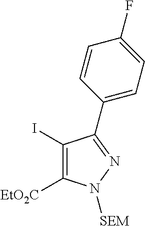 [SMILES: CCOC(=O)c1c(I)c(-c2ccc(F)cc2)nn1COCC[Si](C)(C)C]

Intermediate 89C was synthesized by reacting intermediate 89B with SEM-Cl and by employing the experimental procedure described for intermediate 8A in Scheme 8 to afford intermediate 89C. LCMS: m/z=491.2 [M+H]⁺; HPLC Ret. Time 3.87 min; (HPLC Method E).

### Intermediate 89D: ethyl 4-(3-cyanoimidazo[1,2-a]pyridin-6-yl)-3-(4-fluorophenyl)-1-((2-(trimethylsilyl)ethoxy)methyl)-1H-pyrazole-5-carboxylate

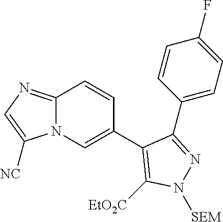 [SMILES: CCOC(=O)c1c(-c2ccc3ncc(C#N)n3c2)c(-c2ccc(F)cc2)nn1COCC[Si](C)(C)C]

Intermediate 89D was synthesized by reacting intermediate 89C with 6-(4,4,5,5-tetramethyl-1,3,2-dioxaborolan-2-yl)imidazo[1,2-a]pyridine-3-carbonitrile and by employing the experimental procedure described for intermediate 1E in Scheme 1. The crude product was purified by silica gel chromatography (24 g RediSep® column, eluting with a gradient of 50-100% EtOAc in petroleum ether). Fractions containing the desired product were combined and evaporated to afford intermediate 89D. LCMS: m/z=506.2 [M+H]⁺; HPLC Ret. Time 3.50 min; (HPLC Method E).

### Example 128: ethyl 4-(3-cyanoimidazo[1,2-a]pyridin-6-yl)-3-(4-fluorophenyl)-1H-pyrazole-5-carboxylate

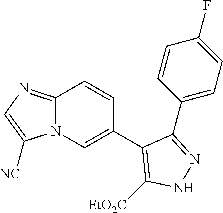 [SMILES: CCOC(=O)c1[nH]nc(-c2ccc(F)cc2)c1-c1ccc2ncc(C#N)n2c1]

Example 128 was synthesized by reacting intermediate 89D with TFA and by employing the experimental procedure described for Example 1 in Scheme 1. The crude product was dissolved in DMF and purified via preparative HPLC (Condition N). Fractions containing the desired product were combined and dried under vacuum to afford Example 128. LCMS: m/z=376.1 [M+H]⁺; HPLC Ret. Time 1.70 min; (HPLC Method C). ¹H NMR (400 MHz, DMSO-d₆) δ ppm 14.23 (s, 1H), 8.66 (br. s., 1H), 8.48 (s, 1H), 7.82 (s, 1H), 7.41-7.47 (m, 2H), 7.35 (dd, J=9.3, 1.7 Hz, 1H), 7.21 (d, J=8.8 Hz, 2H) 4.13-4.22 (m, 2H) 1.09 (t, J=7.1 Hz, 3H).

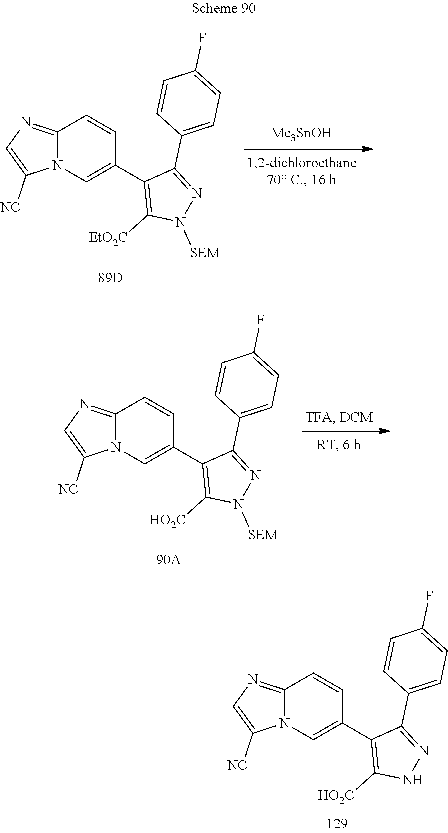

### Intermediate 90A: 4-(3-cyanoimidazo[1,2-a]pyridin-6-yl)-3-(4-fluorophenyl)-1-((2-(trimethylsilyl)ethoxy)methyl)-1H-pyrazole-5-carboxylic acid

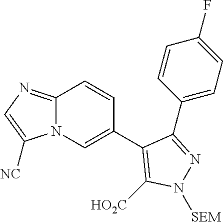 [SMILES: C[Si](C)(C)CCOCn1nc(-c2ccc(F)cc2)c(-c2ccc3ncc(C#N)n3c2)c1C(=O)O]

To a solution of intermediate 89D (0.25 g, 0.494 mmol) in 1,2-dichloroethane (2 mL) was added and Me₃SnOH (0.447 g, 2.472 mmol) and the reaction mixture was stirred at 70° C. for 16 h. The solvent was partially evaporated from the reaction mixture and the mixture was neutralized using 1.5 N HCl solution to pH-7.0. The aqueous layer was back-extracted with ethyl acetate (3×5 mL). The combined the organic layer was washed with brine, dried over Na₂SO₄, filtered and the filtrate was concentrated under reduced pressure to afford intermediate 90A (0.2 g, 85.0% yield). LCMS: m/z=478.2 [M+H]⁺; HPLC Ret. Time 1.63 min. (HPLC Method E).

### Example 129: 4-(3-cyanoimidazo[1,2-b]pyridazin-6-yl)-3-(4-fluorophenyl)-1H-pyrazole-5-carboxylic acid

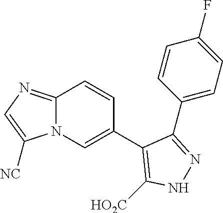 [SMILES: N#Cc1cnc2ccc(-c3c(-c4ccc(F)cc4)n[nH]c3C(=O)O)cn12]

Example 129 was synthesized by reacting intermediate 90A with TFA and by employing the experimental procedure described for Example 1 in Scheme 1. The crude product was dissolved in DMF and purified via preparative HPLC (Condition N). Fractions containing the desired product were combined and dried under vacuum to afford Example 129. LCMS: m/z=348.0 [M+H]⁺; HPLC Ret. Time 1.07 min; (HPLC Method C). ¹H NMR (400 MHz, DMSO-d₆) δ ppm 13.65 (s, 1H), 8.63 (br. s., 1H), 8.45 (s, 1H), 7.76 (d, J=9.5 Hz, 1H), 7.40 (dd, J=8.2, 5.8 Hz, 2H), 7.32 (d, J=9.8 Hz, 1H), 7.15 (br. s., 2H).

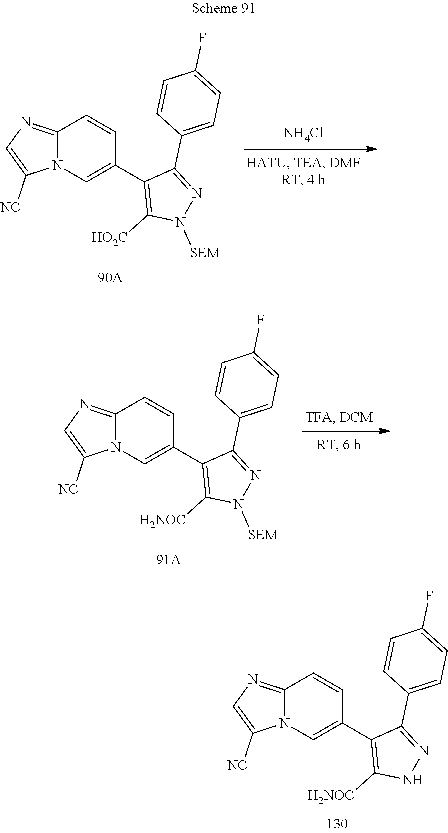

### Intermediate 91A: 4-(3-cyanoimidazo[1,2-a]pyridin-6-yl)-3-(4-fluorophenyl)-1-((2-(trimethylsilyl)ethoxy)methyl)-1H-pyrazole-5-carboxamide

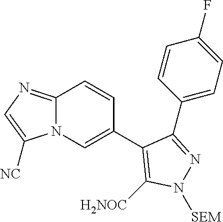 [SMILES: C[Si](C)(C)CCOCn1nc(-c2ccc(F)cc2)c(-c2ccc3ncc(C#N)n3c2)c1C(N)=O]

To a solution of intermediate 90A (0.1 g, 0.209 mmol), ammonium chloride (0.056 g, 1.047 mmol) and TEA (0.175 mL, 1.256 mmol) in DMF (2 mL) was added HATU (0.318 g, 0.838 mmol). The reaction mixture was stirred at RT for 4 h, and partitioned between water and ethyl acetate. The aqueous layer was back-extracted with ethyl acetate. The combined organic layers were washed with brine, dried over Na₂SO₄, filtered and the filtrate was concentrated under reduced pressure to afford intermediate 91A (0.08 g, 80% yield). LCMS: m/z=477.2 [M+H]⁺; HPLC Ret. Time 3.03 min; (HPLC Method E).

### Example 130: 4-(3-cyanoimidazo[1,2-a]pyridin-6-yl)-3-(4-fluorophenyl)-1H-pyrazole-5-carboxamide

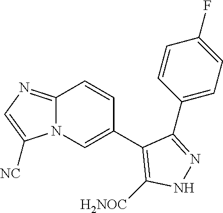 [SMILES: N#Cc1cnc2ccc(-c3c(-c4ccc(F)cc4)n[nH]c3C(N)=O)cn12]

Example 130 was synthesized by reacting intermediate 91B with TFA and by employing the experimental procedure described for Example 1 in Scheme 1. The crude product was dissolved in DMF and purified via preparative HPLC (Condition F). Fractions containing the desired product were combined and dried under vacuum to afford Example 130. LCMS: m/z=347.1 [M+H]⁺; HPLC Ret. Time 1.21 min; (HPLC Method C); ¹H NMR (400 MHz, DMSO-d₆) δ ppm 13.81 (br. s., 1H), 8.56 (s, 1H), 8.45 (s, 1H), 7.76 (d, J=8.8 Hz, 1H), 7.64 (br. s., 1H), 7.42 (dd, J=8.1, 5.6 Hz, 2H), 7.19-7.34 (m, 4H).

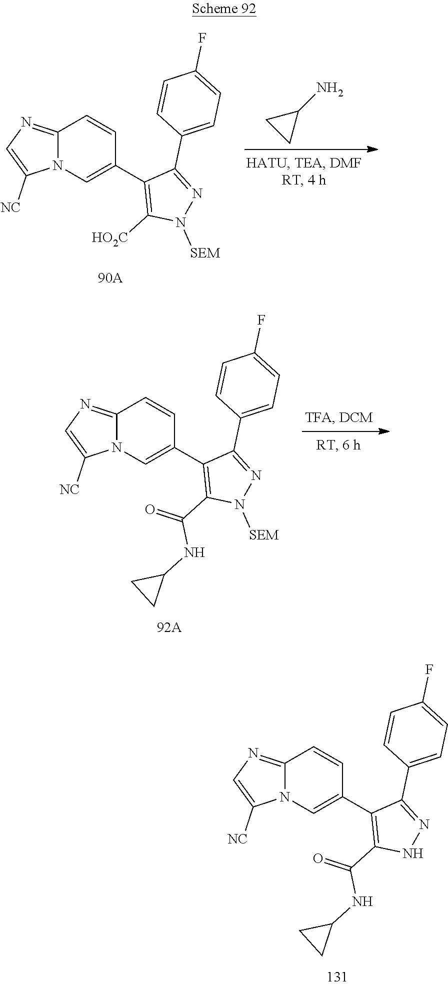

### Intermediate 92A: 4-(3-cyanoimidazo[1,2-a]pyridin-6-yl)-N-cyclopropyl-3-(4-fluorophenyl)-1-((2-(trimethylsilyl)ethoxy)methyl)-1H-pyrazole-5-carboxamide

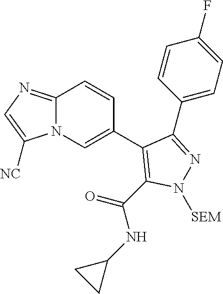 [SMILES: C[Si](C)(C)CCOCn1nc(-c2ccc(F)cc2)c(-c2ccc3ncc(C#N)n3c2)c1C(=O)NC1CC1]

Intermediate 92A was synthesized by reacting Intermediate 90A with cyclopropyl amine and by employing the experimental procedure described for intermediate 66B in Scheme 66 to afford intermediate 92A. LCMS: m/z=517.5 [M+H]⁺; HPLC Ret. Time 1.13 min; (HPLC Method A).

### Example 131: 4-(3-cyanoimidazo[1,2-a]pyridin-6-yl)-N-cyclopropyl-3-(4-fluorophenyl)-1H-pyrazole-5-carboxamide

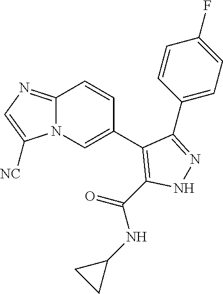 [SMILES: N#Cc1cnc2ccc(-c3c(-c4ccc(F)cc4)n[nH]c3C(=O)NC3CC3)cn12]

Example 131 was synthesized by reacting intermediate 92A with TFA and by employing the experimental procedure described for Example 1 in Scheme 1. The crude product was dissolved in DMF and purified via preparative HPLC (Condition N). Fractions containing the desired product were combined and dried under vacuum to afford Example 131. LCMS: m/z=387.1 [M+H]⁺; HPLC Ret. Time 1.45 min; (HPLC Method C). ¹H NMR (400 MHz, DMSO-d₆) δ ppm 13.79 (br. s., 1H), 8.58 (s, 1H), 8.46 (s, 1H), 8.31 (d, J=4.2 Hz, 1H), 7.77 (d, J=9.3 Hz, 1H), 7.40-7.45 (m, 2H), 7.27 (dd, J=14.9, 9.3 Hz, 3H), 2.70-2.80 (m, 1H), 0.5-0.65 (m, 4H).

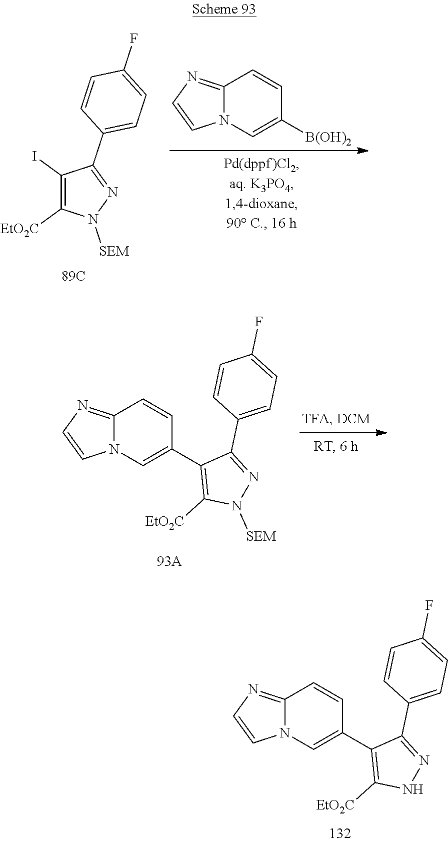

### Intermediate 93A: ethyl 3-(4-fluorophenyl)-4-(imidazo[1,2-a]pyridin-6-yl)-1-((2-(trimethylsilyl)ethoxy)methyl)-1H-pyrazole-5-carboxylate

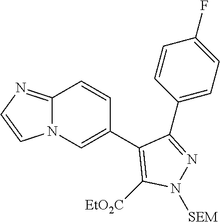 [SMILES: CCOC(=O)c1c(-c2ccc3nccn3c2)c(-c2ccc(F)cc2)nn1COCC[Si](C)(C)C]

Intermediate 93A was synthesized by reacting intermediate 89C with imidazo[1,2-a]pyridin-6-ylboronic acid and by employing the experimental procedure described for intermediate 1E in Scheme 1. The crude product was purified by silica gel chromatography (24 g RediSep® column, eluting with a gradient of 50-100% EtOAc in petroleum ether). Fractions containing the desired product were combined and evaporated under reduced pressure to afford intermediate 93A. LCMS: m/z=481.2 [M+H]⁺; HPLC Ret. Time 3.22 min. (HPLC Method E).

### Example 132: ethyl 3-(4-fluorophenyl)-4-(imidazo[1,2-a]pyridin-6-yl)-1H-pyrazole-5-carboxylate

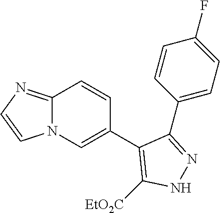 [SMILES: CCOC(=O)c1[nH]nc(-c2ccc(F)cc2)c1-c1ccc2nccn2c1]

Example 132 was synthesized by reacting intermediate 93A with TFA and by employing the experimental procedure described for Example 1 in Scheme 1. The crude product was dissolved in DMF and purified via preparative HPLC (Condition N). Fractions containing the desired product were combined and dried under vacuum to afford Example 132. LCMS: m/z=351.2 [M+H]⁺; HPLC Ret. Time 1.22 min; (HPLC Method C). ¹H NMR (400 MHz, DMSO-d₆) δ ppm 13.88-14.31 (m, 1H), 8.47 (s, 1H), 7.89 (s, 1H), 7.57 (s, 1H), 7.52 (d, J=9.3 Hz, 1H), 7.43 (dd, J=8.3, 5.6 Hz, 2H), 7.19 (t, J=8.7 Hz, 2H), 7.03 (d, J=9.3 Hz, 1H) 4.08-4.21 (m, 2H), 1.08 (t, J=6.97 Hz, 3H).

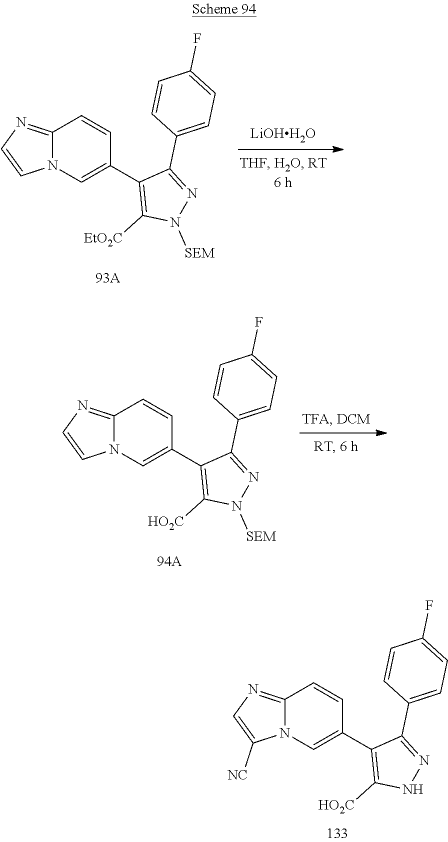

### Intermediate 94A: 3-(4-fluorophenyl)-4-(imidazo[1,2-a]pyridin-6-yl)-1-((2-(trimethylsilyl) ethoxy)methyl)-1H-pyrazole-5-carboxylic acid

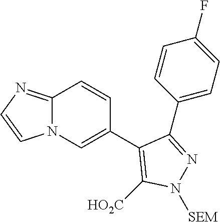 [SMILES: C[Si](C)(C)CCOCn1nc(-c2ccc(F)cc2)c(-c2ccc3nccn3c2)c1C(=O)O]

To a solution of intermediate 93A (0.2 g, 0.416 mmol) in THF (2 mL) and water (1 mL) was added LiOH (0.030 g, 1.248 mmol) and the mixture was stirred at RT for 6 h. The solvent was evaporated from the reaction mixture and the residue was neutralized using 1.5N HCl solution to pH ˜7.0. The aqueous layer was back-extracted with ethyl acetate (3×5 mL). The combined organic layer was washed with brine, dried over Na₂SO₄, filtered and the filtrate was concentrated under reduced pressure to afford intermediate 94A (0.13 g, 69.0% yield). ¹H NMR (400 MHz, DMSO-d₆) δ ppm 8.42 (s, 1H), 7.85 (s, 1H), 7.51 (s, 2H), 7.43 (br. s., 2H), 7.21-7.33 (m, 2H), 6.89 (s, 1H), 5.35 (s, 2H), 3.92 (s, 2H), 0.83 (s, 2H), −0.04 (br. s., 9H).

### Example 133: 3-(4-fluorophenyl)-4-(imidazo[1,2-a]pyridin-6-yl)-1H-pyrazole-5-carboxylic acid

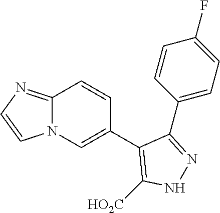 [SMILES: O=C(O)c1[nH]nc(-c2ccc(F)cc2)c1-c1ccc2nccn2c1]

Example 133 was synthesized by reacting intermediate 94A with TFA and by employing the experimental procedure described for Example 1 in Scheme 1. The crude product was dissolved in DMF and purified via preparative HPLC (Condition F). Fractions containing the desired product were combined and dried under vacuum to afford Example 133. LCMS: m/z=323.1 [M+H]⁺; HPLC Ret. Time 0.69 min; (HPLC Method C). ¹H NMR (400 MHz, DMSO-d₆) δ ppm 14.01 (br. s., 1H), 8.47 (br. s., 1H), 7.89 (s, 1H), 7.57 (s, 1H), 7.52 (d, J=9.3 Hz, 1H), 7.37-7.46 (m, 2H), 7.15 (br. s., 2H), 7.04 (d, J=9.3 Hz, 1H).

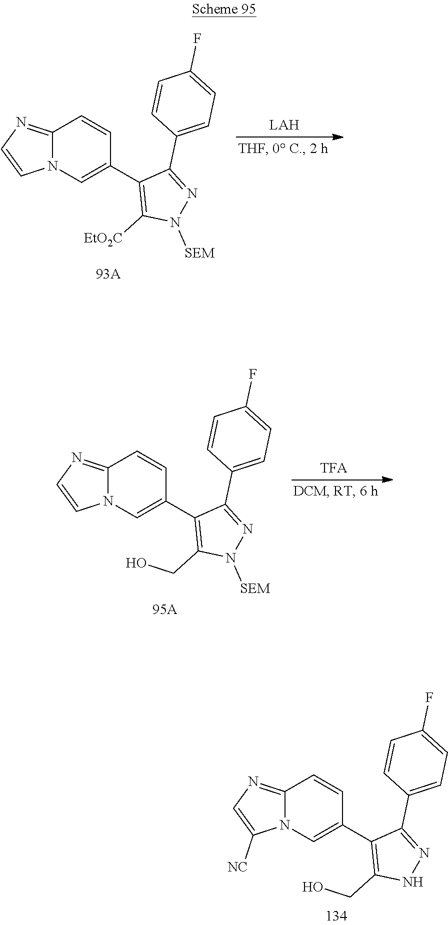 [SMILES: CCOC(=O)c1c(-c2ccc3nccn3c2)c(-c2ccc(F)cc2)nn1COCC[Si](C)(C)C ; C[Si](C)(C)CCOCn1nc(-c2ccc(F)cc2)c(-c2ccc3nccn3c2)c1CO ; N#Cc1cnc2ccc(-c3c(-c4ccc(F)cc4)n[nH]c3CO)cn12]

### Intermediate 95A: (3-(4-fluorophenyl)-4-(imidazo [1,2-a]pyridin-6-yl)-1-((2-(trimethylsilyl) ethoxy)methyl)-1H-pyrazol-5-yl)methanol

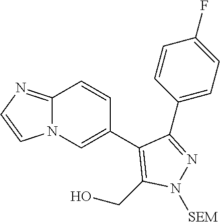 [SMILES: C[Si](C)(C)CCOCn1nc(-c2ccc(F)cc2)c(-c2ccc3nccn3c2)c1CO]

To a solution of intermediate 93A (1.1 g, 2.289 mmol) in THF (10 mL) under nitrogen at 0° C. was added a 2M solution of LAH (1.373 mL, 2.75 mmol) and the reaction mixture was stirred at 0° C. for 2 h. The reaction mixture was quenched with 10% aq. solution of NaOH and the aqueous layer was back-extracted with diethyl ether. The combined organic layers were washed with water and brine, dried over Na₂SO₄, filtered and the filtrate was concentrated to afford intermediate 95A (0.7 g, 69.7% yield). LCMS: m/z=439.5 [M+H]⁺; HPLC Ret. Time 0.85 min; (HPLC Method A).

### Example 134: (3-(4-fluorophenyl)-4-(imidazo[1,2-a]pyridin-6-yl)-1H-pyrazol-5-yl)methanol

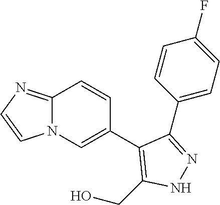 [SMILES: OCc1[nH]nc(-c2ccc(F)cc2)c1-c1ccc2nccn2c1]

Example 134 was synthesized by reacting intermediate 95A with TFA and by employing the experimental procedure described for Example 1 in Scheme 1. The crude product was dissolved in DMF and purified via preparative HPLC (Condition F). Fractions containing the desired product were combined and dried under vacuum to afford Example 134. LCMS: m/z=309.1 [M+H]⁺; HPLC Ret. Time 1.13 min; (HPLC Method C). ¹H NMR (400 MHz, DMSO-d₆) δ ppm 13.30 (s, 1H), 8.80 (s, 1H), 8.09 (d, J 2.0 Hz, 1H), 7.86 (s, 1H), 7.54 (d, J=9.8 Hz, 1H), 7.38-7.45 (m, 2H), 7.10-7.20 (m, 3H), 5.38 (br. s., 1H), 4.47 (s, 2H).

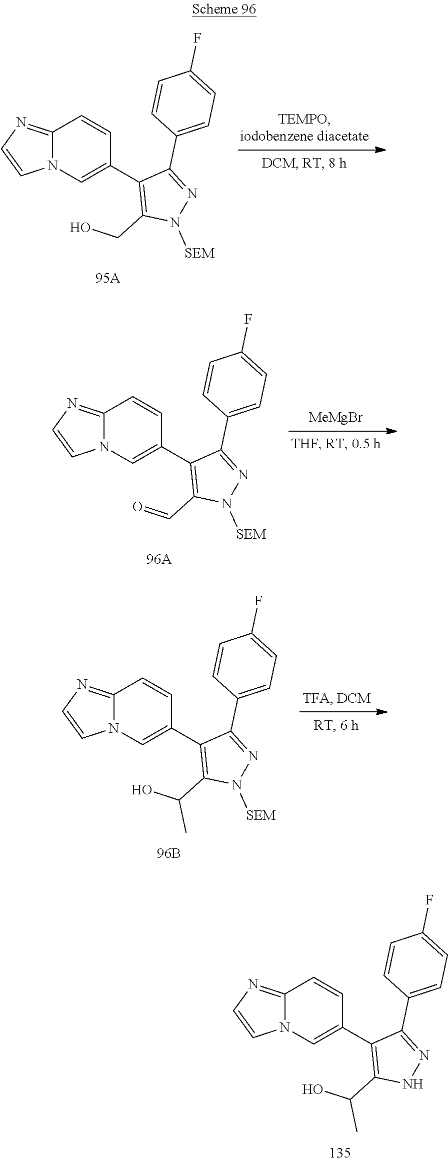

### Intermediate 96A: 3-(4-fluorophenyl)-4-(imidazo[1,2-a]pyridin-6-yl)-1-((2-(trimethylsilyl) ethoxy)methyl)-1H-pyrazole-5-carbaldehyde

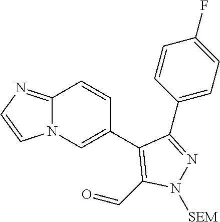 [SMILES: C[Si](C)(C)CCOCn1nc(-c2ccc(F)cc2)c(-c2ccc3nccn3c2)c1C=O]

To a solution of intermediate 95A (0.5 g, 1.140 mmol) in DCM (2 mL) was added iodobenzene diacetate (0.367 g, 1.140 mmol) and TEMPO (0.025 g, 0.160 mmol). The reaction mixture was stirred at RT for 8 h and filtered through a bed of Celite®. The filtrate was then evaporated to afford a pale yellow residue. The crude product was purified by silica gel chromatography (12 g RediSep® column, eluting with a gradient of 100% EtOAc). Fractions containing the desired product were combined and evaporated under reduced pressure to afford intermediate 96A (0.35 g, 70.0% yield). LCMS: m/z=437.2 [M+H]⁺; HPLC Ret. Time 3.2 min; (HPLC Method E).

### Intermediate 96B: 1-(3-(4-fluorophenyl)-4-(imidazo[1,2-a]pyridin-6-yl)-1-((2-(trimethylsilyl)ethoxy)methyl)-1H-pyrazol-5-yl)ethanol

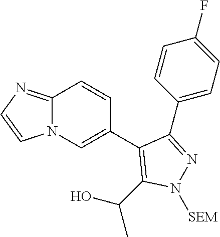 [SMILES: CC(O)c1c(-c2ccc3nccn3c2)c(-c2ccc(F)cc2)nn1COCC[Si](C)(C)C]

Intermediate 96B was synthesized by reacting intermediate 96A with MeMgCl and by employing the experimental procedure described for Example 52 in Scheme 34 to afford intermediate 96B. LCMS: m/z=453.5 [M+H]⁺; HPLC Ret. Time 0.85 min; (HPLC Method B).

### Example 135: 1-(3-(4-fluorophenyl)-4-(imidazo[1,2-a]pyridin-6-yl)-1H-pyrazol-5-yl)ethanol

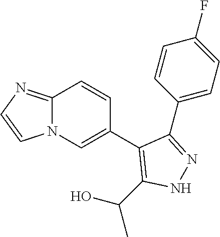 [SMILES: CC(O)c1[nH]nc(-c2ccc(F)cc2)c1-c1ccc2nccn2c1]

Example 135 was synthesized by reacting intermediate 96B with TFA and by employing the experimental procedure described for Example 1 in Scheme 1. The crude product was dissolved in DMF and purified via preparative HPLC (Condition F). Fractions containing the desired product were combined and dried under vacuum to afford Example 135. LCMS: m/z=323.1 [M+H]⁺; HPLC Ret. Time 1.03 min; (HPLC Method C); ¹H NMR (400 MHz, DMSO-d₆) δ ppm 13.32 (br. s., 1H), 8.73 (s, 1H), 8.19 (s, 1H), 7.82-7.87 (m, 1H), 7.81 (s, 1H), 7.67 (dd, J=8.7, 5.8 Hz, 2H), 7.40 (br. s., 2H), 7.25 (d, J=9.5 Hz, 1H), 4.77 (br. s., 2H), 1.39-1.40 (d, 3H).

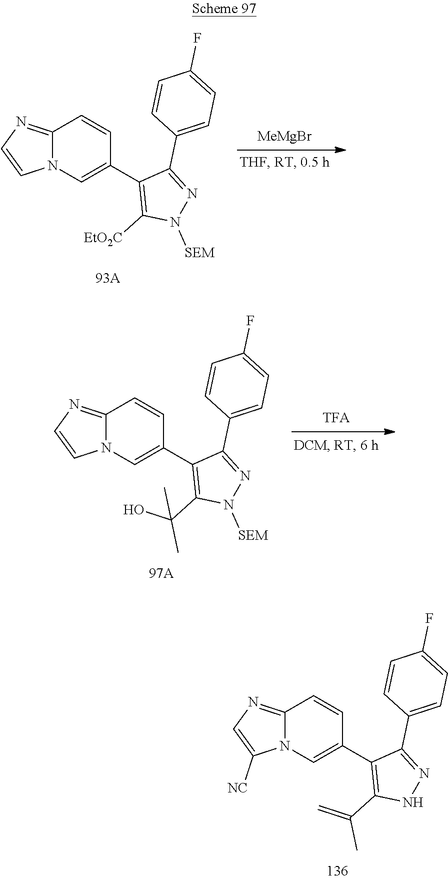 [SMILES: "MeMgBr" ; CCOC(=O)c1c(-c2ccc3nccn3c2)c(-c2ccc(F)cc2)nn1COCC[Si](C)(C)C ; CC(C)(O)c1c(-c2ccc3nccn3c2)c(-c2ccc(F)cc2)nn1COCC[Si](C)(C)C ; C=C(C)c1[nH]nc(-c2ccc(F)cc2)c1-c1ccc2ncc(C#N)n2c1 ; "TFA"]

### Intermediate 97A: 2-(3-(4-fluorophenyl)-4-(imidazo [1,2-a]pyridin-6-yl)-1-((2-(trimethylsilyl) ethoxy)methyl)-1H-pyrazol-5-yl)propan-2-ol

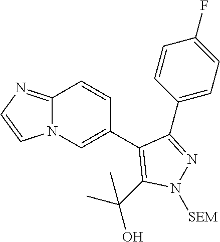 [SMILES: CC(C)(O)c1c(-c2ccc3nccn3c2)c(-c2ccc(F)cc2)nn1COCC[Si](C)(C)C]

Intermediate 97A was synthesized by reacting intermediate 93A with MeMgCl and by employing the experimental procedure described for Example 52 in Scheme 34 to afford intermediate 97A. LCMS: m/z=467.2 [M+H]⁺; HPLC Ret. Time 3.2 min. (HPLC Method E).

### Example 136: 6-(3-(4-fluorophenyl)-5-(prop-1-en-2-yl)-1H-pyrazol-4-yl)imidazo[1,2-a]pyridine

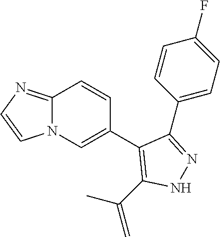 [SMILES: C=C(C)c1[nH]nc(-c2ccc(F)cc2)c1-c1ccc2nccn2c1]

A solution of intermediate 97A (0.05 g, 0.107 mmol) and TFA (0.085 mL, 1.072 mmol) in CH₂Cl₂ (3 mL) was stirred at RT for 3 h. The reaction mixture was evaporated under reduced pressure, the obtained residue was basified using 10% aq. NaHCO₃ solution and the solid thus obtained was filtered through a Buchner funnel to afford the crude product. The crude product was dissolved in DMF and purified via preparative HPLC (Condition N). Fractions containing the desired product were combined and dried under vacuum to afford Example 136 (5.0 mg, 14.04% yield). LCMS: m/z=319.1 [M+H]⁺; HPLC Ret. Time 1.51 min; (HPLC Method C). ¹H NMR (400 MHz, DMSO-d₆) δ ppm 13.28 (br. s., 1H), 8.39-8.42 (m, 1H), 7.89 (s, 1H), 7.54-7.61 (m, 2H), 7.39 (dd, J=8.5, 5.5 Hz, 2H), 7.15 (t, J=8.5 Hz, 2H), 7.07 (dd, J=9.3, 1.8 Hz, 1H), 4.91-5.10 (m, 2H), 1.98 (br. s., 3H).

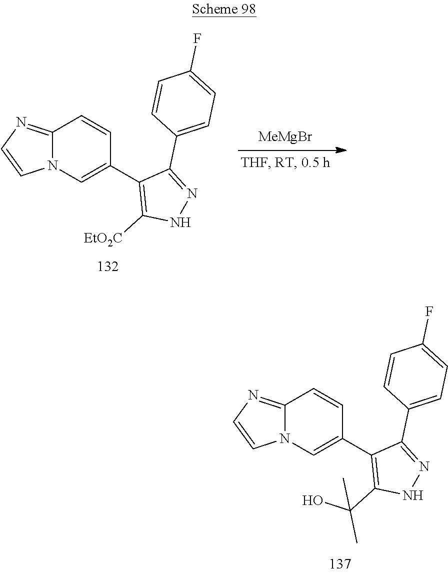 [SMILES: "MeMgBr" ; CCOC(=O)c1[nH]nc(-c2ccc(F)cc2)c1-c1ccc2nccn2c1 ; CC(C)(O)c1[nH]nc(-c2ccc(F)cc2)c1-c1ccc2nccn2c1]

### Example 137: 2-(3-(4-fluorophenyl)-4-(imidazo[1,2-a]pyridin-6-yl)-1H-pyrazol-5-yl)propan-2-ol

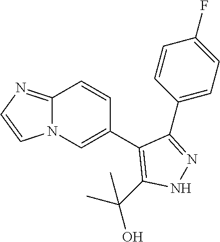 [SMILES: CC(C)(O)c1[nH]nc(-c2ccc(F)cc2)c1-c1ccc2nccn2c1]

Example 136 was synthesized by reacting Example 132 with MeMgCl and by employing the experimental procedure described for Example 52 in Scheme 34. The crude product was dissolved in DMF and purified via preparative HPLC (Condition N). Fractions containing the desired product were combined and dried under vacuum to afford Example 137. LCMS: m/z=337.2 [M+H]⁺; HPLC Ret. Time 1.18 min; (HPLC Method C). ¹H NMR (400 MHz, DMSO-d₆) δ ppm 12.80 (br. s., 1H), 8.41 (dd, J=1.7, 0.98 Hz, 1H), 7.89 (s, 1H), 7.46-7.60 (m, 2H), 7.30-7.40 (m, 2H), 7.07 (d, J=8.8 Hz, 3H), 5.35 (br. s., 1H), 1.35 (s, 6H).

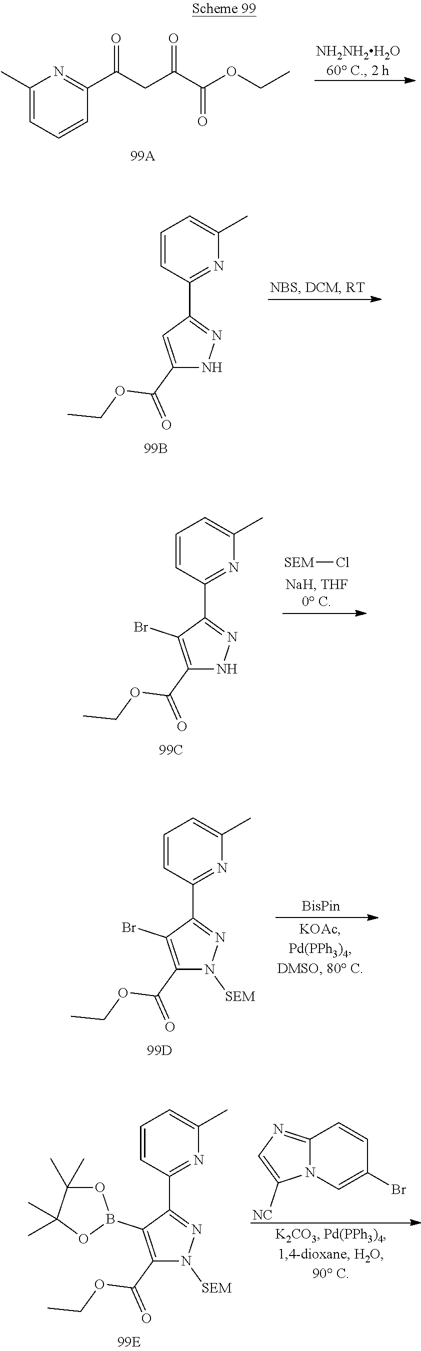

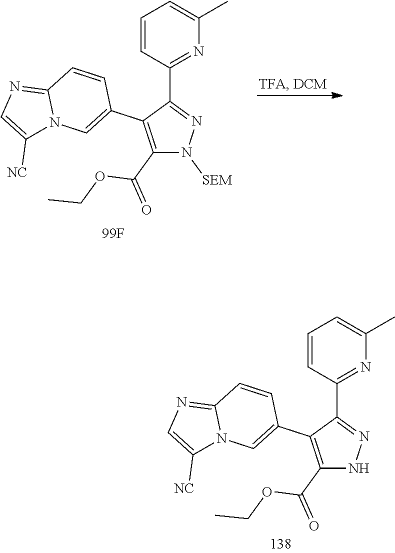 [SMILES: CCOC(=O)c1c(-c2ccc3ncc(C#N)n3c2)c(-c2cccc(C)n2)nn1COCC[Si](C)(C)C ; CCOC(=O)c1[nH]nc(-c2cccc(C)n2)c1-c1ccc2ncc(C#N)n2c1]

### Intermediate 99B: ethyl 3-(6-methylpyridin-2-yl)-1H-pyrazole-5-carboxylate

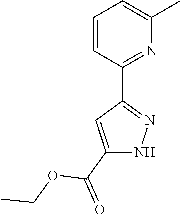 [SMILES: CCOC(=O)c1cc(-c2cccc(C)n2)n[nH]1]

Intermediate 99B was synthesized by reacting commercially available 99A with hydrazine and by employing the experimental procedure described for intermediate 53B in Scheme 53. The crude product was purified by silica gel chromatography (24 g RediSep® column, eluting with a gradient of 0-60% EtOAc in petroleum ether). Fractions containing the desired product were combined and evaporated to afford intermediate 99B. LCMS: m/z=232.5 [M+H]⁺; HPLC Ret. Time 0.51 min (HPLC Method A).

### Intermediate 99C: ethyl 4-bromo-3-(6-methylpyridin-2-yl)-1H-pyrazole-5-carboxylate

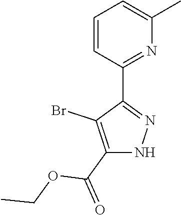 [SMILES: CCOC(=O)c1[nH]nc(-c2cccc(C)n2)c1Br]

Intermediate 99C was synthesized by reacting intermediate 99B with NBS and by employing the experimental procedure described for intermediate 1D in Scheme 1 to afford intermediate 99C. LCMS: m/z=312 [M+H]⁺; HPLC Ret. Time 1.19 min (HPLC Method B).

### Intermediate 99D: ethyl 4-bromo-3-(6-methylpyridin-2-yl)-1-((2-(trimethylsilyl) ethoxy)methyl)-1H-pyrazole-5-carboxylate

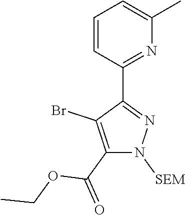 [SMILES: CCOC(=O)c1c(Br)c(-c2cccc(C)n2)nn1COCC[Si](C)(C)C]

Intermediate 99D was synthesized by reacting intermediate 99C with SEM-Cl and by employing the experimental procedure described for intermediate 8A in Scheme 8 to afford intermediate 99D. The crude compound was used in the next step without further purification. LCMS: m/z=442.0 [M+H]⁺; HPLC Ret. Time 1.10 min. (HPLC Method A).

### Intermediate 99E: ethyl 3-(6-methylpyridin-2-yl)-4-(4,4,5,5-tetramethyl-1,3,2-dioxaborolan-2-yl)-1-((2-(trimethylsilyl)ethoxy)methyl)-1H-pyrazole-5-carboxylate

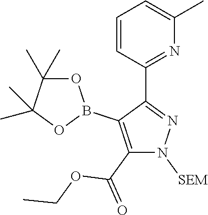

Intermediate 99E was synthesized by reacting intermediate 99D with 4,4,4′,4′,5,5,5′,5′-octamethyl-2,2′-bi(1,3,2-dioxaborolane) and by employing the experimental procedure described for intermediate 8B in Scheme 8. The crude product was purified by silica gel chromatography (40 g RediSep® column, eluting with a gradient of 0-70% EtOAc in petroleum ether). Fractions containing the desired product were combined and evaporated under reduced pressure to afford intermediate 99E. LCMS: m/z=406.1 [M-81, pinacol]⁺; HPLC Ret. Time 1.08 min. (HPLC Method A).

### Intermediate 99F: ethyl 4-(3-cyanoimidazo[1,2-a]pyridin-6-yl)-3-(6-methylpyridin-2-yl)-1-((2-(trimethylsilyl)ethoxy)methyl)-1H-pyrazole-5-carboxylate

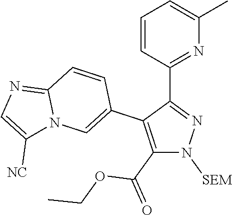 [SMILES: CCOC(=O)c1c(-c2ccc3ncc(C#N)n3c2)c(-c2cccc(C)n2)nn1COCC[Si](C)(C)C]

Intermediate 99F was synthesized by reacting intermediate 99E with 6-bromoimidazo[1,2-a]pyridine-3-carbonitrile and by employing the experimental procedure described for Example 34A in Scheme 34 to afford intermediate 99F. LCMS: m/z=504.3 [M+2H]⁺; HPLC Ret. Time 1.08 min. (HPLC Method A).

### Example 138: ethyl 4-(3-cyanoimidazo[1,2-a]pyridin-6-yl)-3-(6-methylpyridin-2-yl)-1H-pyrazole-5-carboxylate

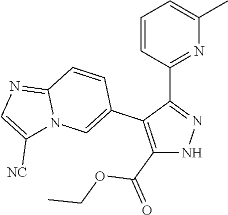 [SMILES: CCOC(=O)c1[nH]nc(-c2cccc(C)n2)c1-c1ccc2ncc(C#N)n2c1]

Example 138 was synthesized by reacting intermediate 99F with TFA and by employing the experimental procedure described for Example 1 in Scheme 1. The crude product was dissolved in DMF and purified via preparative HPLC (Condition N). Fractions containing the desired product were combined and dried under vacuum to afford Example 138. LCMS: m/z=373.1 [M+H]⁺; HPLC Ret. Time 1.523 min. (HPLC Method C); ¹H NMR (400 MHz, DMSO-d₆) δ ppm 8.74-8.85 (m, 1H), 8.49 (s, 1H), 7.48-7.86 (m, 3H), 7.11-7.27 (m, 2H), 4.16 (d, J=6.5 Hz, 2H), 2.37 (s, 3H), 1.11 (d, J=7.5 Hz, 3H).

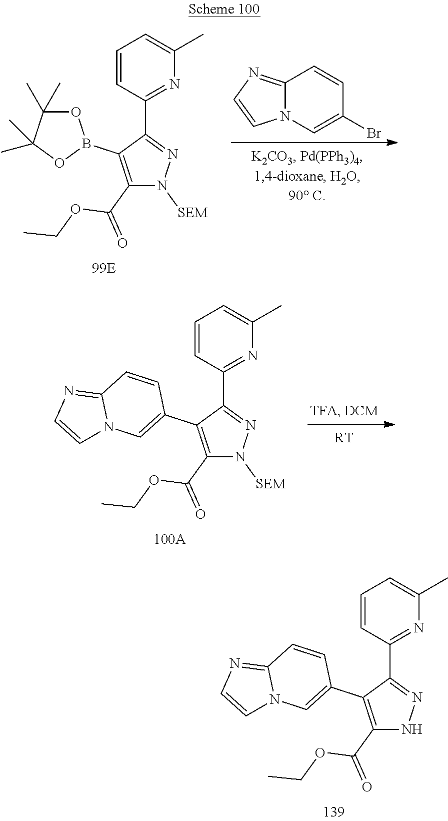

### Intermediate 100A: ethyl 4-(imidazo[1,2-a]pyridin-6-yl)-3-(6-methylpyridin-2-yl)-1-((2-(trimethylsilyl)ethoxy)methyl)-1H-pyrazole-5-carboxylate

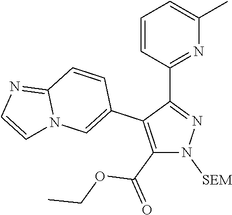 [SMILES: CCOC(=O)c1c(-c2ccc3nccn3c2)c(-c2cccc(C)n2)nn1COCC[Si](C)(C)C]

Intermediate 100A was synthesized by reacting intermediate 99E with 6-bromoimidazo[1,2-a]pyridine and by employing the experimental procedure described for intermediate 1E in Scheme 1. The crude product was purified by silica gel chromatography (40 g RediSep® column, eluting with a gradient of 10-60% EtOAc in petroleum ether). Fractions containing the desired product were combined and evaporated under reduced pressure to afford intermediate 100A. LCMS: m/z=478.2 [M+H]⁺; HPLC Ret. Time 1.00 min. (HPLC Method A).

### Example 139: ethyl 4-(imidazo[1,2-a]pyridin-6-yl)-3-(6-methylpyridin-2-yl)-1H-pyrazole-5-carboxylate

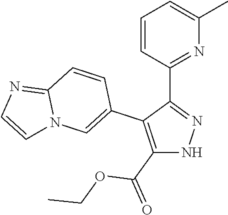 [SMILES: CCOC(=O)c1[nH]nc(-c2cccc(C)n2)c1-c1ccc2nccn2c1]

Example 139 was synthesized by reacting intermediate 100A with TFA and by employing the experimental procedure described for Example 1 in Scheme 1. The crude product was dissolved in DMF and purified via preparative HPLC (Condition F). Fractions containing the desired product were combined and dried under vacuum to afford Example 139. LCMS: m/z=348.1 [M+H]⁺; HPLC Ret. Time 1.343 min. (HPLC Method C); ¹H NMR (400 MHz, DMSO-d₆) δ ppm 8.59 (br. s., 1H), 7.96 (br. s., 1H), 7.53-7.71 (m, 3H), 7.20 (br. s., 2H), 7.00 (d, J=5.6 Hz, 1H), 4.14 (d, J=6.1 Hz, 2H), 2.43-2.47 (m, 3H), 1.09 (t, J=6.7 Hz, 3H).

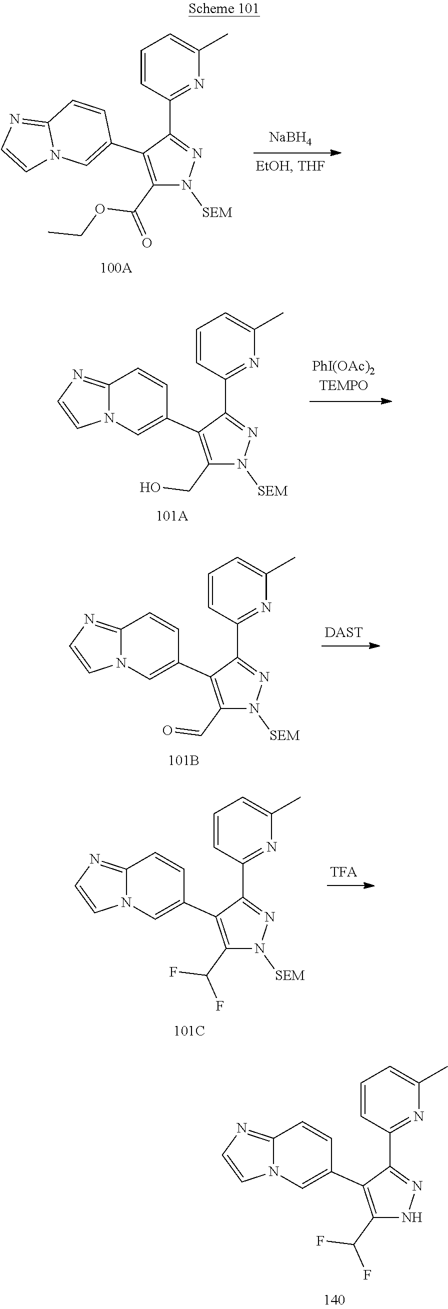

### Intermediate 101A: (4-(imidazo[1,2-a]pyridin-6-yl)-3-(6-methylpyridin-2-yl)-1-((2-(trimethylsilyl)ethoxy)methyl)-1H-pyrazol-5-yl)methanol

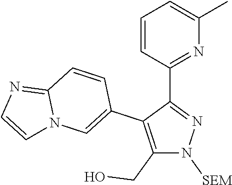 [SMILES: Cc1cccc(-c2nn(COCC[Si](C)(C)C)c(CO)c2-c2ccc3nccn3c2)n1]

To a solution of 100A (250 mg, 0.523 mmol) in ethanol (2 mL), THF (2 mL) was added NaBH₄ (396 mg, 10.47 mmol). The reaction mixture was stirred at RT for 16 h. The reaction mixture was partitioned between water and ethyl acetate. The aqueous layer was back-extracted with ethyl acetate (2×20 mL). The combined organic layers ware washed with brine, dried over Na₂SO₄, filtered and the filtrate was concentrated under reduced pressure to afford intermediate 101A (150 mg, 11.2% yield) LCMS: m/z=436.2 [M+H]⁺; HPLC Ret. Time 0.54 min. (HPLC Method A).

### Intermediate 101B: 4-(imidazo[1,2-a]pyridin-6-yl)-3-(6-methylpyridin-2-yl)-1-((2-(trimethylsilyl)ethoxy)methyl)-1H-pyrazole-5-carbaldehyde

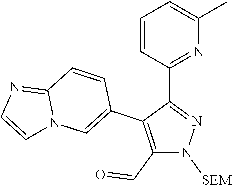 [SMILES: Cc1cccc(-c2nn(COCC[Si](C)(C)C)c(C=O)c2-c2ccc3nccn3c2)n1]

Intermediate 101B was synthesized by reacting intermediate 101A with TEMPO and by employing the experimental procedure described for intermediate 96A in Scheme 96 to afford intermediate 101B. LCMS: m/z=434.5 [M+H]⁺; HPLC Ret. Time 0.68 min. (HPLC Method A).

### Intermediate 101C: 6-(5-(difluoromethyl)-3-(6-methylpyridin-2-yl)-1-((2-(trimethylsilyl) ethoxy)methyl)-1H-pyrazol-4-yl)imidazo[1,2-a]pyridine

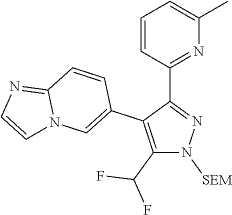 [SMILES: Cc1cccc(-c2nn(COCC[Si](C)(C)C)c(C(F)F)c2-c2ccc3nccn3c2)n1]

To a solution of 101B (120 mg, 0.277 mmol) in DCM (5 mL) was added DAST (0.154 mL, 1.107 mmol) at 0° C. The reaction mixture was warmed to RT, stirred for 4 h, and partitioned between water and DCM. The aqueous layer was back-extracted with DCM (2×20 mL). The combined organic layers were washed with brine, dried over Na₂SO₄, filtered and the filtrate was concentrated under reduced pressure to afford compound 101C (110 mg, 13.96% yield). LCMS: m/z=456.5 [M+H]⁺; HPLC Ret. Time 0.69 min. (HPLC Method A).

### Example 140: 6-(5-(difluoromethyl)-3-(6-methylpyridin-2-yl)-1H-pyrazol-4-yl)imidazo[1,2-a]pyridine

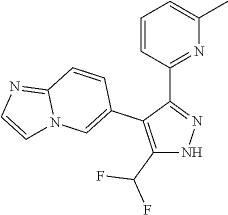 [SMILES: Cc1cccc(-c2n[nH]c(C(F)F)c2-c2ccc3nccn3c2)n1]

Example 140 was synthesized by reacting intermediate 101C with TFA and by employing the experimental procedure described for Example 1 in Scheme 1. The crude product was dissolved in DMF and purified via preparative HPLC (Condition L). Fractions containing the desired product were combined and dried under vacuum to afford Example 140. LCMS: m/z=326.1 [M+H]⁺; HPLC Ret. Time 1.332 min. (HPLC Method C); ¹H NMR (400 MHz, DMSO-d₆) δ ppm 13.99 (s, 1H), 8.59 (s, 1H), 7.99 (s, 3H), 7.55-7.67 (m, 4H), 2.48 (s, 3H).

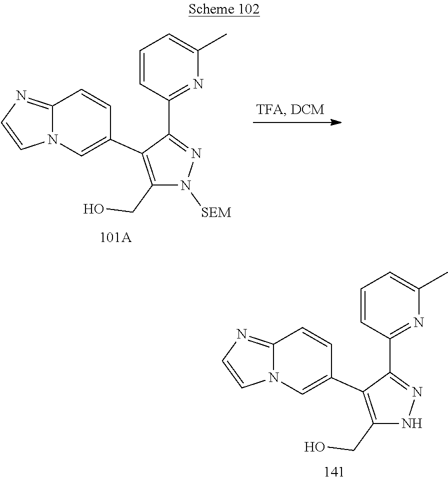

### Example 141: (4-(imidazo[1,2-a]pyridin-6-yl)-3-(6-methylpyridin-2-yl)-1H-pyrazol-5-yl)methanol

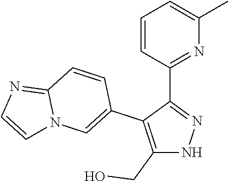 [SMILES: Cc1cccc(-c2n[nH]c(CO)c2-c2ccc3nccn3c2)n1]

Example 141 was synthesized by reacting intermediate 101A with TFA and by employing the experimental procedure described for Example 1 in Scheme 1. The crude product was dissolved in DMF and purified via preparative HPLC (Condition F). Fractions containing the desired product were combined and dried under vacuum to afford Example 141. LCMS: m/z=306.1 [M+H]⁺; HPLC Ret. Time 0.807 min. (HPLC Method C); ¹H NMR (400 MHz, DMSO-d₆) δ ppm 8.88 (s, 1H), 8.26 (s, 1H), 8.07 (s, 1H), 7.73-7.87 (m, 2H), 7.64-7.72 (m, 1H), 7.29-7.62 (m, 1H), 7.08-7.28 (m, 1H), 6.84 (d, J=9.3 Hz, 1H), 5.40 (br. s., 1H), 4.50 (s, 2H), 2.28 (br. s., 3H).

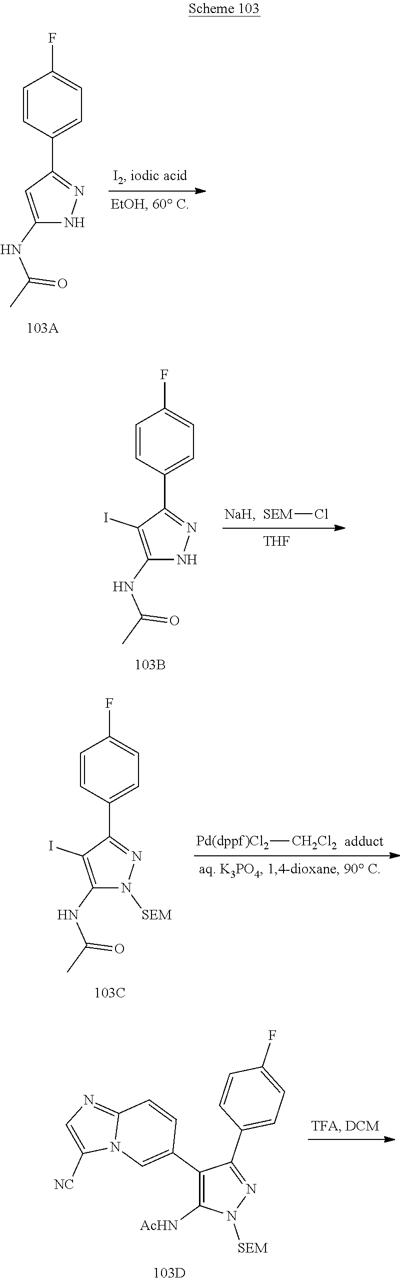

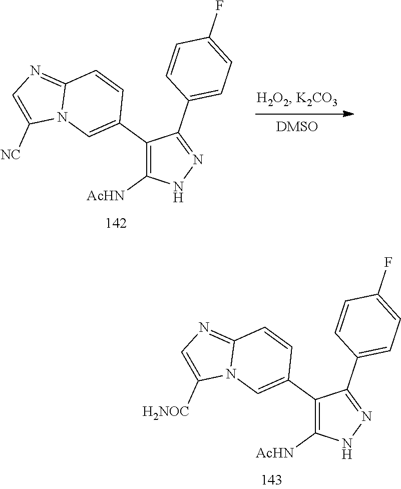 [SMILES: CC(=O)Nc1[nH]nc(-c2ccc(F)cc2)c1-c1ccc2ncc(C#N)n2c1 ; CC(=O)Nc1[nH]nc(-c2ccc(F)cc2)c1-c1ccc2ncc(C(N)=O)n2c1]

### Intermediate 103B: N-(3-(4-fluorophenyl)-4-iodo-1H-pyrazol-5-yl)acetamide

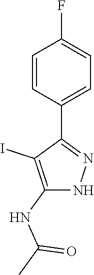 [SMILES: CC(=O)Nc1[nH]nc(-c2ccc(F)cc2)c1I]

To a solution of 103A (700 mg, 3.19 mmol) in ethanol (20 mL) was added iodic acid (140 mg, 0.798 mmol) and iodine (405 mg, 1.597 mmol). The reaction mixture was heated at 60° C. for 1 h. The reaction mixture was cooled to RT and ethanol was evaporated, crude compound was suspended in DCM and washed with a 2M aq. Na₂S₂O₃ solution, dried under dried over Na₂SO₄, filtered and the filtrate was concentrated under reduced pressure to afford intermediate 103B (200 mg, 18.15% yield) as a brown solid. LCMS: m/z=345.9 [M+H]⁺; HPLC Ret. Time 0.96 min. (HPLC Method A).

### Intermediate 103C: N-(3-(4-fluorophenyl)-4-iodo-1-((2-(trimethylsilyl)ethoxy)methyl)-1H-pyrazol-5-yl)acetamide

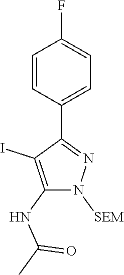 [SMILES: CC(=O)Nc1c(I)c(-c2ccc(F)cc2)nn1COCC[Si](C)(C)C]

Intermediate 103C was synthesized by reacting intermediate 103B with SEM-Cl and by employing the experimental procedure described for intermediate 8A in Scheme 8. The crude product was purified by silica gel chromatography (4 g RediSep® column, eluting with a gradient of 20-50% EtOAc in petroleum ether). Fractions containing the desired product were combined and evaporated to afford intermediate 103C. LCMS: m/z=476.4 [M+H]⁺; HPLC Ret. Time 1.51 min. (HPLC Method B).

### Intermediate 103D: N-(4-(3-cyanoimidazo[1,2-a]pyridin-6-yl)-3-(4-fluorophenyl)-1-((2-(trimethylsilyl)ethoxy)methyl)-1H-pyrazol-5-yl)acetamide

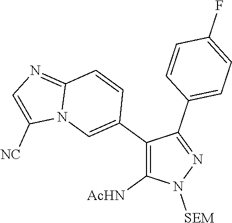 [SMILES: CC(=O)Nc1c(-c2ccc3ncc(C#N)n3c2)c(-c2ccc(F)cc2)nn1COCC[Si](C)(C)C]

Intermediate 103D was synthesized by reacting intermediate 103C with 6-(4,4,5,5-tetramethyl-1,3,2-dioxaborolan-2-yl)imidazo[1,2-a]pyridine-3-carbonitrile and by employing the experimental procedure described for Intermediate 1E in Scheme 1. The crude product was purified by silica gel chromatography (12 g RediSep® column, eluting with a gradient of 5-10% MeOH in chloroform). Fractions containing the desired product were combined and evaporated to afford intermediate 103D (40 mg, 64.6% yield). LCMS: m/z=491.1 [M+H]⁺; HPLC Ret. Time 0.91 min. (HPLC Method A).

### Example 142: N-(4-(3-cyanoimidazo[1,2-a]pyridin-6-yl)-3-(4-fluorophenyl)-1H-pyrazol-5-yl)acetamide

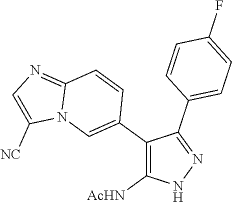 [SMILES: CC(=O)Nc1[nH]nc(-c2ccc(F)cc2)c1-c1ccc2ncc(C#N)n2c1]

Example 142 was synthesized by reacting intermediate 103D with TFA and by employing the experimental procedure described for Example 1 in Scheme 1. The crude product was dissolved in DMF and purified via preparative HPLC (Condition I). Fractions containing the desired product were combined and dried under vacuum to afford Example 142 as a TFA salt. LCMS: m/z=361.1 [M+H]⁺; HPLC Ret. Time 1.20 min. (HPLC Method C); ¹H NMR (400 MHz, DMSO-d₆) δ ppm 13.26 (br. s., 1H), 9.79 (br. s., 1H), 8.39-8.50 (m, 2H), 7.78 (d, J=9.1 Hz, 1H), 7.46 (dd, J=8.6, 5.4 Hz, 2H), 7.17-7.22 (m, 3H) 1.98 (br. s., 3H).

### Example 143: 6-(5-acetamido-3-(4-fluorophenyl)-1H-pyrazol-4-yl)imidazo[1,2-a]pyridine-3-carboxamide

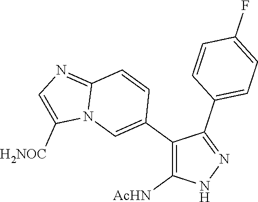 [SMILES: CC(=O)Nc1[nH]nc(-c2ccc(F)cc2)c1-c1ccc2ncc(C(N)=O)n2c1]

Example 143 was synthesized by reacting Example 142 with H₂O₂/K₂CO₃ and by employing the experimental procedure described for Example 2 in Scheme 1. The crude product was dissolved in DMF and purified via preparative HPLC (Condition N). Fractions containing the desired product were combined and dried under vacuum to afford Example 143. LCMS: m/z=379.1 [M+H]⁺; HPLC Ret. Time 1.02 min. (HPLC Method C); HPLC Ret. Time 0.92 min. (HPLC Method D); ¹H NMR (400 MHz, DMSO-d₆) δ ppm 13.11-13.23 (m, 1H), 9.64-10.03 (m, 1H), 9.36 (s, 1H), 8.29-8.36 (m, 1H), 7.63-7.97 (m, 2H), 7.42 (dd, J=8.8, 5.4 Hz, 2H), 7.19-7.29 (m, 2H), 6.94-7.18 (m, 2H), 1.87 (s, 3H).

### Biological Assays

Assays are conducted in 1536-well plates and 2 μL reactions are prepared from addition of HIS-TGFβR1 T204D or HIS-TGFβR2 WT, anti-HIS detection antibody, a labeled small molecule probe (K_{d}=<100 nM; koff=<0.001 s⁻¹) and test compounds in assay buffer (20 mM HEPES pH 7.4, 10 mM MgCl₂, 0.015% Brij35, 4 mM DTT, and 0.05 mg/ml BSA). The reaction is incubated for 1 hour at room temperature and the HTRF signal was measured on an Envision plate reader (Ex: 340 nm; Em: 520 nm/495 nm). Inhibition data were calculated by comparison to no enzyme control reactions for 100% inhibition and vehicle-only reactions for 0% inhibition. The final concentration of reagents in the assay are 1 nM HIS-TGFβR1 T204D or HIS-TGFβR2 WT, 0.2 nM anti-HIS detection antibody, labeled small molecule probe (at K_{d}) and 0.5% DMSO. Dose response curves were generated to determine the concentration required inhibiting 50% of kinase activity (IC₅₀). Compounds were dissolved at 10 mM in dimethylsulfoxide (DMSO) and evaluated at eleven concentrations. IC₅₀ values were derived by non-linear regression analysis.

The table shows the TGFβR1 and TGFβR2 IC₅₀ values for the Examples of this invention.

| Example # | TGFβR2 (μM) | TGFβR1 (μM) |
|---|---|---|
| 1 | | 0.001 |
| 2 | 1.828 | 0.001 |
| 3 | 15.000 | 0.001 |
| 4 | 1.109 | 0.001 |
| 5 | 0.193 | 0.000 |
| 6 | 0.259 | 0.000 |
| 7 | 15.000 | 0.008 |
| 8 | 15.000 | 0.006 |
| 9 | 15.000 | 0.024 |
| 10 | 8.874 | 0.001 |
| 11 | 5.421 | 0.001 |
| 12 | 0.396 | 0.000 |
| 13 | 0.101 | 0.007 |
| 14 | 0.885 | 0.004 |
| 15 | 12.663 | 0.502 |
| 16 | 15.000 | 0.187 |
| 17 | | |
| 18 | 0.752 | 0.000 |
| 19 | 0.277 | 0.000 |
| 20 | 1.448 | 0.001 |
| 21 | 0.205 | 0.000 |
| 22 | 7.552 | 0.035 |
| 23 | 2.478 | 0.001 |
| 24 | 0.627 | 0.001 |
| 25 | 9.737 | 0.003 |
| 26 | | |
| 27 | 11.365 | 0.003 |
| 28 | 0.075 | 0.001 |
| 29 | 1.105 | 0.003 |
| 30 | 10.286 | 0.591 |
| 31 | 9.169 | 0.112 |
| 32 | 15.000 | 0.465 |
| 33 | 7.058 | 0.811 |
| 34 | 5.017 | 0.014 |
| 35 | 0.221 | 0.011 |
| 36 | 0.696 | 0.002 |
| 37 | 0.165 | 0.042 |
| 38 | 1.544 | 0.002 |
| 39 | 1.652 | 0.004 |
| 40 | 4.966 | 0.003 |
| 41 | 0.098 | 0.001 |
| 42 | 0.330 | 0.001 |
| 43 | 0.463 | 0.001 |
| 44 | 0.369 | 0.002 |
| 45 | 4.226 | 0.112 |
| 46 | 0.214 | 0.092 |
| 47 | 0.709 | 0.004 |
| 48 | 4.119 | 0.002 |
| 49 | 0.104 | 0.001 |
| 50 | 0.368 | 0.000 |
| 51 | 2.231 | 0.001 |
| 52 | 15.000 | 0.007 |
| 53 | 15.000 | 0.030 |
| 54 | 15.000 | 0.079 |
| 55 | 4.929 | 0.006 |
| 56 | 0.533 | 0.007 |
| 57 | 3.709 | 0.003 |
| 58 | 15.000 | 0.025 |
| 59 | 5.337 | 0.241 |
| 60 | 15.000 | 0.012 |
| 61 | 2.283 | 0.007 |
| 62 | 0.518 | 0.000 |
| 63 | | |
| 64 | 0.012 | 0.001 |
| 65 | 0.194 | 0.001 |
| 66 | 0.277 | 0.001 |
| 67 | 11.140 | 0.004 |
| 68 | 0.176 | 0.001 |
| 69 | 15.000 | 0.007 |
| 70 | 6.102 | 0.001 |
| 71 | 15.000 | 0.145 |
| 72 | 0.963 | 0.002 |
| 73 | 15.000 | 9.863 |
| 74 | 15.000 | 0.374 |
| 75 | 15.000 | 0.054 |
| 76 | 6.591 | 0.001 |
| 77 | 15.000 | 0.144 |
| 78 | 6.670 | 0.001 |
| 79 | 0.136 | 0.001 |
| 80 | 15.000 | 0.001 |
| 81 | 15.000 | 0.006 |
| 82 | | |
| 83 | 15.000 | 0.017 |
| 84 | 2.680 | 0.003 |
| 85 | 0.143 | 0.000 |
| 86 | 14.565 | 0.004 |
| 87 | 6.899 | 0.002 |
| 88 | | |
| 89 | 15.000 | 0.010 |
| 90 | 15.000 | 0.004 |
| 91 | 15.000 | 0.008 |
| 92 | 2.654 | 0.001 |
| 93 | | |
| 94 | 0.806 | 0.001 |
| 95 | 15.000 | 0.058 |
| 96 | 15.000 | 0.070 |
| 97 | 6.360 | 0.001 |
| 98 | 15.000 | 0.050 |
| 99 | 2.980 | 0.001 |
| 100 | 0.094 | 0.001 |
| 101 | 15.000 | 0.001 |
| 102 | 15.000 | 0.014 |
| 103 | | |
| 104 | 15.000 | 2.240 |
| 105 | 15.000 | 0.041 |
| 106 | 6.655 | 0.001 |
| 107 | 15.000 | 0.147 |
| 108 | 15.000 | 0.022 |
| 109 | 15.000 | 1.132 |
| 110 | 15.000 | 0.173 |
| 111 | 0.577 | 0.002 |
| 112 | 0.105 | 0.011 |
| 113 | 15.000 | 3.815 |
| 114 | 0.761 | 0.001 |
| 115 | 0.009 | 0.001 |
| 116 | 5.770 | 0.001 |
| 117 | 15.000 | 0.009 |
| 118 | 2.689 | 0.001 |
| 119 | 4.955 | 0.003 |
| 120 | 15.000 | 0.115 |
| 121 | 0.216 | 0.001 |
| 122 | 0.004 | 0.001 |
| 123 | 0.469 | 0.001 |
| 124 | 2.059 | 0.001 |
| 125 | 0.013 | 0.001 |
| 126 | 3.155 | 0.001 |
| 127 | 0.128 | 0.001 |
| 128 | 15.000 | 0.281 |
| 129 | 15.000 | 1.763 |
| 130 | 15.000 | 0.145 |
| 131 | 15.000 | 1.408 |
| 132 | 15.000 | 0.758 |
| 133 | 15.000 | 25.336 |
| 134 | 13.133 | 0.027 |
| 135 | | |
| 136 | 15.000 | 0.023 |
| 137 | 15.000 | 0.153 |
| 138 | 15.000 | 0.002 |
| 139 | 15.000 | 0.002 |
| 140 | 15.000 | 0.001 |
| 141 | 4.468 | 0.001 |
| 142 | 15.000 | 0.274 |
| 143 | 15.000 | 0.027 |

1. A compound having the structure of formula (Ia) or (Ib): 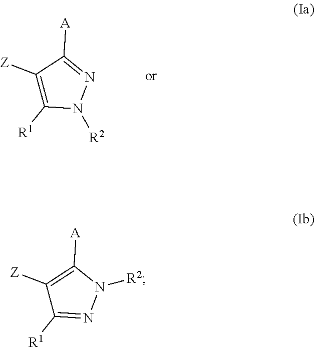
or a pharmaceutically acceptable salt thereof,
wherein
A is phenyl substituted with 0-3 R⁴ groups or pyridyl substituted with 0-3 R⁴ groups;
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
Z is a 10-12 membered heterobicyclic ring substituted with 0-2 R^{Z} groups containing 1-4 heteroatoms selected from —O—, —S— or —N—;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

2. A compound according to claim 1 of formula (Ia): 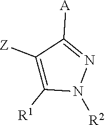
or a pharmaceutically acceptable salt thereof,
wherein
A is phenyl substituted with 0-3 R⁴ groups or pyridyl substituted with 0-3 R⁴ groups;
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
Z is a 10-12 membered heterobicyclic ring substituted with 0-2 R^{Z} groups containing 1-4 heteroatoms selected from —O—, —S— or —N—;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

3. The compound of claim 2, wherein
A is phenyl substituted with 0-2 R⁴ groups or pyridyl substituted with 0-2 R⁴ groups;
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
Z is a 10-12 membered heterobicyclic ring substituted with 0-2 R^{Z} groups containing 1-4 heteroatoms selected from —O—, —S— or —N—;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

4. The compound according to claim 3, wherein
Z is 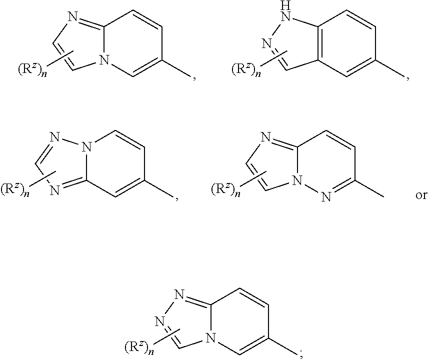
and n is 0, 1 or 2.

5. The compound according to claim 4, wherein
Z is 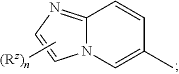
and n is 0, 1 or 2.

6. The compound according to claim 4, wherein
Z is 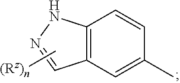
and n is 0, 1 or 2.

7. The compound according to claim 4, wherein
Z is 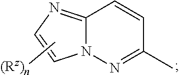
and n is 0, 1 or 2.

8-16. (canceled)

17. The compound as defined in claim 1 of the formula 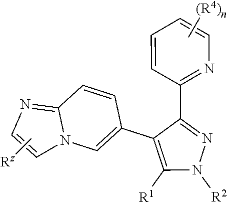
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

18. The compound as defined in claim 1 of the formula 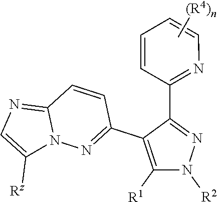
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

19. A compound as defined in claim 1 of the formula 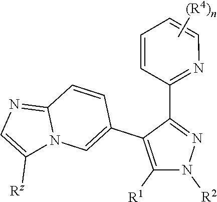
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

20. The compound as defined in claim 1 of the formula 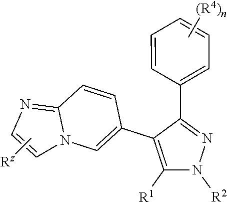
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

21. The compound as defined in claim 1 of the formula 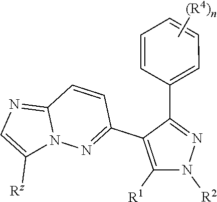
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

22. A compound as defined in claim 1 of the formula 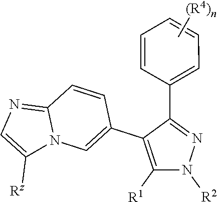
or a pharmaceutically acceptable salt thereof,
wherein
each R⁴ is independently hydrogen, halogen, C₁-C₆ alkyl, C₁-C₆ haloalkyl, C₁-C₆ alkoxy, C₁-C₆ haloalkoxy, —NO₂, or —CN;
each R^{Z} is independently halogen, —CN, C₁-C₆ alkyl, C₁-C₆ haloalkyl, C₁-C₆ alkoxy, C₁-C₆ alkenyl, C₃-C₆ cycloalkyl, C₁-C₆ alkoxy, C₁-C₆ alkanol, —COO C₁-C₆ alkyl, —CONR^{z1}R^{z2}, —NHC(O) C₁-C₆ haloalkyl, —NHC(O) C₁-C₆ alkyl, or —NH C₁-C₆ alkyl;
each R^{z1} and R^{z2} is independently hydrogen, C₁-C₆ alkyl or C₃-C₆ cycloalkyl;
R¹ is hydrogen, C₁-C₆ alkyl, C₁-C₆ alkenyl, C₁-C₆ alkanol, C₁-C₆ haloalkyl, C₁-C₆ alkyloxy, CONR^{1a}R^{1b}, —NHC(O) C₁-C₆ alkyl, —COOH or —COO C₁-C₆ alkyl;
each R^{1a} and R^{1b} is independently hydrogen, C₁-C₆ alkyl or C₃-C₆ cycloalkyl
R² is hydrogen, C₁-C₆ alkyl, C₁-C₆ haloalkyl, C₁-C₆ alkyloxy or C₃-C₈ cycloalkyl;
or R¹ and R² combined with the atoms to which they are attached form a five- to eight-membered ring.

23. A pharmaceutical composition which comprises a compound according to claim 1 or a pharmaceutically acceptable salt thereof and one or more pharmaceutically acceptable carriers, diluents or excipients.

24. (canceled)

25. (canceled)

26. A compound according to claim 1 or a pharmaceutically acceptable salt thereof for use in the treatment of diseases or conditions for which a TGFβR antagonist is indicated.

27. A compound or a pharmaceutically acceptable salt thereof for use according to claim 26, wherein the disease or condition is cancer.

28. The use according to claim 27 wherein the cancer is small cell lung cancer, non-small cell lung cancer, triple-negative breast cancer, ovarian cancer, colorectal cancer, prostate cancer, melanoma, pancreatic cancer, multiple myeloma, T-acute lymphoblastic leukemia or AML.

29-33. (canceled)

34. A method for treating a subject afflicted with cancer comprising administering to the subject a therapeutically effective amount of:
a) a compound according to claim 1, or a pharmaceutically acceptable salt thereof, and
b) an anti-cancer agent which is an antibody or an antigen-binding portion thereof that binds specifically to a Programmed Death-1 (PD-1) receptor and inhibits PD-1 activity.

35. The method of claim 34, wherein the anti-PD-1 antibody is nivolumab.
